(19) United States
(12) Patent Application Publication
(10) Pub. No.: US 2023/0322797 A1
(43) Pub. Date: Oct. 12, 2023

(54) SOLID FORMS, PHARMACEUTICAL COMPOSITIONS AND PREPARATION OF HETEROAROMATIC MACROCYCLIC ETHER COMPOUNDS

(71) Applicant: Nuvalent, Inc., Cambridge, MA (US)

(72) Inventors: Christopher G. F. COOPER, Rehoboth, MA (US); Baudouin GERARD, Arlington, MA (US); Joshua Courtney HORAN, Somerville, MA (US); Jason T. KROPP, Westford, MA (US); Benjamin Stephen LANE, Lynnfield, MA (US); David James PEARSON, Derby (GB); Lauren Ashley MACEACHERN, Dartmouth (CA); Yameng HE, Halifax (CA); Mandeep SINGH, Montreal (CA); Arkesh NARAYANAPPA, Saint-Laurent (CA)

(21) Appl. No.: 18/131,581

(22) Filed: Apr. 6, 2023

Related U.S. Application Data
(60) Provisional application No. 63/328,609, filed on Apr. 7, 2022.

(51) Int. Cl. C07D 491/22; C07D 401/14; A61K 9/20

(52) U.S. Cl. CPC C07D 491/22; C07D 401/14; C07B 2200/13; A61K 9/2009; A61K 9/2013; A61K 9/2054

(57) ABSTRACT

Provided herein are solid forms comprising a compound of formula (I), or a stereoisomer, or a mixture of stereoisomers thereof, or a pharmaceutically acceptable salt thereof. Also provided herein are methods of synthesizing a compound of formula (I), pharmaceutical compositions comprising the same, and methods of treating, preventing, and managing various disorders using the compositions provided herein.

This application claims the benefit of priority to U.S. Ser. No. 63/328,609, filed Apr. 7, 2022, which is incorporated herein by reference in its entirety.

### 1. BACKGROUND

Receptor tyrosine kinases (RTKs) are cell surface enzymes that receive outside signals, such as whether to grow and divide, and transmit those signals in the cell through kinase activity. Many RTKs are proto-oncogenes; aberrant RTK activity can drive cell survival, growth and proliferation leading to cancer and related disorders. This aberrant kinase activity can be caused by mutations such as activating mutations in the kinase domain, gene rearrangements that result in fusion proteins containing the intact kinase domain, amplification and other means. RTK proto-oncogenes include ROS1, anaplastic lymphoma kinase (ALK), NTRK1 (encodes TRKA), NTRK2 (encodes TRKB), and NTRK3 (encodes TRKC).

ROS1 is an RTK proto-oncogene, with ROS1 rearrangements detected in non-small cell lung cancer (NSCLC), glioblastoma, inflammatory myofibroblastic tumor (IMT), cholangiocarcinoma, ovarian cancer, gastric cancer, colorectal cancer, angiosarcoma, and spitzoid melanoma. Oncogenic ROS1 gene fusions contain the kinase domain of ROS1 (3′ region) fused to the 5′ region of a variety of partner genes. Examples of ROS1 fusion partner genes observed in NSCLC include SLC34A2, CD74, TPM3, SDC4, EZR, LRIG3, KDELR2, CEP72, CLTL, CTNND2, GOPC, GPRC6A, LIMA1, LRIG3, MSN, MYO5C, OPRM1, SLC6A17 (putative), SLMAP, SRSF6, TFG, TMEM106B, TPD52L1, ZCCHC8 and CCDC6. Other fusion partners include CAPRIN1, CEP85L, CHCHD3, CLIP1 (putative), EEF1G, KIF21A (putative), KLC1, SART3, ST13 (putative), TRIM24 (putative), ERC1, FIP1L1, HLAA, KIAA1598, MYO5A, PPFIBP1, PWWP2A, FN1, YWHAE, CCDC30, NCOR2, NFKB2, APOB, PLG, RBP4, and GOLGB1.

ALK is an RTK proto-oncogene, with ALK rearrangements detected in many cancers, including NSCLC, anaplastic large cell lymphoma (ALCL), IMT, diffuse large B-cell lymphoma (DLBCL), esophageal squamous cell carcinoma (ESCC), renal medullary carcinoma, renal cell carcinoma, breast cancer, colon cancer, serous ovarian carcinoma, papillary thyroid cancer, and spitzoid tumors, and ALK activating mutations detected in neuroblastoma. Oncogenic ALK gene fusions contain the kinase domain of ALK (3′ region) fused to the 5′ region of more than 20 different partner genes, the most common being EML4 in NSCLC and NPM in ALCL. Other partner genes include TMP1, WDCP, GTF2IRD1, TPM3, TPM4, CLTC, LMNA, PRKAR1A, RANBP2, TFG, FN1, KLC1, VCL, STRN, HIP1, DCTN1, SQSTM1, TPR, CRIM1, PTPN3, FBXO36, ATIC and KIF5B.kinases.

NTRK1, NTRK2 and NTRK3 are RTK proto-oncogenes that encode TRK-family kinases, with NTRK1, NTRK2 and NTRK3 chromosomal rearrangements detected at low frequency in many cancers. For treatment of ROS1-positive or ALK-positive patients, however, TRK inhibition, particularly in the central nervous system (CNS), has been associated with adverse reactions, including dizziness/ataxia/gait disturbance, paraesthesia, weight gain and cognitive changes.

Agents in the prior art used to treat oncogenic ROS1 and ALK have substantial deficiencies. These deficiencies may represent one or more of the following: associated TRK inhibition, limited CNS activity, and inadequate activity against resistance mutations. Treatment of ROS1-positive or ALK-positive patients accompanied by TRK inhibition is associated with adverse reactions, particularly in the CNS, including dizziness/ataxia/gait disturbance, paraesthesia, weight gain and cognitive changes. Additionally, there is a need for CNS-penetrant and TRK-sparing inhibitors of the wild type ROS1 kinase domain and ROS1 with acquired resistance mutations occurring either individually or in combination, including G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. Likewise, there is a need for CNS-penetrant and TRK-sparing inhibitors of ALK with acquired resistance mutations. A variety of ALK drug resistance mutations, occurring either individually or in combination, have been reported, including G1202R, L1196M, G1269A, C1156Y, I1171T, I1171N, I1171S, F1174L, F1174S, V1180L, S1206Y, E1210K, 1151Tins, T1151M, F1174C, G1202del, D1203N, S1206Y, S1206C, L1152R, L1196Q, L1198P, L1198F, R1275Q, L1152P, C1156T, and F1245V.

In addition, for the production of a drug substance intended for use in humans, procedures need to be in place that can control the levels of impurities and ensure that API products are produced, which consistently meet their predetermined specifications. Thus, a need exists for a process to prepare ROS1 and ALK inhibitors suitable for human use, particularly on a commercial scale, that is, inter alia, safe, scalable, efficient, economically viable, and/or having other desirable properties. Among other entities, disclosed herein are crystalline forms and pharmaceutical compositions comprising such crystalline forms to address these needs and provide exemplary advantages.

### 2. SUMMARY

Provided herein are solid forms comprising a compound of formula (I) (also referred as Compound 1) or a stereoisomer, or a mixture of stereoisomers thereof, or a pharmaceutically acceptable salt thereof:

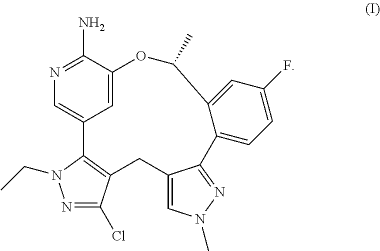 [SMILES: CCn1nc(Cl)c2c1-c1cnc(N)c(c1)O[C@H](C)c1cc(F)ccc1-c1nn(C)cc1C2]

In some embodiments, the solid form is a crystalline form. In other embodiments, the solid form is an amorphous form. In some embodiments, the solid form is a solid form of a compound of formula (I). In some embodiments, the solid form is a solid form of a free base of a compound of formula (I). In some embodiments, the solid form is a crystalline form of a free base of a compound of formula (I).

Also provided herein are methods of preparing the solid forms. In some embodiments, provided herein are methods of preparing solid forms of a free base of a compound of formula (I).

Also provided herein are methods of treating cancer comprising administering a therapeutically effective amount of a solid form of a compound of formula (I) provided herein to a subject in need thereof.

Also provided herein are pharmaceutical compositions comprising a solid form of a compound of formula (I) or a pharmaceutically acceptable salt thereof, and a pharmaceutically acceptable excipient. In some embodiments, the pharmaceutical composition comprises a solid form of a free base of a compound of formula (I). In some embodiments, the pharmaceutical composition comprises a solid form of a pharmaceutically acceptable salt of a compound of formula (I).

Also provided herein are processes of preparing a compound of Formula (II):

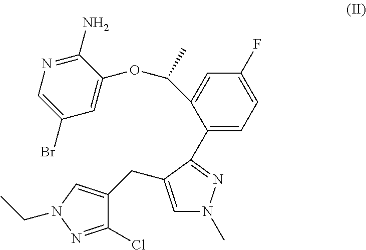 [SMILES: CCn1cc(Cc2cn(C)nc2-c2ccc(F)cc2[C@@H](C)Oc2cc(Br)cnc2N)c(Cl)n1]

or a stereoisomer, or a mixture of stereoisomers thereof, or a pharmaceutically acceptable salt thereof, comprising: (step 2.0) reacting a compound of Formula (III):

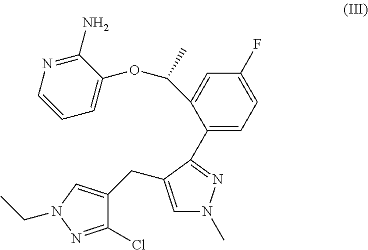 [SMILES: CCn1cc(Cc2cn(C)nc2-c2ccc(F)cc2[C@@H](C)Oc2cccnc2N)c(Cl)n1]

or a stereoisomer, or a mixture of stereoisomers thereof, or a pharmaceutically acceptable salt thereof, with a brominating reagent.

Also provided herein are processes for preparing a compound of Formula (II):

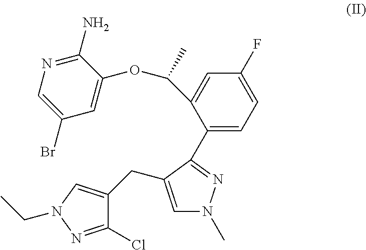 [SMILES: CCn1cc(Cc2cn(C)nc2-c2ccc(F)cc2[C@@H](C)Oc2cc(Br)cnc2N)c(Cl)n1]

or a stereoisomer, or a mixture of stereoisomers thereof, or a pharmaceutically acceptable salt thereof, comprising: (step 2a.1) reacting a compound of Formula (XXIX):

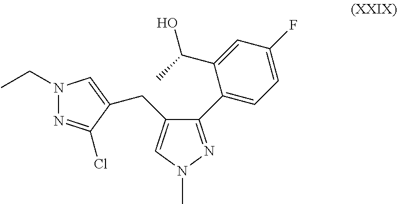 [SMILES: CCn1cc(Cc2cn(C)nc2-c2ccc(F)cc2[C@H](C)O)c(Cl)n1]

or a stereoisomer, or a mixture of stereoisomers thereof, or a pharmaceutically acceptable salt thereof, with a compound of Formula (XXX):

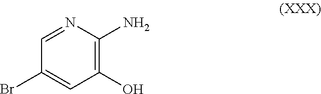

or a pharmaceutically acceptable salt thereof.

In some embodiments, the processes further comprises:

(step 1.0) cyclizing the compound of Formula (II), or a stereoisomer, or a mixture of stereoisomers thereof, or a pharmaceutically acceptable salt thereof, to provide a compound of Formula (I):

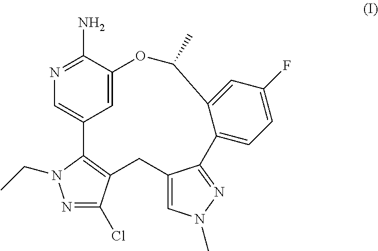 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof.

Also provided herein are methods of treating cancer comprising administering a therapeutically effective amount of a solid form provided herein.

Also provided herein are pharmaceutical compositions comprising Compound 1:

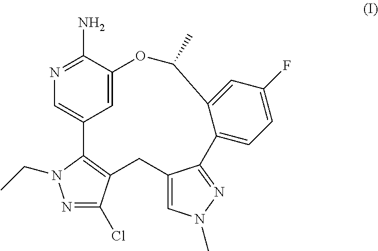 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof, a diluent, a disintegrant, a glidant, a binder, and a lubricant.

Also provided herein are methods of treating cancer comprising administering a therapeutically effective amount of the pharmaceutical composition provided herein.

Also provided herein are salts of a compound of Formula (II):

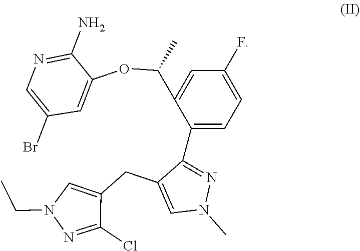 [SMILES: CCn1cc(Cc2cn(C)nc2-c2ccc(F)cc2[C@@H](C)Oc2cc(Br)cnc2N)c(Cl)n1]

Also provided herein are solid forms comprising a salt of a compound of Formula (II):

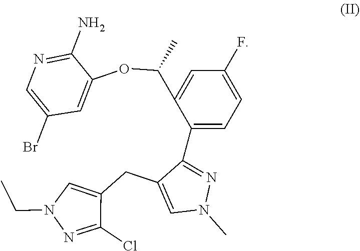 [SMILES: CCn1cc(Cc2cn(C)nc2-c2ccc(F)cc2[C@@H](C)Oc2cc(Br)cnc2N)c(Cl)n1]

### 3. INCORPORATION BY REFERENCE

All publications, patents, and patent applications mentioned in this specification are herein incorporated by reference in their entireties and to the same extent as if each individual publication, patent, or patent application was specifically and individually indicated to be incorporated by reference.

### 4. BRIEF DESCRIPTION OF FIGURES

FIG. 1A is a representative X-ray powder diffraction (XRPD) pattern of Form 1 (2-methyl THF solvate) of free base of Compound 1; FIG. 1B is a representative XRPD pattern of Form 1 (isopropyl acetate solvate) of free base of Compound 1.

FIG. 2 is a representative integrated thermal gravimetric analysis (TGA) and differential scanning calorimetry (DSC) thermograms for Form 1 (2-methyl THF solvate) of free base of Compound 1.

FIG. 3 is a representative integrated thermal TGA and DSC thermograms for Form 1 (isopropyl acetate solvate) of free base of Compound 1.

FIG. 4 is a representative XRPD pattern of Form 2 of free base of Compound 1.

FIG. 5 is a representative DSC thermogram of Form 2 of free base of Compound 1.

FIG. 6 is a representative DVS isotherm of Form 2 of free base of Compound 1.

FIG. 7 is a representative depiction of the unit cell a axis of single-crystal X-ray diffraction studies of Form 2 of free base of Compound 1.

FIG. 8 is a representative XRPD pattern of Form 3 of free base of Compound 1.

FIG. 9 is a representative integrated TGA and DSC thermograms for Form 3 of free base of Compound 1.

FIG. 10 is a representative XRPD pattern of Form 4 of free base of Compound 1.

FIG. 11 is a representative integrated TGA and DSC thermograms for Form 4 of free base of Compound 1.

FIG. 12A is a representative XRPD pattern of Form 5 (t-butanol and isopropanol mixed solvate) of free base of Compound 1; FIG. 12B is a representative XRPD pattern of Form 5 (t-butanol and acetone mixed solvate) of free base of Compound 1; FIG. 12C is a representative XRPD pattern of Form 5 (t-butanol and THF mixed solvate) of free base of Compound 1.

FIG. 13 is a representative integrated TGA and DSC thermograms for Form 5 (t-butanol and isopropanol mixed solvate) of free base of Compound 1.

FIG. 14 is a representative integrated TGA and DSC thermograms for Form 5 (t-butanol and acetone mixed solvate) of free base of Compound 1.

FIG. 15 is a representative integrated TGA and DSC thermograms for Form 5 (t-butanol and THF mixed solvate) of free base of Compound 1.

FIG. 16 is a representative XRPD pattern of Form 6 of free base of Compound 1.

FIG. 17 is a representative XRPD pattern of Form 7 of free base of Compound 1.

FIG. 18 is a representative integrated TGA and DSC thermograms for Form 7 of free base of Compound 1.

FIG. 19 is a representative XRPD pattern of Form 8 of free base of Compound 1.

FIG. 20 is a representative integrated TGA and DSC thermograms for Form 8 of free base of Compound 1.

FIG. 21 is a representative XRPD pattern of Form 9 of free base of Compound 1.

FIG. 22 is a representative integrated TGA and DSC thermograms for Form 9 of free base of Compound 1.

FIG. 23 is a representative XRPD pattern of Form 10 of free base of Compound 1.

FIG. 24 is a representative integrated TGA and DSC thermograms for Form 10 of free base of Compound 1.

FIG. 25 is a representative XRPD pattern of Form 11 of free base of Compound 1.

FIG. 26 is a representative integrated TGA and DSC thermograms for Form 11 of free base of Compound 1.

FIG. 27 is a representative XRPD pattern of Form 12 of free base of Compound 1.

FIG. 28 is a representative integrated TGA and DSC thermograms for Form 12 of free base of Compound 1.

FIG. 29 is a representative XRPD pattern of Form 13 of free base of Compound 1.

FIG. 30 is a representative integrated TGA and DSC thermograms for Form 13 of free base of Compound 1.

FIG. 31 is a representative XRPD pattern of Form 14 of free base of Compound 1.

FIG. 32 is a representative integrated TGA and DSC thermograms for Form 14 of free base of Compound 1.

FIG. 33 is a representative XRPD pattern of Form 15 of free base of Compound 1.

FIG. 34 is a representative integrated TGA and DSC thermograms for Form 15 of free base of Compound 1.

FIG. 35 is a representative XRPD pattern of Form A of mesylate salt of Compound 2.

FIG. 36 is a representative DSC thermogram of Form A of mesylate salt of Compound 2.

FIG. 37 is a representative TGA thermogram of Form A of mesylate salt of Compound 2.

FIG. 38 is a representative XRPD pattern of Form A of camsylate salt of Compound 2.

FIG. 39 is a representative DSC thermogram of Form A of camsylate salt of Compound 2.

FIG. 40 is a representative TGA thermogram of Form A of camsylate salt of Compound 2.

FIG. 41 is a representative DVS isotherm of Form A of camsylate salt of Compound 2.

FIG. 42 is a representative XRPD pattern of Form A of esylate salt of Compound 2.

FIG. 43 is a representative DSC thermogram of Form A of esylate salt of Compound 2.

FIG. 44 is a representative TGA thermogram of Form A of esylate salt of Compound 2.

FIG. 45 is a representative DVS isotherm of Form A of esylate salt of Compound 2.

FIG. 46 is a representative XRPD pattern of Form A of sulfate salt of Compound 2.

FIG. 47 is a representative DSC thermogram of Form A of sulfate salt of Compound 2.

FIG. 48 is a representative TGA thermogram of Form A of sulfate salt of Compound 2.

FIG. 49 is a representative DVS isotherm of Form A of sulfate salt of Compound 2.

FIG. 50 is a representative XRPD pattern of Form A of tosylate salt of Compound 2.

FIG. 51 is a representative DSC thermogram of Form A of tosylate salt of Compound 2.

FIG. 52 is a representative XRPD pattern of Form A of besylate salt of Compound 2.

FIG. 53 is a representative DSC thermogram of Form A of besylate salt of Compound 2.

FIG. 54 is a representative XRPD pattern of Form B of besylate salt of Compound 2.

FIG. 55 is a representative XRPD pattern of Form A of 2-naphthalenesulfonate salt of Compound 2.

FIG. 56 is a representative DSC thermogram of Form A of 2-naphthalenesulfonate salt of Compound 2.

FIG. 57 shows the dissolution profile of tablets of Compound 1.

FIG. 58 is a representative XRPD pattern of Form A of salicylate salt of Compound 1.

FIG. 59 is a representative XRPD pattern of Form A of maleate salt of Compound 1.

### 5. DETAILED DESCRIPTION

### 5.1 Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In some embodiments, chemical structures are disclosed with a corresponding chemical name. In case of conflict, the chemical structure controls the meaning, rather than the name.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

Unless specifically stated or obvious from context, as used herein, the term “or” is understood to be inclusive. Unless specifically stated or obvious from context otherwise, as used herein, the terms “a”, “an”, and “the” are understood to be singular or plural. For example, when a compound provided herein is administered to “a patient”, it includes administering the compound to an individual patient or a patient population.

As used herein and unless otherwise specified, “stereoisomers” refer to the various stereoisomeric forms of a compound that comprises one or more asymmetric centers or stereohindrance in the structure. In some embodiments, a stereoisomer is an enantiomer, a mixture of enantiomers, an atropisomer, or a tautomer thereof. For example, the compounds described herein can be in the form of an individual enantiomer, diastereomer or geometric isomer (e.g. an atropisomer), or can be in the form of a mixture of stereoisomers, including racemic mixtures and mixtures enriched in one or more stereoisomer. In some embodiments, compounds provided herein may be atropisomers. In certain embodiments, atropisomers are stereoisomers arising because of hindered rotation about a single bond, where energy differences due to steric strain or other contributors create a barrier to rotation that is high enough to allow for isolation of individual conformers. Stereoisomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In certain embodiments, compounds provided herein may be racemic. In certain embodiments, compounds provided herein may be enriched in one enantiomer. For example, a compound provided herein may have greater than about 30% ee, about 40% ee, about 50% ee, about 60% ee, about 70% ee, about 80% ee, about 90% ee, or even about 95% or greater ee. In certain embodiments, compounds provided herein may have more than one stereocenter. In certain such embodiments, compounds provided herein may be enriched in one or more diastereomer. For example, a compound provided herein may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound. An enantiomerically enriched mixture may comprise, for example, at least about 60 mol percent of one enantiomer, or more particularly at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound. A diastereomerically enriched mixture may comprise, for example, at least about 60 mol percent of one diastereomer, or more particularly at least about 75, about 90, about 95, or even about 99 mol percent.

In some embodiments, a moiety in a compound exists as a mixture of tautomers. A “tautomer” is a structural isomer of a moiety or a compound that readily interconverts with another structural isomer. For example, a pyrazole ring has two tautomers:

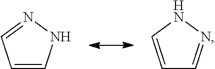 [SMILES: c1cn[nH]c1 ; c1cn[nH]c1]

which differ in the positions of the pi-bonds and a hydrogen atom. Unless explicitly stated otherwise, a drawing of one tautomer of a moiety or a compound encompasses all of the possible tautomers.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In certain embodiments, the subject is a human. In certain embodiments, the subject is a human adult at least of 40 years old. In certain embodiments, the subject is a human adult at least of 50 years old. In certain embodiments, the subject is a human adult at least of 60 years old. In certain embodiments, the subject is a human adult at least of 70 years old. In certain embodiments, the subject is a human adult at least of 18 years old or at least of 12 years old. As used herein and unless otherwise specified, a human subject to which administration of a therapeutic (e.g., a compound as described herein) is contemplated in order to treat, prevent or manage a disease, disorder, or condition, or symptoms thereof, is also called a “patient”.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. These effects are also called “prophylactic” effects. Thus, as used herein and unless otherwise specified, the terms “prevention” and “preventing” refer to an approach for obtaining beneficial or desired results including, but not limited, to prophylactic benefit. For prophylactic benefit, a therapeutic can be administered to a patient at risk of developing a particular disease, or to a patient reporting one or more of the physiological symptoms of a disease, even though a diagnosis of this disease may not have been made. In one embodiment, a therapeutic is administered prior to clinical manifestation of the unwanted condition (e.g., disease or other unwanted state of the subject) for prophylactic benefit (e.g., it protects the subject against developing the unwanted condition).

As used herein and unless otherwise specified, the terms “treatment” and “treating” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment. In one embodiment, “treatment” comprises administration of a therapeutic after manifestation of the unwanted condition (i.e., it is intended to diminish, ameliorate, or stabilize the existing unwanted condition or side effects thereof).

As used herein and unless otherwise specified, “cancer” refers to any malignant and/or invasive growth or tumor caused by abnormal cell growth, including solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. Cancer includes, but not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein and unless otherwise specified, “abnormal cell growth” refers to cell growth that is independent of normal regulatory mechanisms (e.g., loss of contact inhibition). Abnormal cell growth may be benign (not cancerous), or malignant (cancerous). In some embodiments of the methods provided herein, the abnormal cell growth is cancer.

In some embodiments, the abnormal cell growth is cancer mediated by an anaplastic lymphoma kinase (ALK). In some such embodiments, the ALK is a genetically altered ALK. In other embodiments, the abnormal cell growth is cancer mediated by ROS1 kinase. In some such embodiments, the ROS1 kinase is a genetically altered ROS1 kinase. In some embodiments, the abnormal cell growth is cancer, in particular NSCLC. In some such embodiments, the NSCLC is mediated by ALK or ROS1. In specific embodiments, the cancer is NSCLC is mediated by genetically altered ALK or genetically altered ROS1.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

An “effective amount”, as used herein, refers to an amount that is sufficient to achieve a desired biological effect. A “therapeutically effective amount”, as used herein, refers to an amount that is sufficient to achieve a desired therapeutic effect. For example, a therapeutically effective amount can refer to an amount that is sufficient to improve at least one sign or symptom of cancer.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein and unless otherwise indicated, the term “relapsed” refers to a disorder, disease, or condition that responded to prior treatment (e.g., achieved a complete response) then had progression. The prior treatment can include one or more lines of therapy.

As used herein and unless otherwise indicated, the term “refractory” refers to a disorder, disease, or condition that has not responded to prior treatment that can include one or more lines of therapy.

“Crystalline,” as used herein, refers to a homogeneous solid formed by a repeating, three-dimensional pattern of atoms, ions or molecules having fixed distances between constituent parts. The unit cell is the simplest repeating unit in this pattern. Notwithstanding the homogenous nature of an ideal crystal, a perfect crystal rarely, if ever, exists. “Crystalline,” as used herein, encompasses crystalline forms that include crystalline defects, for example, crystalline defects commonly formed by manipulating (e.g., preparing, purifying) the crystalline forms described herein. A person skilled in the art is capable of determining whether a sample of a compound is crystalline notwithstanding the presence of such defects. Crystalline forms can be characterized by analytical methods such as x-ray powder diffraction (XRPD), differential scanning calorimetry (DSC), thermogravimetric analysis (TGA), nuclear magnetic resonance spectroscopy (NMR), single crystal x-ray diffraction, Raman spectroscopy, Fourier transform infrared spectroscopy (FTIR) and/or any other suitable analytical techniques.

As used herein “solvate” refers to a crystalline form of a molecule, atom, and/or ions that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. Solvates may occur as dimers or oligomers comprising more than one molecule or Compound ABC within the crystalline lattice structure.

As used herein “amorphous” refers to a solid form of a molecule, atom, and/or ions that is not crystalline. In particular, the term “amorphous form” describes a disordered solid form, i.e., a solid form lacking long range crystalline order. An amorphous solid does not display a definitive X-ray diffraction pattern. In certain embodiments, an amorphous form of a substance may be substantially pure of other amorphous forms and/or crystal forms.

As used herein and unless otherwise specified, the term “solid form” and related terms refer to a physical form which is not predominantly in a liquid or a gaseous state. Solid forms may be crystalline, amorphous or mixtures thereof. As used herein and unless otherwise specified, the term “crystal forms” and related terms refer to solid forms that are crystalline. Crystal forms include, but are not limited to, non-solvates, non-hydrates, solvates, hydrates, and other molecular complexes, as well as salts, solvates of salts, hydrates of salts, and other molecular complexes of salts thereof. In certain embodiments, a solid form or crystal form of a substance may be substantially free of amorphous forms and/or other solid forms and/or crystal forms. In certain embodiments, a solid form and/or crystal form of a substance may contain less than about 1%, 2%, 3%, 4%, 5%, 6%, 7%, 8%, 9%, 10%, 15%, 20%, 25%, 30%, 35%, 40%, 45% or 50% of one or more amorphous forms and/or other solid forms and/or crystal forms on a weight basis. In certain embodiments, a solid form or crystal form of a substance may be physically and/or chemically pure. In certain embodiments, a solid form or crystal form of a substance may be about 99%, 98%, 97%, 96%, 95%, 94%, 93%, 92%, 91% or 90% physically and/or chemically pure. In certain embodiments, a solid form or crystal form may be substantially chemically pure and/or substantially physically pure.

“Substantially pure,” when used without further qualification, means the compound has a purity greater than about 90 weight percent, for example, greater than about 90, 91, 92, 93, 94, 95, 96, 97, 98, or 99 weight percent, and also including a purity equal to about 100 weight percent, based on the weight of the compound. The remaining material may comprise other form(s) of the compound and/or reaction impurities and/or processing impurities arising from its preparation. Purity can be assessed using techniques known in the art, for example, using an HPLC assay.

“Substantially pure” can also be qualified. If the compound is “substantially pure” with respect to the presence of chemical impurities (e.g. reaction impurities and/or processing impurities arising from its preparation), it can be referred to as “substantially chemically pure”. If the compound is “substantially pure” with respect to the presence of the other enantiomer, it can be referred to as “substantially enantiomerically pure”. In some embodiments, the compound (e.g. Compound 1) is substantially enantiomerically pure with the other enantiomer (e.g. the S enantiomer) present less than about 10%, less than about 5%, less than about 3%, less than about 1%, less than about 0.5%, or less than about 0.1% by weight. If the compound is “substantially pure” with respect to the presence of other physical forms of the compound having the indicated structure, it can be referred to as “substantially physically pure”. When qualified, “substantially pure” means that the indicated compound contains less than about 10%, less than about 5%, less than about 3%, less than about 1%, less than about 0.5%, or less than about 0.1% by weight of the indicated impurity. In certain embodiments, the solid form of Compound 1 is substantially pure (e.g. having the purity of at least about 90 wt %, at least about 95 wt %, at least about 96 wt %, at least about 97 wt %, at least about 98 wt %, or at least about 99 wt %). In certain embodiments, the solid form of Compound 1 has the purity of at least about 95 wt %. In certain embodiments, the solid form of Compound 1 is substantially enantiomerically pure (e.g. having the enantiomeric purity of at least about 98.0 wt %, at least about 99.0 wt %, at least about 99.5 wt %, or at least about 99.9 wt %). In certain embodiments, the solid form of Compound 1 has the enantiomeric purity of at least about 99.5 wt %. In certain embodiments, the pharmaceutical composition comprising Compound 1 has the purity of at least about 95 wt %. In certain embodiments, the pharmaceutical composition comprising Compound 1 has the purity of at least about 96 wt %. In certain embodiments, the pharmaceutical composition comprising Compound 1 has the purity of at least about 97 wt %. In certain embodiments, the pharmaceutical composition comprising Compound 1 has the purity of at least about 98 wt %. In certain embodiments, the pharmaceutical composition comprising Compound 1 has the purity of at least about 99 wt %. In certain embodiments, the pharmaceutical composition comprising Compound 1 has the purity of at least about 95 wt % over 12 months.

Solid forms may exhibit distinct physical characterization data that are unique to a particular solid form, such as the crystal forms described herein. These characterization data may be obtained by various techniques known to those skilled in the art. The data provided by these techniques may be used to identify a particular solid form. For example, an XRPD pattern, DSC thermogram or TGA thermal curve that “matches” or, interchangeably, is “substantially in accordance” with one or more figures herein showing an XRPD pattern or DSC thermogram or TGA thermal curve, respectively, is one that would be considered by one skilled in the art to represent the same single crystalline form of the compound as the sample of the compound that provided the pattern or thermogram or thermal curve of one or more figures provided herein. Thus, an XRPD pattern or DSC thermogram or TGA thermal curve that matches or is substantially in accordance may be identical to that of one of the figures or, more likely, may be somewhat different from one or more of the figures. For example, an XRPD pattern that is somewhat different from one or more of the figures may not necessarily show each of the lines of the diffraction pattern presented herein and/or may show a slight change in appearance or intensity of the lines or a shift in the position of the lines. These differences typically result from differences in the conditions involved in obtaining the data or differences in the purity of the sample used to obtain the data. A person skilled in the art is capable of determining if a sample of a crystalline compound is of the same form as or a different form from a form disclosed herein by comparison of the XRPD pattern or DSC thermogram or TGA thermal curve of the sample and the corresponding XRPD pattern or DSC thermogram or TGA thermal curve disclosed herein.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein and unless otherwise specified, the terms “about” and “approximately,” when used in connection with a numeric value or a range of values which is provided to characterize a particular solid form, e.g., a specific temperature or temperature range, such as, for example, that describing a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of XRPD peak position may vary by up to ±0.2 degrees 2θ while still describing the particular XRPD peak. In one embodiment, the value of XRPD peak position may vary by up to ±0.1 degrees 2θ. In one embodiment, the value of XRPD peak position may vary by up to ±0.05 degrees 2θ.

The term “between” includes the endpoint numbers on both limits of the range. For example, the range described by “between 3 and 5” is inclusive of the numbers “3” and “5”.

As used herein and unless otherwise specified, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. In certain embodiments, pharmaceutically acceptable salts include, but are not limited to, alkyl, dialkyl, trialkyl or tetra-alkyl ammonium salts. In certain embodiments, pharmaceutically acceptable salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, pharmaceutically acceptable salts include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Pharmaceutically acceptable anionic salts include, but are not limited to, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate.

As used herein, and unless otherwise specified, the term “enantiomerically pure” refers to a composition comprising an enantiomeric excess of at least about 50%, at least about 75%, at least about 90%, at least about 95%, or at least about 99% of one enantiomer of a compound having one or more chiral center(s). In some embodiments, the composition may be “substantially enantiomerically pure”, which refers to preparations of compositions which have at least about 85% by weight of one enantiomer relative to the other enantiomer of a compound, such as at least about 90% by weight, and further such as at least 95% by weight. In certain embodiments, the compositions provided herein comprise an enantiomeric excess of at least about 90% by weight of one enantiomer of the compound. In other embodiments, the compositions comprises an enantiomeric excess of at least about 95%, at least about 98%, or at least about 99% by weight of one enantiomer of the compound.

As used herein and unless otherwise indicated, the term “process(es)” provided herein refers to the methods provided herein which are useful for preparing a compound as described herein or a solid form thereof (e.g. a crystalline form, partically crystalline form, or an amorphous form) provided herein. Modifications to the methods provided herein (e.g., starting materials, reagents, protecting groups, solvents, temperatures, reaction times, purification) are also provided herein. In general, the technical teaching of one embodiment provided herein can be combined with that disclosed in any other embodiments provided herein.

As used herein, and unless otherwise indicated, the term “adding,” “reacting,” “treating,” or the like means contacting one reactant, reagent, solvent, catalyst, reactive group or the like with another reactant, reagent, solvent, catalyst, reactive group or the like. Reactants, reagents, solvents, catalysts, reactive group or the like can be added individually, simultaneously or separately and can be added in any order. Reactants, reagents, solvents, catalysts, reactive group or the like can each respectively be added in one portion, which may be delivered all at once or over a period of time, or in discrete portions, which also may be delivered all at once or over a period of time. They can be added in the presence or absence of heat and can optionally be added under an inert atmosphere. “Reacting” can refer to in situ formation or intramolecular reaction where the reactive groups are in the same molecule.

As used herein, the term “combining” refers to bringing one or more chemical entities into association with another one or more chemical entities. Combining includes the processes of adding one or more compounds to a solid, liquid or gaseous mixture of one or more compounds (the same or other chemical entities), or a liquid solution or multiphasic liquid mixture. The act of combining includes the process or processes of one or more compounds reacting (e.g., bond formation or cleavage; salt formation, solvate formation, chelation, or other non-bond altering association) with one or more compounds (the same or other chemical entities). The act of combining can include alteration of one or more compounds, such as by isomerization (e.g., tautomerization, resolution of one isomer from another, or racemization).

As used herein, and unless otherwise indicated, the term “transforming” refers to subjecting the compound at hand to reaction conditions suitable to effect the formation of the desired compound at hand.

As used herein, the term “recovering” includes, but is not limited to, the action of obtaining one or more compounds by collection during and/or after a process step as disclosed herein, and the action of obtaining one or more compounds by separation of one or more compounds from one or more other chemical entities during and/or after a process step as disclosed herein. The term “collection” refers to any action(s) known in the art for this purpose, including, but not limited to, filtration, decanting a mother liquor from a solid to obtain one or more compounds, and evaporation of liquid media in a solution or other mixture to afford a solid, oil, or other residue that includes one or more compounds. The solid can be crystalline, acrystalline, partially crystalline, or amorphous, a powder, granular, of varying particle sizes, of uniform particle size, among other characteristics known in the art. An oil can vary in color and viscosity, and include one or more solid forms as a heterogeneous mixture, among other characteristics known in the art. The term “separation” refers to any action(s) known in the art for this purpose, including, but not limited to, isolating one or more compounds from a solution or mixture using, for example, seeded or seedless crystallization or other precipitation techniques (e.g., adding an anti-solvent to a solution to induce compound precipitation; heating a solution, then cooling to induce compound precipitation; scratching the surface of a solution with an implement to induce compound precipitation), and distillation techniques. Recovering one or more compounds can involve preparation of a salt, solvate, hydrate, chelate or other complexes of the same, then collecting or separating as described above.

As used herein, the term “catalyst precursor” refers to a chemical composition wherein one or more components of an active catalyst (e.g. metal center and supporting ligand) are added to the reaction mixture such that formation of an active catalyst occurs in situ. For example, a cataCXium A ligated palladium catalyst may be formed in situ by adding a catalyst precursor comprising a palladium source (e.g. Pd(OAc)₂) and a source of cataCXium A (e.g. cataCXium A). Those skilled in the art will recognize that even when the metal source and supporting ligand are added to a reaction mixture in the form of a single chemical entity (e.g. Pd(dppf)Cl₂), further activation and/or reaction in situ may be required to produce an active catalyst. Notwithstanding, as used herein, the term “catalyst” includes, but is not limited to a chemical composition wherein more than one component of an active catalyst (e.g. metal center and supporting ligand) is added to a reaction mixture in the form of a single chemical entity (e.g. Pd(dppf)Cl₂), even if further activation and/or reaction in situ is required to produce an active catalyst.

Although most embodiments and examples provided herein are directed to one enantiomer of a compound, it is to be understood that the opposite enantiomer of a compound can be prepared by the provided processes when the stereochemistry of chiral reactant, reagent, solvent, catalyst, ligand or the like is reversed.

As used herein, and unless otherwise specified, the terms “solvent,” “organic solvent,” or “inert solvent” each mean a solvent inert under the conditions of the reaction being described. Unless specified to the contrary, for each gram of a limiting reagent, one cc (or mL) of solvent constitutes a volume equivalent (or “vol.”).

The disclosure can be understood more fully by reference to the following detailed description and illustrative examples, which are intended to exemplify non-limiting embodiments.

### 5.2 Solid Forms

Potential pharmaceutical solids include crystalline solids and amorphous solids. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability (see, e.g., S. R. Vippagunta et al., Adv. Drug. Deliv. Rev., (2001) 48:3-26; L. Yu, Adv. Drug. Deliv. Rev., (2001) 48:27-42). A change in solid form may affect a variety of physical and chemical properties, which may provide benefits or drawbacks in processing, formulation, stability and bioavailability, among other important pharmaceutical characteristics.

Whether crystalline or amorphous, potential solid forms of a pharmaceutical compound may include single-component and multiple-component solids. Single-component solids consist essentially of the pharmaceutical compound in the absence of other compounds. Variety among single-component crystalline materials may potentially arise from the phenomenon of polymorphism, wherein multiple three-dimensional arrangements exist for a particular pharmaceutical compound (see, e.g., S. R. Byrn et al., Solid State Chemistry of Drugs, (1999) SSCI, West Lafayette).

Additional diversity among the potential solid forms of a pharmaceutical compound may arise from the possibility of multiple-component solids. Crystalline solids comprising two or more ionic species are termed salts (see, e.g., Handbook of Pharmaceutical Salts: Properties, Selection and Use, P. H. Stahl and C. G. Wermuth, Eds., (2002), Wiley, Weinheim). Additional types of multiple-component solids that may potentially offer other property improvements for a pharmaceutical compound or salt thereof include, e.g., hydrates, solvates, co-crystals and clathrates, among others (see, e.g., S. R. Byrn et al., Solid State Chemistry of Drugs, (1999) SSCI, West Lafayette). Multiple-component crystal forms may potentially be susceptible to polymorphism, wherein a given multiple-component composition may exist in more than one three-dimensional crystalline arrangement. The discovery of solid forms is of great importance in the development of a safe, effective, stable and marketable pharmaceutical compound.

The solid forms provided herein are useful as active pharmaceutical ingredients for the preparation of formulations for use in animals or humans. Thus, embodiments herein encompass the use of these solid forms as a final drug product. Certain embodiments provide solid forms useful in making final dosage forms with improved properties, e.g., powder flow properties, compaction properties, tableting properties, stability properties, and excipient compatibility properties, among others, that are needed for manufacturing, processing, formulation and/or storage of final drug products. Certain embodiments herein provide pharmaceutical compositions comprising a single-component crystal form, and/or a multiple-component crystal form comprising the compound of formula (I) and a pharmaceutically acceptable excipient.

Solid form and related terms refer to a physical form which is not predominantly in a liquid or a gaseous state. Solid forms may be crystalline or mixtures of crystalline and amorphous forms. A “single-component” solid form comprising a particular compound consists essentially of that compound. A “multiple-component” solid form comprising a particular compound comprises that compound and a significant quantity of one or more additional species, such as ions and/or molecules, within the solid form. The solid forms provided herein may be crystalline or an intermediate form (e.g., a mixture of crystalline and amorphous forms). The crystal forms described herein, therefore, may have varying degrees of crystallinity or lattice order. The solid forms described herein are not limited to any particular degree of crystallinity or lattice order, and may be 0-100% crystalline. Methods of determining the degree of crystallinity are known to those of ordinary skill in the art, such as those described in Suryanarayanan, R., X-Ray Powder Diffractometry, Physical Characterization of Pharmaceutical Solids, H. G. Brittain, Editor, Marcel Dekker, Murray Hill, N.J., 1995, pp. 187-199, which is incorporated herein by reference in its entirety. In some embodiments, the solid forms described herein are about 0, 5, 10, 15, 20, 25, 30, 35, 40, 45, 50, 55, 60, 65, 70, 75, 80, 85, 90, 95 or 100% crystalline.

Solid forms may exhibit distinct physical characterization data that are unique to a particular solid form, such as the crystal forms described herein. These characterization data may be obtained by various techniques known to those skilled in the art, including for example X-ray powder diffraction, differential scanning calorimetry, thermal gravimetric analysis, and nuclear magnetic resonance spectroscopy. The data provided by these techniques may be used to identify a particular solid form. One skilled in the art can determine whether a solid form is one of the forms described herein by performing one of these characterization techniques and determining whether the resulting data is “substantially similar” to the reference data provided herein, which is identified as being characteristic of a particular solid form. Characterization data that is “substantially similar” to those of a reference solid form is understood by those skilled in the art to correspond to the same solid form as the reference solid form. In analyzing whether data is “substantially similar,” a person of ordinary skill in the art understands that particular characterization data points may vary to a reasonable extent while still describing a given solid form, due to, for example, experimental error and routine sample-to-sample analysis.

In some embodiments, provided herein are solid forms comprising a compound of formula (I), or a pharmaceutically acceptable salt thereof:

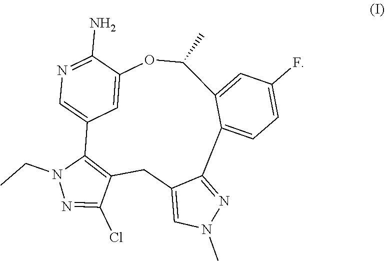 [SMILES: CCn1nc(Cl)c2c1-c1cnc(N)c(c1)O[C@H](C)c1cc(F)ccc1-c1nn(C)cc1C2]

In one embodiment, the solid form comprising a compound of formula (I) can be a crystalline form, a partially crystalline form, or a mixture of crystalline form(s), or amorphous form(s). In one embodiment, provided herein is a solid form comprising a crystalline form of a compound of formula (I). In one embodiment, the solid form comprises a salt, solvate (e.g., hydrate), or solvate of a salt thereof, or a mixture thereof. In another embodiment, the solid form is an amorphous form. In one embodiment, the solid form is substantially pure. In one embodiment, the solid form is substantially chemically pure. In one embodiment, the solid form is substantially physically pure. In one embodiment, the solid form is substantially enantiomerically pure. In one embodiment, the solid form (e.g. Form 2) has an enantiomeric purity of at least about 98% (e.g. about 99% or about 99.5%). The compound of formula (I) is described in international patent application No. PCT/US2021/030940, the entirety of which is incorporated herein by reference.

5.2.1. Solid Forms of Free Base of Compound 1

Provided herein is a solid form comprising a compound of Formula (I):

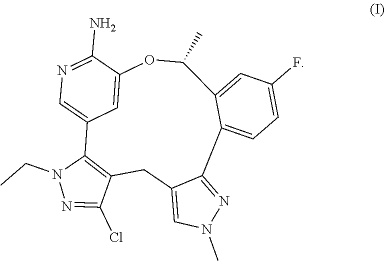 [SMILES: CCn1nc(Cl)c2c1-c1cnc(N)c(c1)O[C@H](C)c1cc(F)ccc1-c1nn(C)cc1C2]

In one embodiment, provided herein is a solid form comprising a free base of Compound 1. In one embodiment, provided herein is a solid form comprising an anhydrous free base of Compound 1. In one embodiment, provided herein is a solid form comprising a solvate of a free base of Compound 1. In one embodiment, provided herein is a solid form comprising a hydrate of a free base of Compound 1. In one embodiment, provided herein is a solid form comprising a 2-MeTHF, isopropyl acetate, 1,4-dioxane, 2-propanol, acetone, THF, t-butanol, MIBK, cyclohexanone, MEK, methylcyclohexane, or cyclohexane solvate of a free base of Compound 1.

As used herein, “Compound 1,” “free base of Compound 1,” “free base of a compound of formula (I),” and “Compound 1 free base” are used interchangeably.

It is contemplated that Compound 1, or a stereoisomer, or a mixture of stereoisomers thereof, or a pharmaceutically acceptable salt thereof, can exist in a variety of solid forms. Such solid forms include crystalline solids (e.g., crystalline forms of anhydrous Compound 1, crystalline forms of hydrates of Compound 1, and crystalline forms of solvates of Compound 1), amorphous solids, or mixtures of crystalline and amorphous solids. In one embodiment, the solid form is substantially crystalline. In one embodiment, the solid form is crystalline.

In some embodiments, the molar ratio of Compound 1 to the solvent (e.g., water) in the solid form ranges from about 10:1 to about 1:10. In some embodiments, the molar ratio of Compound 1 to the solvent (e.g., water) in the solid form ranges from about 5:1 to about 1:5. In some embodiments, the molar ratio of Compound 1 to the solvent (e.g., water) in the solid form ranges from about 3:1 to about 1:3. In some embodiments, the molar ratio of Compound 1 to the solvent (e.g., water) in the solid form ranges from about 2:1 to about 1:2. In one embodiment, the molar ratio is about 1:2 (i.e., bis-solvate or dihydrate). In another embodiment, the molar ratio is about 1:1 (i.e., mono-solvate or mono-hydrate). In yet another embodiment, the molar ratio is about 2:1 (i.e., hemi-solvate or hemi-hydrate).

5.2.1.1 Form 1 of Compound 1

In one embodiment, provided herein is a Form 1 of Compound 1.

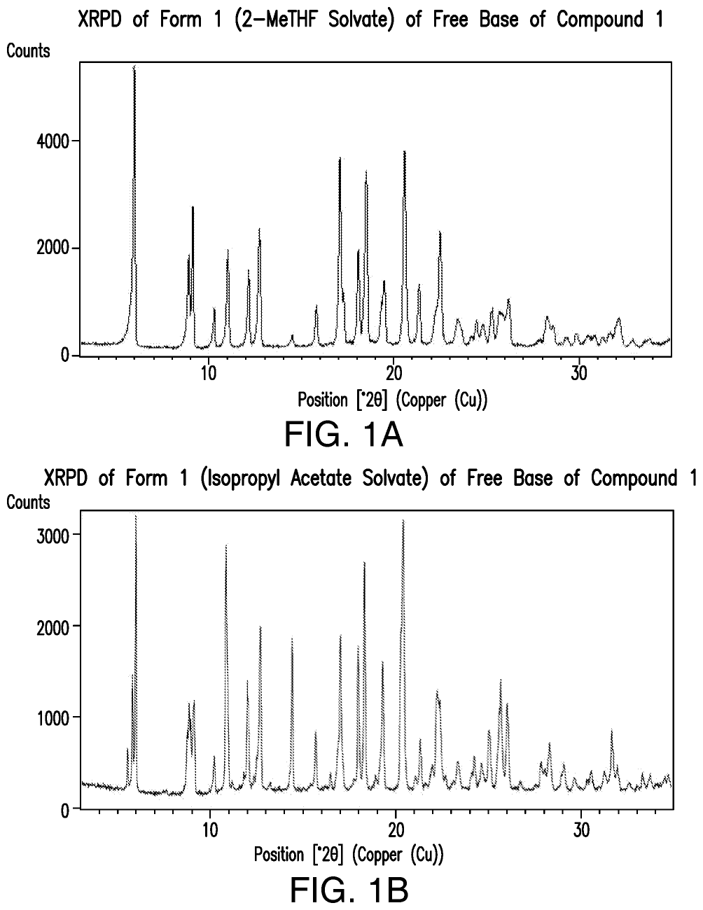

A representative XRPD pattern of Form 1 of Compound 1 is provided in FIG. 1A.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, 23 or all of the XRPD peaks located at approximately the following positions (e.g., degrees 2θ±0.2) when measured using Cu Kα radiation: 6.0, 8.9, 9.2, 10.3, 11.1, 12.2, 12.8, 15.8, 17.1, 17.3, 18.1, 18.5, 19.3, 19.5, 20.6, 21.4, 22.2, 22.5, 23.4, 24.5, 25.3, 25.7, 26.2 and 28.2° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a free base of Compound 1, characterized by an XRPD pattern, when measured using Cu Kα radiation, comprising at least three peaks selected from the group consisting of approximately (e.g., ±0.2°) 6.0, 8.9, 9.2, 11.1, 12.2, 12.8, 17.1, 18.1, 18.5, 20.6, and 22.5° 2θ. In one embodiment, the solid form is characterized by an XRPD pattern comprising at least four peaks selected from the group consisting of approximately (e.g., ±0.2°) 6.0, 8.9, 9.2, 11.1, 12.2, 12.8, 17.1, 18.1, 18.5, 20.6, and 22.5° 2θ. In one embodiment, the solid form is characterized by an XRPD pattern comprising at least five peaks selected from the group consisting of approximately (e.g., ±0.2°) 6.0, 8.9, 9.2, 11.1, 12.2, 12.8, 17.1, 18.1, 18.5, 20.6, and 22.5° 2θ.

In one embodiment, provided herein is a solid form (e.g. a crystalline form) comprising a free base of Compound 1, characterized by an XRPD pattern comprising peaks at approximately (e.g., ±0.2°) 6.0, 18.5 and 20.6° 2θ. In one embodiment, the XRPD pattern further comprises peaks at approximately (e.g., ±0.2°) 12.8 and 17.1° 2θ. In one embodiment, the XRPD pattern further comprises peaks at approximately (e.g., ±0.2°) 9.2 and 22.5° 2θ. In one embodiment, the XRPD pattern comprises peaks at approximately (e.g., ±0.2°) 6.0, 8.9, 9.2, 11.1, 12.8, 17.1, 18.5, 20.6 and 22.5° 2θ.

In one embodiment, provided herein is a solid form comprising a free base of Compound 1, characterized by an XRPD pattern that matches the XRPD pattern depicted in FIG. 1A. In one embodiment, the Form 1 that provides FIG. 1A is a 2-MeTHF solvate.

Another representative XRPD pattern of Form 1 of Compound 1 is provided in FIG. 1B.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, 23, 24, 25, or all of the XRPD peaks located at approximately the following positions (e.g., degrees 2θ±0.2) when measured using Cu Kα radiation: 5.8, 6.0, 8.7, 8.8, 9.1, 10.8, 10.9, 12.0, 12.1, 12.7, 14.4, 15.7, 17.0, 18.0, 18.3, 19.3, 20.3, 20.4, 21.3, 22.2, 22.4, 25.0, 25.7, 26.0, 28.3, and 31.7° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a free base of Compound 1, characterized by an XRPD pattern, when measured using Cu Kα radiation, comprising at least three peaks selected from the group consisting of approximately (e.g., ±0.2°) 6.0, 10.8, 12.7, 14.4, 17.0, 18.0, 18.3, 19.3, and 20.4° 2θ. In one embodiment, the solid form is characterized by an XRPD pattern comprising at least four peaks selected from the group consisting of approximately (e.g., ±0.2°) 6.0, 10.8, 12.7, 14.4, 17.0, 18.0, 18.3, 19.3, and 20.4° 2θ. In one embodiment, the solid form is characterized by an XRPD pattern comprising at least five peaks selected from the group consisting of approximately (e.g., ±0.2°) 6.0, 10.8, 12.7, 14.4, 17.0, 18.0, 18.3, 19.3, and 20.4° 2θ.

In one embodiment, provided herein is a solid form (e.g. a crystalline form) comprising a free base of Compound 1, characterized by an XRPD pattern comprising peaks at approximately (e.g., ±0.2°) 6.0, 10.8, and 20.4° 2θ. In one embodiment, the XRPD pattern further comprises peaks at approximately (e.g., ±0.2°) 12.7 and 18.3° 2θ. In one embodiment, the XRPD pattern further comprises peaks at approximately (e.g., ±0.2°) 14.4 and 17.0° 2θ. In one embodiment, the XRPD pattern comprises peaks at approximately (e.g., ±0.2°) 6.0, 10.8, 12.7, 14.4, 17.0, 18.0, 18.3, 19.3, and 20.4° 2θ.

In one embodiment, provided herein is a solid form comprising a free base of Compound 1, characterized by an XRPD pattern that matches the XRPD pattern depicted in FIG. 1B. In one embodiment, the Form 1 that provides FIG. 1B is an isopropy acetate solvate.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

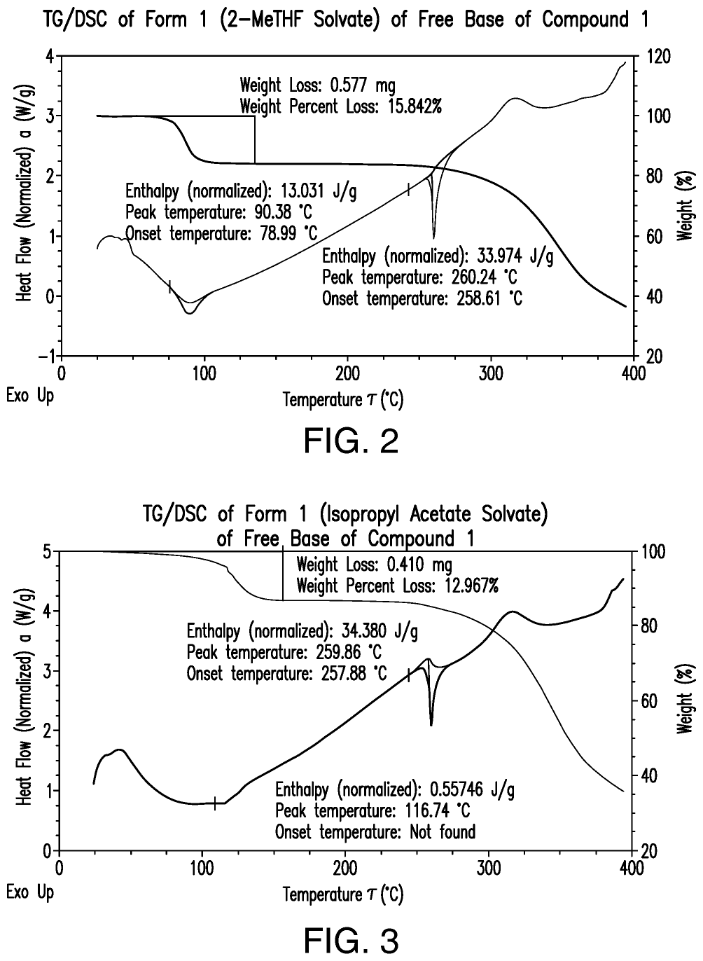

A representative overlay of TGA/DSC thermograms of Form 1 is provided in FIG. 2. In one embodiment, provided herein is a solid form comprising a free base of Compound 1, which exhibits, as characterized by DSC, a thermal (endo) event with an onset temperature of about 79° C. (e.g. ±2°). In one embodiment, the thermal event has a peak temperature of about 90° C. (e.g. ±2°). In one embodiment, the solid form is characterized by a DSC thermogram that matches the DSC thermogram depicted in FIG. 2. In one embodiment, the DSC thermogram is as measured by DSC using a scanning rate of about 10° C./minute. In one embodiment, provided herein is a solid form comprising a free base of Compound 1, which exhibits a weight loss of about 15.8% upon heating from about 60° C. to about 110° C. In one embodiment, the solid form is characterized by a TGA thermogram that matches the TGA thermogram depicted in FIG. 2. In one embodiment, the TGA thermogram is as measured using a heating rate of about 10° C./minute. In one embodiment, the Form 1 that provides FIG. 2 is a 2-MeTHF solvate.

Another representative overlay of TGA/DSC thermograms of Form 1 is provided in FIG. 3. In one embodiment, provided herein is a solid form comprising a free base of Compound 1, which exhibits, as characterized by DSC, a thermal (endo) event with a peak temperature of about 117° C. (e.g. ±2°). In one embodiment, the thermal event has an onset temperature of about 110° C. (e.g. ±2°). In one embodiment, the solid form is characterized by a DSC thermogram that matches the DSC thermogram depicted in FIG. 3. In one embodiment, the DSC thermogram is as measured by DSC using a scanning rate of about 10° C./minute. In one embodiment, provided herein is a solid form comprising a free base of Compound 1, which exhibits a weight loss of about 13.0% upon heating from about 25° C. to about 150° C. In one embodiment, the solid form is characterized by a TGA thermogram that matches the TGA thermogram depicted in FIG. 3. In one embodiment, the TGA thermogram is as measured using a heating rate of about 10° C./minute. In one embodiment, the Form 1 that provides FIG. 3 is an isopropyl acetate solvate.

In some embodiments, provided herein is a solid form comprising a free base of Compound 1, which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1 which is an isostructural solvate. In one embodiment, provided herein is a solid form comprising a free base of Compound 1, wherein the molar ratio of Compound 1 to the solvent ranges from about 1:0.5 to about 1:1. In one embodiment, the solid form is a 2-MeTHF solvate of a free base of Compound 1. In another embodiment, the solid form is an isopropyl acetate solvate of a free base of Compound 1.

In one embodiment, provided herein is a solid form comprising Form 1 of a free base of Compound 1 and amorphous free base of Compound 1. In one embodiment, provided herein is a solid form comprising Form 1 of a free base Compound 1 and one or more other crystalline forms of a free base of Compound 1 provided herein.

All of the combinations of the above embodiments are encompassed by this application.

5.2.1.2 Form 2 of Compound 1

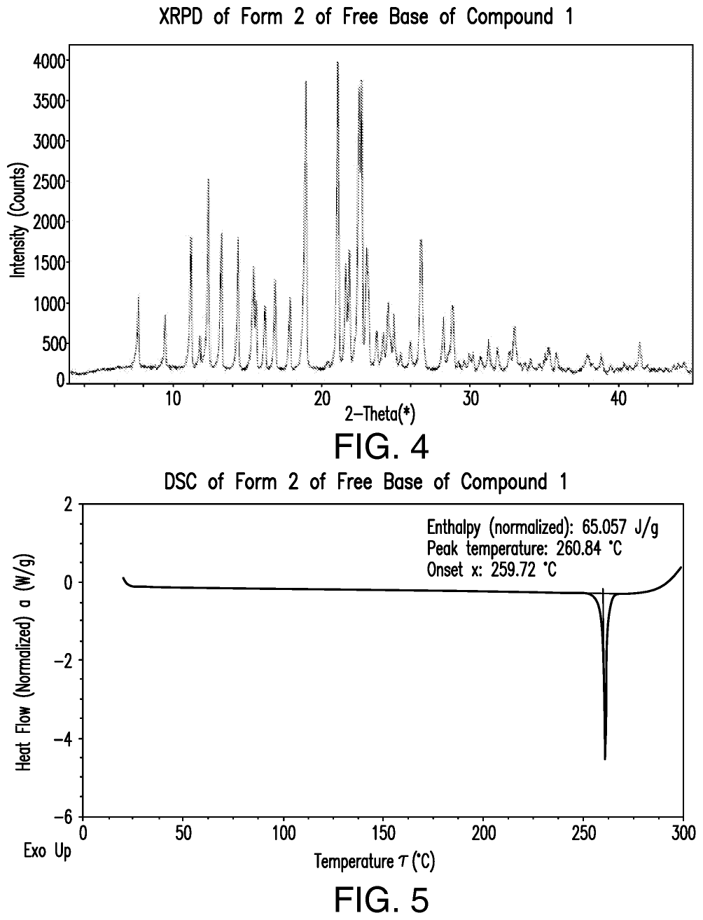

In one embodiment, provided herein is a Form 2 of Compound 1. A representative XRPD pattern of Form 2 of Compound 1 is provided in FIG. 4.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21 or all of the XRPD peaks located at approximately the following positions (e.g., degrees 2θ±0.2) when measured using Cu Kα radiation: 7.6, 9.4, 11.2, 12.4, 13.2, 14.3, 15.4, 15.6, 16.2, 16.9, 17.9, 18.9, 21.1, 21.6, 21.8, 22.5, 22.7, 23.0, 24.5, 24.9, 27.0, and 28.8° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form or substantially crystalline form) comprising a free base of Compound 1, characterized by an XRPD pattern, when measured using Cu Kα radiation, comprising at least three peaks selected from the group consisting of approximately (e.g., ±0.2°) 11.2, 12.4, 13.2, 14.3, 18.9, 21.1, 21.6, 21.8, 22.5, 22.7, 23.0, and 27.0° 2θ. In one embodiment, the solid form is characterized by an XRPD pattern comprising at least four peaks selected from the group consisting of approximately (e.g., ±0.2°) 11.2, 12.4, 13.2, 14.3, 18.9, 21.1, 21.6, 21.8, 22.5, 22.7, 23.0, and 27.0° 2θ. In one embodiment, the solid form is characterized by an XRPD pattern comprising at least five peaks selected from the group consisting of approximately (e.g., ±0.2°) 11.2, 12.4, 13.2, 14.3, 18.9, 21.1, 21.6, 21.8, 22.5, 22.7, 23.0, and 27.0° 2θ.

In one embodiment, provided herein is a solid form (e.g. a crystalline form) comprising a free base of Compound 1, characterized by an XRPD pattern comprising peaks at approximately (e.g., ±0.2°) 12.4, 18.9, and 21.1° 2θ. In one embodiment, the XRPD pattern further comprises a peak at approximately (e.g., ±0.2°) 13.2 and 22.5° 2θ. In one embodiment, the XRPD pattern further comprises peaks at approximately (e.g., ±0.2°) 11.2 and 22.7° 2θ. In one embodiment, the XRPD pattern comprises peaks at approximately (e.g., ±0.2°) 11.2, 12.4, 13.2, 14.3, 18.9, 21.1, 21.8, 22.5, 22.7, 23.0, and 27.0° 2θ.

In one embodiment, provided herein is a solid form comprising a free base of Compound 1, characterized by an XRPD pattern that matches the XRPD pattern depicted in FIG. 4.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

A representative DSC thermogram of Form 2 is provided in FIG. 5. In one embodiment, provided herein is a solid form comprising a free base of Compound 1, which exhibits, as characterized by DSC, a thermal event (endothermic) with an onset temperature of about 260° C. (e.g. ±2°). In one embodiment, the thermal event has a peak temperature of about 261° C. (e.g. ±2°). In one embodiment, without being bound by a particular theory, the thermal event corresponds to melting. In one embodiment, the solid form is characterized by a DSC thermogram that matches the DSC thermogram depicted in FIG. 5. In one embodiment, the DSC thermogram is as measured by DSC using a scanning rate of about 10° C./minute.

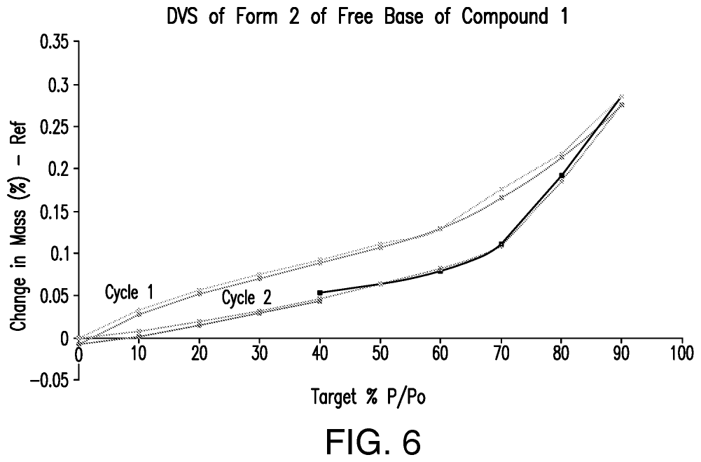

A representative DVS isotherm of Form 2 is provided in FIG. 6. In one embodiment, provided herein is a solid form comprising a free base of Compound 1, which exhibits a weight increase of about 0.3% (e.g. ±0.05%) when subjected to an increase in relative humidity from about 0 to about 90% relative humidity. In one embodiment, the solid form is characterized by a DVS isotherm that matches the DVS isotherm depicted in FIG. 6. In one embodiment, the DVS isotherm is as measured at about 25° C.

In one embodiment, provided herein is a solid form comprising a free base of Compound 1 having approximately unit cell dimensions of: a=8.2 Å, b=14.8 Å, c=18.7 Å, α=90°, β=90°, and γ=90°. In one embodiment, Form 2 has approximately unit cell dimensions of: a=8.17 Å, b=14.75 Å, c=18.69 Å, α=90°, β=90°, and γ=90°. In one embodiment, Form 2 has approximately unit cell dimensions of: a=8.169 Å, b=14.750 Å, c=18.694 Å, α=90°, β=90°, and γ=90°. In one embodiment, Form 2 has a unit cell of a space group of P2₁2₁2₁. In one embodiment, Form 2 has a volume of about 2252.4 Å³/cell. In one embodiment, Form 2 has a Z value of 4. In one embodiment, Form 2 has a density of about 1.336 g/cm³.

In one embodiment, provided herein is a solid form comprising a free base of Compound 1 which is anhydrous. In one embodiment, the solid form is a crystalline anhydrous free base of Compound 1. In one embodiment, the solid form is substantially free of amorphous Compound 1. In one embodiment, the solid form is substantially free of other crystalline forms of Compound 1. In one embodiment, the solid form is substantially free of salts of Compound 1. In one embodiment, the solid form is not solvated. In one embodiment, one or more residual solvent may be present in the solid form, but the residual solvent does not form a solvate of Compound 1. In one embodiment, the solid form is substantially pure. In one embodiment, the solid form is substantially chemically pure. In one embodiment, the solid form is about over 95 wt % chemically pure. In one embodiment, the solid form is about over 96 wt % chemically pure. In one embodiment, the solid form is about over 97 wt % chemically pure. In one embodiment, the solid form is about over 98 wt % chemically pure. In one embodiment, the solid form is about over 99 wt % chemically pure. In one embodiment, the solid form is substantially enantiomerically pure. In one embodiment, the solid form is about at least 98% enantiomerically pure. In one embodiment, the solid form is about at least 99% enantiomerically pure. In one embodiment, the solid form is about at least 99.5% enantiomerically pure. In one embodiment, the solid form is substantially physically pure.

In one embodiment, Form 2 is substantially non-hygroscopic. In one embodiment, Form 2 is non-hygroscopic. In one embodiment, Form 2 is stable after storage at 30° C.±2° C./65%±5% RH for at least 12 months. In one embodiment, Form 2 is stable after storage at 40° C.±2° C./75%±5% RH for at least 6 months. In one embodiment, Form 2 stored at 30° C.±2° C./65%±5% RH for at least 12 months or at 40° C.±2° C./75%±5% RH for at least 6 months is at least 97 wt % chemically pure. In one embodiment, Form 2 stored at 30° C.±2° C./65%±5% RH for at least 12 months or at 40° C.±2° C./75%±5% RH for at least 6 months is at least 99% enantiomerically pure. In one embodiment, Form 2 stored at 30° C.±2° C./65%±5% RH for at least 12 months or at 40° C.±2° C./75%±5% RH for at least 6 months is at least 99 wt % physically pure (e.g. substantially free of amorphous Compound 1 or other solid forms of Compound 1).

In one embodiment, provided herein is a solid form comprising Form 2 of a free base of Compound 1 and amorphous free base of Compound 1. In one embodiment, provided herein is a solid form comprising Form 2 of a free base Compound 1 and one or more other crystalline forms of a free base of Compound 1 provided herein.

All of the combinations of the above embodiments are encompassed by this application.

5.2.1.3 Form 3 of Compound 1

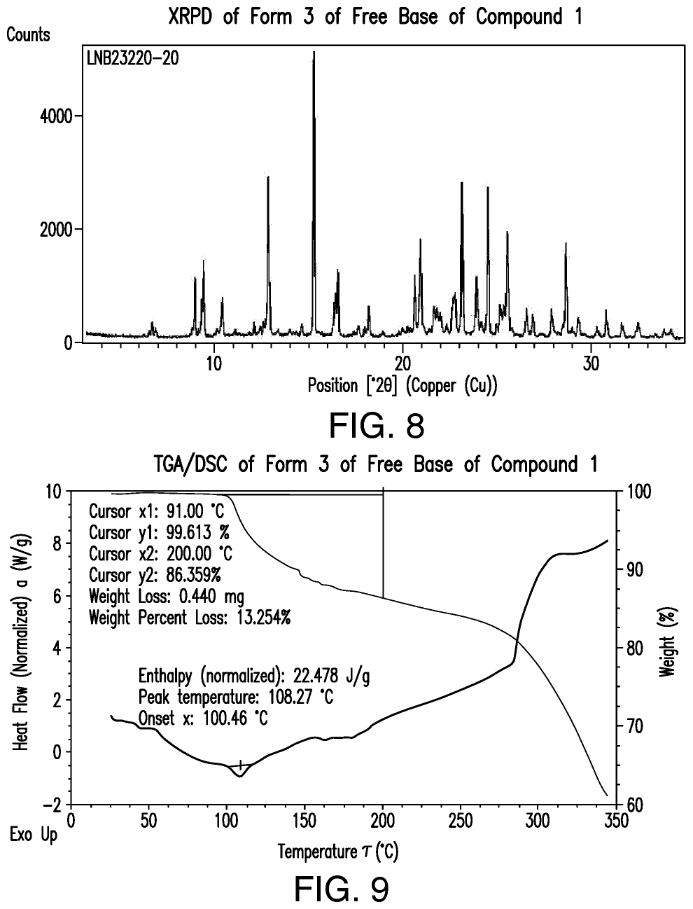

In one embodiment, provided herein is a Form 3 of Compound 1. A representative XRPD pattern of Form 3 of Compound 1 is provided in FIG. 8.

In one embodiment, provided herein is a solid form comprising a free base of Compound 1, characterized by 1, 2, 3, 4, 5, 6, 7, 8, or all of the XRPD peaks located at approximately the following positions (e.g., degrees 2θ±0.2) when measured using Cu Kα radiation: 9.0, 9.4, 10.4, 12.8, 15.3, 16.4, 16.6, 18.2, and 20.6° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 9.0, 9.4, 10.4, 12.8, 15.3, 16.4, 16.6, 18.2, and 20.6° 2θ. In one embodiment, the solid form is characterized by an XRPD pattern comprising at least four peaks selected from the group consisting of approximately (e.g., ±0.2°) 9.0, 9.4, 10.4, 12.8, 15.3, 16.4, 16.6, 18.2, and 20.6° 2θ. In one embodiment, the solid form is characterized by an XRPD pattern comprising at least five peaks selected from the group consisting of approximately (e.g., ±0.2°) 9.0, 9.4, 10.4, 12.8, 15.3, 16.4, 16.6, 18.2, and 20.6° 2θ.

In one embodiment, provided herein is a solid form comprising a free base of Compound 1, characterized by an XRPD pattern comprising peaks at approximately (e.g., ±0.2°) 9.4, 12.8, and 15.3° 2θ. In one embodiment, the solid form is characterized by an XRPD pattern further comprising peaks at approximately (e.g., ±0.2°) 16.6 and 20.6° 2θ. In one embodiment, the XRPD pattern further comprises peaks at approximately (e.g., ±0.2°) 9.0 and 16.4° 2θ. In one embodiment, the XRPD pattern further comprises a peak at approximately (e.g., ±0.2°) 10.4° 2θ. In one embodiment, the XRPD pattern further comprises a peak at approximately (e.g., ±0.2°) 18.2° 2θ.

In one embodiment, provided herein is a solid form comprising a free base of Compound 1, characterized by an XRPD pattern that matches the XRPD pattern depicted in FIG. 8.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

A representative overlay of TGA/DSC thermograms of Form 3 is provided in FIG. 9. In one embodiment, provided herein is a solid form comprising a free base of Compound 1, which exhibits, as characterized by DSC, a thermal (endo) event with an onset temperature of about 100° C. (e.g. ±2°). In one embodiment, the thermal event has a peak temperature at about 108° C. (e.g. ±2°). In one embodiment, the solid form is characterized by a DSC thermogram that matches the DSC thermogram depicted in FIG. 9. In one embodiment, the DSC thermogram is as measured by DSC using a scanning rate of about 10° C./minute. In one embodiment, the Form 3 that provides FIG. 9 is a 2-MeTHF solvate.

In one embodiment, provided herein is a solid form comprising a free base of Compound 1, which exhibits a weight loss of about 13.3% upon heating from about 50° C. to about 200° C. In one embodiment, the solid form is characterized by a TGA thermogram that matches the TGA thermogram depicted in FIG. 9. In one embodiment, the TGA thermogram is as measured using a heating rate of about 10° C./minute.

In some embodiments, provided herein is a solid form comprising a free base of Compound 1, which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent(s) ranges from about 1:1 to about 1:8. In one embodiment, the molar ratio of Compound 1 to the solvent(s) ranges from about 1:4 to about 1:5. In one embodiment, the molar ratio of Compound 1 to water ranges from about 1:1 to about 1:6. In one embodiment, the molar ratio of Compound 1 to water ranges from about 1:3 to about 1:5. In one embodiment, the molar ratio of Compound 1 to the organic solvent ranges from about 1:0.5 to about 1:1.5. In one embodiment, the molar ratio of Compound 1 to the organic solvent ranges from about 1:0.8 to about 1:1.1. In one embodiment, the solid form is a 2-MeTHF solvate of free base of Compound 1. In one embodiment, the molar ratio of Compound 1 to 2-MeTHF is about 1:0.7. In one embodiment, the molar ratio of Compound 1 to 2-MeTHF is about 1:0.8. In one embodiment, the molar ratio of Compound 1 to 2-MeTHF is about 1:1.

In one embodiment, provided herein is a solid form comprising Form 3 of a free base of Compound 1 and amorphous free base of Compound 1. In one embodiment, provided herein is a solid form comprising Form 3 of a free base Compound 1 and one or more other crystalline forms of a free base of Compound 1 provided herein.

All of the combinations of the above embodiments are encompassed by this application.

5.2.1.4 Form 4 of Compound 1

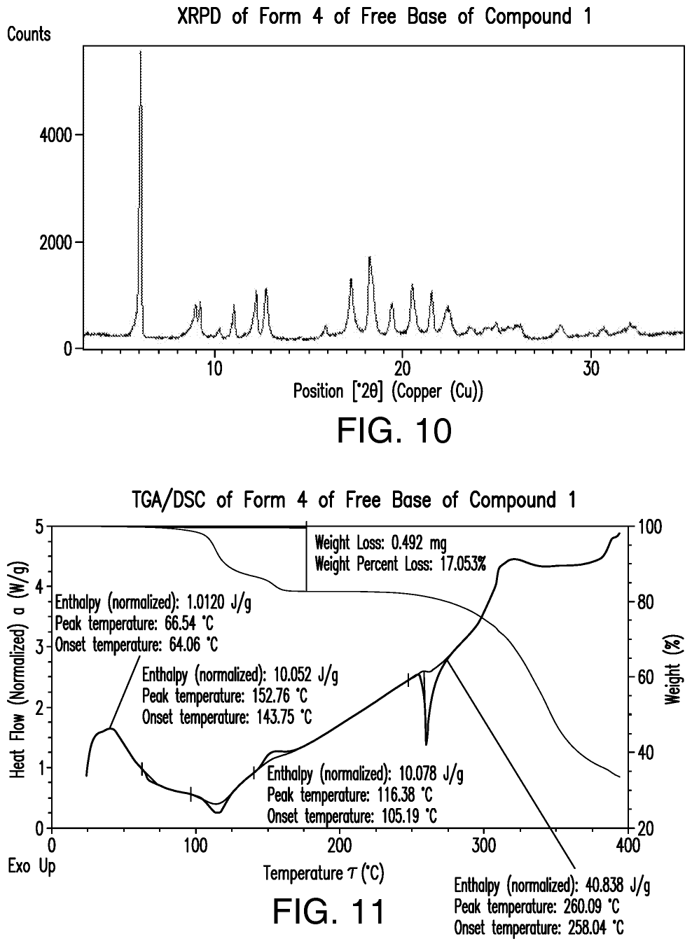

In one embodiment, provided herein is a Form 4 of Compound 1. A representative XRPD pattern of Form 4 of Compound 1 is provided in FIG. 10.

In one embodiment, provided herein is a solid form comprising a free base of Compound 1, characterized by 1, 2, 3, 4, 5, 6, 7, 8, 9, 10, 11, 12, 13, or all of the XRPD peaks located at approximately the following positions (e.g., degrees 2θ±0.2) when measured using Cu Kα radiation: 6.0, 6.1, 9.0, 9.2, 11.0, 12.2, 12.7, 17.2, 18.2, 18.4, 19.4, 20.5, 21.5, and 22.5° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, solid form is characterized by at least 11 of the peaks. In one embodiment, solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.1, 9.2, 11.0, 12.2, 12.7, 17.2, 18.2, 20.5 and 21.5° 2θ. In one embodiment, the solid form is characterized by an XRPD pattern comprising at least four peaks selected from the group consisting of approximately (e.g., ±0.2°) 6.1, 9.2, 11.0, 12.2, 12.7, 17.2, 18.2, 20.5 and 21.5° 2θ. In one embodiment, the solid form is characterized by an XRPD pattern comprising at least five peaks selected from the group consisting of approximately (e.g., ±0.2°) 6.1, 9.2, 11.0, 12.2, 12.7, 17.2, 18.2, 20.5 and 21.5° 2θ.

In one embodiment, provided herein is a solid form comprising a free base of Compound 1, characterized by an XRPD pattern comprising peaks at approximately (e.g., ±0.2°) 6.1, 17.2, and 18.2° 2θ. In one embodiment, the solid form is characterized by an XRPD pattern further comprising peaks at approximately (e.g., ±0.2°) 12.7 and 20.5° 2θ. In one embodiment, the solid form is characterized by an XRPD pattern further comprising peaks at approximately (e.g., ±0.2°) 12.2 and 21.5° 2θ. In one embodiment, the XRPD pattern comprises peaks at approximately (e.g., ±0.2°) 6.1, 9.2, 11.0, 12.2, 12.7, 17.2, 18.2, 20.5, and 21.5° 2θ.

In one embodiment, provided herein is a solid form comprising a free base of Compound 1, characterized by an XRPD pattern that matches the XRPD pattern depicted in FIG. 10.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

A representative overlay of TGA/DSC thermograms of Form 4 is provided in FIG. 11. In one embodiment, provided herein is a solid form comprising a free base of Compound 1, which exhibits, as characterized by DSC, a thermal event (endo) with an onset temperature of about 64° C. (e.g. ±2°). In one embodiment the thermal event has a peak temperature of about 67° C. (e.g. ±2°). In one embodiment, the solid form is characterized by a DSC thermogram that matches the DSC thermogram depicted in FIG. 11. In one embodiment, the DSC thermogram is as measured by DSC using a scanning rate of about 10° C./minute. In one embodiment, the Form 4 that provides FIG. 11 is a 1,4-dioxane solvate.

In one embodiment, provided herein is a solid form comprising a free base of Compound 1, which exhibits a weight loss of about 17.1% upon heating from about 75° C. to about 165° C. In one embodiment, the solid form is characterized by a TGA thermogram that matches the TGA thermogram depicted in FIG. 11. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.6 to about 1:2. In one embodiment, the solid form is an 1,4-dioxane solvate of free base of Compound 1. In one embodiment, the molar ratio of Compound 1 to 1,4-dioxane is about 1:0.8. In one embodiment, the molar ratio of Compound 1 to 1,4-dioxane is about 1:1.1.

In one embodiment, provided herein is a solid form comprising Form 4 of a free base of Compound 1 and amorphous free base of Compound 1. In one embodiment, provided herein is a solid form comprising Form 4 of a free base Compound 1 and one or more other crystalline forms of a free base of Compound 1 provided herein.

All of the combinations of the above embodiments are encompassed by this application.

5.2.1.5 Form 5 of Compound 1

In one embodiment, provided herein is a Form 5 of Compound 1.

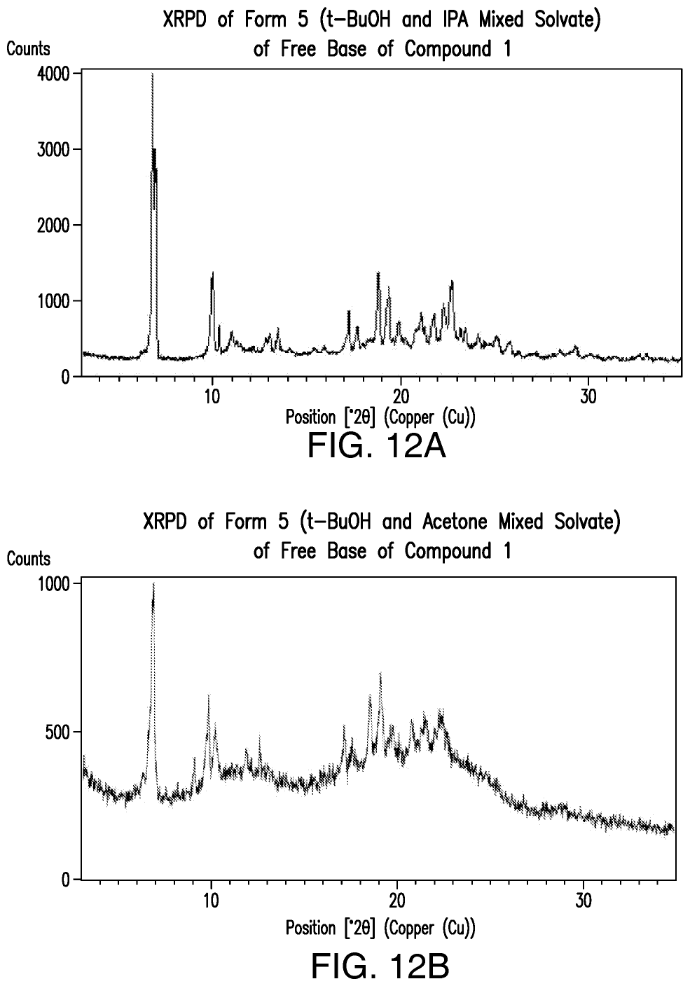

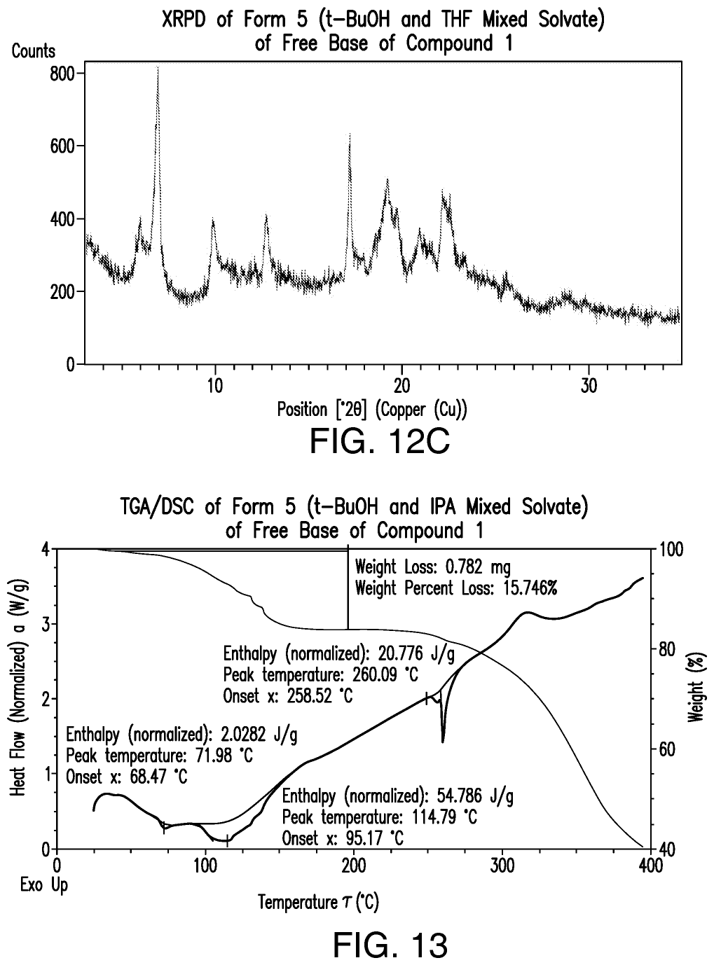

A representative XRPD pattern of Form 5 of Compound 1 is provided in FIG. 12A.

In one embodiment, provided herein is a solid form comprising a free base of Compound 1, characterized by 1, 2, 3, 4, 5, 6, 7, 8, 9, 10, 11, 12, 13, 14, 15, 16, or all of the XRPD peaks located at approximately the following positions (e.g., degrees 2θ±0.2) when measured using Cu Kα radiation: 6.8, 7.0, 10.0, 10.3, 11.0, 17.2, 17.7, 18.9, 19.4, 20.0, 21.1, 21.3, 21.8, 22.2, 22.8, 23.2, and 23.5° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.8, 7.0, 10.0, 17.2, 18.9, 19.4, 21.1, 22.2, and 22.8° 2θ. In one embodiment, the solid form is characterized by an XRPD pattern comprising at least four peaks selected from the group consisting of approximately (e.g., ±0.2°) 6.8, 7.0, 10.0, 17.2, 18.9, 19.4, 21.1, 22.2, and 22.8° 2θ. In one embodiment, the solid form is characterized by an XRPD pattern comprising at least five peaks selected from the group consisting of approximately (e.g., ±0.2°) 6.8, 7.0, 10.0, 17.2, 18.9, 19.4, 21.1, 22.2, and 22.8° 2θ.

In one embodiment, provided herein is a solid form comprising a free base of Compound 1, characterized by an XRPD pattern comprising peaks at approximately (e.g., ±0.2°) 6.8, 10.0, and 18.9° 2θ. In one embodiment, the solid form is characterized by an XRPD pattern further comprising peaks at approximately (e.g., ±0.2°) 19.4 and 22.8° 2θ. In one embodiment, the solid form is characterized by an XRPD pattern further comprising peaks at approximately (e.g., ±0.2°) 7.0 and 22.2° 2θ. In one embodiment, the XRPD pattern comprises peaks at approximately (e.g., ±0.2°) 6.8, 7.0, 10.0, 17.2, 18.9, 19.4, 21.1, 22.2, and 22.8° 2θ.

In one embodiment, provided herein is a solid form comprising a free base of Compound 1, characterized by an XRPD pattern that matches the XRPD pattern depicted in FIG. 12A. In one embodiment, the Form 5 that provides FIG. 12A is a 2-propanol and t-butanol mixed solvate.

Another representative XRPD pattern of Form 5 of Compound 1 is provided in FIG. 12B.

In one embodiment, provided herein is a solid form comprising a free base of Compound 1, characterized by 1, 2, 3, 4, 5, 6, 7, 8, 9, 10, 11, 12, or all of the XRPD peaks located at approximately the following positions (e.g., degrees 2θ±0.2) when measured using Cu Kα radiation: 6.9, 9.1, 9.9, 10.2, 11.9, 12.6, 17.2, 18.6, 19.1, 19.7, 20.8, 21.5, and 22.4° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.9, 9.9, 10.2, 12.6, 18.6, 19.1, 20.8, 21.5, and 22.4° 2θ. In one embodiment, the solid form is characterized by an XRPD pattern comprising at least four peaks selected from the group consisting of approximately (e.g., ±0.2°) 6.9, 9.9, 10.2, 12.6, 18.6, 19.1, 20.8, 21.5, and 22.4° 2θ. In one embodiment, the solid form is characterized by an XRPD pattern comprising at least five peaks selected from the group consisting of approximately (e.g., ±0.2°) 6.9, 9.9, 10.2, 12.6, 18.6, 19.1, 20.8, 21.5, and 22.4° 2θ.

In one embodiment, provided herein is a solid form comprising a free base of Compound 1, characterized by an XRPD pattern comprising peaks at approximately (e.g., ±0.2°) 6.9, 9.9, and 19.1° 2θ. In one embodiment, the solid form is characterized by an XRPD pattern further comprising peaks at approximately (e.g., ±0.2°) 10.2 and 18.6° 2θ. In one embodiment, the solid form is characterized by an XRPD pattern further comprising peaks at approximately (e.g., ±0.2°) 20.8 and 22.4° 2θ. In one embodiment, the XRPD pattern comprises peaks at approximately (e.g., ±0.2°) 6.9, 9.9, 10.2, 12.6, 18.6, 19.1, 20.8, 21.5, and 22.4° 2θ.

In one embodiment, provided herein is a solid form comprising a free base of Compound 1, characterized by an XRPD pattern that matches the XRPD pattern depicted in FIG. 12B. In one embodiment, the Form 5 that provides FIG. 12B is a t-butanol and acetone mixed solvate.

Another representative XRPD pattern of Form 5 of Compound 1 is provided in FIG. 12C.

In one embodiment, provided herein is a solid form comprising a free base of Compound 1, characterized by 1, 2, 3, 4, 5, 6, 7, 8, 9, 10, 11, or all of the XRPD peaks located at approximately the following positions (e.g., degrees 2θ±0.2) when measured using Cu Kα radiation: 3.2, 6.0, 6.9, 9.8, 12.7, 17.2, 19.2, 19.7, 20.9, 22.2, 25.6, and 28.8° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.0, 6.9, 9.8, 12.7, 17.2, 19.2, 19.7, 20.9, and 22.2° 2θ. In one embodiment, the solid form is characterized by an XRPD pattern comprising at least four peaks selected from the group consisting of approximately (e.g., ±0.2°) 6.0, 6.9, 9.8, 12.7, 17.2, 19.2, 19.7, 20.9, and 22.2° 2θ. In one embodiment, the solid form is characterized by an XRPD pattern comprising at least five peaks selected from the group consisting of approximately (e.g., ±0.2°) 6.0, 6.9, 9.8, 12.7, 17.2, 19.2, 19.7, 20.9, and 22.2° 2θ.

In one embodiment, provided herein is a solid form comprising a free base of Compound 1, characterized by an XRPD pattern comprising peaks at approximately (e.g., ±0.2°) 6.9, 17.2, and 19.2° 2θ. In one embodiment, the solid form is characterized by an XRPD pattern further comprising peaks at approximately (e.g., ±0.2°) 6.0 and 22.2° 2θ. In one embodiment, the solid form is characterized by an XRPD pattern further comprising peaks at approximately (e.g., ±0.2°) 9.8 and 19.7° 2θ. In one embodiment, the XRPD pattern comprises peaks at approximately (e.g., ±0.2°) 6.0, 6.9, 9.8, 12.7, 17.2, 19.2, 19.7, 20.9, and 22.2° 2θ.

In one embodiment, provided herein is a solid form comprising a free base of Compound 1, characterized by an XRPD pattern that matches the XRPD pattern depicted in FIG. 12C. In one embodiment, the Form 5 that provides FIG. 12C is a t-butanol and THF mixed solvate.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

A representative overlay of TGA/DSC thermograms of Form 5 is provided in FIG. 13. In one embodiment, provided herein is a solid form comprising a free base of Compound 1, which exhibits, as characterized by DSC, a thermal event (endo) with an onset temperature of about 68° C. (e.g. ±2°). In one embodiment the thermal event has a peak temperature of about 72° C. (e.g. ±2°). In one embodiment, the solid form is characterized by a DSC thermogram that matches the DSC thermogram depicted in FIG. 13. In one embodiment, the DSC thermogram is as measured by DSC using a scanning rate of about 10° C./minute. In one embodiment, provided herein is a solid form comprising a free base of Compound 1, which exhibits a weight loss of about 15.7% upon heating from about 25° C. to about 170° C. In one embodiment, the solid form is characterized by a TGA thermogram that matches the TGA thermogram depicted in FIG. 13. In one embodiment, the TGA thermogram is as measured using a heating rate of about 10° C./minute. In one embodiment, the Form 5 that provides FIG. 13 is a 2-propanol and t-butanol mixed solvate.

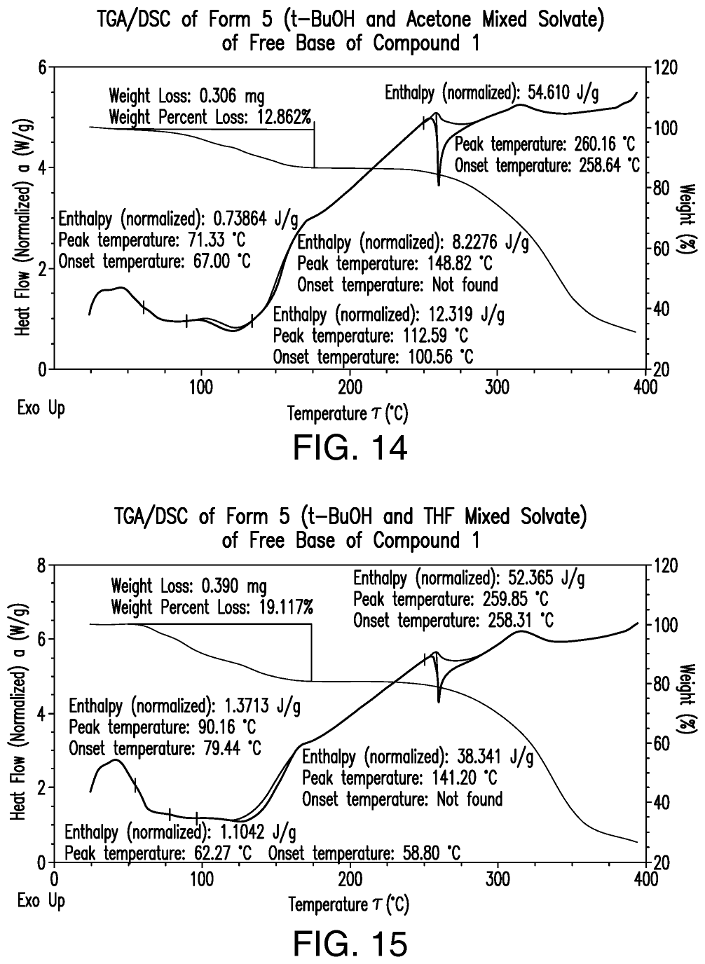

A representative overlay of TGA/DSC thermograms of Form 5 is provided in FIG. 14. In one embodiment, provided herein is a solid form comprising a free base of Compound 1, which exhibits, as characterized by DSC, a thermal event (endo) with an onset temperature of about 67° C. (e.g. ±2°). In one embodiment the thermal event has a peak temperature of about 71° C. (e.g. ±2°). In one embodiment, the solid form is characterized by a DSC thermogram that matches the DSC thermogram depicted in FIG. 14. In one embodiment, the DSC thermogram is as measured by DSC using a scanning rate of about 10° C./minute. In one embodiment, provided herein is a solid form comprising a free base of Compound 1, which exhibits a weight loss of about 12.9% upon heating from about 25° C. to about 170° C. In one embodiment, the solid form is characterized by a TGA thermogram that matches the TGA thermogram depicted in FIG. 14. In one embodiment, the TGA thermogram is as measured using a heating rate of about 10° C./minute. In one embodiment, the Form 5 that provides FIG. 14 is an acetone and t-butanol mixed solvate.

A representative overlay of TGA/DSC thermograms of Form 5 is provided in FIG. 15. In one embodiment, provided herein is a solid form comprising a free base of Compound 1, which exhibits, as characterized by DSC, a thermal event (endo) with an onset temperature of about 59° C. (e.g. ±2°). In one embodiment, the thermal event has a peak temperature of about 62° C. (e.g. ±2°). In one embodiment, the solid form is characterized by a DSC thermogram that matches the DSC thermogram depicted in FIG. 15. In one embodiment, the DSC thermogram is as measured by DSC using a scanning rate of about 10° C./minute. In one embodiment, provided herein is a solid form comprising a free base of Compound 1, which exhibits a weight loss of about 19.1% upon heating from about 25° C. to about 170° C. In one embodiment, the solid form is characterized by a TGA thermogram that matches the TGA thermogram depicted in FIG. 15. In one embodiment, the TGA thermogram is as measured using a heating rate of about 10° C./minute. In one embodiment, the Form 5 that provides FIG. 15 is a THF and t-butanol mixed solvate.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1 which is an isostructural solvate. In one embodiment, provided herein is a solid form comprising a free base of Compound 1 which is a mixed solvate. In one embodiment, the solid form is a 2-propanol and t-butanol mixed solvate. In another embodiment, the solid form is an acetone and t-butanol mixed solvate. In another embodiment, the solid form is a THF and t-butanol mixed solvate. In one embodiment, provided herein is a solid form comprising a free base of Compound 1, wherein the molar ratio of Compound 1 to a solvent ranges from about 1:0.1 to about 1:1.1. In one embodiment, the molar ratio of Compound 1 to a solvent (e.g., acetone) is about 1:0.1. In one embodiment, the molar ratio of Compound 1 to a solvent (e.g., 2-propanol) is about 1:0.4. In one embodiment, the molar ratio of Compound 1 to a solvent (e.g., t-butanol) is about 1:0.6. In one embodiment, the molar ratio of Compound 1 to a solvent (e.g., THF) is about 1:0.7. In one embodiment, the molar ratio of Compound 1 to a solvent is about 1:0.7. In one embodiment, the molar ratio of Compound 1 to a mixture of two solvents is about 1:0.5 to 1:1.9. In one embodiment, the molar ratio of Compound 1 to a mixture of two solvents (e.g. 2-propanol and t-butanol) is about 1:1.0. In one embodiment, the molar ratio of Compound 1 to a mixture of two solvents (e.g. acetone and t-butanol) is about 1:0.8. In one embodiment, the molar ratio of Compound 1 to a mixture of two solvents (e.g. THF and t-butanol) is about 1:1.3.

In one embodiment, provided herein is a solid form comprising Form 5 of a free base of Compound 1 and amorphous free base of Compound 1. In one embodiment, provided herein is a solid form comprising Form 5 of a free base Compound 1 and one or more other crystalline forms of a free base of Compound 1 provided herein.

All of the combinations of the above embodiments are encompassed by this application.

5.2.1.6 Form 6 of Compound 1

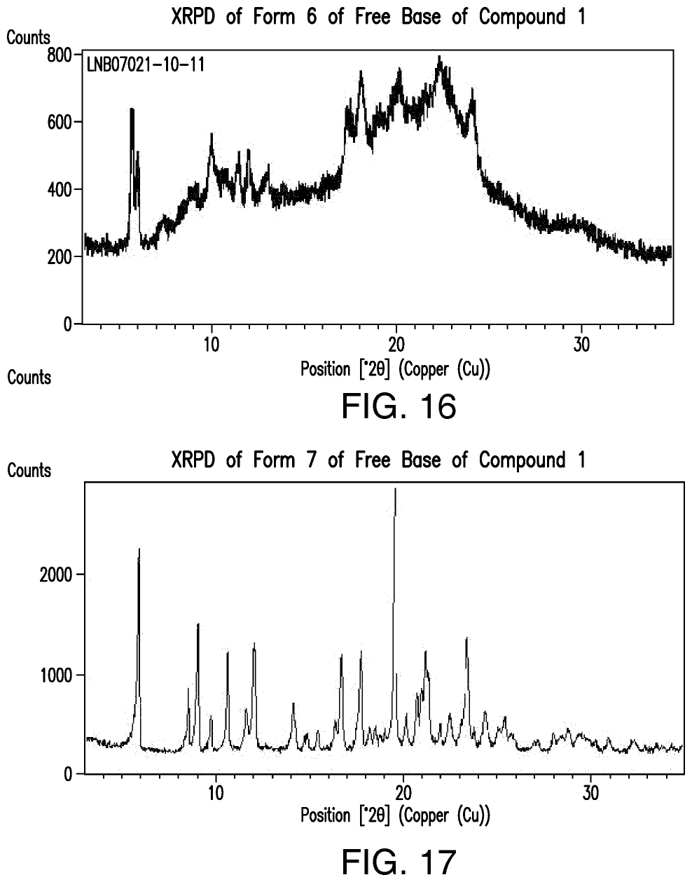

In one embodiment, provided herein is a Form 6 of Compound 1. A representative XRPD pattern of Form 6 of Compound 1 is provided in FIG. 16.

In one embodiment, provided herein is a solid form comprising a free base of Compound 1, characterized by 1, 2, 3, 4, 5, 6, 7, 8, 9, 10, 11, 12, or all of the XRPD peaks located at approximately the following positions (e.g., degrees 2θ±0.2) when measured using Cu Kα radiation: 5.8, 6.0, 9.0, 10.0, 11.5, 12.0, 17.3, 18.0, 19.0, 20.1, 21.5, 22.4, and 24.1° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.8, 6.0, 10.0, 18.1, 20.1, 22.4, and 24.1° 2θ. In one embodiment, the solid form is characterized by an XRPD pattern comprising at least four peaks selected from the group consisting of approximately (e.g., ±0.2°) 5.8, 6.0, 10.0, 18.1, 20.1, 22.4, and 24.1° 2θ. In one embodiment, the solid form is characterized by an XRPD pattern comprising at least five peaks selected from the group consisting of approximately (e.g., ±0.2°) 5.8, 6.0, 10.0, 18.1, 20.1, 22.4, and 24.1° 2θ.

In one embodiment, provided herein is a solid form comprising a free base of Compound 1, characterized by an XRPD pattern comprising peaks at approximately (e.g., ±0.2°) 5.8, 10.0, and 18.1° 2θ. In one embodiment, the solid from is characterized by an XRPD pattern further comprising peaks at approximately (e.g., ±0.2°) 6.0, and 22.4° 2θ. In one embodiment, the solid form is characterized by an XRPD pattern further comprising peaks at approximately (e.g., ±0.2°) 20.1 and 24.1° 2θ. In one embodiment, the XRPD pattern further comprises peaks at approximately (e.g., ±0.2°) 11.5 and 12.0° 2θ.

In one embodiment, provided herein is a solid form comprising a free base of Compound 1, characterized by an XRPD pattern that matches the XRPD pattern depicted in FIG. 16.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising Form 6 of a free base of Compound 1 and amorphous free base of Compound 1. In one embodiment, provided herein is a solid form comprising Form 6 of a free base Compound 1 and one or more other crystalline forms of a free base of Compound 1 provided herein.

All of the combinations of the above embodiments are encompassed by this application.

5.2.1.7 Form 7 of Compound 1

In one embodiment, provided herein is a Form 7 of Compound 1. A representative XRPD pattern of Form 7 of Compound 1 is provided in FIG. 17.

In one embodiment, provided herein is a solid form comprising a free base of Compound 1, characterized by 1, 2, 3, 4, 5, 6, 7, 8, 9, 10, 11, 12, 13, or all of the XRPD peaks located at approximately the following positions (e.g., degrees 2θ±0.2) when measured using Cu Kα radiation: 5.9, 8.6, 9.1, 10.6, 12.0, 12.1, 16.7, 17.8, 19.6, 20.7, 21.0, 21.2, 21.4, and 23.4° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.9, 9.1, 10.6, 12.0, 16.7, 17.8, 19.6, 21.2, and 23.4° 2θ. In one embodiment, the solid form is characterized by an XRPD pattern comprising at least four peaks selected from the group consisting of approximately (e.g., ±0.2°) 5.9, 9.1, 10.6, 12.0, 16.7, 17.8, 19.6, 21.2, and 23.4° 2θ. In one embodiment, the solid form is characterized by an XRPD pattern comprising at least five peaks selected from the group consisting of approximately (e.g., ±0.2°) 5.9, 9.1, 10.6, 12.0, 16.7, 17.8, 19.6, 21.2, and 23.4° 2θ.

In one embodiment, provided herein is a solid form comprising a free base of Compound 1, characterized by an XRPD pattern comprising peaks at approximately (e.g., ±0.2°) 5.9, 9.1, and 19.6° 2θ. In one embodiment, the solid form is characterized by an XRPD pattern further comprising peaks at approximately (e.g., ±0.2°) 12.0 and 23.4° 2θ. In one embodiment, the solid form is characterized by an XRPD pattern further comprising peaks at approximately (e.g., ±0.2°) 10.6 and 21.2° 2θ. In one embodiment, the XRPD pattern comprises peaks at approximately (e.g., ±0.2°) 5.9, 9.1, 10.6, 12.0, 16.7, 17.8, 19.6, 21.2, and 23.4° 2θ.

In one embodiment, provided herein is a solid form comprising a free base of Compound 1, characterized by an XRPD pattern that matches the XRPD pattern depicted in FIG. 17.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

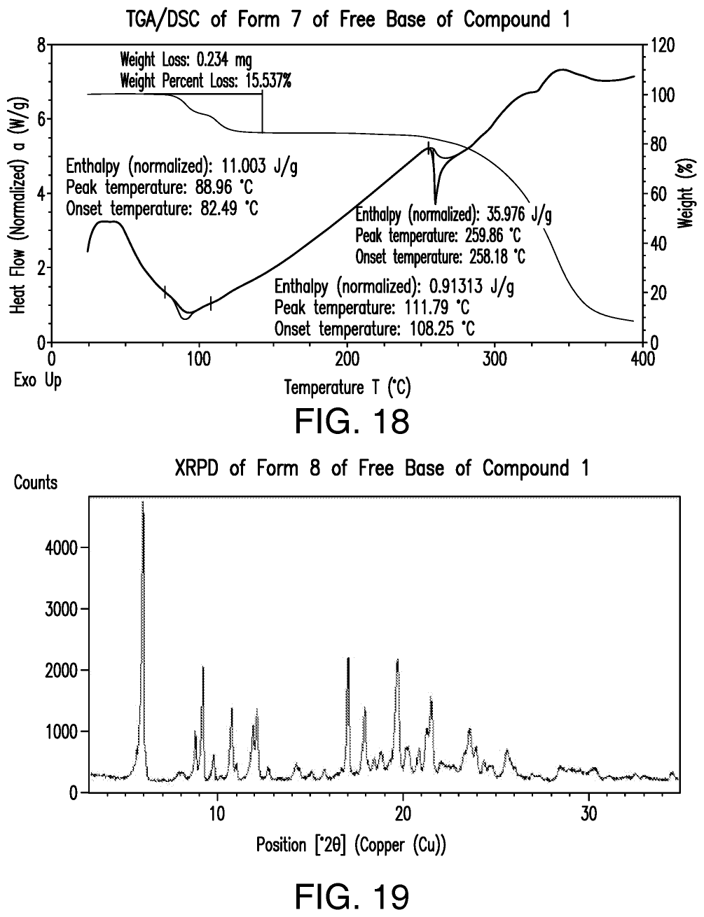

A representative overlay of TGA/DSC thermograms of Form 7 is provided in FIG. 18. In one embodiment, provided herein is a solid form comprising a free base of Compound 1, which exhibits, as characterized by DSC, a thermal event (endo) with an onset temperature of about 83° C. (e.g. ±2°). In one embodiment the thermal event has a peak temperature of about 89° C. (e.g. ±2°). In one embodiment, the solid form is characterized by a DSC thermogram that matches the DSC thermogram depicted in FIG. 18. In one embodiment, the DSC thermogram is as measured by DSC using a scanning rate of about 10° C./minute. In one embodiment, the Form 7 that provides FIG. 18 is a MIBK solvate.

In one embodiment, provided herein is a solid form comprising a free base of Compound 1, which exhibits a weight loss of about 15.5% upon heating from about 85° C. to about 135° C. In one embodiment, the solid form is characterized by a TGA thermogram that matches the TGA thermogram depicted in FIG. 18. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.6 to about 1:1.1. In one embodiment, the solid form is an MIBK solvate of free base of Compound 1. In one embodiment, the molar ratio of Compound 1 to MIBK is about 1:0.7. In one embodiment, the molar ratio of Compound 1 to MIBK is about 1:0.8.

In one embodiment, provided herein is a solid form comprising Form 7 of a free base of Compound 1 and amorphous free base of Compound 1. In one embodiment, provided herein is a solid form comprising Form 7 of a free base Compound 1 and one or more other crystalline forms of a free base of Compound 1 provided herein.

All of the combinations of the above embodiments are encompassed by this application.

5.2.1.8 Form 8 of Compound 1

In one embodiment, provided herein is a Form 8 of Compound 1. A representative XRPD pattern of Form 8 of Compound 1 is provided in FIG. 19.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, or all of the XRPD peaks located at approximately the following positions (e.g., degrees 2θ±0.2) when measured using Cu Kα radiation: 6.0, 6.1, 8.8, 9.2, 9.8, 10.8, 11.9, 12.1, 17.0, 18.0, 18.9, 19.7, 20.1, 20.3, 20.9, 21.3, 21.5, 21.6, 23.6, 24.0, and 25.6° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.0, 9.2, 10.8, 11.9, 12.1, 17.0, 18.0, 19.7, and 21.5° 2θ. In one embodiment, the solid form is characterized by an XRPD pattern comprising at least four peaks selected from the group consisting of approximately (e.g., ±0.2°) 6.0, 9.2, 10.8, 11.9, 12.1, 17.0, 18.0, 19.7, and 21.5° 2θ. In one embodiment, the solid form is characterized by an XRPD pattern comprising at least five peaks selected from the group consisting of approximately (e.g., ±0.2°) 6.0, 9.2, 10.8, 11.9, 12.1, 17.0, 18.0, 19.7, and 21.5° 2θ.

In one embodiment, provided herein is a solid form comprising a free base of Compound 1, characterized by an XRPD pattern comprising peaks at approximately (e.g., ±0.2°) 6.0, 17.0, and 19.7° 2θ. In one embodiment, the solid form is characterized by an XRPD pattern further comprising peaks at approximately (e.g., ±0.2°) 9.2 and 21.5° 2θ. In one embodiment, the solid form is characterized by an XRPD pattern further comprising peaks at approximately (e.g., ±0.2°) 10.8 and 18.0° 2θ. In one embodiment, the XRPD pattern comprises peaks at approximately (e.g., ±0.2°) 6.0, 9.2, 10.8, 11.9, 12.1, 17.0, 18.0, 19.7, and 21.5° 2θ.

In one embodiment, provided herein is a solid form comprising a free base of Compound 1, characterized by an XRPD pattern that matches the XRPD pattern depicted in FIG. 19.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

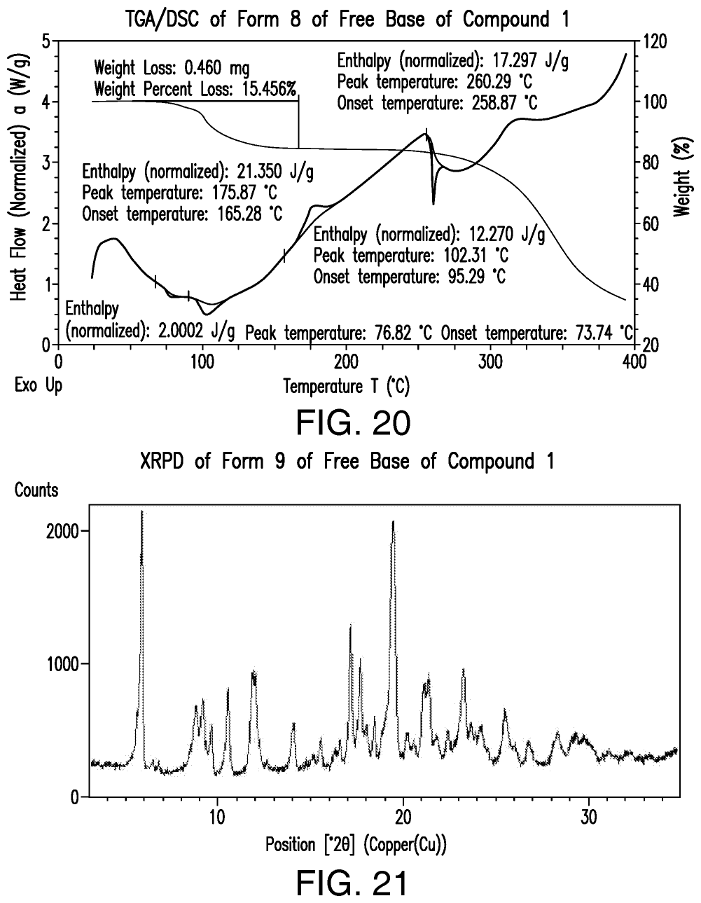

A representative overlay of TGA/DSC thermograms of Form 8 is provided in FIG. 20. In one embodiment, provided herein is a solid form comprising a free base of Compound 1, which exhibits, as characterized by DSC, a thermal event (endo) with an onset temperature of about 74° C. (e.g. ±2°). In one embodiment the thermal event has a peak temperature of about 77° C. (e.g. ±2°). In one embodiment, the solid form is characterized by a DSC thermogram that matches the DSC thermogram depicted in FIG. 20. In one embodiment, the DSC thermogram is as measured by DSC using a scanning rate of about 10° C./minute. In one embodiment, the Form 8 that provides FIG. 20 is a THF solvate.

In one embodiment, provided herein is a solid form comprising a free base of Compound 1, which exhibits a weight loss of about 15.5% upon heating from about 70° C. to about 150° C. In one embodiment, the solid form is characterized by a TGA thermogram that matches the TGA thermogram depicted in FIG. 20. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:2. In one embodiment, the molar ratio of Compound 1 to the solvent ranges from about 1:1 to about 1:1.7. In one embodiment, the solid form is a THF solvate of free base of Compound 1. In one embodiment, the molar ratio of Compound 1 to THF is about 1:1.1. In one embodiment, the molar ratio of Compound 1 to THF is about 1:1.6.

In one embodiment, provided herein is a solid form comprising Form 8 of a free base of Compound 1 and amorphous free base of Compound 1. In one embodiment, provided herein is a solid form comprising Form 8 of a free base Compound 1 and one or more other crystalline forms of a free base of Compound 1 provided herein.

All of the combinations of the above embodiments are encompassed by this application.

5.2.1.9 Form 9 of Compound 1

In one embodiment, provided herein is a Form 9 of Compound 1. A representative XRPD pattern of Form 9 of Compound 1 is provided in FIG. 21.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, or all of the XRPD peaks located at approximately the following positions (e.g., degrees 2θ±0.2) when measured using Cu Kα radiation: 5.9, 8.7, 8.8, 9.2, 9.7, 10.5, 11.9, 12.0, 14.1, 17.2, 17.7, 18.0, 19.1, 19.4, 19.6, 21.1, 21.4, 23.3, 23.7, and 25.5° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.9, 10.5, 11.9, 12.0, 17.2, 17.7, 19.4, 19.6, 21.4, and 23.3° 2θ. In one embodiment, the solid form is characterized by an XRPD pattern comprising at least four peaks selected from the group consisting of approximately (e.g., ±0.2°) 5.9, 10.5, 11.9, 12.0, 17.2, 17.7, 19.4, 19.6, 21.4, and 23.3° 2θ. In one embodiment, the solid form is characterized by an XRPD pattern comprising at least five peaks selected from the group consisting of approximately (e.g., ±0.2°) 5.9, 10.5, 11.9, 12.0, 17.2, 17.7, 19.4, 19.6, 21.4, and 23.3° 2θ.

In one embodiment, provided herein is a solid form comprising a free base of Compound 1, characterized by an XRPD pattern comprising peaks at approximately (e.g., ±0.2°) 5.9, 17.2, and 19.4° 2θ. In one embodiment, the solid form is characterized by an XRPD pattern further comprising peaks at approximately (e.g., ±0.2°) 17.7 and 19.6° 2θ. In one embodiment, the solid form is characterized by an XRPD pattern further comprising peaks at approximately (e.g., ±0.2°) 11.9 and 23.3° 2θ. In one embodiment, the XRPD pattern comprises peaks at approximately (e.g., ±0.2°) 5.9, 10.5, 11.9, 12.0, 17.2, 17.7, 19.4, 19.6, 21.4, and 23.3° 2θ.

In one embodiment, provided herein is a solid form comprising a free base of Compound 1, characterized by an XRPD pattern that matches the XRPD pattern depicted in FIG. 21.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

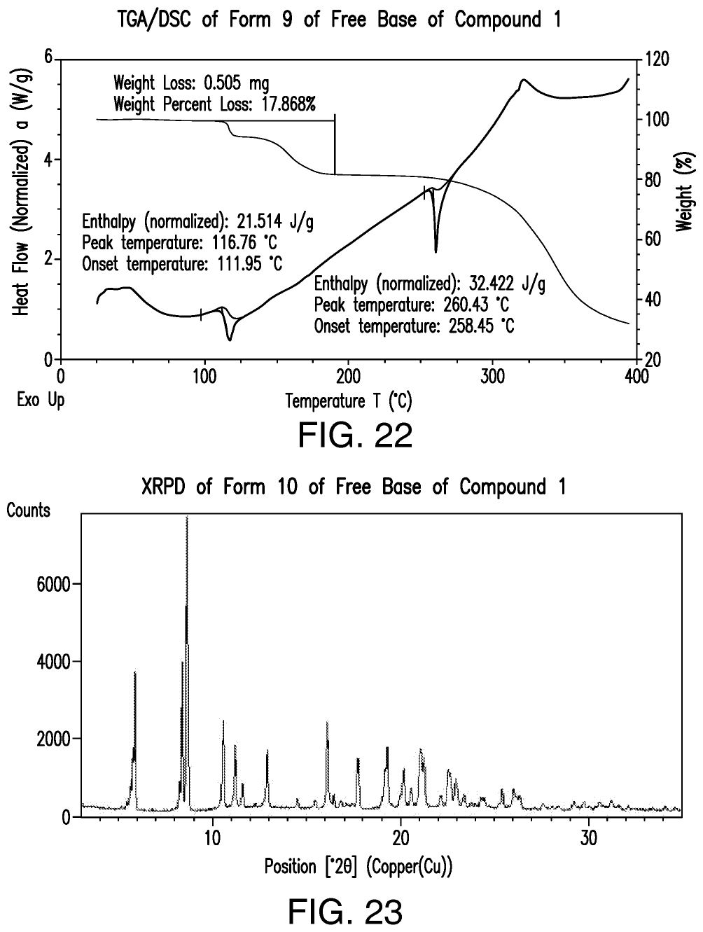

A representative overlay of TGA/DSC thermograms of Form 9 is provided in FIG. 22. In one embodiment, provided herein is a solid form comprising a free base of Compound 1, which exhibits, as characterized by DSC, a thermal event (endo) with an onset temperature of about 112° C. (e.g. ±2°). In one embodiment, the thermal event has a peak temperature of about 117° C. (e.g. ±2°). In one embodiment, the solid form is characterized by a DSC thermogram that matches the DSC thermogram depicted in FIG. 22. In one embodiment, the DSC thermogram is as measured by DSC using a scanning rate of about 10° C./minute. In one embodiment, the Form 9 that provides FIG. 22 is a cyclohexanone solvate.

In one embodiment, provided herein is a solid form comprising a free base of Compound 1, which exhibits a weight loss of about 17.9% upon heating from about 110° C. to about 180° C. In one embodiment, the solid form is characterized by a TGA thermogram that matches the TGA thermogram depicted in FIG. 22. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.8 to about 1:1.2. In one embodiment, the solid form is a cyclohexanone solvate of free base of Compound 1. In one embodiment, the molar ratio of Compound 1 to cyclohexanone is about 1:1.

In one embodiment, provided herein is a solid form comprising Form 9 of a free base of Compound 1 and amorphous free base of Compound 1. In one embodiment, provided herein is a solid form comprising Form 9 of a free base Compound 1 and one or more other crystalline forms of a free base of Compound 1 provided herein.

All of the combinations of the above embodiments are encompassed by this application.

5.2.1.10 Form 10 of Compound 1

In one embodiment, provided herein is a Form 10 of Compound 1. A representative XRPD pattern of Form 10 of Compound 1 is provided in FIG. 23.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, or all of the XRPD peaks located at approximately the following positions (e.g., degrees 2θ±0.2) when measured using Cu Kα radiation: 5.7, 5.8, 5.9, 8.2, 8.4, 8.6, 10.6, 11.2, 12.9, 16.1, 17.7, 19.2, 19.3, 20.2, 21.0, 21.1, 21.3, 22.5, 22.7, and 22.9° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.9, 8.4, 8.6, 10.6, 11.2, 12.9, 16.1, 19.3, and 21.1° 2θ. In one embodiment, the solid form is characterized by an XRPD pattern comprising at least four peaks selected from the group consisting of approximately (e.g., ±0.2°) 5.9, 8.4, 8.6, 10.6, 11.2, 12.9, 16.1, 19.3, and 21.1° 2θ. In one embodiment, the solid form is characterized by an XRPD pattern comprising at least five peaks selected from the group consisting of approximately (e.g., ±0.2°) 5.9, 8.4, 8.6, 10.6, 11.2, 12.9, 16.1, 19.3, and 21.1° 2θ.

In one embodiment, provided herein is a solid form comprising a free base of Compound 1, characterized by an XRPD pattern comprising peaks at approximately (e.g., ±0.2°) 5.9, 8.4, and 8.6° 2θ. In one embodiment, the solid form is characterized by an XRPD pattern further comprising peaks at approximately (e.g., ±0.2°) 10.6 and 16.1° 2θ. In one embodiment, the solid form is characterized by an XRPD pattern further comprising peaks at approximately (e.g., ±0.2°) 11.2 and 19.3° 2θ. In one embodiment, the XRPD pattern comprises peaks at approximately (e.g., ±0.2°) 5.9, 8.4, 8.6, 10.6, 11.2, 12.9, 16.1, 19.3, and 21.1° 2θ.

In one embodiment, provided herein is a solid form comprising a free base of Compound 1, characterized by an XRPD pattern that matches the XRPD pattern depicted in FIG. 23.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

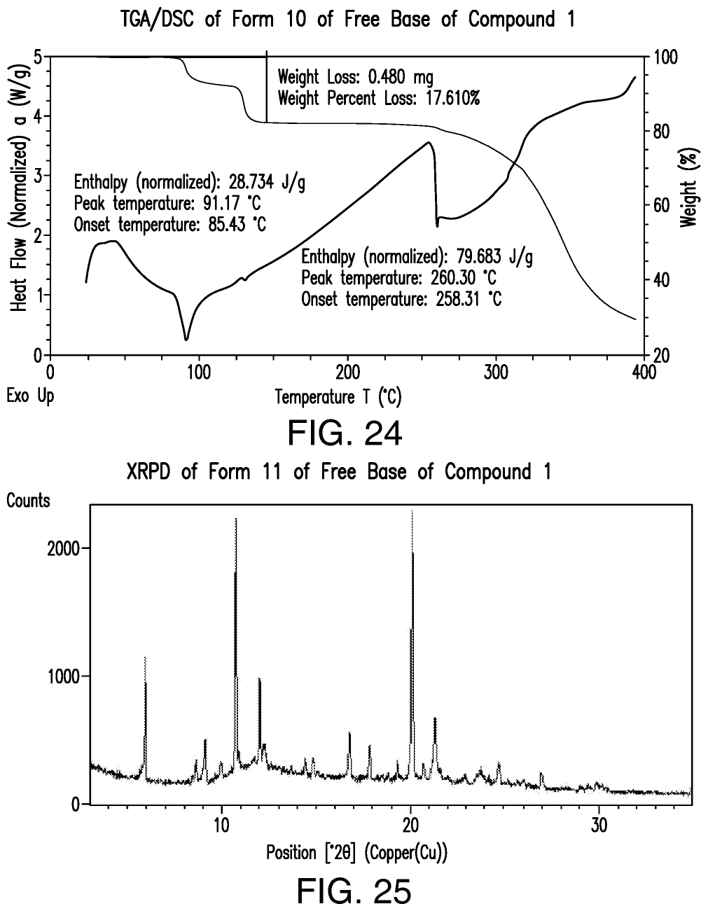

A representative overlay of TGA/DSC thermograms of Form 10 is provided in FIG. 24. In one embodiment, provided herein is a solid form comprising a free base of Compound 1, which exhibits, as characterized by DSC, a thermal event (endo) with an onset temperature of about 85° C. (e.g. ±2°). In one embodiment, the thermal event has a peak temperature of about 91° C. (e.g. ±2°). In one embodiment, the solid form is characterized by a DSC thermogram that matches the DSC thermogram depicted in FIG. 24. In one embodiment, the DSC thermogram is as measured by DSC using a scanning rate of about 10° C./minute. In one embodiment, the Form 10 that provides FIG. 24 is a MIBK solvate.

In one embodiment, provided herein is a solid form comprising a free base of Compound 1, which exhibits a weight loss of about 17.6% upon heating from about 85° C. to about 120° C. In one embodiment, the solid form is characterized by a TGA thermogram that matches the TGA thermogram depicted in FIG. 24. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.8 to about 1:1.2. In one embodiment, the solid form is a MIBK solvate of free base of Compound 1. In one embodiment, the molar ratio of Compound 1 to MIBK is about 1:1.

In one embodiment, provided herein is a solid form comprising Form 10 of a free base of Compound 1 and amorphous free base of Compound 1. In one embodiment, provided herein is a solid form comprising Form 10 of a free base Compound 1 and one or more other crystalline forms of a free base of Compound 1 provided herein.

All of the combinations of the above embodiments are encompassed by this application.

5.2.1.11 Form 11 of Compound 1

In one embodiment, provided herein is a Form 11 of Compound 1. A representative XRPD pattern of Form 11 of Compound 1 is provided in FIG. 25.

In one embodiment, provided herein is a solid form comprising a free base of Compound 1, characterized by 1, 2, 3, 4, 5, 6, 7, 8, 9, 10, 11, 12, 13, 14, 15, 16, 17, or all of the XRPD peaks located at approximately the following positions (e.g., degrees 2θ±0.2) when measured using Cu Kα radiation: 5.9, 8.6, 9.1, 10.0, 10.7, 10.9, 11.7, 12.0, 12.2, 14.4, 14.8, 16.7, 17.8, 19.3, 20.1, 20.7, 21.3, and 24.7° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.9, 9.1, 10.7, 12.0, 12.2, 16.7, 17.8, 20.1, and 21.3° 2θ. In one embodiment, the solid form is characterized by an XRPD pattern comprising at least four peaks selected from the group consisting of approximately (e.g., ±0.2°) 5.9, 9.1, 10.7, 12.0, 12.2, 16.7, 17.8, 20.1, and 21.3° 2θ. In one embodiment, the solid form is characterized by an XRPD pattern comprising at least five peaks selected from the group consisting of approximately (e.g., ±0.2°) 5.9, 9.1, 10.7, 12.0, 12.2, 16.7, 17.8, 20.1, and 21.3° 2θ.

In one embodiment, provided herein is a solid form comprising a free base of Compound 1, characterized by an XRPD pattern comprising peaks at approximately (e.g., ±0.2°) 5.9, 10.7, and 20.1° 2θ. In one embodiment, the solid form is characterized by an XRPD pattern further comprising peaks at approximately (e.g., ±0.2°) 12.0 and 23.1° 2θ. In one embodiment, the solid form is characterized by an XRPD pattern further comprising peaks at approximately (e.g., ±0.2°) 9.1 and 16.7° 2θ. In one embodiment, the XRPD pattern comprises peaks at approximately (e.g., ±0.2°) 5.9, 9.1, 10.7, 12.0, 12.2, 16.7, 17.8, 20.1, and 21.3° 2θ.

In one embodiment, provided herein is a solid form comprising a free base of Compound 1, characterized by an XRPD pattern that matches the XRPD pattern depicted in FIG. 25.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

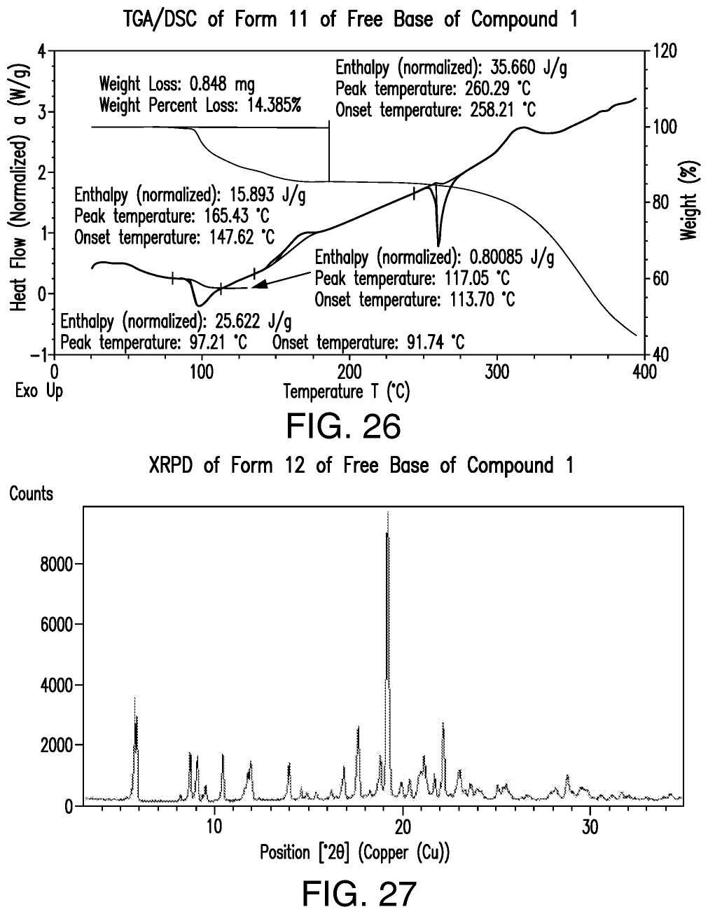

A representative overlay of TGA/DSC thermograms of Form 11 is provided in FIG. 26. In one embodiment, provided herein is a solid form comprising a free base of Compound 1, which exhibits, as characterized by DSC, a thermal event (endo) with an onset temperature of about 92° C. (e.g. ±2°). In one embodiment the thermal event has a peak temperature of about 97° C. (e.g. ±2°). In one embodiment, the solid form is characterized by a DSC thermogram that matches the DSC thermogram depicted in FIG. 26. In one embodiment, the DSC thermogram is as measured by DSC using a scanning rate of about 10° C./minute. In one embodiment, the Form 11 that provides FIG. 26 is an MEK solvate.

In one embodiment, provided herein is a solid form comprising a free base of Compound 1, which exhibits a weight loss of about 14.4% upon heating from about 90° C. to about 150° C. In one embodiment, the solid form is characterized by a TGA thermogram that matches the TGA thermogram depicted in FIG. 26. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.7 to about 1:1.2. In one embodiment, the solid form is a MEK solvate of free base of Compound 1. In one embodiment, the molar ratio of Compound 1 to MEK is about 1:0.8. In one embodiment, the molar ratio of Compound 1 to MEK is about 1:1.

In one embodiment, provided herein is a solid form comprising Form 11 of a free base of Compound 1 and amorphous free base of Compound 1. In one embodiment, provided herein is a solid form comprising Form 11 of a free base Compound 1 and one or more other crystalline forms of a free base of Compound 1 provided herein.

All of the combinations of the above embodiments are encompassed by this application.

5.2.1.12 Form 12 of Compound 1

In one embodiment, provided herein is a Form 12 of Compound 1. A representative XRPD pattern of Form 12 of Compound 1 is provided in FIG. 27.

In one embodiment, provided herein is a solid form comprising a free base of Compound 1, characterized by 1, 2, 3, 4, 5, 6, 7, 8, 9, 10, 11, 12, 13, 14, 15, 16, 17, 18, or all of the XRPD peaks located at approximately the following positions (e.g., degrees 2θ±0.2) when measured using Cu Kα radiation: 5.8, 5.9, 8.7, 9.1, 10.5, 11.8, 11.9, 14.0, 16.9, 17.7, 18.8, 19.2, 19.9, 20.4, 20.8, 21.1, 21.7, 22.1, and 23.0° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.8, 5.9, 8.7, 9.1, 17.7, 18.8, 19.2, 21.1, and 22.1° 2θ. In one embodiment, the solid form is characterized by an XRPD pattern comprising at least four peaks selected from the group consisting of approximately (e.g., ±0.2°) 5.8, 5.9, 8.7, 9.1, 17.7, 18.8, 19.2, 21.1, and 22.1° 2θ. In one embodiment, the solid form is characterized by an XRPD pattern comprising at least five peaks selected from the group consisting of approximately (e.g., ±0.2°) 5.8, 5.9, 8.7, 9.1, 17.7, 18.8, 19.2, 21.1, and 22.1° 2θ.

In one embodiment, provided herein is a solid form comprising a free base of Compound 1, characterized by an XRPD pattern comprising peaks at approximately (e.g., ±0.2°) 5.8, 19.2, and 22.1° 2θ. In one embodiment, the solid form is characterized by an XRPD pattern further comprising peaks at approximately (e.g., ±0.2°) 5.9 and 17.7° 2θ. In one embodiment, the solid form is characterized by an XRPD pattern further comprising peaks at approximately (e.g., ±0.2°) 8.7 and 18.8° 2θ. In one embodiment, the XRPD pattern comprises peaks at approximately (e.g., ±0.2°) 5.8, 5.9, 8.7, 9.1, 17.7, 18.8, 19.2, 21.1, and 22.1° 2θ.

In one embodiment, provided herein is a solid form comprising a free base of Compound 1, characterized by an XRPD pattern that matches the XRPD pattern depicted in FIG. 27.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

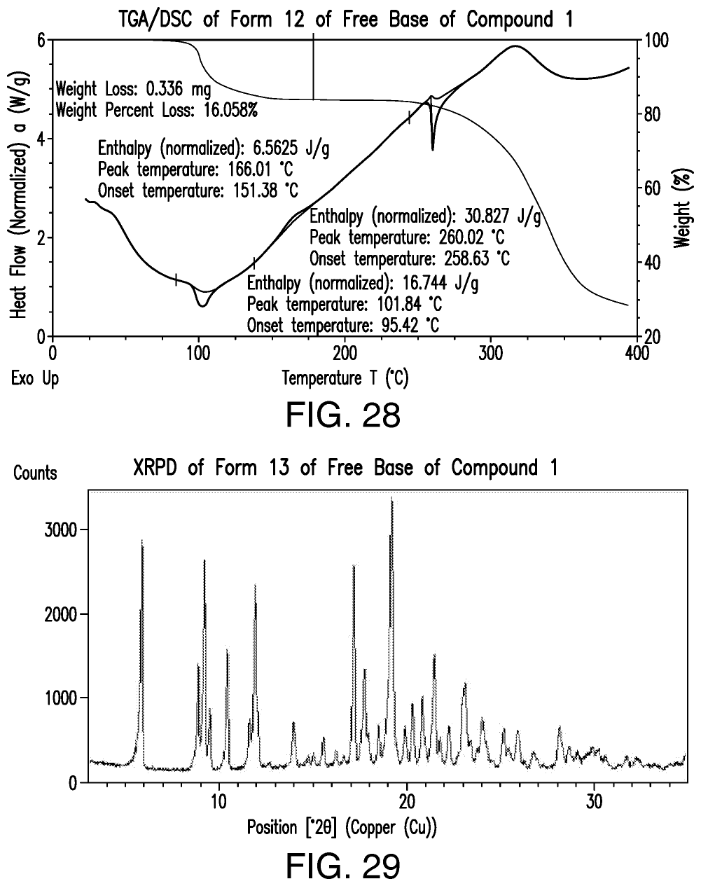

A representative overlay of TGA/DSC thermograms of Form 12 is provided in FIG. 28. In one embodiment, provided herein is a solid form comprising a free base of Compound 1, which exhibits, as characterized by DSC, a thermal event (endo) with an onset temperature of about 95° C. (e.g. ±2°). In one embodiment the thermal event has a peak temperature of about 102° C. (e.g. ±2°). In one embodiment, the solid form is characterized by a DSC thermogram that matches the DSC thermogram depicted in FIG. 28. In one embodiment, the DSC thermogram is as measured by DSC using a scanning rate of about 10° C./minute. In one embodiment, the Form 12 that provides FIG. 28 is a methylcyclohexane solvate.

In one embodiment, provided herein is a solid form comprising a free base of Compound 1, which exhibits a weight loss of about 16.1% upon heating from about 95° C. to about 150° C. In one embodiment, the solid form is characterized by a TGA thermogram that matches the TGA thermogram depicted in FIG. 28. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.7 to about 1:1.1. In one embodiment, the solid form is a methylcyclohexane solvate of free base of Compound 1. In one embodiment, the molar ratio of Compound 1 to methylcyclohexane is about 1:0.8. In one embodiment, the molar ratio of Compound 1 to methylcyclohexane is about 1:0.9.

In one embodiment, provided herein is a solid form comprising Form 12 of a free base of Compound 1 and amorphous free base of Compound 1. In one embodiment, provided herein is a solid form comprising Form 12 of a free base Compound 1 and one or more other crystalline forms of a free base of Compound 1 provided herein.

All of the combinations of the above embodiments are encompassed by this application.

5.2.1.13 Form 13 of Compound 1

In one embodiment, provided herein is a Form 13 of Compound 1. A representative XRPD pattern of Form 13 of Compound 1 is provided in FIG. 29.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, or all of the XRPD peaks located at approximately the following positions (e.g., degrees 2θ±0.2) when measured using Cu Kα radiation: 5.9, 8.9, 9.2, 9.5, 10.4, 11.6, 11.9, 12.1, 13.9, 15.6, 17.2, 17.8, 18.5, 19.2, 19.9, 20.3, 20.8, 21.4, 22.2, 22.9, 23.1, and 24.0° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 5.9, 8.9, 9.2, 10.4, 11.9, 17.2, 17.8, 19.2, 21.4, and 23.1° 2θ. In one embodiment, the solid form is characterized by an XRPD pattern comprising at least four peaks selected from the group consisting of approximately (e.g., ±0.2°) 5.9, 8.9, 9.2, 10.4, 11.9, 17.2, 17.8, 19.2, 21.4, and 23.1° 2θ. In one embodiment, the solid form is characterized by an XRPD pattern comprising at least five peaks selected from the group consisting of approximately (e.g., ±0.2°) 5.9, 8.9, 9.2, 10.4, 11.9, 17.2, 17.8, 19.2, 21.4, and 23.1° 2θ.

In one embodiment, provided herein is a solid form comprising a free base of Compound 1, characterized by an XRPD pattern comprising peaks at approximately (e.g., ±0.2°) 5.9, 9.2, and 19.2° 2θ. In one embodiment, the solid form is characterized by an XRPD pattern further comprising peaks at approximately (e.g., ±0.2°) 11.9 and 17.2° 2θ. In one embodiment, the solid form is characterized by an XRPD pattern further comprising peaks at approximately (e.g., ±0.2°) 10.4, 21.4, and 23.1° 2θ. In one embodiment, the XRPD pattern comprises peaks at approximately (e.g., ±0.2°) 5.9, 8.9, 9.2, 10.4, 11.9, 17.2, 17.8, 19.2, 21.4, and 23.1° 2θ.

In one embodiment, provided herein is a solid form comprising a free base of Compound 1, characterized by an XRPD pattern that matches the XRPD pattern depicted in FIG. 29.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

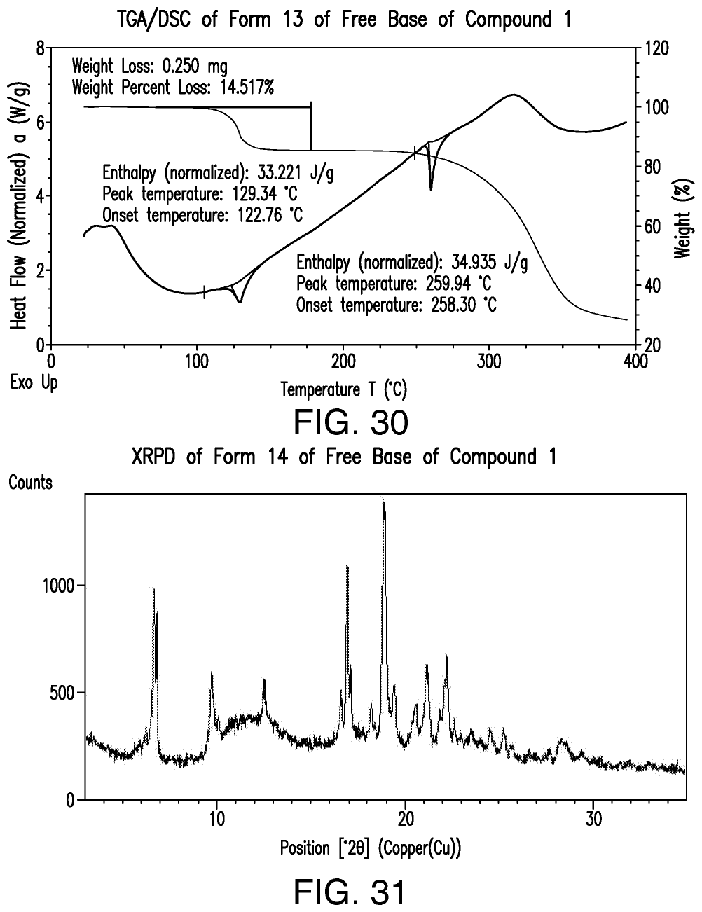

A representative overlay of TGA/DSC thermograms of Form 13 is provided in FIG. 30. In one embodiment, provided herein is a solid form comprising a free base of Compound 1, which exhibits, as characterized by DSC, a thermal event (endo) with an onset temperature of about 123° C. (e.g. ±2°). In one embodiment, the thermal event has a peak temperature of about 129° C. (e.g. ±2°). In one embodiment, the solid form is characterized by a DSC thermogram that matches the DSC thermogram depicted in FIG. 30. In one embodiment, the DSC thermogram is as measured by DSC using a scanning rate of about 10° C./minute. In one embodiment, the Form 13 that provides FIG. 30 is a cyclohexane solvate.

In one embodiment, provided herein is a solid form comprising a free base of Compound 1, which exhibits a weight loss of about 14.5% upon heating from about 120° C. to about 150° C. In one embodiment, the solid form is characterized by a TGA thermogram that matches the TGA thermogram depicted in FIG. 30. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.5. In one embodiment, the molar ratio of Compound 1 to the solvent ranges from about 1:0.6 to about 1:1.1. In one embodiment, the solid form is a cyclohexane solvate of free base of Compound 1. In one embodiment, the molar ratio of Compound 1 to cyclohexane is about 1:0.7. In one embodiment, the molar ratio of Compound 1 to cyclohexane is about 1:0.9.

In one embodiment, provided herein is a solid form comprising Form 13 of a free base of Compound 1 and amorphous free base of Compound 1. In one embodiment, provided herein is a solid form comprising Form 13 of a free base Compound 1 and one or more other crystalline forms of a free base of Compound 1 provided herein.

All of the combinations of the above embodiments are encompassed by this application.

5.2.1.14 Form 14 of Compound 1

In one embodiment, provided herein is a Form 14 of Compound 1. A representative XRPD pattern of Form 14 of Compound 1 is provided in FIG. 31.

In one embodiment, provided herein is a solid form comprising a free base of Compound 1, characterized by 1, 2, 3, 4, 5, 6, 7, 8, 9, 10, 11, 12, 13, 14, 15, 16, 17, 18, or all of the XRPD peaks located at approximately the following positions (e.g., degrees 2θ±0.2) when measured using Cu Kα radiation: 6.7, 6.8, 9.7, 10.1, 12.5, 16.6, 16.9, 17.1, 18.2, 18.9, 19.4, 20.6, 21.2, 21.8, 22.2, 22.6, 23.5, 24.5, and 25.2° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.7, 6.8, 9.7, 12.5, 16.9, 17.1, 18.9, 21.2, and 22.2° 2θ. In one embodiment, the solid form is characterized by an XRPD pattern comprising at least four peaks selected from the group consisting of approximately (e.g., ±0.2°) 6.7, 6.8, 9.7, 12.5, 16.9, 17.1, 18.9, 21.2, and 22.2° 2θ. In one embodiment, the solid form is characterized by an XRPD pattern comprising at least five peaks selected from the group consisting of approximately (e.g., ±0.2°) 6.7, 6.8, 9.7, 12.5, 16.9, 17.1, 18.9, 21.2, and 22.2° 2θ.

In one embodiment, provided herein is a solid form comprising a free base of Compound 1, characterized by an XRPD pattern comprising peaks at approximately (e.g., ±0.2°) 6.7, 16.9, and 18.9° 2θ. In one embodiment, the solid form is characterized by an XRPD pattern further comprising peaks at approximately (e.g., ±0.2°) 6.8 and 22.2° 2θ. In one embodiment, the solid form is characterized by an XRPD pattern further comprising peaks at approximately (e.g., ±0.2°) 9.7 and 21.2° 2θ. In one embodiment, the XRPD pattern comprises peaks at approximately (e.g., ±0.2°) 6.7, 6.8, 9.7, 12.5, 16.9, 17.1, 18.9, 21.2, and 22.2° 2θ.

In one embodiment, provided herein is a solid form comprising a free base of Compound 1, characterized by an XRPD pattern that matches the XRPD pattern depicted in FIG. 31.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

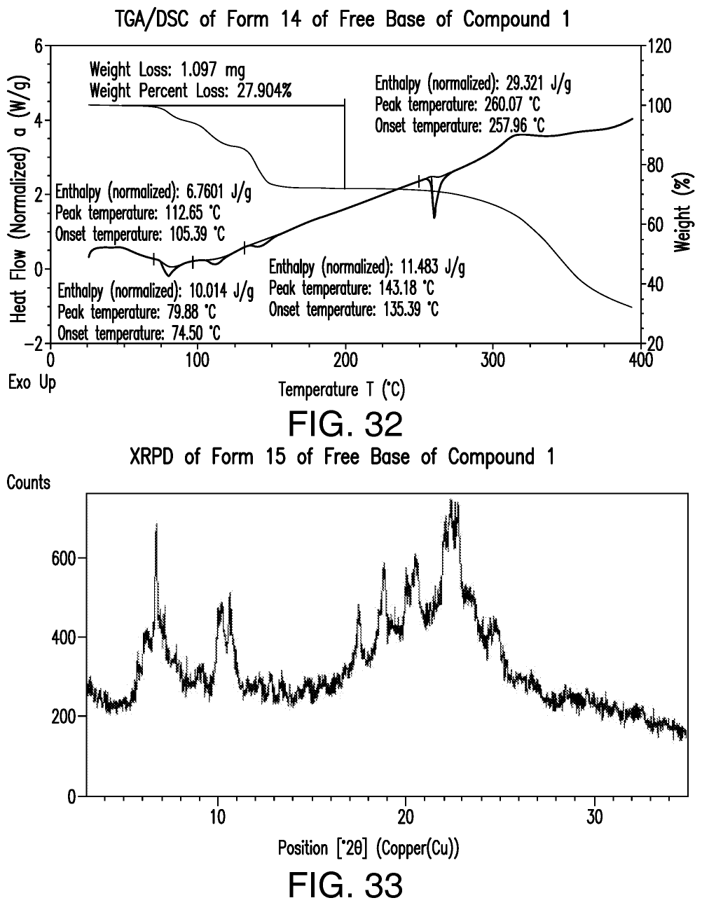

A representative overlay of TGA/DSC thermograms of Form 14 is provided in FIG. 32. In one embodiment, provided herein is a solid form comprising a free base of Compound 1, which exhibits, as characterized by DSC, a thermal event (endo) with an onset temperature of about 75° C. (e.g. ±2°). In one embodiment the thermal event has a peak temperature of about 80° C. (e.g. ±2°). In one embodiment, the solid form is characterized by a DSC thermogram that matches the DSC thermogram depicted in FIG. 32. In one embodiment, the DSC thermogram is as measured by DSC using a scanning rate of about 10° C./minute. In one embodiment, the Form 14 that provides FIG. 32 is a mixed solvate of cyclohexanone and t-butanol.

In one embodiment, provided herein is a solid form comprising a free base of Compound 1, which exhibits a weight loss of about 27.9% upon heating from about 75° C. to about 150° C. In one embodiment, the solid form is characterized by a TGA thermogram that matches the TGA thermogram depicted in FIG. 32. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1 which is a mixed solvate. In one embodiment, the solid form is a cyclohexanone and t-butanol mixed solvate. In one embodiment, provided herein is a solid form comprising a free base of Compound 1, wherein the molar ratio of Compound 1 to the solvent ranges from about 1:0.9 to about 1:2. In one embodiment, the molar ratio of Compound 1 to the solvent is about 1:1.4.

In one embodiment, provided herein is a solid form comprising Form 14 of a free base of Compound 1 and amorphous free base of Compound 1. In one embodiment, provided herein is a solid form comprising Form 14 of a free base Compound 1 and one or more other crystalline forms of a free base of Compound 1 provided herein.

All of the combinations of the above embodiments are encompassed by this application.

5.2.1.15 Form 15 of Compound 1

In one embodiment, provided herein is a Form 15 of Compound 1. A representative XRPD pattern of Form 15 of Compound 1 is provided in FIG. 33.

In one embodiment, provided herein is a solid form comprising a free base of Compound 1, characterized by 1, 2, 3, 4, 5, 6, 7, 8, 9, 10, 11, 12, 13, 14, 15, 16, 17, 18, or all of the XRPD peaks located at approximately the following positions (e.g., degrees 2θ±0.2) when measured using Cu Kα radiation: 5.7, 6.1, 6.7, 7.1, 7.7, 9.1, 10.0, 10.6, 12.8, 16.7, 17.5, 18.8, 19.3, 20.0, 20.5, 22.0, 22.8, 23.4, and 24.8° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form comprising a free base of Compound 1, characterized by an XRPD pattern, when measured using Cu Kα radiation, comprising at least three peaks selected from the group consisting of approximately (e.g., ±0.2°) 6.7, 7.1, 10.0, 10.6, 18.8, 20.0, 20.5, 22.0, and 22.8° 2θ. In one embodiment, the solid form is characterized by an XRPD pattern comprising at least four peaks selected from the group consisting of approximately (e.g., ±0.2°) 6.7, 7.1, 10.0, 10.6, 18.8, 20.0, 20.5, 22.0, and 22.8° 2θ. In one embodiment, the solid form is characterized by an XRPD pattern comprising at least five peaks selected from the group consisting of approximately (e.g., ±0.2°) 6.7, 7.1, 10.0, 10.6, 18.8, 20.0, 20.5, 22.0, and 22.8° 2θ.

In one embodiment, provided herein is a solid form comprising a free base of Compound 1, characterized by an XRPD pattern comprising peaks at approximately (e.g., ±0.2°) 6.7, 22.0, and 22.8° 2θ. In one embodiment, the solid form is characterized by an XRPD pattern further comprising peaks at approximately (e.g., ±0.2°) 18.8 and 20.5° 2θ. In one embodiment, the solid form is characterized by an XRPD pattern further comprising peaks at approximately (e.g., ±0.2°) 10.6 and 20.0° 2θ. In one embodiment, the XRPD pattern comprises peaks at approximately (e.g., ±0.2°) 6.7, 7.1, 10.0, 10.6, 18.8, 20.0, 20.5, 22.0, and 22.8° 2θ.

In one embodiment, provided herein is a solid form comprising a free base of Compound 1, characterized by an XRPD pattern that matches the XRPD pattern depicted in FIG. 33.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

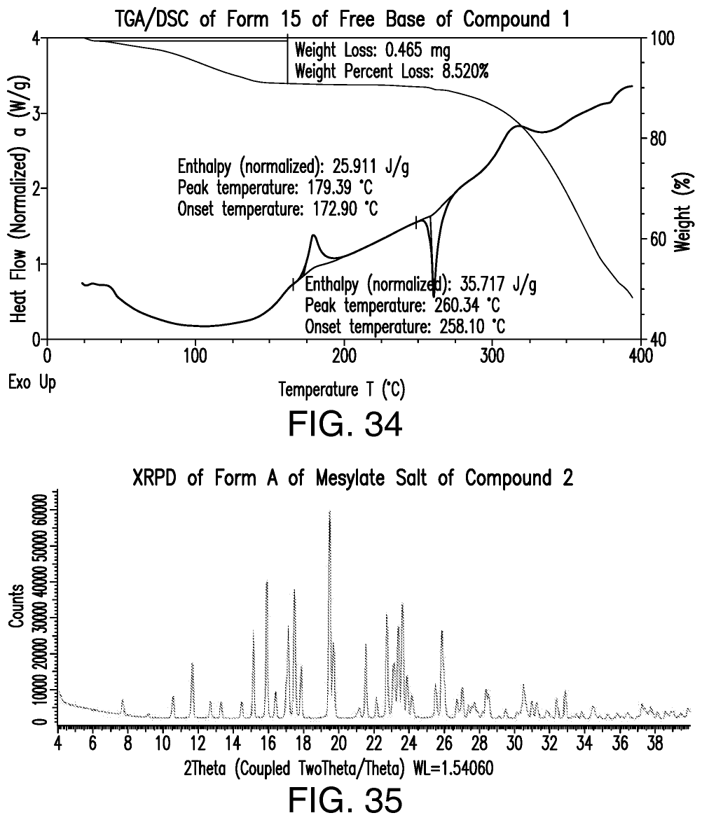

A representative overlay of TGA/DSC thermograms of Form 15 is provided in FIG. 34. In one embodiment, provided herein is a solid form comprising a free base of Compound 1, which exhibits, as characterized by DSC, a thermal event (exo) with an onset temperature of about 173° C. (e.g. ±2°). In one embodiment the thermal event has a peak temperature of about 179° C. (e.g. ±2°). In one embodiment, the solid form is characterized by a DSC thermogram that matches the DSC thermogram depicted in FIG. 34. In one embodiment, the DSC thermogram is as measured by DSC using a scanning rate of about 10° C./minute. In one embodiment, the Form 15 that provides FIG. 34 is an acetone solvate.

In one embodiment, provided herein is a solid form comprising a free base of Compound 1, which exhibits a weight loss of about 8.5% upon heating from about 25° C. to about 150° C. In one embodiment, the solid form is characterized by a TGA thermogram that matches the TGA thermogram depicted in FIG. 34. In one embodiment, the TGA thermogram is as measured using a heating rate of about 10° C./minute.

In some embodiments, provided herein is a solid form comprising a free base of Compound 1 which is a crystalline solvate of free base of Compound 1. In some embodiments, the solid form is substantially free of amorphous Compound 1. In some embodiments, the solid form is substantially free of other solid forms (e.g., crystalline forms) of Compound 1. In some embodiments, the solid form is substantially free of salts of Compound 1. In some embodiments, the solid form is provided as substantially pure. In some embodiments, the solid form is substantially chemically pure. In some embodiments, the solid form is substantially physically pure.

In one embodiment, provided herein is a solid form comprising a free base of Compound 1, wherein the molar ratio of Compound 1 to the solvent ranges from about 1:0.5 to about 1:1.2. In one embodiment, the molar ratio of Compound 1 to the solvent ranges from about 1:0.6 to about 1:1.1. In one embodiment, the solid form is an acetone solvate of free base of Compound 1. In one embodiment, the molar ratio of Compound 1 to acetone is about 1:0.7. In one embodiment, the molar ratio of Compound 1 to acetone is about 1:1.

In one embodiment, provided herein is a solid form comprising Form 15 of a free base of Compound 1 and amorphous free base of Compound 1. In one embodiment, provided herein is a solid form comprising Form 15 of a free base Compound 1 and one or more other crystalline forms of a free base of Compound 1 provided herein.

All of the combinations of the above embodiments are encompassed by this application.

5.2.2. Process of Preparing Solid Forms of Compound 1

As used herein and unless otherwise specified, all solvents ratios are meant for volume ratios.

In one embodiment, provided herein is a process for preparing Form 1 of a compound of Formula (I), comprising:
(i) exposing a composition comprising at least one non-Form 1 solid form of a compound of Formula (I) to one or more solvent for a period of time sufficient to convert at least about 50% of the total amount of the non-Form 1 solid form(s) into Form 1; and
(ii) recovering said Form 1.

In one embodiment, the non-Form 1 solid form is exposed to one solvent. In one embodiment, the non-Form 1 solid form is exposed to a mixture of two solvents. In one embodiment, the non-Form 1 solid form is exposed to one or more solvents. In one embodiment, the solvent is an organic solvent. In one embodiment, the solvent is 2-MeTHF, isopropyl acetate, heptane, or a mixture thereof. In one embodiment, the solvent is 2-MeTHF. In one embodiment, the solvent is isopropyl acetate. In one embodiment, the solvent is a mixture of 2-MeTHF and heptane. In one embodiment, the ratio of 2-MeTHF to heptane is from about 1:2 to about 1:6. In one embodiment, the solvent is a mixture of isopropyl acetate and heptane. In one embodiment, the ratio of isopropyl acetate to heptane is from about 1:2 to about 1:6. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the solvent is 2-MeTHF and the anti-solvent is heptane. In one embodiment, the solvent is isopropyl acetate and the anti-solvent is heptane. In one embodiment, the final ratio of solvent to anti-solvent is from about 1:2 to about 1:6. In one embodiment, the non-Form 1 solid form is exposed to the solvent and/or the anti-solvent at room temperature. In one embodiment, the non-Form 1 solid form is exposed to the solvent and/or the anti-solvent at a temperature above room temperature. In one embodiment, the non-Form 1 solid form is exposed to the solvent and/or the anti-solvent at a temperature from about 25° C. to about 60° C.

In one embodiment, the non-Form 1 solid form is an amorphous solid form of a compound of Formula (I). In one embodiment, the non-Form 1 solid form is any one of Form 2 to Form 15 of a compound of Formula (I). In one embodiment, the period of time sufficient to convert at least about 50% of the total amount of the non-Form 1 solid form into Form 1 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 1 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 1 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is 2-MeTHF. In one embodiment, the solvent is isopropyl acetate.

In one embodiment, Form 1 of a compound of Formula (I) is prepared by crystallization or recrystallization of a compound of Formula (I) from a solvent comprising a mixture of two solvents. In one embodiment, the mixture of two solvents is a mixture of 2-MeTHF and heptane. In one embodiment, the volume ratio of 2-MeTHF to heptane is from about 1:10 to about 1:2. In one embodiment, the mixture of two solvents is a mixture of isopropyl acetate and heptane. In one embodiment, the volume ratio of isopropyl acetate to heptane is from about 1:10 to about 1:2. In one embodiment, the volume ratio of isopropyl acetate to heptane is about 1:1.

In one embodiment, Form 1 of a compound of Formula (I) is prepared by evaporating a solution of the compound in 2-MeTHF. In one embodiment, the evaporation is conducted at about 20° C. In one embodiment, the evaporation is slow evaporation (e.g., for about 7 days).

In one embodiment, Form 1 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 2 of a compound of Formula (I), comprising:
(i) exposing a composition comprising at least one non-Form 2 solid form of a compound of Formula (I) to one or more solvent for a period of time sufficient to convert at least about 50% of the total amount of the non-Form 2 solid form(s) into Form 2; and
(ii) recovering said Form 2.

In one embodiment, the non-Form 2 solid form is exposed to one solvent. In one embodiment, the non-Form 2 solid form is exposed to a mixture of two solvents. In one embodiment, the non-Form 2 solid form is exposed to one or more solvents. In one embodiment, the solvent is an organic solvent. In one embodiment, the solvent is ethanol, acetonitrile, t-butyl methyl ether, isobutyl acetate, cyclopentyl methyl ether, isopropyl acetate, ethyl acetate, 1-butanol, 2-butanol, cyclohexane, THF, cyclopentyl methyl (CPME), diisopropyl ether (DIIPE), methyl ether Ketone (MEK), methylisobutyl Ketone (MIBK), 1-propanol, 2-propanol, t-amyl alcohol, n-butyl acetate, methanol, toluene, dichloride methane, ethyl tert-butyl ether (tBME), cyclopentyl methyl ether, methyl cyclohexane, acetone, 2-Methyl THF, 2-ethoxyethanol, anisole, DMSO, 1,4-dioxane, heptane, or a mixture thereof. In one embodiment, the solvent is ethanol. In one embodiment, the solvent is a mixture of ethanol and heptane. In one embodiment, the volume ratio of ethanol to heptane is from about 1:2 to about 1:15. In one embodiment, the volume ratio of ethanol to heptane is from about 1:6 to about 1:10. In one embodiment, the solvent is ethyl acetate. In one embodiment, the solvent is a mixture of ethyl acetate and heptane. In one embodiment, the volume ratio of ethyl acetate to heptane is from about 1:2 to about 1:15. In one embodiment, the volume ratio of ethyl acetate to heptane is from about 1:6 to about 1:10. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the final volume ratio of solvent to anti-solvent is from about 1:1 to about 1:15. In one embodiment, the final volume ratio of solvent to anti-solvent is from about 1:6 to about 1:10. In one embodiment, the non-Form 2 solid form is exposed to the solvent and/or the anti-solvent at room temperature. In one embodiment, the non-Form 2 solid form is exposed to the solvent and/or the anti-solvent at a temperature above room temperature. In one embodiment, the non-Form 2 solid form is exposed to the solvent and/or the anti-solvent at a temperature from about 25° C. to about 60° C. In one embodiment, the non-Form 2 solid form is exposed to the solvent and/or the anti-solvent at a temperature from about 35° C. to about 55° C.

In one embodiment, the non-Form 2 solid form is an amorphous solid form of a compound of Formula (I). In one embodiment, the non-Form 2 solid form is any one of Form 1 or Form 3 to Form 15 of a compound of Formula (I). In one embodiment, the period of time sufficient to convert at least about 50% of the total amount of the non-Form 2 solid form into Form 2 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 2 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 2 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is ethanol. In one embodiment, the solvent is ethyl acetate.

In one embodiment, Form 2 of a compound of Formula (I) is prepared by crystallization or recrystallization of a compound of Formula (I) from a solvent comprising a mixture of two solvents. In one embodiment, the mixture of two solvents is a mixture of ethanol and heptane. In one embodiment, the volume ratio of ethanol to heptane is from about 1:15 to about 1:2. In one embodiment, the mixture of two solvents is a mixture of ethyl acetate and heptane. In one embodiment, the volume ratio of ethyl acetate to heptane is from about 1:15 to about 1:2. In one embodiment, the volume ratio of ethyl acetate to heptane is about 1:8. In one embodiment, the weight ratio of EtOAc to heptane is from about 1:3 to about 1:10. In one embodiment, the weight ratio of EtOAc to heptane is about 1:6.2.

In one embodiment, Form 2 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 2 of a compound of Formula (I), comprising
(i) dissolving the compound of Formula (I) in a solvent;
(ii) adding an anti-solvent; and
(iii) recovering said Form 2.

In one embodiment, the solvent is ethanol. In one embodiment, the anti-solvent is heptane. In one embodiment, the solvent is ethanol and the anti-solvent is heptane.

In another embodiment, the solvent is ethyl acetate. In one embodiment, the anti-solvent is heptane. In one embodiment, the solvent is ethyl acetate and the anti-solvent is heptane.

In one embodiment, provided herein is a process for preparing Form 2 of a compound of Formula (I), comprising
(i) desolvating at least one non-Form 2 solid form of a compound of Formula (I) for a period of time sufficient to convert at least about 50% of the total amount of the non-Form 2 solid form(s) into Form 2; and
(ii) recovering said Form 2.

In one embodiment, the at least one non-Form 2 solid form is desolvated at a temperature above room temperature. In one embodiment, the temperature is from about 30 to about 60° C. In one embodiment, the temperature is from about 40-60° C. In one embodiment, the temperature is from about 50-60° C. In one embodiment, the temperature is from about 100 to about 200° C. In one embodiment, the at least one non-Form 2 solid form is desolvated under vacuum. In one embodiment, the at least one non-Form 2 solid form is an amorphous solid form of a compound of Formula (I). In one embodiment, the at least one non-Form 2 solid form is any one of Form 1 or Form 3 to Form 15 of a compound of Formula (I). In one embodiment, the at least one non-Form 2 solid form is Form 1 of a compound of Formula (I). In one embodiment, the at least one non-Form 2 solid form is Form 3 of a compound of Formula (I). In one embodiment, the period of time sufficient to convert at least about 50% of the total amount of the non-Form 2 solid form into Form 2 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

In one embodiment, Form 2 of a compound of Formula (I) is prepared by a process comprising: (i) concentrating a solution comprising Compound 1 in a mixture of EtOH and heptane (volume ratio of EtOH:heptane about 1:0.2 to about 1:0.7); (ii) adding heptane of about 3 times to about 15 times of solution volume (e.g. 6 to 8 times); and (iii) heating the solution from about 45 to about 55° C. In one embodiment, the process further comprises after step (iii): stirring the solution at about 10-15° C.

In one embodiment, Form 2 of a compound of Formula (I) is prepared by a process comprising: (i) concentrating a solution comprising a compound of Formula (I) in EtOAc; (ii) adding heptane to form a solution; and (iii) heating the solution from about 45 to about 55° C. In one embodiment, the concentration is conducted at about below 50° C. In one embodiment, the process further comprises after step (ii): adding a seed amount of Form 2. In certain embodiments, the seed amount is about 0.5 wt % to about 15 wt % of the compound of Formula (I). In certain embodiments, the seed amount is about 1 wt % to about 10 wt % of the compound of Formula (I). In certain embodiments, the seed amount is about 5 wt % of the compound of Formula (I). In certain embodiments, the seed amount is about 4 wt % of the compound of Formula (I). In certain embodiments, the seed amount is about 3 wt % of the compound of Formula (I). In certain embodiments, the seed amount is about 2 wt % of the compound of Formula (I). In certain embodiments, the seed amount is about 1 wt % of the compound of Formula (I).

In one embodiment, provided herein is a process for preparing Form 3 of a compound of Formula (I), comprising:
(i) exposing a composition comprising at least one non-Form 3 solid form of a compound of Formula (I) to one or more solvent for a period of time sufficient to convert at least about 50% of the total amount of the non-Form 3 solid form(s) into Form 3; and
(ii) recovering said Form 3.

In one embodiment, the non-Form 3 solid form is exposed to one solvent. In one embodiment, the non-Form 3 solid form is exposed to a mixture of two solvents. In one embodiment, the non-Form 3 solid form is exposed to one or more solvents. In one embodiment, the solvent is an organic solvent. In one embodiment, the solvent is 2-MeTHF. In one embodiment, the non-Form 3 solid form is exposed to the solvent at room temperature. In one embodiment, the non-Form 3 solid form is exposed to the solvent at a temperature above room temperature. In one embodiment, the non-Form 3 solid form is exposed to the solvent at a temperature from about 25° C. to about 60° C.

In one embodiment, the non-Form 3 solid form is an amorphous solid form of a compound of Formula (I). In one embodiment, the non-Form 3 solid form is any one of Form 1 to Form 2 or Form 4 to Form 15 of a compound of Formula (I). In one embodiment, the period of time sufficient to convert at least about 50% of the total amount of the non-Form 3 solid form into Form 3 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 3 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 3 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is 2-MeTHF.

In one embodiment, Form 3 of a compound of Formula (I) is prepared by a process comprising evaporating a solution of the compound in 2-MeTHF. In one embodiment, the evaporation is conducted at about 20° C. In one embodiment, the evaporation is conducted at about 50° C. In one embodiment, the evaporation is slow evaporation (e.g., for about 3 days or about 7 days).

In one embodiment, Form 3 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 4 of a compound of Formula (I), comprising:
(i) exposing a composition comprising at least one non-Form 4 solid form of a compound of Formula (I) to one or more solvent for a period of time sufficient to convert at least about 50% of the total amount of the non-Form 4 solid form(s) into Form 4; and
(ii) recovering said Form 4.

In one embodiment, the non-Form 4 solid form is exposed to one solvent. In one embodiment, the non-Form 4 solid form is exposed to a mixture of two solvents. In one embodiment, the non-Form 4 solid form is exposed to one or more solvents. In one embodiment, the solvent is an organic solvent. In one embodiment, the solvent is 1,4-dioxane, water, heptane, or a mixture thereof. In one embodiment, the solvent is 1,4-dioxane. In one embodiment, the solvent is a mixture of 1,4-dioxane and heptane. In one embodiment, the volume ratio of 1,4-dioxane to heptane is from about 1:2 to about 1:6. In one embodiment, the solvent is a mixture of 1,4-dioxane and water. In one embodiment, the volume ratio of 1,4-dioxane to water is from about 1:2 to about 10:1. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the anti-solvent is water. In one embodiment, the solvent is 1,4-dioxane and the anti-solvent is heptane. In one embodiment, the solvent is 1,4-dioxane and the anti-solvent is water. In one embodiment, the final ratio of solvent to anti-solvent is about 1:2. In one embodiment, the final ratio of solvent to anti-solvent is from about 1:1 to about 10:1. In one embodiment, the final ratio of solvent to anti-solvent is about 8:1. In one embodiment, the non-Form 4 solid form is exposed to the solvent and/or the anti-solvent at room temperature. In one embodiment, the non-Form 4 solid form is exposed to the solvent and/or the anti-solvent at a temperature above room temperature. In one embodiment, the non-Form 4 solid form is exposed to the solvent and/or the anti-solvent at a temperature from about 25° C. to about 60° C.

In one embodiment, the non-Form 4 solid form is an amorphous solid form of a compound of Formula (I). In one embodiment, the non-Form 4 solid form is any one of Form 1 to Form 3 or Form 5 to Form 15 of a compound of Formula (I). In one embodiment, the period of time sufficient to convert at least about 50% of the total amount of the non-Form 4 solid form into Form 4 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 4 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 4 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is 1,4-dioxane.

In one embodiment, Form 4 of a compound of Formula (I) is prepared by crystallization or recrystallization of a compound of Formula (I) from a solvent comprising a mixture of two solvents. In one embodiment, the mixture of two solvents is a mixture of 1,4-dioxane and heptane. In one embodiment, the volume ratio of 1,4-dioxane to heptane is from about 1:10 to about 1:1. In one embodiment, the mixture of two solvents is a mixture of 1,4-dioxane and water. In one embodiment, the volume ratio of 1,4-dioxane to water is from about 1:10 to about 10:1. In one embodiment, the volume ratio of 1,4-dioxane to water is about 1:1. In one embodiment, the volume ratio of 1,4-dioxane to water is about 8:1.

In one embodiment, Form 4 of a compound of Formula (I) is prepared by a process comprising slurrying and/or agitating the compound in a mixture of 1,4-dioxane and water. In one embodiment, the mixture has a volume ratio of 1,4-dioxane to water of about 1:2. In one embodiment, the mixture has a volume ratio of 1,4-dioxane to water of about 8:1. In one embodiment, the slurrying and/or agitating is conducted at about 20° C. In one embodiment, the slurring and/or agitating is conducted for at least 12 hours.

In one embodiment, Form 4 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 5 of a compound of Formula (I), comprising:
(i) exposing a composition comprising at least one non-Form 5 solid form of a compound of Formula (I) to one or more solvent for a period of time sufficient to convert at least about 50% of the total amount of the non-Form 5 solid form(s) into Form 5; and
(ii) recovering said Form 5.

In one embodiment, the non-Form 5 solid form is exposed to one solvent. In one embodiment, the non-Form 5 solid form is exposed to a mixture of two solvents. In one embodiment, the non-Form 5 solid form is exposed to one or more solvents. In one embodiment, the solvent is an organic solvent. In one embodiment, the solvent is 2-propanol, t-butanol, THF, acetone, heptane, or a mixture thereof. In one embodiment, the solvent is 2-propanol. In one embodiment, the solvent is t-butanol. In one embodiment, the solvent is THF. In one embodiment, the solvent is acetone. In one embodiment, the solvent is a mixture of 2-propanol and t-butanol. In one embodiment, the solvent is a mixture of acetone and t-butanol. In one embodiment, the solvent is a mixture of THF and t-butanol. In one embodiment, the non-Form 5 solid form is exposed to the solvent at room temperature. In one embodiment, the non-Form 5 solid form is exposed to the solvent at a temperature above room temperature. In one embodiment, the non-Form 5 solid form is exposed to the solvent at a temperature from about 25° C. to about 60° C.

In one embodiment, the non-Form 5 solid form is an amorphous solid form of a compound of Formula (I). In one embodiment, the non-Form 5 solid form is any one of Form 1 to Form 4 or Form 6 to Form 15 of a compound of Formula (I). In one embodiment, the period of time sufficient to convert at least about 50% of the total amount of the non-Form 5 solid form into Form 5 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 5 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 5 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is 2-propanol, t-butanol, THF, acetone, heptane, or a mixture thereof. In one embodiment, the solvent is 2-propanol. In one embodiment, the solvent is t-butanol. In one embodiment, the solvent is THF. In one embodiment, the solvent is acetone.

In one embodiment, Form 5 of a compound of Formula (I) is prepared by crystallization or recrystallization of a compound of Formula (I) from a solvent comprising a mixture of two solvents. In one embodiment, the solvent is a mixture of 2-propanol and t-butanol. In one embodiment, the solvent is a mixture of acetone and t-butanol. In one embodiment, the solvent is a mixture of THF and t-butanol.

In one embodiment, Form 5 of a compound of Formula (I) is prepared by a process comprising milling the compound in t-butanol (e.g., with steel beads). In one embodiment, the compound is milled with the beads at 6000 RPM. In one embodiment, the milling is conducted in cycles, for example, 90 second cycles with a pause of 10 seconds per cycle. In one embodiment, 40 cycles are conducted.

In one embodiment, Form 5 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 6 of a compound of Formula (I), comprising:
(i) exposing a composition comprising at least one non-Form 6 solid form of a compound of Formula (I) to one or more solvent for a period of time sufficient to convert at least about 50% of the total amount of the non-Form 6 solid form(s) into Form 6; and
(ii) recovering said Form 6.

In one embodiment, the non-Form 6 solid form is exposed to one solvent. In one embodiment, the non-Form 6 solid form is exposed to a mixture of two solvents. In one embodiment, the non-Form 6 solid form is exposed to one or more solvents. In one embodiment, the solvent is an organic solvent. In one embodiment, the solvent is acetone. In one embodiment, the non-Form 6 solid form is exposed to the solvent at room temperature. In one embodiment, the non-Form 6 solid form is exposed to the solvent at a temperature above room temperature. In one embodiment, the non-Form 6 solid form is exposed to the solvent at a temperature from about 25° C. to about 60° C.

In one embodiment, the non-Form 6 solid form is an amorphous solid form of a compound of Formula (I). In one embodiment, the non-Form 6 solid form is any one of Form 1 to Form 5 or Form 7 to Form 15 of a compound of Formula (I). In one embodiment, the period of time sufficient to convert at least about 50% of the total amount of the non-Form 6 solid form into Form 6 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 6 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 6 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is acetone.

In one embodiment, Form 6 of a compound of Formula (I) is prepared by a process comprising milling the compound in acetone (e.g., with steel beads). In one embodiment, the compound is milled with the beads at 6000 RPM. In one embodiment, the milling is conducted in cycles, for example, 90 second cycles with a pause of 10 seconds per cycle. In one embodiment, 40 cycles are conducted.

In one embodiment, Form 6 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 7 of a compound of Formula (I), comprising:
(i) exposing a composition comprising at least one non-Form 7 solid form of a compound of Formula (I) to one or more solvent for a period of time sufficient to convert at least about 50% of the total amount of the non-Form 7 solid form(s) into Form 7; and
(ii) recovering said Form 7.

In one embodiment, the non-Form 7 solid form is exposed to one solvent. In one embodiment, the non-Form 7 solid form is exposed to a mixture of two solvents. In one embodiment, the non-Form 7 solid form is exposed to one or more solvents. In one embodiment, the solvent is an organic solvent. In one embodiment, the solvent is MIBK, heptane, or a mixture thereof. In one embodiment, the solvent is MIBK. In one embodiment, the solvent is a mixture of MIBK and heptane. In one embodiment, the ratio of MIBK to heptane is from about 1:1 to about 1:6. In one embodiment, the ratio of MIBK to heptane is about 1:2. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the solvent is MIBK and the anti-solvent is heptane. In one embodiment, the final ratio of solvent to anti-solvent is from about 1:1 to about 1:6. In one embodiment, the final ratio of solvent to anti-solvent is about 1:2. In one embodiment, the non-Form 7 solid form is exposed to the solvent and/or the anti-solvent at room temperature. In one embodiment, the non-Form 7 solid form is exposed to the solvent and/or the anti-solvent at a temperature above room temperature. In one embodiment, the non-Form 7 solid form is exposed to the solvent and/or the anti-solvent at a temperature from about 25° C. to about 60° C.

In one embodiment, the non-Form 7 solid form is an amorphous solid form of a compound of Formula (I). In one embodiment, the non-Form 7 solid form is any one of Form 1 to Form 6 or Form 8 to Form 15 of a compound of Formula (I). In one embodiment, the period of time sufficient to convert at least about 50% of the total amount of the non-Form 7 solid form into Form 7 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 7 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 7 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is MIBK.

In one embodiment, Form 7 of a compound of Formula (I) is prepared by crystallization or recrystallization of a compound of Formula (I) from a solvent comprising a mixture of two solvents. In one embodiment, the mixture of two solvents is a mixture of MIBK and heptane. In one embodiment, the volume ratio of MIBK to heptane is from about 1:10 to about 1:1. In one embodiment, the volume ratio of MIBK to heptane is about 1:2.

In one embodiment, Form 7 of a compound of Formula (I) is prepared by a process comprising slurrying and/or agitating the compound in a mixture of MIBK and heptane. In one embodiment, the mixture has a volume ratio of MIBK to heptane of about 1:2. In one embodiment, the slurrying and/or agitating is conducted at about 20° C. In one embodiment, the slurring and/or agitating is conducted for at least 12 hours.

In one embodiment, Form 7 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 8 of a compound of Formula (I), comprising:
(i) exposing a composition comprising at least one non-Form 8 solid form of a compound of Formula (I) to one or more solvent for a period of time sufficient to convert at least about 50% of the total amount of the non-Form 8 solid form(s) into Form 8; and
(ii) recovering said Form 8.

In one embodiment, the non-Form 8 solid form is exposed to one solvent. In one embodiment, the non-Form 8 solid form is exposed to a mixture of two solvents. In one embodiment, the non-Form 8 solid form is exposed to one or more solvents. In one embodiment, the solvent is an organic solvent. In one embodiment, the solvent is THF, heptane, or a mixture thereof. In one embodiment, the solvent is THF. In one embodiment, the solvent is a mixture of THF and heptane. In one embodiment, the volume ratio of THF to heptane is from about 1:1 to about 1:6. In one embodiment, the volume ratio of THF to heptane is about 1:1. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the solvent is THF and the anti-solvent is heptane. In one embodiment, the final volume ratio of solvent to anti-solvent is from about 1:1 to about 1:6. In one embodiment, the final volume ratio of solvent to anti-solvent is about 1:1. In one embodiment, the non-Form 8 solid form is exposed to the solvent and/or the anti-solvent at room temperature. In one embodiment, the non-Form 8 solid form is exposed to the solvent and/or the anti-solvent at a temperature above room temperature. In one embodiment, the non-Form 8 solid form is exposed to the solvent and/or the anti-solvent at a temperature from about 25° C. to about 60° C.

In one embodiment, the non-Form 8 solid form is an amorphous solid form of a compound of Formula (I). In one embodiment, the non-Form 8 solid form is any one of Form 1 to Form 7 or Form 9 to Form 15 of a compound of Formula (I). In one embodiment, the period of time sufficient to convert at least about 50% of the total amount of the non-Form 8 solid form into Form 8 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 8 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 8 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is THF.

In one embodiment, Form 8 of a compound of Formula (I) is prepared by crystallization or recrystallization of a compound of Formula (I) from a solvent comprising a mixture of two solvents. In one embodiment, the mixture of two solvents is a mixture of THF and heptane. In one embodiment, the volume ratio of THF to heptane is from about 1:10 to about 1:1. In one embodiment, the volume ratio of THF to heptane is about 1:1.

In one embodiment, Form 8 of a compound of Formula (I) is prepared by a process comprising slurrying and/or agitating the compound in a mixture of THF and heptane. In one embodiment, the mixture has a volume ratio of THF to heptane of about 1:2. In one embodiment, the slurrying and/or agitating is conducted at about 20° C. In one embodiment, the slurring and/or agitating is conducted for at least 24 hours.

In one embodiment, Form 8 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 9 of a compound of Formula (I), comprising:
(i) exposing a composition comprising at least one non-Form 9 solid form of a compound of Formula (I) to one or more solvent for a period of time sufficient to convert at least about 50% of the total amount of the non-Form 9 solid form(s) into Form 9; and
(ii) recovering said Form 9.

In one embodiment, the non-Form 9 solid form is exposed to one solvent. In one embodiment, the non-Form 9 solid form is exposed to a mixture of two solvents. In one embodiment, the non-Form 9 solid form is exposed to one or more solvents. In one embodiment, the solvent is an organic solvent. In one embodiment, the solvent is cyclohexanone, heptane, or a mixture thereof. In one embodiment, the solvent is cyclohexanone. In one embodiment, the solvent is cyclohexanone. In one embodiment, the ratio of cyclohexanone to heptane is from about 1:1 to about 1:6. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the solvent is cyclohexanone and the anti-solvent is heptane. In one embodiment, the final ratio of solvent to anti-solvent is from about 1:1 to about 1:6. In one embodiment, the non-Form 9 solid form is exposed to the solvent and/or the anti-solvent at room temperature. In one embodiment, the non-Form 9 solid form is exposed to the solvent and/or the anti-solvent at a temperature above room temperature. In one embodiment, the non-Form 9 solid form is exposed to the solvent and/or the anti-solvent at a temperature from about 25° C. to about 60° C.

In one embodiment, the non-Form 9 solid form is an amorphous solid form of a compound of Formula (I). In one embodiment, the non-Form 9 solid form is any one of Form 1 to Form 8 or Form 10 to Form 15 of a compound of Formula (I). In one embodiment, the period of time sufficient to convert at least about 50% of the total amount of the non-Form 9 solid form into Form 9 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 9 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 9 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is cyclohexanone.

In one embodiment, Form 9 of a compound of Formula (I) is prepared by crystallization or recrystallization of a compound of Formula (I) from a solvent comprising a mixture of two solvents. In one embodiment, the mixture of two solvents is a mixture of cyclohexanone and heptane. In one embodiment, the volume ratio of cyclohexanone to heptane is from about 1:10 to about 1:1.

In one embodiment, Form 9 of a compound of Formula (I) is prepared by evaporating a solution of the compound in cyclohexanone. In one embodiment, the evaporation is conducted at about 20° C. In one embodiment, the evaporation is slow evaporation (e.g., for about 7 days).

In one embodiment, Form 9 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 10 of a compound of Formula (I), comprising:
(i) exposing a composition comprising at least one non-Form 10 solid form of a compound of Formula (I) to one or more solvent for a period of time sufficient to convert at least about 50% of the total amount of the non-Form 10 solid form(s) into Form 10; and
(ii) recovering said Form 10.

In one embodiment, the non-Form 10 solid form is exposed to one solvent. In one embodiment, the non-Form 10 solid form is exposed to a mixture of two solvents. In one embodiment, the non-Form 10 solid form is exposed to one or more solvents. In one embodiment, the solvent is an organic solvent. In one embodiment, the solvent is MIBK. In one embodiment, the non-Form 10 solid form is exposed to the solvent at room temperature. In one embodiment, the non-Form 10 solid form is exposed to the solvent at a temperature above room temperature. In one embodiment, the non-Form 10 solid form is exposed to the solvent at a temperature from about 25° C. to about 60° C.

In one embodiment, the non-Form 10 solid form is an amorphous solid form of a compound of Formula (I). In one embodiment, the non-Form 10 solid form is any one of Form 1 to Form 9 or Form 11 to Form 15 of a compound of Formula (I). In one embodiment, the period of time sufficient to convert at least about 50% of the total amount of the non-Form 10 solid form into Form 10 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 10 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 10 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is MIBK. In one embodiment, the solvent mixture is MIBK and water. In one embodiment, the solvent mixture is MIBK and water with the volume ratio of about 1:3 to 3:1. In one embodiment, the solvent mixture is MIBK and water with the volume ratio of about 1:1.

In one embodiment, Form 10 of a compound of Formula (I) is prepared by evaporating a solution of the compound in MIBK. In one embodiment, the evaporation is conducted at about 20° C. In one embodiment, the evaporation is slow evaporation (e.g., for about 7 days).

In one embodiment, Form 10 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 11 of a compound of Formula (I), comprising:
(i) exposing a composition comprising at least one non-Form 11 solid form of a compound of Formula (I) to one or more solvent for a period of time sufficient to convert at least about 50% of the total amount of the non-Form 11 solid form(s) into Form 11; and
(ii) recovering said Form 11.

In one embodiment, the non-Form 11 solid form is exposed to one solvent. In one embodiment, the non-Form 11 solid form is exposed to a mixture of two solvents. In one embodiment, the non-Form 11 solid form is exposed to one or more solvents. In one embodiment, the solvent is an organic solvent. In one embodiment, the solvent is MEK, heptane, or a mixture thereof. In one embodiment, the solvent is MEK. In one embodiment, the solvent is a mixture of MEK and heptane. In one embodiment, the volume ratio of MEK to heptane is from about 1:1 to about 1:6. In one embodiment, the volume ratio of MEK to heptane is about 1:1. In one embodiment, the solvent is a mixture of MEK and water. In one embodiment, the solvent is a mixture of MEK and water with a volume ratio of about 10:1 to 1:1. In one embodiment, the solvent is a mixture of MEK and water with a volume ratio of about 5:1. In one embodiment, an anti-solvent is added to the solvent. In one embodiment, the anti-solvent is a non-polar organic solvent. In one embodiment, the non-polar organic solvent is a hydrocarbon solvent. In one embodiment, the anti-solvent is heptane. In one embodiment, the solvent is MEK and the anti-solvent is heptane. In one embodiment, the final volume ratio of solvent to anti-solvent is from about 1:1 to about 1:6. In one embodiment, the non-Form 11 solid form is exposed to the solvent and/or the anti-solvent at room temperature. In one embodiment, the non-Form 11 solid form is exposed to the solvent and/or the anti-solvent at a temperature above room temperature. In one embodiment, the non-Form 11 solid form is exposed to the solvent and/or the anti-solvent at a temperature from about 25° C. to about 60° C.

In one embodiment, the non-Form 11 solid form is an amorphous solid form of a compound of Formula (I). In one embodiment, the non-Form 11 solid form is any one of Form 1 to Form 10 or Form 12 to Form 15 of a compound of Formula (I). In one embodiment, the period of time sufficient to convert at least about 50% of the total amount of the non-Form 11 solid form into Form 11 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 11 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 11 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is MEK.

In one embodiment, Form 11 of a compound of Formula (I) is prepared by crystallization or recrystallization of a compound of Formula (I) from a solvent comprising a mixture of two solvents. In one embodiment, the mixture of two solvents is a mixture of MEK and heptane. In one embodiment, the volume ratio of MEK to heptane is from about 1:10 to about 1:1.

In one embodiment, Form 11 of a compound of Formula (I) is prepared by a process comprising slurrying and/or agitating the compound in a mixture of MEK and heptane. In one embodiment, the mixture has a volume ratio of MEK to heptane of about 1:1. In one embodiment, the slurrying and/or agitating is conducted at about 20° C. In one embodiment, the slurring and/or agitating is conducted for at least 12 hours.

In one embodiment, Form 11 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 12 of a compound of Formula (I), comprising:
(i) exposing a composition comprising at least one non-Form 12 solid form of a compound of Formula (I) to one or more solvent for a period of time sufficient to convert at least about 50% of the total amount of the non-Form 12 solid form(s) into Form 12; and
(ii) recovering said Form 12.

In one embodiment, the non-Form 12 solid form is exposed to one solvent. In one embodiment, the non-Form 12 solid form is exposed to a mixture of two solvents. In one embodiment, the non-Form 12 solid form is exposed to one or more solvents. In one embodiment, the solvent is an organic solvent. In one embodiment, the solvent is methylcyclohexane. In one embodiment, the non-Form 12 solid form is exposed to the solvent at room temperature. In one embodiment, the non-Form 12 solid form is exposed to the solvent at a temperature above room temperature. In one embodiment, the non-Form 12 solid form is exposed to the solvent at a temperature from about 25° C. to about 60° C.

In one embodiment, the non-Form 12 solid form is an amorphous solid form of a compound of Formula (I). In one embodiment, the non-Form 12 solid form is any one of Form 1 to Form 11 or Form 13 to Form 15 of a compound of Formula (I). In one embodiment, the period of time sufficient to convert at least about 50% of the total amount of the non-Form 12 solid form into Form 12 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 12 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 12 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is methylcyclohexane.

In one embodiment, Form 12 of a compound of Formula (I) is prepared by a process comprising slurrying and/or agitating the compound in methylcyclohexane. In one embodiment, the slurrying and/or agitating is conducted at about 20° C. In one embodiment, the slurring and/or agitating is conducted for at least 24 hours.

In one embodiment, Form 12 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 13 of a compound of Formula (I), comprising:
(i) exposing a composition comprising at least one non-Form 13 solid form of a compound of Formula (I) to one or more solvent for a period of time sufficient to convert at least about 50% of the total amount of the non-Form 13 solid form(s) into Form 13; and
(ii) recovering said Form 13.

In one embodiment, the non-Form 13 solid form is exposed to one solvent. In one embodiment, the non-Form 13 solid form is exposed to a mixture of two solvents. In one embodiment, the non-Form 13 solid form is exposed to one or more solvents. In one embodiment, the solvent is an organic solvent. In one embodiment, the solvent is cyclohexane. In one embodiment, the non-Form 13 solid form is exposed to the solvent at room temperature. In one embodiment, the non-Form 13 solid form is exposed to the solvent at a temperature above room temperature. In one embodiment, the non-Form 13 solid form is exposed to the solvent at a temperature from about 25° C. to about 60° C.

In one embodiment, the non-Form 13 solid form is an amorphous solid form of a compound of Formula (I). In one embodiment, the non-Form 13 solid form is any one of Form 1 to Form 12 or Form 14 to Form 15 of a compound of Formula (I). In one embodiment, the period of time sufficient to convert at least about 50% of the total amount of the non-Form 13 solid form into Form 13 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 13 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 13 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is cyclohexane.

In one embodiment, Form 13 of a compound of Formula (I) is prepared by a process comprising slurrying and/or agitating the compound in cyclohexane. In one embodiment, the slurrying and/or agitating is conducted at about 20° C. In one embodiment, the slurring and/or agitating is conducted for at least 24 hours.

In one embodiment, Form 13 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 14 of a compound of Formula (I), comprising:
(i) exposing a composition comprising at least one non-Form 14 solid form of a compound of Formula (I) to one or more solvent for a period of time sufficient to convert at least about 50% of the total amount of the non-Form 14 solid form(s) into Form 14; and
(ii) recovering said Form 14.

In one embodiment, the non-Form 14 solid form is exposed to one solvent. In one embodiment, the non-Form 14 solid form is exposed to a mixture of two solvents. In one embodiment, the non-Form 14 solid form is exposed to one or more solvents. In one embodiment, the solvent is an organic solvent. In one embodiment, the solvent is cyclohexanone, t-butanol, or a mixture thereof. In one embodiment, the solvent is cyclohexanone. In one embodiment, the non-Form 14 solid form is exposed to the solvent at room temperature. In one embodiment, the non-Form 14 solid form is exposed to the solvent at a temperature above room temperature. In one embodiment, the non-Form 14 solid form is exposed to the solvent at a temperature from about 25° C. to about 60° C.

In one embodiment, the non-Form 14 solid form is an amorphous solid form of a compound of Formula (I). In one embodiment, the non-Form 14 solid form is any one of Form 1 to Form 13 or Form 15 of a compound of Formula (I). In one embodiment, the period of time sufficient to convert at least about 50% of the total amount of the non-Form 14 solid form into Form 14 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 14 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 14 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is cyclohexanone, t-butanol, or a mixture thereof.

In one embodiment, Form 14 of a compound of Formula (I) is prepared by a process comprising milling the compound in cyclohexanone (e.g., with steel beads). In one embodiment, the compound is milled with the beads at 6000 RPM. In one embodiment, the milling is conducted in cycles, for example, 90 second cycles with a pause of 10 seconds per cycle. In one embodiment, 40 cycles are conducted.

In one embodiment, Form 14 of a compound of Formula (I) is prepared by crystallization or recrystallization as described in the experiments provided herein, including but not limited to evaporation, anti-solvent addition, slow cooling, or crash cooling.

In one embodiment, provided herein is a process for preparing Form 15 of a compound of Formula (I), comprising:
(i) exposing a composition comprising at least one non-Form 15 solid form of a compound of Formula (I) to one or more solvent for a period of time sufficient to convert at least about 50% of the total amount of the non-Form 15 solid form(s) into Form 15; and
(ii) recovering said Form 15.

In one embodiment, the non-Form 15 solid form is exposed to one solvent. In one embodiment, the non-Form 15 solid form is exposed to a mixture of two solvents. In one embodiment, the non-Form 15 solid form is exposed to one or more solvents. In one embodiment, the solvent is an organic solvent. In one embodiment, the solvent is acetone. In one embodiment, the non-Form 15 solid form is exposed to the solvent at room temperature. In one embodiment, the non-Form 15 solid form is exposed to the solvent at a temperature above room temperature. In one embodiment, the non-Form 15 solid form is exposed to the solvent at a temperature from about 25° C. to about 60° C.

In one embodiment, the non-Form 15 solid form is an amorphous solid form of a compound of Formula (I). In one embodiment, the non-Form 15 solid form is any one of Form 1 to Form 14 of a compound of Formula (I). In one embodiment, the period of time sufficient to convert at least about 50% of the total amount of the non-Form 15 solid form into Form 15 is about 1 hr, about 2 hr, about 5 hr, about 10 hr, about 12 hr, about 20 hr, about 24 hr, about 30 hr, about 40 hr, about 48 hr, about 72 hr, about 97 hours, about 121 hours, or greater than 121 hours.

Form 15 of a compound of Formula (I) may be prepared by exposing a composition comprising a compound of Formula (I) to one or more solvent as described in the experiments provided herein, including but not limited to evaporation, anti-solvent addition, slow cooling, crash cooling, temperature cycling, slurrying, bead milling, or solvent drop grinding.

In one embodiment, Form 15 of a compound of Formula (I) is prepared by crystallization or recrystallization of a compound of Formula (I), or a stereoisomer, or a mixture of stereoisomers thereof, or a pharmaceutically acceptable salt thereof, from one or more solvents. In one embodiment, the solvent is an organic solvent. In one embodiment, the solvent is acetone.

In one embodiment, Form 15 of a compound of Formula (I) is prepared by a process comprising slurrying and/or agitating Form 2 of the compound in acetone. In one embodiment, the slurrying and/or agitating is conducted at about 20° C. In one embodiment, the slurring and/or agitating is conducted for at least 36 hours.

In one embodiment, Form 15 of a compound of Formula (I) is prepared by crystallization or recrystallization as described in the experiments provided herein, including but not limited to evaporation, anti-solvent addition, slow cooling, or crash cooling.

5.2.3. Salts of a Compound of Formula (I)

In certain embodiments, provided herein is a solid form comprising a salt of a compound of Formula (I):

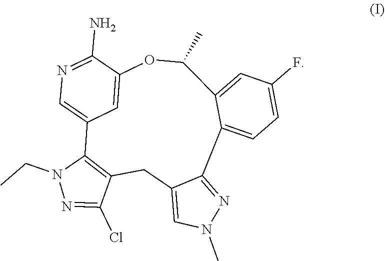 [SMILES: CCn1nc(Cl)c2c1-c1cnc(N)c(c1)O[C@H](C)c1cc(F)ccc1-c1nn(C)cc1C2]

In one embodiment, provided herein is a hydrochloric acid salt (hydrochloride salt), methane sulfonic acid salt (mesylate salt), benzene sulfonic acid salt (besylate salt), maleic acid salt (maleate salt), phosphoric acid salt (phosphate salt), citric acid salt (citrate salt), L-tartaric acid salt (L-tartarate salt), fumaric acid salt (fumarate salt), toluenesulfonic acid (tosylate), or salicylic acid salt (salicylate salt) of Compound 1. In one embodiment, provided herein is a hydrochloric acid salt (hydrochloride salt), methane sulfonic acid salt (mesylate salt), maleic acid salt (maleate salt), phosphoric acid salt (phosphate salt), citric acid salt (citrate salt), L-tartaric acid salt (L-tartarate salt), fumaric acid salt (fumarate salt), toluenesulfonic acid (tosylate), or salicylic acid salt (salicylate salt) of Compound 1.

The molar ratio of Compound 1 to a counterion of the salt of Compound 1 may be about 1:1, about 1:2, about 1:3, or about 1:4. In one embodiment, the molar ratio of Compound 1 to counterion is about 1:1. In one embodiment, the molar ratio of Compound 1 to a counterion is about 1:2. In one embodiment, the molar ratio of Compound 1 to a counterion is about 1:3. In one embodiment, the molar ratio of Compound 1 to a counterion is about 1:4.

In one embodiment, the counterion is chloride, mesylate, besylate, maleate, phosphate, citrate, L-tartarate, fumarate, tosylate, or salicylate. In one embodiment, the salt of Compound 1 is a hydrochloric acid salt (hydrochloride salt) of Compound 1. In one embodiment, the salt of Compound 1 is a methane sulfonic acid salt (mesylate salt) of Compound 1. In one embodiment, the salt of Compound 1 is a benzene sulfonic acid salt (besylate salt) of Compound 1. In one embodiment, the salt of Compound 1 is a maleic acid salt (maleate salt) of Compound 1. In one embodiment, the salt of Compound 1 is a phosphoric acid salt (phosphate salt) of Compound 1. In one embodiment, the salt of Compound 1 is a citric acid salt (citrate salt) of Compound 1. In one embodiment, the salt of Compound 1 is a L-tartaric acid salt (L-tartarate salt). In one embodiment, the salt of Compound 1 is a fumaric acid salt (fumarate salt). In one embodiment, the salt of Compound 1 is a toluenesulfonic acid (tosylate). In one embodiment, the salt of Compound 1 is a salicylic acid salt (salicylate salt).

In one embodiment, provided herein a solid form comprising a salt of Compound 1. In one embodiment, the salt of Compound 1 provided herein is amorphous.

5.2.4. Solid Forms of Salts of Compound of Formula (I)

Provided herein are solid forms comprising a salt of a compound of Formula (I):

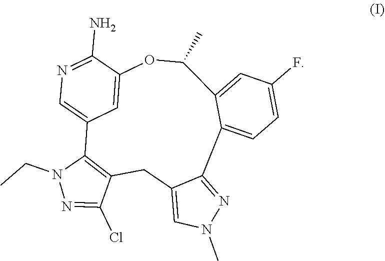 [SMILES: CCn1nc(Cl)c2c1-c1cnc(N)c(c1)O[C@H](C)c1cc(F)ccc1-c1nn(C)cc1C2]

In one embodiment, provided herein is a solid form comprising a salt of Compound 1. In one embodiment, the solid form comprises a salicylic acid salt (salicylate salt) or maleic acid salt (maleate salt) of Compound 1. In one embodiment, provided herein is a solid form comprising an anhydrous salt of Compound 1.

It is contemplated that salts of Compound 1 can exist in a variety of solid forms. Such solid forms include crystalline solids (e.g., crystalline forms of an anhydrous salt of Compound 1), amorphous solids, or mixtures of crystalline and amorphous solids. In one embodiment, the solid form is substantially crystalline. In one embodiment, the solid form is crystalline.

5.2.4.1 Form A of a Salicylate Salt of Compound 1

In one embodiment, provided herein is a Form A of a salicylate salt of Compound 1. In one embodiment, Form A is a mono-salicylate salt of Compound 1.

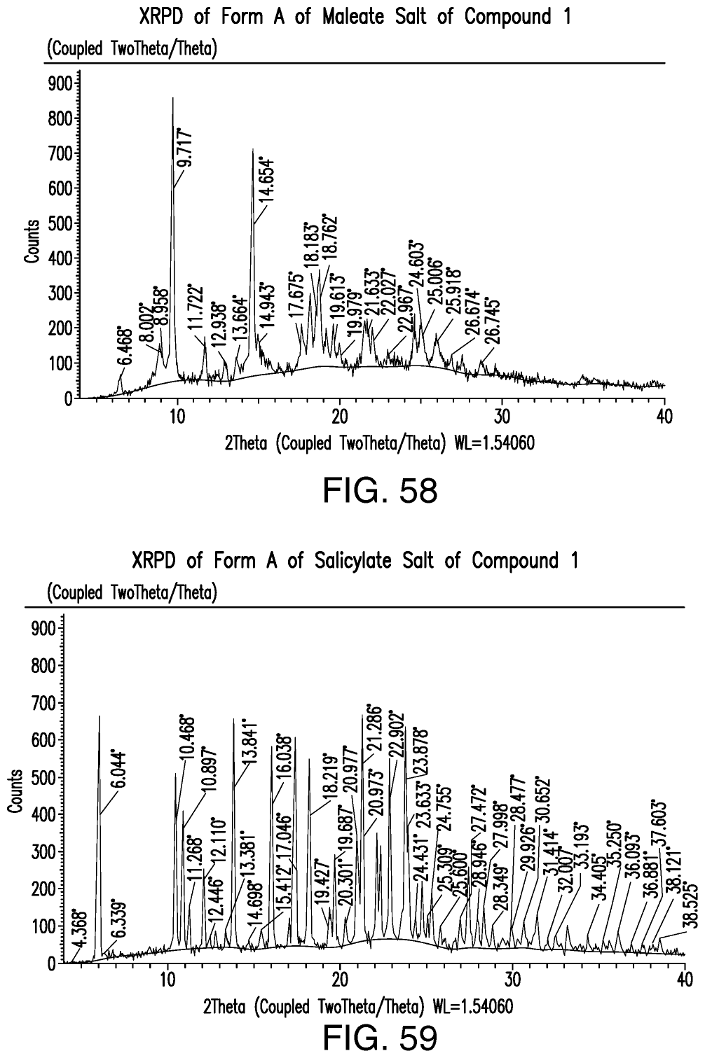

A representative XRPD pattern of Form A of a salicylate salt of Compound 1 is provided in FIG. 58.

In one embodiment, provided herein is a solid form comprising a salicylate salt of Compound 1, characterized by 1, 2, 3, 4, 5, 6, 7, 8, 9, 10, 11, 12, 13, 14, 15, 16, 17, 18, 19, or all of the XRPD peaks located at approximately the following positions (e.g., degrees 2θ±0.2) when measured using Cu Kα radiation: 6.5, 8.5, 9.0, 9.7, 11.7, 12.9, 13.7, 14.7, 14.9, 17.7, 18.2, 18.8, 19.6, 20.0, 21.6, 22.0, 23.0, 24.6, 25.0, and 25.9° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a salicylate salt of Compound 1, characterized by an XRPD pattern, when measured using Cu Kα radiation, comprising at least three peaks selected from the group consisting of approximately (e.g., ±0.2°) 6.5, 8.5, 9.7, 11.7, 14.7, 17.7, and 18.8° 2θ. In one embodiment, the solid form is characterized by an XRPD pattern comprising at least four peaks selected from the group consisting of approximately (e.g., ±0.2°) 6.5, 8.5, 9.7, 11.7, 14.7, 17.7, and 18.8° 2θ. In one embodiment, the solid form is characterized by an XRPD pattern comprising at least five peaks selected from the group consisting of approximately (e.g., ±0.2°) 6.5, 8.5, 9.7, 11.7, 14.7, 17.7, and 18.8° 2θ.

In one embodiment, provided herein is a solid form (e.g. a crystalline form) comprising a salicylate salt of Compound 1, characterized by an XRPD pattern comprising peaks at approximately (e.g., ±0.2°) 9.7, 11.7, and 14.7° 2θ. In one embodiment, the XRPD pattern further comprises peaks at approximately (e.g., ±0.2°) 6.5 and 18.8° 2θ. In one embodiment, the XRPD pattern further comprises peaks at approximately (e.g., ±0.2°) 8.5 and 17.7° 2θ. In one embodiment, the XRPD pattern comprises peaks at approximately (e.g., ±0.2°) 6.5, 8.5, 9.7, 11.7, 14.7, 17.7, 18.2, 18.8, and 19.6° 2θ.

In one embodiment, provided herein is a solid form comprising a salicylate salt of Compound 1, characterized by an XRPD pattern that matches the XRPD pattern depicted in FIG. 58. In one embodiment, the Form A that provides FIG. 58 is anhydrous.

In one embodiment, an XRPD pattern described herein is obtained using Cu Kα radiation. In one embodiment, the XRPD pattern is measured by XRPD using Cu Kα radiation comprising Kα₁ radiation having a wavelength of 1.5406 Å and Kα₂ radiation having a wavelength of 1.5444 Å.

In some embodiments, provided herein is a solid form comprising a salicylate salt of Compound 1, which is a crystalline anhydrous salicylate salt of Compound 1. In some embodiments, the solid form is substantially free of amorphous salicylate salt of Compound 1. In some embodiments, the solid form is substantially free of other solid forms (e.g., crystalline forms) of salicylate salt of Compound 1. In some embodiments, the solid form is substantially free of the free base form of Compound 1. In some embodiments, the solid form is substantially free of other salts of Compound 1. In some embodiments, the solid form is provided as substantially pure. In some embodiments, the solid form is substantially chemically pure. In some embodiments, the solid form is substantially enantiomerically pure. In some embodiments, the solid form is substantially physically pure.

In some embodiments, also provided herein is a process for preparing a salicylate salt of Compound 1, comprising exposing a composition comprising a free base of Compound 1 to salicylic acid. In one embodiment, the free base of Compound 1 is exposed to salicylic acid in an organic solvent, such as isopropyl acetate. In some embodiments, the organic solvent is isopropyl acetate. In some embodiments, the organic solvent is ethyl acetate.

All of the combinations of the above embodiments are encompassed by this application.

5.2.4.2 Form A of a Maleate Salt of Compound 1

In one embodiment, provided herein is a Form A of a maleate salt of Compound 1. In one embodiment, Form A is a mono-maleate salt of Compound 1.

A representative XRPD pattern of Form A of a maleate salt of Compound 1 is provided in FIG. 59.

In one embodiment, provided herein is a solid form comprising a maleate salt of Compound 1, characterized by 1, 2, 3, 4, 5, 6, 7, 8, 9, 10, 11, 12, 13, 14, 15, 16, 17, 18, 19, 20, 21, or all of the XRPD peaks located at approximately the following positions (e.g., degrees 2θ±0.2) when measured using Cu Kα radiation: 6.0, 10.5, 10.9, 11.3, 12.1, 13.8, 16.0, 17.4, 18.2, 19.7, 21.0, 21.3, 22.1, 22.3, 22.9, 23.9, 24.8, 25.3, 26.9, 27.5, 28.3, and 28.8° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a maleate salt of Compound 1, characterized by an XRPD pattern, when measured using Cu Kα radiation, comprising at least three peaks selected from the group consisting of approximately (e.g., ±0.2°) 6.0, 10.5, 10.9, 11.3, 12.1, 13.8, 16.0, 17.4, 18.2, 19.7, 21.0, 21.3, 22.1, 22.3, 22.9, 23.9, 24.8, 25.3, 26.9, 27.5, 28.3, and 28.8° 2θ. In one embodiment, the solid form is characterized by an XRPD pattern comprising at least four peaks selected from the group consisting of approximately (e.g., ±0.2°) 6.0, 10.5, 10.9, 11.3, 12.1, 13.8, 16.0, 17.4, 18.2, 19.7, 21.0, 21.3, 22.1, 22.3, 22.9, 23.9, 24.8, 25.3, 26.9, 27.5, 28.3, and 28.8° 2θ. In one embodiment, the solid form is characterized by an XRPD pattern comprising at least five peaks selected from the group consisting of approximately (e.g., ±0.2°) 6.0, 10.5, 10.9, 11.3, 12.1, 13.8, 16.0, 17.4, 18.2, 19.7, 21.0, 21.3, 22.1, 22.3, 22.9, 23.9, 24.8, 25.3, 26.9, 27.5, 28.3, and 28.8° 2θ.

In one embodiment, provided herein is a solid form (e.g. a crystalline form) comprising a maleate salt of Compound 1, characterized by an XRPD pattern comprising peaks at approximately (e.g., ±0.2°) 6.0, 13.8, and 21.3° 2θ. In one embodiment, the XRPD pattern further comprises peaks at approximately (e.g., ±0.2°) 16.0 and 17.4° 2θ. In one embodiment, the XRPD pattern further comprises peaks at approximately (e.g., ±0.2°) 18.2 and 22.9° 2θ. In one embodiment, the XRPD pattern comprises peaks at approximately (e.g., ±0.2°) 6.0, 10.5, 10.9, 12.1, 13.8, 16.0, 17.4, 18.2, 21.3, and 22.9° 2θ.

In one embodiment, provided herein is a solid form comprising a maleate salt of Compound 1, characterized by an XRPD pattern that matches the XRPD pattern depicted in FIG. 59. In one embodiment, the Form A that provides FIG. 59 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

In some embodiments, provided herein is a solid form comprising a maleate salt of Compound 1, which is a crystalline anhydrous maleate salt of Compound 1. In some embodiments, the solid form is substantially free of amorphous maleate salt of Compound 1. In some embodiments, the solid form is substantially free of other solid forms (e.g., crystalline forms) of maleate salt of Compound 1. In some embodiments, the solid form is substantially free of the free base form of Compound 1. In some embodiments, the solid form is substantially free of other salts of Compound 1. In some embodiments, the solid form is provided as substantially pure. In some embodiments, the solid form is substantially chemically pure. In some embodiments, the solid form is substantially enantiomerically pure. In some embodiments, the solid form is substantially physically pure.

In some embodiments, also provided herein is a process for preparing a maleate salt of Compound 1, comprising exposing a composition comprising a free base of Compound 1 to maleic acid. In one embodiment, the free base of Compound 1 is exposed to maleic acid in an organic solvent, such as isopropyl acetate. In some embodiments, the organic solvent is isopropyl acetate. In some embodiments, the organic solvent is ethyl acetate.

All of the combinations of the above embodiments are encompassed by this application.

5.2.5. Salts of a Compound of Formula (II)

In certain embodiments, provided herein is a salt of a compound of Formula (II):

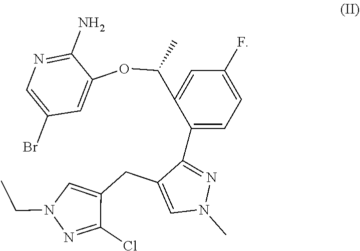 [SMILES: CCn1cc(Cc2cn(C)nc2-c2ccc(F)cc2[C@@H](C)Oc2cc(Br)cnc2N)c(Cl)n1]

In one embodiment, provided herein is a benzenesulfonic acid, ethanedisulfonic acid, citric acid, fumaric acid, hydrochloric acid, L-malic acid, maleic acid, methanesulfonic acid, naphthalene-1,5-disulfonic acid, sulfuric acid, succinic acid, L-tartaric acid, phosphoric acid, toluenesulfonic acid, oxalic acid, camphorsulfonic acid, ethanesulfonic acid, 2-naphthalenesulfonic acid, 2-hydroxyethanesulfonic acid, trifluoroacetic acid, or hydrobromic acid salt of the compound of Formula (II). In one embodiment, provided herein is a methanesulfonic acid (mesylate), toluenesulfonic acid (tosylate), camphorsulfonic acid (camsylate), ethanesulfonic acid (esylate), benzenesulfonic acid (besylate), 2-naphthalenesulfonic acid (2-naphthalenesulfonate), or sulfuric acid (sulfate) salt of the compound of Formula (II).

The molar ratio of the compound of Formula (II) (also referred to herein as “Compound 2”) to a counterion of the salt of Compound 2 may be about 1:1, about 1:2, about 1:3, about 2:1. In one embodiment, the molar ratio of Compound 2 to counterion is about 1:1. In one embodiment, the molar ratio of Compound 2 to a counterion is about 1:2.

In one embodiment, the counterion is besylate, mesylate, tosylate, camsylate, esylate, sulfate, or 2-naphthalenesulfonate. In one embodiment, the salt of Compound 2 is a benzenesulfonic acid salt (besylate salt) of Compound 2. In one embodiment, the salt of Compound 2 is a mono-besylate salt of Compound 2. In one embodiment, the salt of Compound 2 is a methanesulfonic acid salt (mesylate salt) of Compound 2. In one embodiment, the salt of Compound 2 is a mono-mesylate salt of Compound 2. In one embodiment, the salt of Compound 2 is a toluenesulfonic acid salt (tosylate salt) of Compound 2. In one embodiment, the salt of Compound 2 is a mono-tosylate salt of Compound 2. In one embodiment, the salt of Compound 2 is a camphorsulfonic acid salt (camsylate salt) of Compound 2. In one embodiment, the salt of Compound 2 is a mono-camsylate salt of Compound 2. In one embodiment, the salt of Compound 2 is an ethanesulfonic acid salt (esylate salt) of Compound 2. In one embodiment, the salt of Compound 2 is a mono-esylate salt of Compound 2. In one embodiment, the salt of Compound 2 is a sulfate salt of Compound 2. In one embodiment, the salt of Compound 2 is a hemi-sulfate salt (e.g. about 0.5 molar equiv. sulfate) of Compound 2. In one embodiment, the salt of Compound 2 is a 2-naphthalenesulfonic acid salt (2-naphthalenesulfonate salt) of Compound 2. In one embodiment, the salt of Compound 2 is a mono-2-naphthalenesulfonate salt of Compound 2.

In one embodiment, provided herein a solid form comprising a salt of Compound 2. In one embodiment, the salt of Compound 2 provided herein is amorphous. In one embodiment, the salt of Compound 2 provided herein is a crystalline solid form.

5.2.6. Solid Forms of Salts of Compound of Formula (II)

Provided herein are solid forms comprising a salt of a compound of Formula (II):

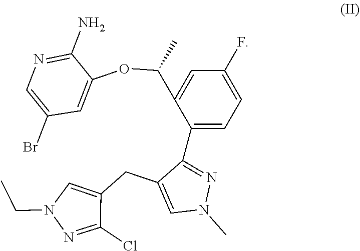 [SMILES: CCn1cc(Cc2cn(C)nc2-c2ccc(F)cc2[C@@H](C)Oc2cc(Br)cnc2N)c(Cl)n1]

In one embodiment, provided herein is a solid form comprising a salt of Compound 2. In one embodiment, the solid form comprises a benzenesulfonic acid, ethanedisulfonic acid, citric acid, fumaric acid, hydrochloric acid, L-malic acid, maleic acid, methanesulfonic acid, naphthalene-1,5-disulfonic acid, sulfuric acid, succinic acid, L-tartaric acid, phosphoric acid, toluenesulfonic acid, oxalic acid, camphorsulfonic acid, ethanesulfonic acid, 2-naphthalenesulfonic acid, 2-hydroxyethanesulfonic acid, trifluoroacetic acid, or hydrobromic acid salt of Compound 2. In one embodiment, the solid form comprises a a methanesulfonic acid (mesylate), toluenesulfonic acid (tosylate), camphorsulfonic acid (camsylate), ethanesulfonic acid (esylate), benzenesulfonic acid (besylate), 2-naphthalenesulfonic acid (2-naphthalenesulfonate), or sulfuric acid (sulfate) salt of Compound 2. In one embodiment, provided herein is a solid form comprising an anhydrous salt of Compound 2.

It is contemplated that salts of Compound 2 can exist in a variety of solid forms. Such solid forms include crystalline solids (e.g., crystalline forms of an anhydrous salt of Compound 2), amorphous solids, or mixtures of crystalline and amorphous solids. In one embodiment, the solid form is substantially crystalline. In one embodiment, the solid form is crystalline.

5.2.6.1 Form A of a Mesylate Salt of Compound 2

In one embodiment, provided herein is a Form A of a mesylate salt of Compound 2. In one embodiment, Form A is a mono-mesylate salt of Compound 2.

A representative XRPD pattern of Form A of a mesylate salt of Compound 2 is provided in FIG. 35.

In one embodiment, provided herein is a solid form comprising a mesylate salt of Compound 2, characterized by 1, 2, 3, 4, 5, 6, 7, 8, 9, 10, 11, 12, 13, 14, 15, 16, 17, 18, 19, 20, 21, 22, 23, 24, or all of the XRPD peaks located at approximately the following positions (e.g., degrees 2θ±0.2) when measured using Cu Kα radiation: 7.7, 10.6, 11.7, 15.1, 15.9, 16.4, 17.1, 17.5, 17.9, 19.5, 19.7, 21.5, 22.7, 23.1, 23.4, 23.6, 23.9, 24.2, 25.5, 25.9, 27.0, 28.4, 28.5, 30.5, and 32.9° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a mesylate salt of Compound 2, characterized by an XRPD pattern, when measured using Cu Kα radiation, comprising at least three peaks selected from the group consisting of approximately (e.g., ±0.2°) 7.7, 10.6, 11.7, 15.1, 15.9, 17.1, 17.5, 19.5, 19.7, 21.5, 22.7, 23.4, 23.6, and 25.9° 2θ. In one embodiment, the solid form is characterized by an XRPD pattern comprising at least four peaks selected from the group consisting of approximately (e.g., ±0.2°) 7.7, 10.6, 11.7, 15.1, 15.9, 17.1, 17.5, 19.5, 19.7, 21.5, 22.7, 23.4, 23.6, and 25.9° 2θ. In one embodiment, the solid form is characterized by an XRPD pattern comprising at least five peaks selected from the group consisting of approximately (e.g., ±0.2°) 7.7, 10.6, 11.7, 15.1, 15.9, 17.1, 17.5, 19.5, 19.7, 21.5, 22.7, 23.4, 23.6, and 25.9° 2θ.

In one embodiment, provided herein is a solid form (e.g. a crystalline form) comprising a mesylate salt of Compound 2, characterized by an XRPD pattern comprising peaks at approximately (e.g., ±0.2°) 15.9, 17.5, and 19.5° 2θ. In one embodiment, the XRPD pattern further comprises peaks at approximately (e.g., ±0.2°) 10.6 and 11.7° 2θ. In one embodiment, the XRPD pattern further comprises peaks at approximately (e.g., ±0.2°) 21.5 and 22.7° 2θ. In one embodiment, the XRPD pattern comprises peaks at approximately (e.g., ±0.2°) 7.7, 10.6, 11.7, 15.9, 17.5, 19.5, 21.5, and 22.7° 2θ.

In one embodiment, provided herein is a solid form comprising a mesylate salt of Compound 2, characterized by an XRPD pattern that matches the XRPD pattern depicted in FIG. 35. In one embodiment, the Form A that provides FIG. 35 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

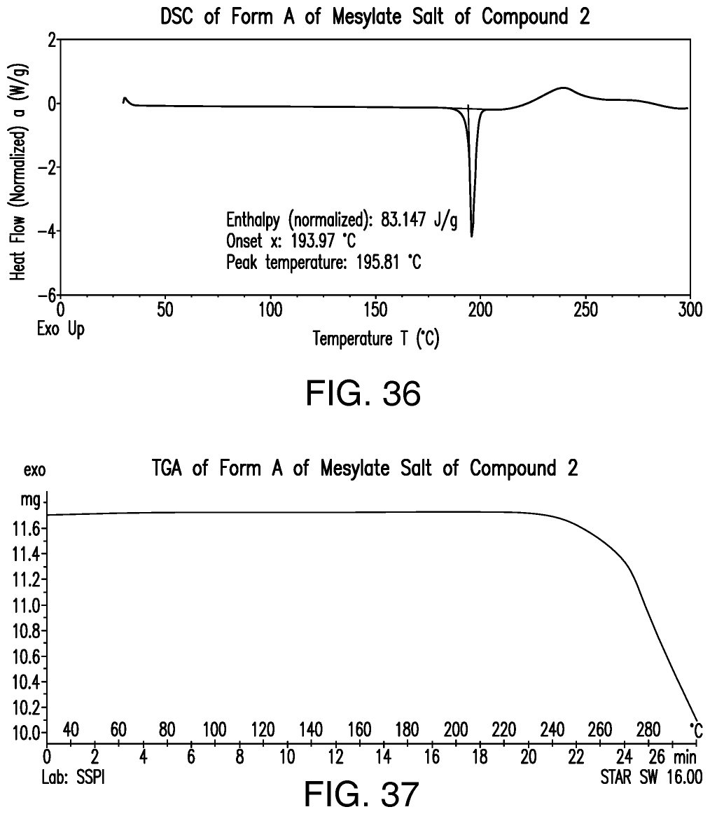

Representative DSC and TGA thermograms of Form A of a mesylate salt of Compound 2 are provided in FIG. 36 and FIG. 37, respectively. In one embodiment, provided herein is a solid form comprising a mesylate salt of Compound 2, which exhibits, as characterized by DSC, a thermal (endo) event with an onset temperature of about 194° C. (e.g. ±2°). In one embodiment, the thermal event has a peak temperature of about 196° C. (e.g. ±2°). In one embodiment, the solid form is characterized by a DSC thermogram that matches the DSC thermogram depicted in FIG. 36. In one embodiment, the DSC thermogram is as measured by DSC using a scanning rate of about 10° C./minute. In one embodiment, provided herein is a solid form comprising a mesylate salt of Compound 2, which exhibits a weight loss of about 0% upon heating from about 30° C. to about 210° C. In one embodiment, the solid form is characterized by a TGA thermogram that matches the TGA thermogram depicted in FIG. 37. In one embodiment, the TGA thermogram is as measured using a heating rate of about 10° C./minute. In one embodiment, the Form A that provides FIG. 36 and FIG. 37 is an anhydrous mesylate salt of Compound 2.

In some embodiments, provided herein is a solid form comprising a mesylate salt of Compound 2, which is a crystalline anhydrous mesylate salt of Compound 2. In some embodiments, the solid form is substantially free of amorphous mesylate salt of Compound 2. In some embodiments, the solid form is substantially free of other solid forms (e.g., crystalline forms) of mesylate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially enantiomerically pure. In some embodiments, the solid form is substantially physically pure.

In some embodiments, also provided herein is a process for preparing a mesylate salt of Compound 2, comprising exposing a composition comprising a free base of Compound 2 to methanesulfonic acid. In one embodiment, the free base of Compound 2 is exposed to methanesulfonic acid in an organic solvent, such as acetonitrile, ethyl acetate, THF, isopropyl acetate, or a mixture of ethyl acetate and heptane. In some embodiments, the organic solvent is acetonitrile. In some embodiments, the organic solvent is ethyl acetate. In some embodiments, the organic solvent is isopropyl acetate. In some embodiments, the organic solvent is a mixture of ethyl acetate and heptane. In some embodiments, the organic solvent is a mixture of isopropyl acetate and heptane.

All of the combinations of the above embodiments are encompassed by this application.

5.2.6.2 Form A of a Camsylate Salt of Compound 2

In one embodiment, provided herein is a Form A of a camsylate salt of Compound 2. In one embodiment, Form A is a mono-camsylate salt of Compound 2.

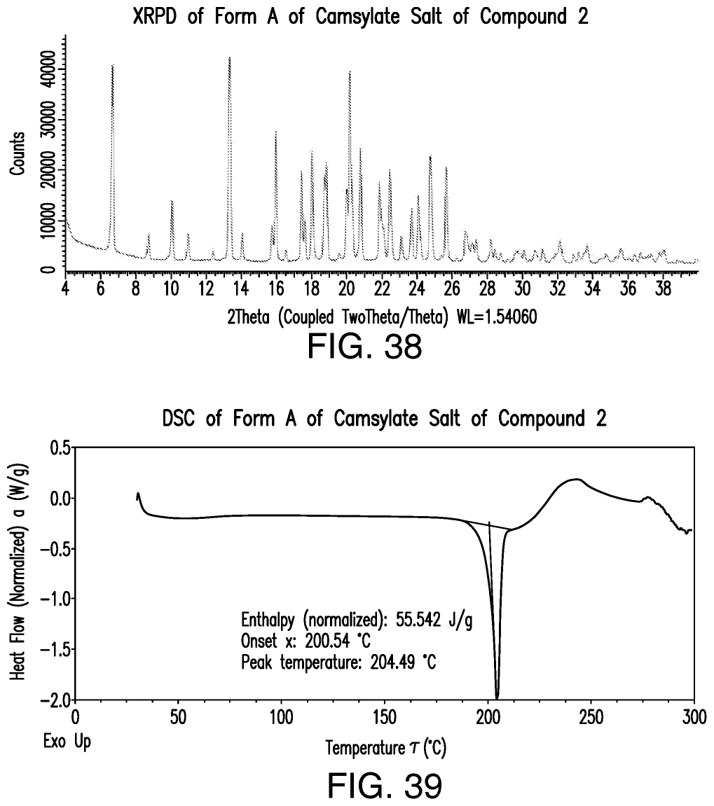

A representative XRPD pattern of Form A of a camsylate salt of Compound 2 is provided in FIG. 38.

In one embodiment, provided herein is a solid form comprising a camsylate salt of Compound 2, characterized by 1, 2, 3, 4, 5, 6, 7, 8, 9, 10, 11, 12, 13, 14, 15, 16, 17, 18, 19, 20, 21, 22, 23, 24 or all of the XRPD peaks located at approximately the following positions (e.g., degrees 2θ±0.2) when measured using Cu Kα radiation: 6.7, 8.7, 10.1, 11.0, 13.3, 14.1, 15.7, 16.0, 17.4, 17.6, 18.0, 18.7, 18.8, 20.0, 20.2, 20.8, 21.9, 22.1, 22.5, 23.7, 24.1, 24.8, 25.7, 26.8, and 32.2° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a camsylate salt of Compound 2, characterized by an XRPD pattern, when measured using Cu Kα radiation, comprising at least three peaks selected from the group consisting of approximately (e.g., ±0.2°) 6.7, 8.7, 10.1, 11.0, 13.3, 16.0, 17.4, 18.0, 18.8, 20.2, 20.8, 22.5, 24.8, and 25.7° 2θ. In one embodiment, the solid form is characterized by an XRPD pattern comprising at least four peaks selected from the group consisting of approximately (e.g., ±0.2°) 6.7, 8.7, 10.1, 11.0, 13.3, 16.0, 17.4, 18.0, 18.8, 20.2, 20.8, 22.5, 24.8, and 25.7° 2θ. In one embodiment, the solid form is characterized by an XRPD pattern comprising at least five peaks selected from the group consisting of approximately (e.g., ±0.2°) 6.7, 8.7, 10.1, 11.0, 13.3, 16.0, 17.4, 18.0, 18.8, 20.2, 20.8, 22.5, 24.8, and 25.7° 2θ.

In one embodiment, provided herein is a solid form (e.g. a crystalline form) comprising a camsylate salt of Compound 2, characterized by an XRPD pattern comprising peaks at approximately (e.g., ±0.2°) 6.7, 13.3, and 20.2° 2θ. In one embodiment, the XRPD pattern further comprises peaks at approximately (e.g., ±0.2°) 10.1 and 16.0° 2θ. In one embodiment, the XRPD pattern further comprises peaks at approximately (e.g., ±0.2°) 8.7 and 18.0° 2θ. In one embodiment, the XRPD pattern comprises peaks at approximately (e.g., ±0.2°) 6.7, 8.7, 10.1, 11.0, 13.3, 14.1, 16.0, 18.0, and 20.2° 2θ.

In one embodiment, provided herein is a solid form comprising a camsylate salt of Compound 2, characterized by an XRPD pattern that matches the XRPD pattern depicted in FIG. 38. In one embodiment, the Form A that provides FIG. 38 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

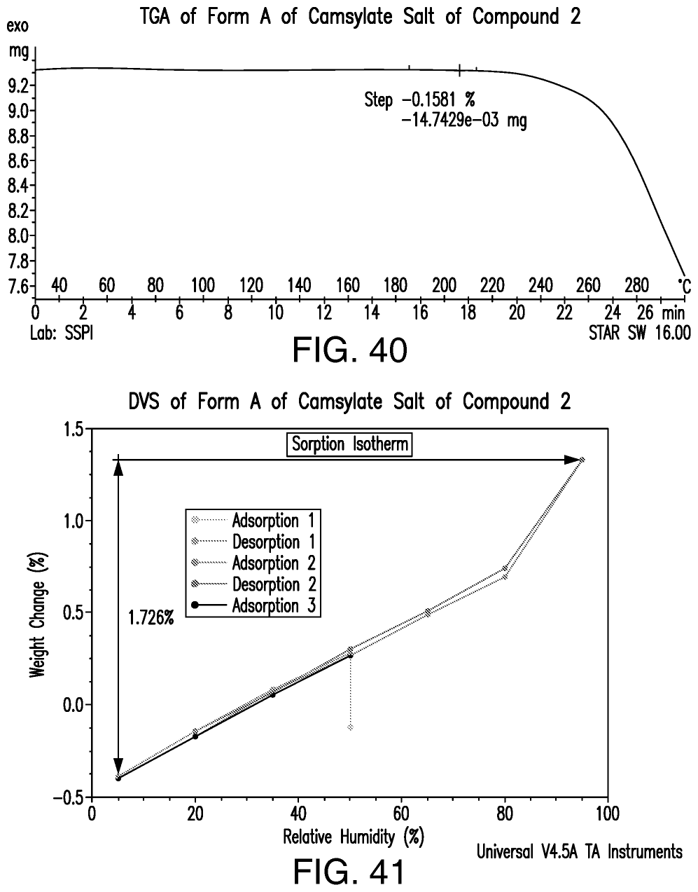

Representative DSC and TGA thermograms of Form A of a camsylate salt of Compound 2 are provided in FIG. 39 and FIG. 40, respectively. In one embodiment, provided herein is a solid form comprising a camsylate salt of Compound 2, which exhibits, as characterized by DSC, a thermal (endo) event with an onset temperature of about 201° C. (e.g. ±2°). In one embodiment, the thermal event has a peak temperature of about 204° C. (e.g. ±2°). In one embodiment, the solid form is characterized by a DSC thermogram that matches the DSC thermogram depicted in FIG. 39. In one embodiment, the DSC thermogram is as measured by DSC using a scanning rate of about 10° C./minute. In one embodiment, provided herein is a solid form comprising a camsylate salt of Compound 2, which exhibits a weight loss of about 0.2% upon heating from about 185° C. to about 215° C. In one embodiment, the solid form is characterized by a TGA thermogram that matches the TGA thermogram depicted in FIG. 40. In one embodiment, the TGA thermogram is as measured using a heating rate of about 10° C./minute. In one embodiment, the Form A that provides FIG. 39 and FIG. 40 is an anhydrous camsylate salt of Compound 2.

In some embodiments, provided herein is a solid form comprising a camsylate salt of Compound 2, which is a crystalline anhydrous camsylate salt of Compound 2. In some embodiments, the solid form is substantially free of amorphous camsylate salt of Compound 2. In some embodiments, the solid form is substantially free of other solid forms (e.g., crystalline forms) of camsylate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially enantiomerically pure. In some embodiments, the solid form is substantially chemically pure. In some embodiments, the solid form is substantially physically pure.

In some embodiments, also provided herein is a process for preparing a camsylate salt of Compound 2, comprising exposing a composition comprising a free base of Compound 2 to camphorsulfonic acid. In one embodiment, the free base of Compound 2 is exposed to camphorsulfonic acid in an organic solvent, such as a mixture of methyl acetate and heptane, a mixture of ethyl acetate and heptane, or a mixture of isopropyl acetate and heptane. In certain embodiments, the organic solvent is about 1:1 methyl acetate and heptane. In certain embodiments, the organic solvent is about 1:1 ethyl acetate and heptane. In certain embodiments, the organic solvent is about 1:1 isopropyl acetate and heptane.

All of the combinations of the above embodiments are encompassed by this application.

5.2.6.3 Form A of an Esylate Salt of Compound 2

In one embodiment, provided herein is a Form A of an esylate salt of Compound 2. In one embodiment, Form A is a mono-esylate salt of Compound 2.

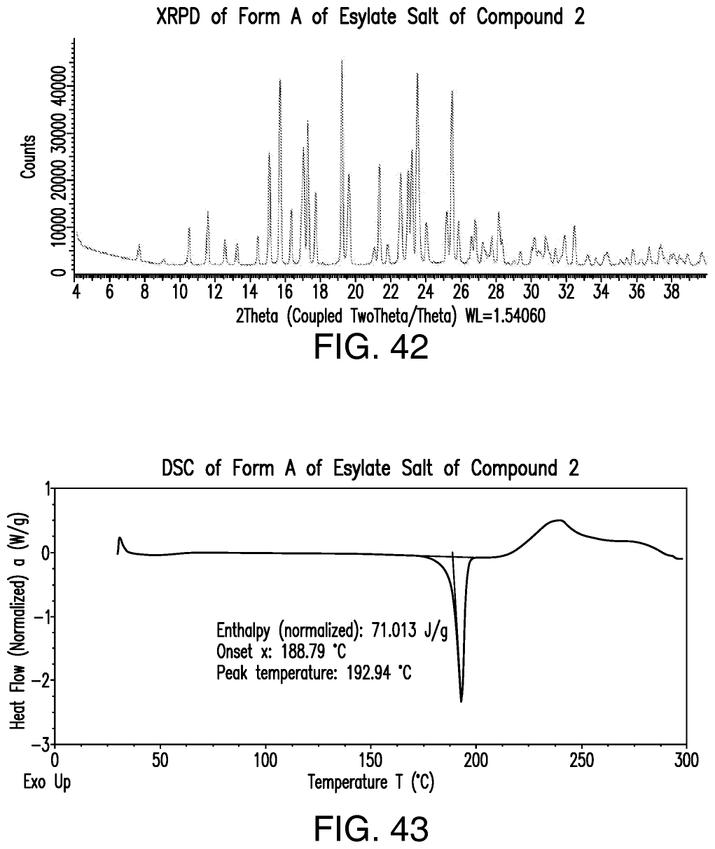

A representative XRPD pattern of Form A of an esylate salt of Compound 2 is provided in FIG. 42.

In one embodiment, provided herein is a solid form comprising an esylate salt of Compound 2, characterized by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or all of the XRPD peaks located at approximately the following positions (e.g., degrees 2θ±0.2) when measured using Cu Kα radiation: 7.8, 10.5, 11.6, 12.6, 13.2, 14.4, 15.1, 15.7, 16.3, 17.0, 17.3, 17.7, 19.2, 19.6, 21.4, 22.6, 23.0, 23.2, 23.5, 24.0, 25.2, 25.5, 25.9, 26.6, 26.8, 27.8, 28.2, 28.4, 30.2, 30.9, 31.9, and 32.5° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising an esylate salt of Compound 2, characterized by an XRPD pattern, when measured using Cu Kα radiation, comprising at least three peaks selected from the group consisting of approximately (e.g., ±0.2°) 8.7, 10.5, 11.6, 12.6, 13.2, 14.4, 15.1, 15.7, 17.0, 17.3, 19.2, 19.6, 21.4, 22.6, 23.0, 23.2, 23.5, and 25.5° 2θ. In one embodiment, the solid form is characterized by an XRPD pattern comprising at least four peaks selected from the group consisting of approximately (e.g., ±0.2°) 8.7, 10.5, 11.6, 12.6, 13.2, 14.4, 15.1, 15.7, 17.0, 17.3, 19.2, 19.6, 21.4, 22.6, 23.0, 23.2, 23.5, and 25.5° 2θ. In one embodiment, the solid form is characterized by an XRPD pattern comprising at least five peaks selected from the group consisting of approximately (e.g., ±0.2°) 8.7, 10.5, 11.6, 12.6, 13.2, 14.4, 15.1, 15.7, 17.0, 17.3, 19.2, 19.6, 21.4, 22.6, 23.0, 23.2, 23.5, and 25.5° 2θ.

In one embodiment, provided herein is a solid form (e.g. a crystalline form) comprising an esylate salt of Compound 2, characterized by an XRPD pattern comprising peaks at approximately (e.g., ±0.2°) 11.6, 15.7, and 19.2° 2θ. In one embodiment, the XRPD pattern further comprises peaks at approximately (e.g., ±0.2°) 10.5 and 17.3° 2θ. In one embodiment, the XRPD pattern further comprises peaks at approximately (e.g., ±0.2°) 14.4 and 15.1° 2θ. In one embodiment, the XRPD pattern comprises peaks at approximately (e.g., ±0.2°) 7.8, 10.5, 11.6, 12.6, 13.2, 14.4, 15.1, 15.7, 17.3, and 19.2° 2θ.

In one embodiment, provided herein is a solid form comprising an esylate salt of Compound 2, characterized by an XRPD pattern that matches the XRPD pattern depicted in FIG. 42. In one embodiment, the Form A that provides FIG. 42 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

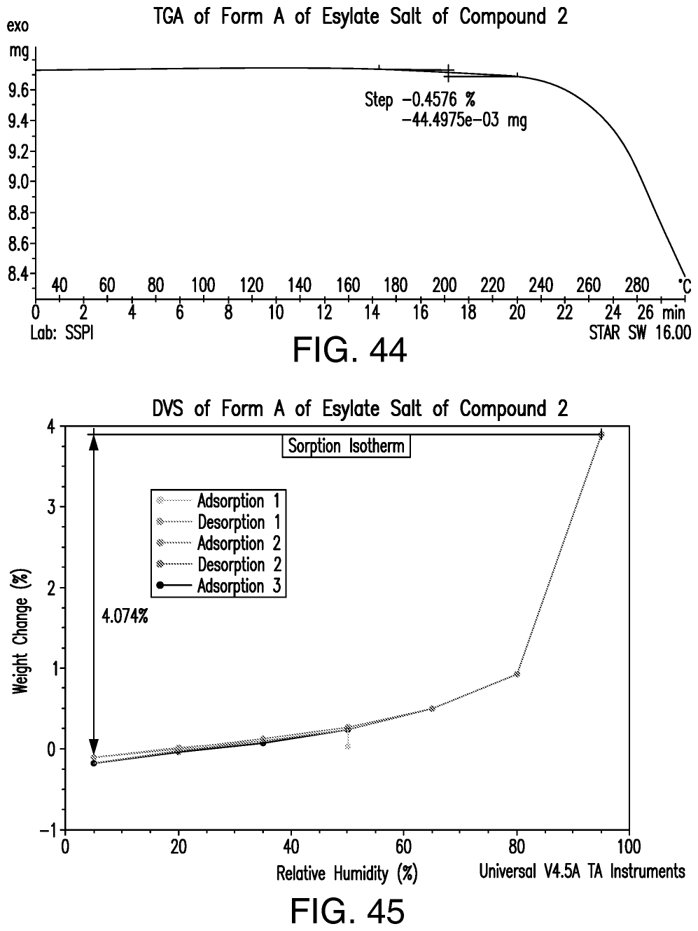

Representative DSC and TGA thermograms of Form A of an esylate salt of Compound 2 are provided in FIG. 43 and FIG. 44, respectively. In one embodiment, provided herein is a solid form comprising an esylate salt of Compound 2, which exhibits, as characterized by DSC, a thermal (endo) event with an onset temperature of about 189° C. (e.g. ±2°). In one embodiment, the thermal event has a peak temperature of about 193° C. (e.g. ±2°). In one embodiment, the solid form is characterized by a DSC thermogram that matches the DSC thermogram depicted in FIG. 43. In one embodiment, the DSC thermogram is as measured by DSC using a scanning rate of about 10° C./minute. In one embodiment, provided herein is a solid form comprising an esylate salt of Compound 2, which exhibits a weight loss of about 0.5% upon heating from about 170° C. to about 235° C. In one embodiment, the solid form is characterized by a TGA thermogram that matches the TGA thermogram depicted in FIG. 44. In one embodiment, the TGA thermogram is as measured using a heating rate of about 10° C./minute. In one embodiment, the Form A that provides FIG. 43 and FIG. 44 is an anhydrous esylate salt of Compound 2.

In some embodiments, provided herein is a solid form comprising an esylate salt of Compound 2, which is a crystalline anhydrous esylate salt of Compound 2. In some embodiments, the solid form is substantially free of amorphous esylate salt of Compound 2. In some embodiments, the solid form is substantially free of other solid forms (e.g., crystalline forms) of esylate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially physically pure.

In some embodiments, also provided herein is a process for preparing an esylate salt of Compound 2, comprising exposing a composition comprising a free base of Compound 2 to ethanesulfonic acid. In one embodiment, the free base of Compound 2 is exposed to ethanesulfonic acid in an organic solvent, such as a 1:1 mixture of ethyl acetate and heptane.

All of the combinations of the above embodiments are encompassed by this application.

5.2.6.4 Form A of a Sulfate Salt of Compound 2

In one embodiment, provided herein is a Form A of a sulfate salt of Compound 2. In one embodiment, Form A is a hemi-sulfate (e.g. about 0.5 molar equiv. sulfate) salt of Compound 2.

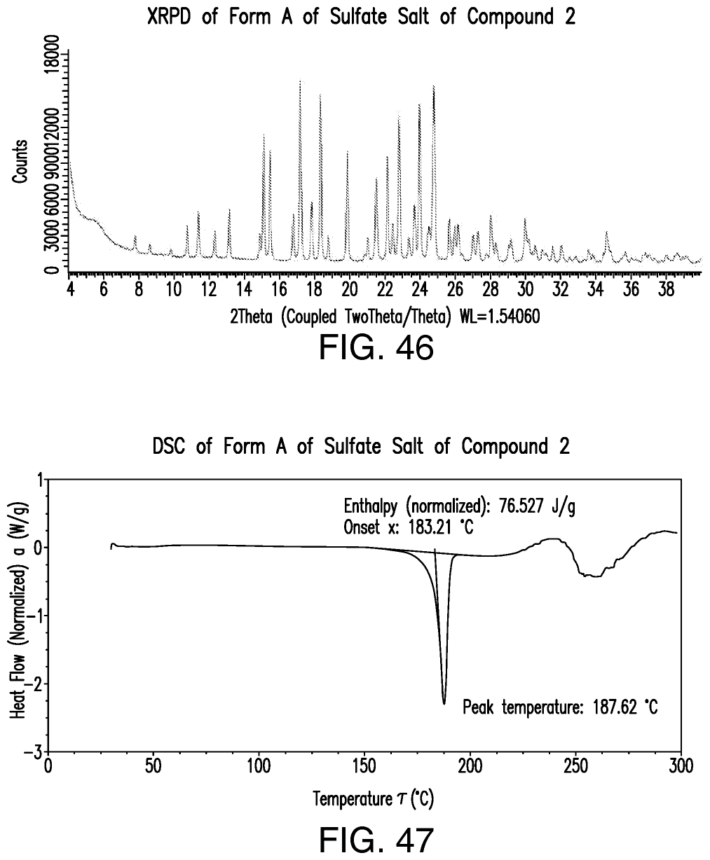

A representative XRPD pattern of Form A of a sulfate salt of Compound 2 is provided in FIG. 46.

In one embodiment, provided herein is a solid form comprising a sulfate salt of Compound 2, characterized by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or all of the XRPD peaks located at approximately the following positions (e.g., degrees 2θ±0.2) when measured using Cu Kα radiation: 10.8, 11.4, 12.3, 13.1, 14.9, 15.1, 15.5, 16.8, 17.2, 17.8, 18.3, 18.8, 19.9, 21.0, 21.5, 22.1, 22.5, 22.8, 23.4, 23.7, 24.0, 24.5, 24.8, 25.7, 26.0, 26.2, 27.0, 27.3, 28.0, 29.2, 30.0, and 34.6° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a sulfate salt of Compound 2, characterized by an XRPD pattern, when measured using Cu Kα radiation, comprising at least three peaks selected from the group consisting of approximately (e.g., ±0.2°) 10.8, 11.4, 12.3, 13.1, 15.1, 15.5, 17.2, 18.3, 19.9, 21.5, 22.1, 22.8, 24.0, and 24.8° 2θ. In one embodiment, the solid form is characterized by an XRPD pattern comprising at least four peaks selected from the group consisting of approximately (e.g., ±0.2°) 10.8, 11.4, 12.3, 13.1, 15.1, 15.5, 17.2, 18.3, 19.9, 21.5, 22.1, 22.8, 24.0, and 24.8° 2θ. In one embodiment, the solid form is characterized by an XRPD pattern comprising at least five peaks selected from the group consisting of approximately (e.g., ±0.2°) 10.8, 11.4, 12.3, 13.1, 15.1, 15.5, 17.2, 18.3, 19.9, 21.5, 22.1, 22.8, 24.0, and 24.8° 2θ.

In one embodiment, provided herein is a solid form (e.g. a crystalline form) comprising a sulfate salt of Compound 2, characterized by an XRPD pattern comprising peaks at approximately (e.g., ±0.2°) 13.1, 17.2, and 18.3° 2θ. In one embodiment, the XRPD pattern further comprises peaks at approximately (e.g., ±0.2°) 10.8 and 11.4° 2θ. In one embodiment, the XRPD pattern further comprises peaks at approximately (e.g., ±0.2°) 15.1 and 19.9° 2θ. In one embodiment, the XRPD pattern comprises peaks at approximately (e.g., ±0.2°) 10.8, 11.4, 12.3, 13.1, 15.1, 15.5, 16.8, 17.2, 18.3, 19.9° 2θ.

In one embodiment, provided herein is a solid form comprising a sulfate salt of Compound 2, characterized by an XRPD pattern that matches the XRPD pattern depicted in FIG. 46. In one embodiment, the Form A that provides FIG. 46 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

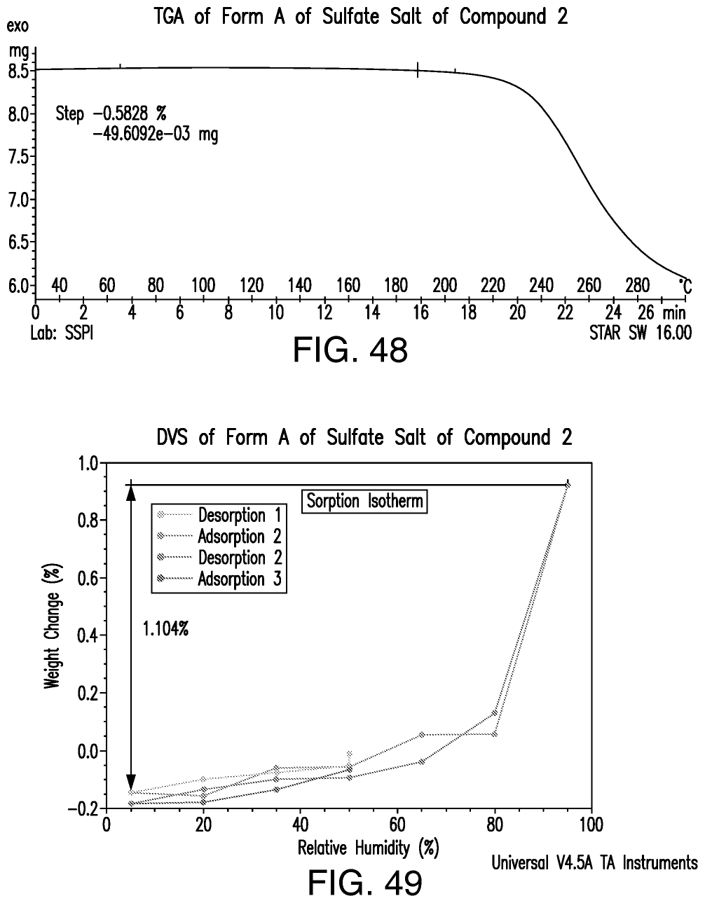

Representative DSC and TGA thermograms of Form A of a sulfate salt of Compound 2 are provided in FIG. 47 and FIG. 48, respectively. In one embodiment, provided herein is a solid form comprising a sulfate salt of Compound 2, which exhibits, as characterized by DSC, a thermal (endo) event with an onset temperature of about 183° C. (e.g. ±2°). In one embodiment, the thermal event has a peak temperature of about 188° C. (e.g. ±2°). In one embodiment, the solid form is characterized by a DSC thermogram that matches the DSC thermogram depicted in FIG. 47. In one embodiment, the DSC thermogram is as measured by DSC using a scanning rate of about 10° C./minute. In one embodiment, provided herein is a solid form comprising a sulfate salt of Compound 2, which exhibits a weight loss of about 0.6% upon heating from about 30° C. to about 200° C. In one embodiment, the solid form is characterized by a TGA thermogram that matches the TGA thermogram depicted in FIG. 48. In one embodiment, the TGA thermogram is as measured using a heating rate of about 10° C./minute. In one embodiment, the Form A that provides FIG. 47 and FIG. 48 is an anhydrous sulfate salt of Compound 2.

In some embodiments, provided herein is a solid form comprising a sulfate salt of Compound 2, which is a crystalline anhydrous sulfate salt of Compound 2. In some embodiments, the solid form is substantially free of amorphous sulfate salt of Compound 2. In some embodiments, the solid form is substantially free of other solid forms (e.g., crystalline forms) of sulfate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially physically pure.

In some embodiments, also provided herein is a process for preparing a sulfate salt of Compound 2, comprising exposing a composition comprising a free base of Compound 2 to sulfuric acid. In one embodiment, the free base of Compound 2 is exposed to sulfuric acid in an organic solvent, such as a 1:1 mixture of ethanol and heptane.

All of the combinations of the above embodiments are encompassed by this application.

5.2.6.5 Form A of a Tosylate Salt of Compound 2

In one embodiment, provided herein is a Form A of a tosylate salt of Compound 2. In one embodiment, Form A is a mono-tosylate salt of Compound 2.

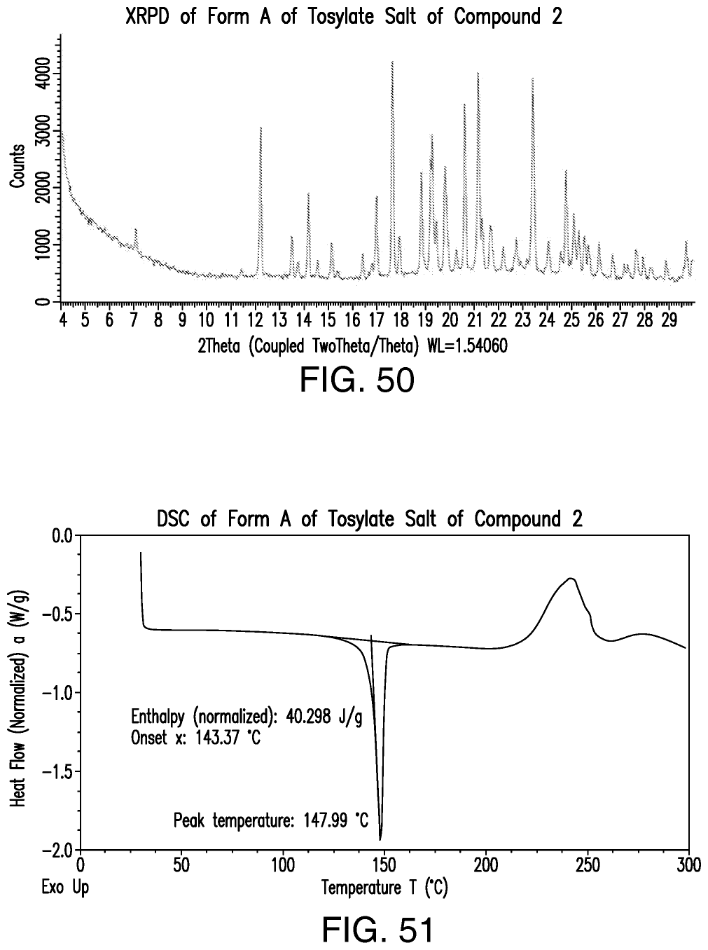

A representative XRPD pattern of Form A of a tosylate salt of Compound 2 is provided in FIG. 50.

In one embodiment, provided herein is a solid form comprising a tosylate salt of Compound 2, characterized by 1, 2, 3, 4, 5, 6, 7, 8, 9, 10, 11, 12, 13, 14, 15, 16, 17, 18, 19, or all of the XRPD peaks located at approximately the following positions (e.g., degrees 2θ±0.2) when measured using Cu Kα radiation: 7.0, 12.2, 13.5, 14.2, 15.1, 17.0, 17.6, 18.8, 19.2, 19.4, 19.8, 20.6, 21.2, 21.3, 21.7, 23.4, 24.8, 25.1, 25.3, and 25.5° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a tosylate salt of Compound 2, characterized by an XRPD pattern, when measured using Cu Kα radiation, comprising at least three peaks selected from the group consisting of approximately (e.g., ±0.2°) 12.2, 13.5, 14.2, 15.1, 17.0, 17.6, 18.8, 19.2, 19.4, 19.8, 20.6, 21.2, 21.3, 21.7, 23.4, 24.8, 25.1, 25.3, and 25.5° 2θ. In one embodiment, the solid form is characterized by an XRPD pattern comprising at least four peaks selected from the group consisting of approximately (e.g., ±0.2°) 12.2, 13.5, 14.2, 15.1, 17.0, 17.6, 18.8, 19.2, 19.4, 19.8, 20.6, 21.2, 21.3, 21.7, 23.4, 24.8, 25.1, 25.3, and 25.5° 2θ. In one embodiment, the solid form is characterized by an XRPD pattern comprising at least five peaks selected from the group consisting of approximately (e.g., ±0.2°) 12.2, 13.5, 14.2, 15.1, 17.0, 17.6, 18.8, 19.2, 19.4, 19.8, 20.6, 21.2, 21.3, 21.7, 23.4, 24.8, 25.1, 25.3, and 25.5° 2θ.

In one embodiment, provided herein is a solid form (e.g. a crystalline form) comprising a tosylate salt of Compound 2, characterized by an XRPD pattern comprising peaks at approximately (e.g., ±0.2°) 12.2, 14.2, and 17.6° 2θ. In one embodiment, the XRPD pattern further comprises peaks at approximately (e.g., ±0.2°) 19.2 and 20.6° 2θ. In one embodiment, the XRPD pattern further comprises peaks at approximately (e.g., ±0.2°) 19.8 and 21.2° 2θ. In one embodiment, the XRPD pattern comprises peaks at approximately (e.g., ±0.2°) 7.0, 12.2, 13.5, 14.2, 15.1, 17.6, 19.2, 19.8, 20.6, and 21.2° 2θ.

In one embodiment, provided herein is a solid form comprising a tosylate salt of Compound 2, characterized by an XRPD pattern that matches the XRPD pattern depicted in FIG. 50. In one embodiment, the Form A that provides FIG. 50 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

A representative DSC thermogram of Form A of a tosylate salt of Compound 2 are provided in FIG. 51. In one embodiment, provided herein is a solid form comprising a tosylate salt of Compound 2, which exhibits, as characterized by DSC, a thermal (endo) event with an onset temperature of about 143° C. (e.g. ±2°). In one embodiment, the thermal event has a peak temperature of about 148° C. (e.g. ±2°). In one embodiment, the solid form is characterized by a DSC thermogram that matches the DSC thermogram depicted in FIG. 51. In one embodiment, the DSC thermogram is as measured by DSC using a scanning rate of about 10° C./minute. In one embodiment, the Form A that provides FIG. 51 is an anhydrous tosylate salt of Compound 2.

In some embodiments, provided herein is a solid form comprising a tosylate salt of Compound 2, which is a crystalline anhydrous tosylate salt of Compound 2. In some embodiments, the solid form is substantially free of amorphous tosylate salt of Compound 2. In some embodiments, the solid form is substantially free of other solid forms (e.g., crystalline forms) of tosylate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially physically pure.

In some embodiments, also provided herein is a process for preparing a tosylate salt of Compound 2, comprising exposing a composition comprising a free base of Compound 2 to toluenesulfonic acid. In one embodiment, the free base of Compound 2 is exposed to toluenesulfonic acid in an organic solvent, such as a 1:1 mixture of ethyl acetate and heptane.

All of the combinations of the above embodiments are encompassed by this application.

5.2.6.6 Form A of a Besylate Salt of Compound 2

In one embodiment, provided herein is a Form A of a besylate salt of Compound 2. In one embodiment, Form A is a mono-besylate salt of Compound 2.

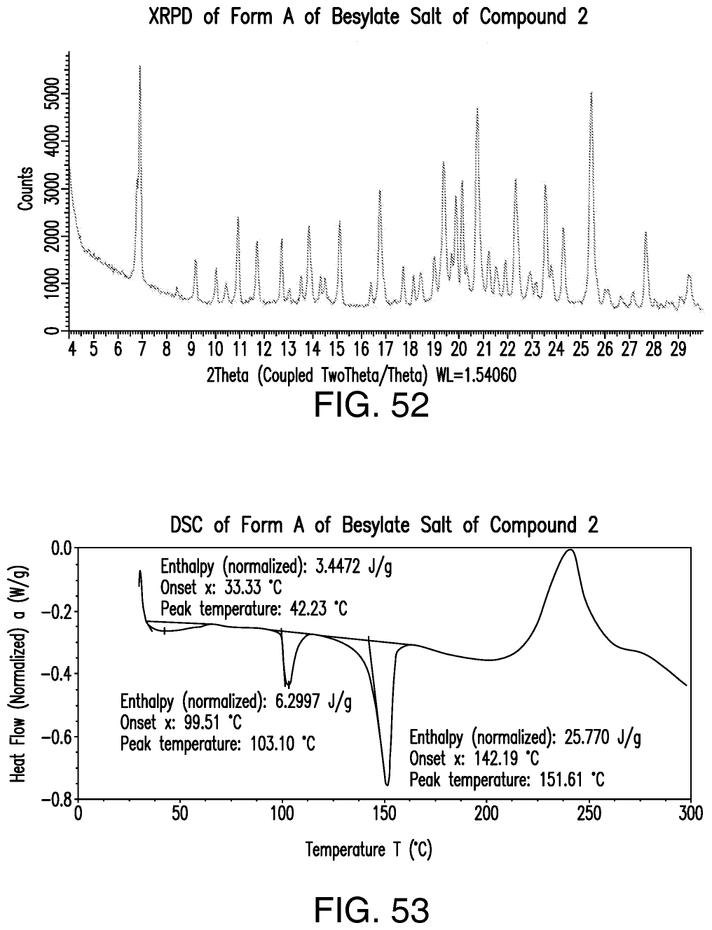

A representative XRPD pattern of Form A of a besylate salt of Compound 2 is provided in FIG. 52.

In one embodiment, provided herein is a solid form comprising a besylate salt of Compound 2, characterized by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or all of the XRPD peaks located at approximately the following positions (e.g., degrees 2θ±0.2) when measured using Cu Kα radiation: 6.8, 6.9, 9.2, 10.0, 10.9, 11.7, 12.7, 13.5, 13.8, 14.3, 14.5, 15.1, 16.8, 17.7, 18.1, 18.4, 19.0, 19.4, 19.7, 19.9, 20.1, 20.3, 20.8, 21.2, 21.5, 21.9, 22.3, 22.9, 23.6, 23.8, 24.3, and 25.4° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a besylate salt of Compound 2, characterized by an XRPD pattern, when measured using Cu Kα radiation, comprising at least three peaks selected from the group consisting of approximately (e.g., ±0.2°) 6.9, 9.2, 10.0, 10.9, 11.7, 12.7, 13.5, 13.8, 14.3, 14.5, 15.1, 16.8, 17.7, 18.1, 18.4, 19.0, 19.4, 19.7, 19.9, 20.1, 20.3, and 20.8° 2θ. In one embodiment, the solid form is characterized by an XRPD pattern comprising at least four peaks selected from the group consisting of approximately (e.g., ±0.2°) 6.9, 9.2, 10.0, 10.9, 11.7, 12.7, 13.5, 13.8, 14.3, 14.5, 15.1, 16.8, 17.7, 18.1, 18.4, 19.0, 19.4, 19.7, 19.9, 20.1, 20.3, and 20.8° 2θ. In one embodiment, the solid form is characterized by an XRPD pattern comprising at least five peaks selected from the group consisting of approximately (e.g., ±0.2°) 6.9, 9.2, 10.0, 10.9, 11.7, 12.7, 13.5, 13.8, 14.3, 14.5, 15.1, 16.8, 17.7, 18.1, 18.4, 19.0, 19.4, 19.7, 19.9, 20.1, 20.3, and 20.8° 2θ.

In one embodiment, provided herein is a solid form (e.g. a crystalline form) comprising a besylate salt of Compound 2, characterized by an XRPD pattern comprising peaks at approximately (e.g., ±0.2°) 6.9, 10.9, and 16.8° 2θ. In one embodiment, the XRPD pattern further comprises peaks at approximately (e.g., ±0.2°) 13.8 and 15.1° 2θ. In one embodiment, the XRPD pattern further comprises peaks at approximately (e.g., ±0.2°) 11.7 and 12.7° 2θ° 2θ. In one embodiment, the XRPD pattern comprises peaks at approximately (e.g., ±0.2°) 6.9, 9.2, 10.0, 10.9, 11.7, 12.7, 13.5, 13.8, 15.1, 16.8, 19.4, and 20.8° 2θ.

In one embodiment, provided herein is a solid form comprising a besylate salt of Compound 2, characterized by an XRPD pattern that matches the XRPD pattern depicted in FIG. 52. In one embodiment, the Form A that provides FIG. 52 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

A representative DSC thermogram of Form A of a besylate salt of Compound 2 are provided in FIG. 53. In one embodiment, provided herein is a solid form comprising a besylate salt of Compound 2, which exhibits, as characterized by DSC, a first thermal (endo) event with an onset temperature of about 33° C. (e.g. ±2°). In one embodiment, the first thermal event has a peak temperature of about 42° C. (e.g. ±2°). In one embodiment, provided herein is a solid form comprising a besylate salt of Compound 2, which exhibits, as characterized by DSC, a second thermal (endo) event with an onset temperature of about 100° C. (e.g. ±2°). In one embodiment, the second thermal event has a peak temperature of about 103° C. (e.g. ±2°). In one embodiment, provided herein is a solid form comprising a besylate salt of Compound 2, which exhibits, as characterized by DSC, a third thermal (endo) event with an onset temperature of about 142° C. (e.g. ±2°). In one embodiment, the third thermal event has a peak temperature of about 152° C. (e.g. ±2°). In one embodiment, the solid form is characterized by a DSC thermogram that matches the DSC thermogram depicted in FIG. 53. In one embodiment, the DSC thermogram is as measured by DSC using a scanning rate of about 10° C./minute. In one embodiment, the Form A that provides FIG. 53 is an anhydrous besylate salt of Compound 2.

In some embodiments, provided herein is a solid form comprising a besylate salt of Compound 2, which is a crystalline anhydrous besylate salt of Compound 2. In some embodiments, the solid form is substantially free of amorphous besylate salt of Compound 2. In some embodiments, the solid form is substantially free of other solid forms (e.g., crystalline forms) of besylate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially physically pure.

In some embodiments, also provided herein is a process for preparing a Form A of a besylate salt of Compound 2, comprising exposing a composition comprising a free base of Compound 2 to benzenesulfonic acid. In one embodiment, the free base of Compound 2 is exposed to benzenesulfonic acid in an organic solvent, such as a 1:1 mixture of methyl acetate and heptane. In one embodiment, the process further comprises drying the besylate salt of Compound 2 under vacuum.

All of the combinations of the above embodiments are encompassed by this application.

5.2.6.7 Form B of a Besylate Salt of Compound 2

In one embodiment, provided herein is a Form B of a besylate salt of Compound 2. In one embodiment, Form B is a mono-besylate salt of Compound 2.

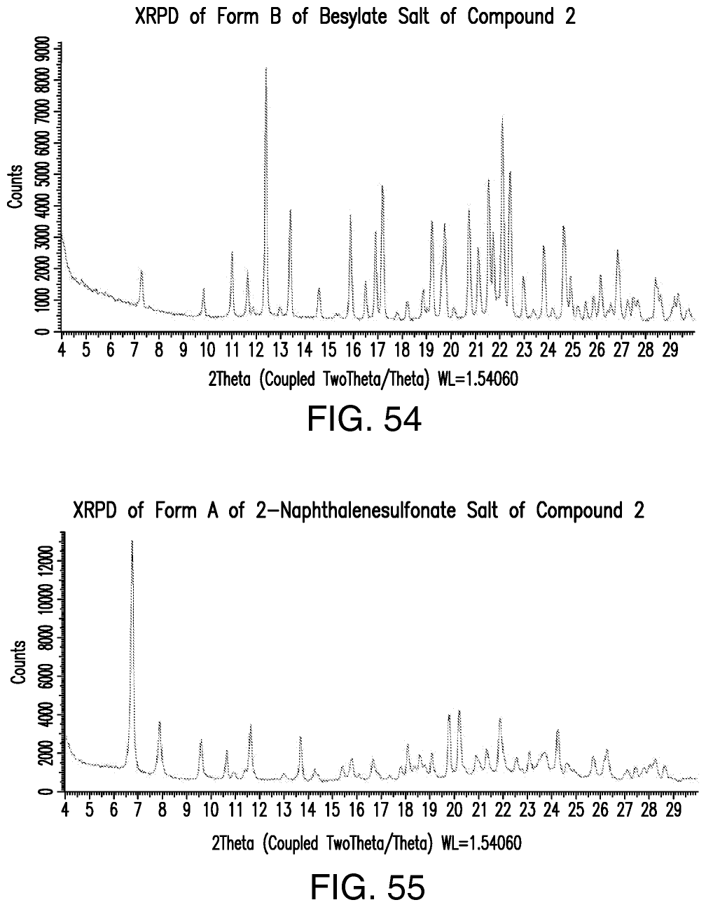

A representative XRPD pattern of Form B of a besylate salt of Compound 2 is provided in FIG. 54.

In one embodiment, provided herein is a solid form comprising a besylate salt of Compound 2, characterized by 1, 2, 3, 4, 5, 6, 7, 8, 9, 10, 11, 12, 13, 14, 15, 16, 17, 18, 19, 20, 21, or all of the XRPD peaks located at approximately the following positions (e.g., degrees 2θ±0.2) when measured using Cu Kα radiation: 7.3, 9.8, 11.0, 11.7, 12.4, 13.4, 15.9, 16.9, 17.2, 19.2, 19.8, 20.8, 21.1, 21.6, 21.7, 22.1, 22.4, 23.0, 23.8, 24.6, 24.9, and 26.9° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a besylate salt of Compound 2, characterized by an XRPD pattern, when measured using Cu Kα radiation, comprising at least three peaks selected from the group consisting of approximately (e.g., ±0.2°) 11.0, 11.7, 12.4, 13.4, 15.9, 16.9, 17.2, 19.2, 19.8, 20.8, 21.1, 21.6, 21.7, 22.1, 22.4, 23.0, 23.8, 24.6, 24.9, and 26.9° 2θ. In one embodiment, the solid form is characterized by an XRPD pattern comprising at least four peaks selected from the group consisting of approximately (e.g., ±0.2°) 11.0, 11.7, 12.4, 13.4, 15.9, 16.9, 17.2, 19.2, 19.8, 20.8, 21.1, 21.6, 21.7, 22.1, 22.4, 23.0, 23.8, 24.6, 24.9, and 26.9° 2θ. In one embodiment, the solid form is characterized by an XRPD pattern comprising at least five peaks selected from the group consisting of approximately (e.g., ±0.2°) 11.0, 11.7, 12.4, 13.4, 15.9, 16.9, 17.2, 19.2, 19.8, 20.8, 21.1, 21.6, 21.7, 22.1, 22.4, 23.0, 23.8, 24.6, 24.9, and 26.9° 2θ.

In one embodiment, provided herein is a solid form (e.g. a crystalline form) comprising a besylate salt of Compound 2, characterized by an XRPD pattern comprising peaks at approximately (e.g., ±0.2°) 11.0, 12.4, and 13.4° 2θ. In one embodiment, the XRPD pattern further comprises peaks at approximately (e.g., ±0.2°) 17.2 and 20.7° 2θ. In one embodiment, the XRPD pattern further comprises peaks at approximately (e.g., ±0.2°) 15.9 and 19.2° 2θ. In one embodiment, the XRPD pattern comprises peaks at approximately (e.g., ±0.2°) 7.3, 9.8, 11.0, 11.7, 12.4, 13.4, 15.9, 17.2, 19.2, and 20.7° 2θ.

In one embodiment, provided herein is a solid form comprising a besylate salt of Compound 2, characterized by an XRPD pattern that matches the XRPD pattern depicted in FIG. 54.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

In some embodiments, provided herein is a solid form comprising a besylate salt of Compound 2, which is a crystalline besylate salt of Compound 2. In some embodiments, the solid form is substantially free of amorphous besylate salt of Compound 2. In some embodiments, the solid form is substantially free of other solid forms (e.g., crystalline forms) of besylate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially physically pure.

In some embodiments, also provided herein is a process for preparing Form B of a besylate salt of Compound 2, comprising exposing Form A of a besylate salt of Compound 2 to a humidified environment, such as 95% RH.

All of the combinations of the above embodiments are encompassed by this application.

5.2.6.8 Form A of a 2-Naphthalenesulfonate Salt of Compound 2

In one embodiment, provided herein is a Form A of a 2-naphthalenesulfonate salt of Compound 2. In one embodiment, Form A is a mono-2-naphthalenesulfonate salt of Compound 2.

A representative XRPD pattern of Form A of a 2-naphthalenesulfonate salt of Compound 2 is provided in FIG. 55.

In one embodiment, provided herein is a solid form comprising a 2-naphthalenesulfonate salt of Compound 2, characterized by 1, 2, 3, 4, 5, 6, 7, 8, 9, 10, 11, 12, 13, 14, 15, 16, 17, 18, or all of the XRPD peaks located at approximately the following positions (e.g., degrees 2θ±0.2) when measured using Cu Kα radiation: 6.8, 7.9, 9.6, 10.6, 11.6, 13.7, 18.1, 18.6, 19.1, 19.8, 20.2, 20.9, 21.3, 21.9, 23.1, 23.7, 24.2, 25.7, and 26.3° 2θ. In one embodiment, the solid form is characterized by at least 3 of the peaks. In one embodiment, the solid form is characterized by at least 5 of the peaks. In one embodiment, the solid form is characterized by at least 7 of the peaks. In one embodiment, the solid form is characterized by at least 9 of the peaks. In one embodiment, the solid form is characterized by at least 11 of the peaks. In one embodiment, the solid form is characterized by all of the peaks.

In one embodiment, provided herein is a solid form (e.g. a crystalline form) comprising a 2-naphthalenesulfonate salt of Compound 2, characterized by an XRPD pattern, when measured using Cu Kα radiation, comprising at least three peaks selected from the group consisting of approximately (e.g., ±0.2°) 6.8, 7.9, 9.6, 10.6, 11.6, 13.7, 18.1, 18.6, 19.1, 19.8, 20.2, 20.9, 21.3, 21.9, 23.1, 23.7, 24.2, 25.7, and 26.3° 2θ. In one embodiment, the solid form is characterized by an XRPD pattern comprising at least four peaks selected from the group consisting of approximately (e.g., ±0.2°) 6.8, 7.9, 9.6, 10.6, 11.6, 13.7, 18.1, 18.6, 19.1, 19.8, 20.2, 20.9, 21.3, 21.9, 23.1, 23.7, 24.2, 25.7, and 26.3° 2θ. In one embodiment, the solid form is characterized by an XRPD pattern comprising at least five peaks selected from the group consisting of approximately (e.g., ±0.2°) 6.8, 7.9, 9.6, 10.6, 11.6, 13.7, 18.1, 18.6, 19.1, 19.8, 20.2, 20.9, 21.3, 21.9, 23.1, 23.7, 24.2, 25.7, and 26.3° 2θ.

In one embodiment, provided herein is a solid form (e.g. a crystalline form) comprising a 2-naphthalenesulfonate salt of Compound 2, characterized by an XRPD pattern comprising peaks at approximately (e.g., ±0.2°) 6.8, 7.9, and 9.6° 2θ. In one embodiment, the XRPD pattern further comprises peaks at approximately (e.g., ±0.2°) 11.6 and 13.7° 2θ. In one embodiment, the XRPD pattern further comprises peaks at approximately (e.g., ±0.2°) 10.6 and 19.8° 2θ. In one embodiment, the XRPD pattern comprises peaks at approximately (e.g., ±0.2°) 6.8, 7.9, 9.6, 10.6, 11.6, 13.7, 18.1, 19.8, and 20.2° 2θ.

In one embodiment, provided herein is a solid form comprising a 2-naphthalenesulfonate salt of Compound 2, characterized by an XRPD pattern that matches the XRPD pattern depicted in FIG. 55. In one embodiment, the Form A that provides FIG. 55 is anhydrous.

In one embodiment, an XRPD pattern described herein is obtained using Cu Kα radiation. In one embodiment, the XRPD pattern is measured by XRPD using Cu Kα radiation comprising Kα1 radiation having a wavelength of 1.5406 Å and Kα₂ radiation having a wavelength of 1.5444 Å.

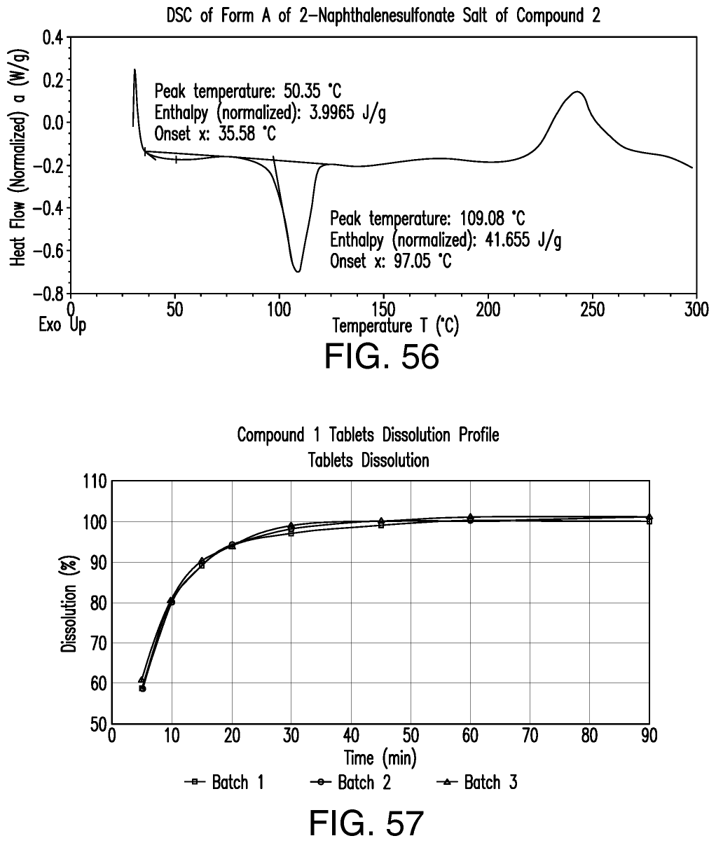

A representative DSC thermogram of Form A of a 2-naphthalenesulfonate salt of Compound 2 are provided in FIG. 56. In one embodiment, provided herein is a solid form comprising a 2-naphthalenesulfonate salt of Compound 2, which exhibits, as characterized by DSC, a first thermal (endo) event with an onset temperature of about 36° C. (e.g. ±2°). In one embodiment, the first thermal event has a peak temperature of about 50° C. (e.g. ±2°). In one embodiment, provided herein is a solid form comprising a 2-naphthalenesulfonate salt of Compound 2, which exhibits, as characterized by DSC, a second thermal (endo) event with an onset temperature of about 97° C. (e.g. ±2°). In one embodiment, the second thermal event has a peak temperature of about 109° C. (e.g. ±2°). In one embodiment, the solid form is characterized by a DSC thermogram that matches the DSC thermogram depicted in FIG. 56. In one embodiment, the DSC thermogram is as measured by DSC using a scanning rate of about 10° C./minute. In one embodiment, the Form A that provides FIG. 56 is an anhydrous 2-naphthalenesulfonate salt of Compound 2.

In some embodiments, provided herein is a solid form comprising a 2-naphthalenesulfonate salt of Compound 2, which is a crystalline anhydrous 2-naphthalenesulfonate salt of Compound 2. In some embodiments, the solid form is substantially free of amorphous 2-naphthalenesulfonate salt of Compound 2. In some embodiments, the solid form is substantially free of other solid forms (e.g., crystalline forms) of 2-naphthalenesulfonate salt of Compound 2. In some embodiments, the solid form is substantially free of the free base form of Compound 2. In some embodiments, the solid form is substantially free of other salts of Compound 2. In some embodiments, the solid form is provided as substantially pure. In some embodiments, the solid form is substantially chemically pure. In some embodiments, the solid form is substantially physically pure.

In some embodiments, also provided herein is a process for preparing a 2-naphthalenesulfonate salt of Compound 2, comprising exposing a composition comprising a free base of Compound 2 to 2-naphthalenesulfonic acid. In one embodiment, the free base of Compound 2 is exposed to 2-naphthalenesulfonic acid in an organic solvent, such as 2-MeTHF.

All of the combinations of the above embodiments are encompassed by this application.

### 5.3 Process for Preparation of Compound 1

In certain embodiments, provided herein is a process of preparing a compound of Formula (II):

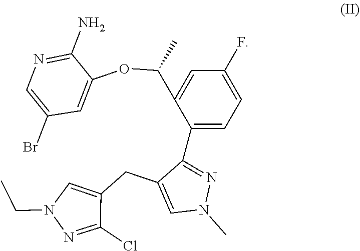 [SMILES: CCn1cc(Cc2cn(C)nc2-c2ccc(F)cc2[C@@H](C)Oc2cc(Br)cnc2N)c(Cl)n1]

or a stereoisomer, or a mixture of stereoisomers thereof, or a pharmaceutically acceptable salt thereof, comprising: (step 2.0) reacting a compound of Formula (III):

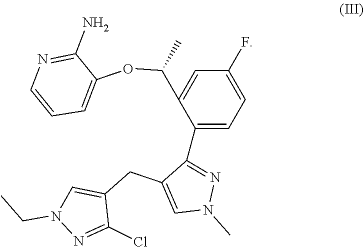 [SMILES: CCn1cc(Cc2cn(C)nc2-c2ccc(F)cc2[C@@H](C)Oc2cccnc2N)c(Cl)n1]

or a stereoisomer, or a mixture of stereoisomers thereof, or a pharmaceutically acceptable salt thereof, with a brominating reagent.

In another embodiment, provided herein is a process of preparing a compound of Formula (II):

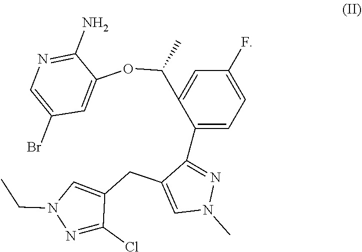 [SMILES: CCn1cc(Cc2cn(C)nc2-c2ccc(F)cc2[C@@H](C)Oc2cc(Br)cnc2N)c(Cl)n1]

or a stereoisomer, or a mixture of stereoisomers thereof, or a pharmaceutically acceptable salt thereof, comprising: (step 2a.1) reacting a compound of Formula (XXIX):

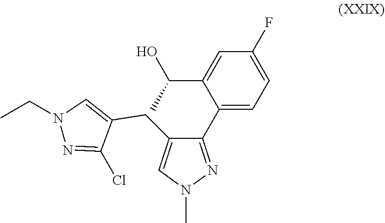 [SMILES: CCn1cc(Cc2cn(C)nc2-c2ccc(F)cc2[C@H](C)O)c(Cl)n1]

or a stereoisomer, or a mixture of stereoisomers thereof, or a pharmaceutically acceptable salt thereof, with a compound of Formula (XXX):

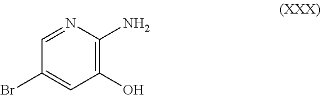

or a pharmaceutically acceptable salt thereof.

In some embodiments, the process further comprises:

(step 1.0) cyclizing the compound of Formula (II), or a stereoisomer, or a mixture of stereoisomers thereof, or a pharmaceutically acceptable salt thereof, to provide a compound of Formula (I):

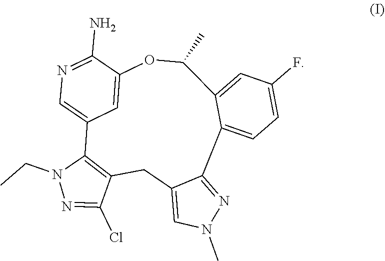 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof.

In some embodiments, a salt of the compound of Formula (II) is cyclized in step 1.0. In some embodiments, a solid form of the salt of the compound of Formula (II) is cyclized in step 1.0. In one embodiment, a camsylate salt of the compound of Formula (II) is cyclized in step 1.0. In one embodiment, a solid form (e.g. Form A) of the camsylate salt of the compound of Formula (II) is cyclized in step 1.0. In another embodiment, wherein step 1.0 comprises:
(step 1.1) converting the camsylate salt of the compound of Formula (II) to a free base of the compound under basic conditions; and
(step 1.2) cyclizing the free base of the compound.

In some embodiments, step 1.0 occurs in the presence of a base. In some embodiments, the base is an organic base. In some embodiments, the organic base is a carboxylate base. In some embodiments, the carboxylate base is lithium acetate, sodium acetate, potassium acetate, lithium pivalate, sodium pivalate, potassium pivalate, cesium acetate, or cesium pivalate. In one embodiment, the base is potassium pivalate.

In some embodiments, the molar ratio of the compound of Formula (II) to base in step 1.0 is from about 1:2 to about 1:6. In one embodiment, the molar ratio of the compound of Formula (II) to base in step 1.0 is about 1:3.

In some embodiments, step 1.0 occurs in the presence of a catalyst precursor. In some embodiments, the catalyst precursor comprises a palladium source. In some embodiments, the palladium source is Pd-G3, Pd₂(dba)₃, PdCl₂(MeCN)₂, Pd(OAc)₂, Pd(PPh₃)₄, PdCl₂(PPh₃)₂, PdCl₂(Pcy₃)₂, PdCl₂(dtbpf), PdCl₂(dppf), PdCl₂(Amphos), {Pd(μ-Br)[P(tBu)₃]}₂, PdCl₂[P(Cy)3]₂, Pd[P(tBu)₃]₂, PdCl₂(dtbpf), Pd[P(Cy)₃]₂, or PdCl₂[P(tBu)(Cy)₂]₂. In one embodiment, the palladium source is Pd(OAc)₂. In one embodiment, the catalyst precursor comprises Pd(OAc)₂. In some embodiments, the catalysts precursor comprises a ligand. In some embodiments, the ligand is a phosphine ligand or bisphosphine ligand. In some embodiments, the ligand is phosphine or bisphosphine ligand commonly used in the art. In one embodiment, the ligand is a cataCXium ligand. In one embodiment, the cataCXium ligand is cataCXium A, cataCXium Abn, cataCXium AHI, cataCXium PintB, cataCXium PICy, cataCXium PtB, cataCXium PomeB, or cataCXium C. In one embodiment, the cataCXium ligand is cataCXium A. In one embodiment, the catalyst precursor comprises cataCXium A. In some embodiments, the catalyst precursor comprises a palladium source and a ligand. In some embodiments, the catalyst precursor and the ligand are pre-formed palladium ligand complexes such as cataCXium A Pd G2, cataCXium A Pd G3, or bis(butyldi-1-adamantylphosphine) palladium diacetate. In one embodiment, the catalyst precursor comprises Pd(OAc)₂ and cataCXium A.

In some embodiments, the molar ratio of the compound of Formula (II) to palladium source (e.g., Pd(OAc)₂) in step 1.0 is from about 1:0.02 (i.e. 2 mol %) to about 1:0.3 (i.e. 30 mol %). In some embodiments, the molar ratio of the compound of Formula (II) to palladium source in step 1.0 is about 1:0.02, about 1:0.03, about 1:0.04, about 1:0.05, about 1:0.06, about 1:0.07, about 1:0.08, about 1:0.09, about 1:0.10, about 1:0.11, about 1:0.12, about 1:0.13, about 1:0.14, about 1:0.15, about 1:0.16, about 1:0.17, about 1:0.18, about 1:0.19, or about 1:0.20. In one embodiment, the molar ratio of the compound of Formula (II) to palladium source in step 1.0 is about 1:0.05 (i.e. 5 mol %). In one embodiment, the molar ratio of the compound of Formula (II) to palladium source in step 1.0 is about 1:0.12 (i.e. 12 mol %). In one embodiment, a palladium loading of less than about 20 mol %, less than about 15 mol %, less than about 10 mol %, or less than about 5 mol %, is employed in step 1.0.

In some embodiments, the molar ratio of the compound of Formula (II) to ligand (e.g., cataCXium ligand) in step 1.0 is from about 1:0.05 (i.e. 5 mol %) to about 1:0.32 (i.e. 32 mol %). In some embodiments, the molar ratio of the compound of Formula (II) to ligand in step 1.0 is about 1:0.05, about 1:0.08, about 1:0.10, about 1:0.16, about 1:0.17, about 1:0.18, about 1:0.19, about 1:0.20, about 1:0.21, about 1:0.22, about 1:0.23, about 1:0.24, about 1:0.25, about 1:0.26, about 1:0.27, about 1:0.28, about 1:0.29, about 1:0.30, about 1:0.31, or about 1:0.32. In one embodiment, the molar ratio of the compound of Formula (II) to ligand in step 1.0 is about 1:0.10 (i.e. 10 mol %). In one embodiment, the molar ratio of the compound of Formula (II) to ligand in step 1.0 is about 1:0.24 (i.e. 24 mol %). In one embodiment, a ligand loading of less than about 30 mol % is employed in step 1.0.

In one embodiment, the molar ratio of the ligand (e.g., cataCXium ligand) to the palladium source (e.g., Pd(OAc)₂) in step 1.0 is from about 5:1 to about 1:5. In one embodiment, the molar ratio of the ligand to the palladium source is from about 2:1 to about 1:2. In one embodiment, the molar ratio of the ligand to the palladium source is from about 2:1 to about 1:1. In one embodiment, the ligand is a monodentate ligand and the molar ratio of the ligand to the palladium source is about 2:1. In one embodiment, the ligand is a monodentate ligand and the molar ratio of the ligand to the palladium source is about 1:1. In one embodiment, the ligand is a bidentate ligand and the molar ratio of the ligand to the palladium source is about 1:1. In one embodiment, the ligand is a bidentate ligand and the molar ratio of the ligand to the palladium source is about 1:2.

Step 1.0 may occur in a solvent suitable for the reaction. In some embodiments, the solvent is an organic solvent or a mixture of organic solvents. In one embodiment, the solvent is a high-boiling solvent, including but not limited to C₄₋₁₂ aliphatic alcohol (branched or unbranched), anisole, 2-MeTHF, DMF, NMP, DMA or tAmOH. In one embodiment, the solvent is an alcohol. In one embodiment, the solvent is t-amyl alcohol (tAmOH). In one embodiment, the solvent is n-BuOH, s-BuOH, or t-BuOH.

In some embodiments, the volume of solvent in step 1.0 is from about 10 vol to about 30 vol. In one embodiment, the volume of the solvent in step 1.0 is about 20 vol.

As used herein, vol refers to the volume (L or mL) of a solvent relevant to the weight (kg or g respectively) of the limiting reagent. In some embodiments, step 1.0 occurs in an inert atmosphere (i.e. under conditions which eliminate or substantially reduce the presence of atmospheric oxygen). In one embodiment, the solvent is sparged with an inert gas (e.g. dinitrogen or argon) in step 1.0.

In some embodiments, step 1.0 occurs at a reaction temperature of from about 90° C. to about 120° C. In one embodiment, the reaction temperature is the boiling temperature of the solvent. In one embodiment, the reaction temperature is from about 100° C. to about 110° C. In one embodiment, the reaction temperature is about 102° C.

In some embodiments, step 1.0 occurs at a reaction time from about 16 hours to about 30 hours. In one embodiment, the reaction time is from about 16 hours to about 20 hours.

In one embodiment, step 1.0 occurs in the presence of potassium pivalate base and a catalyst precursor comprising Pd(OAc)₂ and cataCXium A. In one embodiment, the molar ratios of the compound of Formula (II) to potassium pivalate, Pd(OAc)₂ and cataCXium A are about 1:3, about 1:0.12, and about 1:0.24, respectively. In one embodiment, the molar ratios of the compound of Formula (II) to potassium pivalate, Pd(OAc)₂ and cataCXium A are about 1:0.05, and about 1:0.1, respectively. In one embodiment, step 1.0 occurs in a solvent of t-amyl alcohol and a solvent volume of 20 vol at a reaction temperature of about 100-110° C. In one embodiment, the solvent is sparged with nitrogen gas in step 1.0. In some embodiments, a salt of the compound of Formula (II) is used in step 1.0. In one embodiment, a camsylate salt of the compound of Formula (II) is used in step 1.0. In another embodiment, wherein step 1.0 comprises:
(step 1.1) converting the camsylate salt of the compound of Formula (II) to a free base of the compound under basic conditions; and
(step 1.2) cyclizing the free base of the compound.

In some embodiments, step 1.0 proceeds to greater than about 90%, greater than about 95%, greater than about 96%, greater than about 97%, greater than about 98%, or greater than about 99% conversion within about 18 hours, as determined by HPLC and/or NMR. In some embodiments, step 1.0 provides less than about 10%, less than about 5%, less than about 4%, less than about 3%, less than about 2%, or less than about 1% of an impurity distinct from the compound of Formula (I). Impurities provided in step 1.0 may include, but are not limited to, the compound of Formula (II), the compound of Formula (III), a compound of Formula (V) described herein below, a t-butylcarbonylated species of Formula (SP-1), and/or a dechlorinated species of Formula (SP-2).

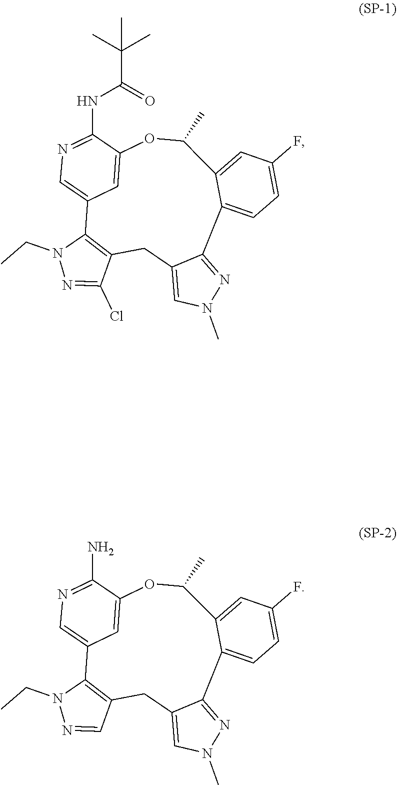 [SMILES: CCn1nc(Cl)c2c1-c1cnc(NC(=O)C(C)(C)C)c(c1)O[C@H](C)c1cc(F)ccc1-c1nn(C)cc1C2 ; CCn1ncc2c1-c1cnc(N)c(c1)O[C@H](C)c1cc(F)ccc1-c1nn(C)cc1C2]

In one embodiment, the total amount of impurities provided in step 1.0 is less than about 10 wt %, less than about 8 wt %, less than about 5 wt %, less than about 4 wt %, less than about 3 wt %, less than about 2 wt %, less than about 1 wt %, less than about 0.5 wt %, less than about 0.1 wt %, less than about 0.05 wt %, less than about 0.03 wt %, or less than about 0.02 wt %.

In some embodiments, step 1.0 further comprises purification of the compound of Formula (I). In certain embodiments, the compound of Formula (I) produced in step 1.0 is purified by chromatography, palladium remediation, and/or (re)crystallization.

In one embodiment, the palladium remediation comprises treatment with a palladium scavenger. In one embodiment, the palladium scavenger is a thiopropyl silica scavenger. In one embodiment, the reaction mixture is stirred with thiopropyl silica scavenger and subsequently filtered off with the filter cake rinsed with t-AmOH. In one embodiment, the reaction mixture is stirred with thiopropyl silica scavenger and subsequently filtered off with the filter cake rinsed with MTBE. In one embodiment, the palladium scavenger is an aqueous solution of L-cysteine. In one embodiment, the combined filtrates from the thiopropyl silica scavenger treatment are concentrated and further treated with L-cysteine. In one embodiment, the palladium remediation occurs at a temperature above room temperature, e.g., from about 40° C. to about 80° C., e.g., about 60° C. In one embodiment, the palladium remediation comprises treatment with a palladium scavenger for a period of about 1 hour, greater than 1 hour, greater than 4 hours, greater than 10 hours, greater than 14 hours, greater than 16 hours, or about 16 hours. In one embodiment, the palladium remediation comprises more than one treatment with a palladium scavenger, e.g., two treatments, three treatments, or four treatments with a palladium scavenger. In one embodiment, the palladium remediation comprises separate treatments with two different palladium scavengers. In one embodiment, the palladium remediation comprises separate treatments with the same palladium scavenger.

In one embodiment, the compound of Formula (I) is crystallized or recrystallized from an organic solvent or a mixture of organic solvents. In one embodiment, crystallization or recrystallization of the compound of Formula (I) provides Form 2 of Compound 1. In one embodiment, the compound of Formula (I) is crystallized or recrystallized from ethanol, ethyl acetate, acetonitrile, TBME, isobutyl acetate, CPME, or a mixture thereof, optionally by addition of an anti-solvent. In one embodiment, the anti-solvent is a non-polar organic solvent. In one embodiment, the non-polar organic solvent is a hydrocarbon solvent. In one embodiment, the anti-solvent is heptane. In one embodiment, the solvent is ethanol and the anti-solvent is heptane. In one embodiment, the solvent is ethyl acetate and the anti-solvent is heptane. In one embodiment, the final volume ratio of solvent to anti-solvent is from about 1:2 to about 1:10. In one embodiment, the final volume ratio of solvent to anti-solvent is from about 1:4 to about 1:10. In one embodiment, the final volume ratio of solvent to anti-solvent is from about 1:6 to about 1:10. In one embodiment, the final volume ratio of solvent to anti-solvent is about 1:8. In one embodiment, the compound of Formula (I) is dissolved or suspended in a mixture of solvent and anti-solvent. In one embodiment, additional anti-solvent is added the solution or suspension of the compound of Formula (I). In one embodiment, the solution or suspension of the compound of Formula (I) is cycled between an elevated temperature and a reduced temperature. In one embodiment, the elevated temperature is a temperature above room temperature, such as, but not limited to a temperature of between about 30° C. and about 60° C., e.g. about 40-45° C. In one embodiment, the reduced temperature is a temperature below room temperature, such as, but not limited to a temperature of between about 0° C. and about 20° C., e.g. about 10-15° C. In one embodiment, the temperature is cycled at least one time, at least two times, at least three times, at least four times, or at least five times. In one embodiment, the crude Compound 1 is dissolved in mixed solvents of EtOH and heptane, treated with additional heptane and a seed amount of Form 2 of Compound 1. In one embodiment, the crude Compound 1 is dissolved in mixed solvents of EtOAc and heptane, treated with additional heptane and a seed amount of Form 2 of Compound 1. In certain embodiment, the seed amount is about 0.01 mol % to about 9.0 mol % of the crude Compound 1. In certain embodiments, the seed amount is about 0.05 mol %, about 0.1 mol %, about 0.5 mol %, about 1.0 mol %, about 2.0 mol %, about 3.0 mol %, about 4.0 mol %, about 5.0 mol %, about 6.0 mol %, about 7.0 mol %, or about 8.0 mol % of the crude Compound 1. In certain embodiments, the seed amount is about 5 mol % of the crude Compound 1. In certain embodiments, the seed amount is about 2 mol % of the crude Compound 1.

In certain embodiments, step 1.0 provides a compound of Formula (I) in a substantially pure form. In certain embodiments, step 1.0 provides a compound of Formula (I) in a substantially chemically pure form (e.g. at least 95 wt %, at least 96 wt %, at least 97 wt %, at least 98 wt %, or at least 99 wt %). In certain embodiments, step 1.0 provides a compound of Formula (I) in a substantially enantiomerically pure form (e.g. at least at least 97 wt %, at least 98 wt %, at least 99 wt %, or at least 99.5%). In certain embodiments, step 1.0 provides a compound of Formula (I) substantially free of impurities. In certain embodiments, step 1.0 provides a composition comprising Compound 1 having a residual palladium content of less than about 200 ppm, less than about 100 ppm, less than about 50 ppm, less than about 40 ppm, less than about 30 ppm, less than about 20 ppm, or less than about 10 ppm. In certain embodiments, step 1.0 provides a compound of Formula (I) in a substantially enantiomerically pure form. In certain embodiments, step 1.0 provides a compound of Formula (I) in a substantially physically pure form. In certain embodiments, step 1.0 provides a compound of Formula (I) in a solid form having a desired morphology (e.g. a specific crystalline form, such as Form 2 of Compound 1) or advantageous rheological properties.

In certain embodiments, step 1.0 further comprises milling Form 2 of Compound 1 after the crystallization or recrystallization. In certain embodiments, the milling is dry milling, jet milling, or wet milling. In certain embodiments, the milling is jet milling. In certain embodiments, the milling is wet milling. In certain embodiments, the milling is wet milling (e.g., at a temperature of about 23-27° C.). In certain embodiments, the particles after the milling are of a consistent size and still of Form 2. In certain embodiments, the particle size ranges from about 1 μm to about 100 μm. In some embodiments, the particle size ranges from about 10 μm to about 80 μm. In some embodiments, the particle size ranges from about 10 μm to about 60 μm. The term “about,” as used herein with respect to particle size, means+/−5 μm.

In some embodiments, at least 90% of a representative sample of the particles after milling has a particle size of no more than about 100, about 80, about 70, about 60, about 50, about 40, about 30, about 20, or about 10 μm. In some embodiments, at least about 90% of a representative sample of the particles after milling has a particle size of no more than about 90 μm. In some embodiments, at least about 90% of a representative sample of the particles after milling has a particle size of no more than about 80 μm. In some embodiments, at least about 90% of a representative sample of the particles after milling has a particle size of no more than about 70 μm. In some embodiments, at least about 90% of a representative sample of the particles after milling has a particle size of no more than about 60 μm. In some embodiments, at least about 90% of a representative sample of the particles after milling has a particle size of no more than about 50 μm. In some embodiments, at least about 90% of a representative sample of the particles after milling has a particle size of no more than about 40 μm. In some embodiments, at least about 90% of a representative sample of the particles after milling has a particle size of no more than about 30 μm. In some embodiments, at least about 90% of a representative sample of the particles after milling has a particle size of no more than about 20 μm. In some embodiments, at least about 90% of a representative sample of the particles after milling has a particle size of no more than about 10 μm. In some embodiments, at least 90% of a representative sample of the particles after milling has a particle size of about 100 μm to about 10 μm. In some embodiments, at least 90% of a representative sample of the particles after milling has a particle size of about 60 μm to about 20 μm. In one embodiment, at least 90% of a representative sample of the particles after milling has a particle size of about 19 μm to about 106 μm.

In some embodiments, about 50% of a representative sample of the particles after milling has a particle size of about 50 μm to about 1 μm. In some embodiments, about 50% of a representative sample of the particles after milling has a particle size of about 30 μm to about 5 μm. In some embodiments, about 50% of a representative sample of the particles after milling has a particle size of about 20 μm to about 5 μm. In some embodiments, about 50% of a representative sample of the particles after milling has a particle size of about 20 μm to about 10 μm. In one embodiment, about 50% of a representative sample of the particles after milling has a particle size of about 47 μm to about 10 μm. In some embodiments, at least 50% of a representative sample of the particles after milling has a particle size of about 50 μm to about 1 μm. In one embodiment, at least 50% of a representative sample of the particles after milling has a particle size of about 47 μm to about 10 μm.

In some embodiments, about 10% of a representative sample of the particles after milling has a particle size of about 40 μm to about 1 μm. In some embodiments, about 10% of a representative sample of the particles after milling has a particle size of about 30 μm to about 1 μm. In some embodiments, about 10% of a representative sample of the particles after milling has a particle size of about 20 μm to about 1 μm. In some embodiments, about 10% of a representative sample of the particles after milling has a particle size of about 10 μm to about 1 μm. In some embodiments, about 10% of a representative sample of the particles after milling has a particle size of about 5 μm to about 1 μm. In one embodiment, about 10% of a representative sample of the particles after milling has a particle size of about 15 μm to about 4 μm. In some embodiments, at least 10% of a representative sample of the particles after milling has a particle size of about 40 μm to about 1 μm. In one embodiment, at least 10% of a representative sample of the particles after milling has a particle size of about 15 μm to about 4 μm.

In some embodiments, the brominating reagent in step 2.0 is bromine (Br₂), N-bromosuccinimide (NBS), phosphorus tribromide (PBr₃), 3-bromo-5,5-dimethylhydantoin, 1,3-dibromo-5,5-dimethylhydantoin, sodium monobromoisocyanurate, N-bromophthalimide, or hydrobromic acid/hydrogen peroxide (HBr/HOOH). In one embodiment, the brominating reagent is N-bromosuccinimide (NBS).

In some embodiments, the molar ratio of the compound of Formula (III) to brominating reagent in step 2.0 is from about 1:0.95 to about 1:2. In one embodiment, the molar ratio of the compound of Formula (III) to brominating reagent is 1:1.05. In one embodiment, the molar ratio of the compound of Formula (III) to brominating reagent is 1:1.03.

Step 2.0 may occur in a solvent suitable for the reaction. In some embodiments, the solvent is an organic solvent or a mixture of organic solvents. In one embodiment, the organic solvent is THF. In one embodiment, the organic solvent is acetonitrile. In one embodiment, the organic solvent is DCM. In one embodiment, the organic solvent is 2-MeTHF. In one embodiment, the organic solvent is EtOAc. In one embodiment, the organic solvent is isopropyl acetate.

In some embodiments, the weight ratio of the solvent to the compound of Formula (III) in step 2.0 is from about 5:1 to about 25:1. In one embodiment, the weight ratio of the solvent to the compound of Formula (III) in step 2.0 is about 9:1.

In some embodiments, step 2.0 occurs in an inert atmosphere (i.e. under conditions which eliminate or substantially reduce the presence of atmospheric oxygen). In one embodiment, the solvent is sparged with an inert gas (e.g. nitrogen or argon) in step 2.0.

In some embodiments, step 2.0 occurs at a reaction temperature of from about −20° C. to about 10° C. In one embodiment, the reaction temperature is from about −10° C. to about 0° C.

In some embodiments, step 2.0 occurs at a reaction time of from about 10 minutes to about 3 hours. In one embodiment, the reaction time is from about 30 minutes to about 1 hour.

In one embodiment, the brominating reagent in step 2.0 is NBS and the molar ratio of the compound of Formula (III) to NBS is about 1:1.03. In one embodiment, step 2.0 occurs in a solvent of THF and a solvent weight of about 9 folds relevant to the compound of Formula (III) at a reaction temperature of about −10° C. to about 0° C. In one embodiment, the solvent is sparged with dinitrogen gas in step 2.0.

In some embodiments, step 2.0 proceeds to greater than 90%, greater than 95%, greater than 96%, greater than 97%, greater than 98%, or greater than 99% conversion within about 1 hour, as determined by HPLC and/or NMR. In some embodiments, step 2.0 provides less than about 10%, less than about 5%, less than about 4%, less than about 3%, less than about 2%, or less than about 1% of an impurity distinct from the compound of Formula (II). Impurities provided in step 2.0 may include, but are not limited to, the compound of Formula (III), the compound of Formula (V), and/or a compound of Formula (SP-3).

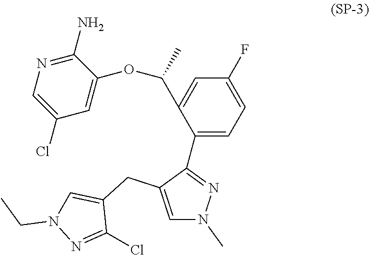 [SMILES: CCn1cc(Cc2cn(C)nc2-c2ccc(F)cc2[C@@H](C)Oc2cc(Cl)cnc2N)c(Cl)n1]

In one embodiment, the total amount of impurities provided in step 2.0 is less than about 10 wt %, less than about 8 wt %, less than about 5 wt %, less than about 4 wt %, less than about 3 wt %, less than about 2 wt %, less than about 1 wt %, less than about 0.5 wt %, less than about 0.1 wt %, or less than about 0.05 wt %.

In some embodiments, step 2.0 further comprises purification of the compound of Formula (II). In certain embodiments, the compound of Formula (II) produced in step 2.0 is purified by quenching with a reducing agent, treatment with activated carbon, and/or treatment with silica. In one embodiment, the reducing agent is an aqueous solution of Na₂S₂O₃.

In certain embodiments, step 2.0 further comprises converting a free base form of the compound of Formula (II) to a salt of the compound. In some embodiments, step 2.0 comprises converting a free base form of the compound of Formula (II) to a camsylate salt of the compound. In one embodiment, in step 2.0, the free base form the compound of Formula (II) is reacted with camphor sulfonic acid to provide the camsylate salt of the compound. In one embodiment, the free base form of the compound of Formula (II) is reacted with camphor sulfonic acid in a solvent comprising MeOAc and/or heptane (e.g. a 1:1 mixture of MeOAc and heptane). In another embodiment, the free base form of the compound of Formula (II) is reacted with camphor sulfonic acid in a solvent comprising isopropyl acetate. In another embodiment, the free base form of the compound of Formula (II) is reacted with camphor sulfonic acid in a solvent comprising ethyl acetate; ethyl acetate and t-amyl alcohol; or ethyl acetate and heptane. In certain embodiments, the camsylate salt of the compound of Formula (II) is isolated as a solid form (e.g. Form A) of the camsylate salt. In certain embodiments, the isolated solid form of the camsylate salt of the compound of Formula (II) has improved chemical and/or physical purity as compared to the free base form of the compound prepared in step 2.0. In certain embodiments, the camsylate salt of the compound of Formula (II) is more easily isolated and/or worked-up than the free base form of the compound prepared in step 2.0.

In certain embodiments, step 2.0 provides a compound of Formula (II) in a substantially pure form. In certain embodiments, step 2.0 provides a compound of Formula (II) in a substantially chemically pure form. In certain embodiments, step 2.0 provides a compound of Formula (II) in a substantially enantiomerically pure form. In certain embodiments, step 2.0 provides a compound of Formula (II) substantially free of impurities and easy scale up. In certain embodiments, step 2.0 reduces, eliminates or minimizes the amount of impurities carried forward into step 1.0.

In certain embodiments, the reaction of the compound of Formula (XXIX) with the compound of Formula (XXX) in step 2a.1 occurs via a Mitsunobu reaction. In certain embodiments, step 2a.1 occurs in the presence of a diazene and a phosphine. In other embodiments, step 2a.1 occurs in the presence of cyanomethylenetributylphosphorane (Tsunoda reagent).

In certain embodiments, the diazene in step 2a.1 is an azodicarboxamide compound (e.g. tetramethylazodicarboxamide or, “TMAD”) or an azodicarboxylate (e.g. diethylazodicarboxylate, “DEAD”). In certain embodiments, the diazene is DIAD (diisopropyl azodicarboxylate), DtBAD (di(t-butyl) azodicarboxylate), ADDP (azodicarbonyl dipiperidine), DCAD (dicyclohexyl azodicarboxylate), or Dibenzyl azodicarboxylate. In one embodiment, the diazene is TMAD.

In certain embodiments, the phosphine in step 2a.1 is triphenylphosphine, tricyclohexylphosphine, or bis(dicyclohexylphosphino)ethane. In certain embodiments, the phosphine in step 2a.1 is a trialkyl phosphine. In one embodiment, the trialkyl phosphine is nBu₃P.

In some embodiments, the molar ratio of the compound of Formula (XXIX) to the compound of Formula (XXX) in step 2a.1 is from about 1:0.95 to about 1:2. In one embodiment, the molar ratio of the compound of Formula (XXIX) to the compound of Formula (XXX) is 1:1.3.

In some embodiments, the molar ratio of the compound of Formula (XXIX) to the diazene in step 2a.1 is from about 1:1 to about 1:2. In one embodiment, the molar ratio of the compound of Formula (XXIX) to the diazene is 1:1.3. In one embodiment, the molar ratio of the compound of Formula (XXIX) to the diazene is about 1:1.6.

In some embodiments, the molar ratio of the compound of Formula (XXIX) to the phosphine in step 2a.1 is from about 1:1 to about 1:2. In one embodiment, the molar ratio of the compound of Formula (XXIX) to the phosphine is 1:1.3. In one embodiment, the molar ratio of the compound of Formula (XXIX) to the phosphine is about 1:1.6.

In certain embodiments, step 2a.1 occurs in the presence of a base. In some embodiments, step 2a.1 occurs in the presence of an organic base. In some embodiments, the organic base is nitrogen containing base. In some embodiments, step 2a.1 occurs in the presence of NH₄OH, triethylamine, diisopropylethylamine (DIEA or DIPEA), pyridine, lutidine, 4-dimethylaminopyridine, imidazole, or 1,8-diazabicyclo[5.4.0]undec-7-ene (DBU). In one embodiment, the base is 1,8-diazabicyclo[5.4.0]undec-7-ene (DBU).

In some embodiments, the molar ratio of the compound of Formula (XXIX) to the base in step 2a.1 is from about 1:1 to about 1:2. In one embodiment, the molar ratio of the compound of Formula (XXIX) to the diazene is 1:1.3. In one embodiment, the molar ratio of the compound of Formula (XXIX) to the base is about 1:1.6.

Step 2a.1 may occur in a solvent suitable for the reaction. In some embodiments, the solvent is an organic solvent or a mixture of organic solvents. In one embodiment, the organic solvent is THF. In one embodiment, the organic solvent is 2-MeTHF.

In some embodiments, step 2a.1 occurs at a reaction temperature of from about 20° C. to about 30° C. In one embodiment, the reaction temperature is room temperature.

In some embodiments, step 2a.1 occurs at a reaction time of from about 30 minutes to about 3 hours.

In one embodiment, the diazene in step 2a.1 is TMAD, the phosphine is nBu₃P, and the base is DBU. In one embodiment, the molar ratios of the compound of Formula (XXIX) to the compound of Formula (XXX), the diazene, the phosphine, and the base are each 1:1.3, respectively. In one embodiment, step 2.0 occurs in a solvent of THF at a room temperature.

In certain embodiments, step 2a.1 further comprises converting a free base form of the compound of Formula (II) to a salt of the compound. In some embodiments, step 2a.1 comprises converting a free base form of the compound of Formula (II) to a camsylate salt of the compound. In one embodiment, in step 2a.1, the free base form the compound of Formula (II) is reacted with camphor sulfonic acid to provide the camsylate salt of the compound. In one embodiment, the free base form of the compound of Formula (II) is reacted with camphor sulfonic acid in a solvent comprising MeOAc and/or heptane (e.g. a 1:1 mixture of MeOAc and heptane). In another embodiment, the free base form of the compound of Formula (II) is reacted with camphor sulfonic acid in a solvent comprising isopropyl acetate. In another embodiment, the free base form of the compound of Formula (II) is reacted with camphor sulfonic acid in a solvent comprising ethyl acetate; ethyl acetate and t-amyl alcohol; or ethyl acetate and heptane. In certain embodiments, the camsylate salt of the compound of Formula (II) is isolated as a solid form (e.g. Form A) of the camsylate salt. In certain embodiments, the isolated solid form of the camsylate salt of the compound of Formula (II) has improved chemical and/or physical purity as compared to the free base form of the compound prepared in step 2a.1. In certain embodiments, the camsylate salt of the compound of Formula (II) is more easily isolated and/or worked-up than the free base form of the compound prepared in step 2a.1.

In certain embodiments, step 2a.1 provides a compound of Formula (II) in a substantially pure form. In certain embodiments, step 2a.1 provides a compound of Formula (II) in a substantially chemically pure form. In certain embodiments, step 2a.1 provides a compound of Formula (II) in a substantially enantiomerically pure form. In certain embodiments, step 2a.1 provides a compound of Formula (II) substantially free of impurities and easy scale up. In certain embodiments, step 2a.1 reduces, eliminates or minimizes the amount of impurities carried forward into step 1.0.

In certain embodiments, also provided herein is a process of preparing a compound of Formula (III), or a stereoisomer, or a mixture of stereoisomers thereof, or a pharmaceutically acceptable salt thereof, comprising:

(step 3.0) reducing a compound of Formula (IV):

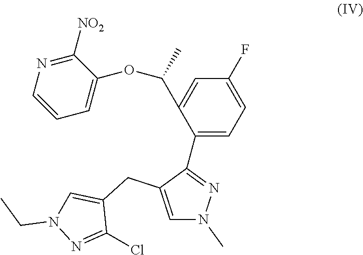 [SMILES: CCn1cc(Cc2cn(C)nc2-c2ccc(F)cc2[C@@H](C)Oc2cccnc2[N+](=O)[O-])c(Cl)n1]

or a stereoisomer, or a mixture of stereoisomers thereof, or a pharmaceutically acceptable salt thereof.

In some embodiments, step 3.0 occurs in the presence of a catalyst. In some embodiments, the catalyst is a platinum catalyst. In some embodiments, the catalyst is Pt/C. In some embodiments, the catalyst is Pt—V/C (platinum-vanadium on carbon).

In some embodiments, step 3.0 occurs in the presence of a reducing agent. In some embodiments, the reducing agent is a metallic reducing agent. In some embodiment, the metallic reducing agent is Fe⁰. In some embodiments, the reducing agent is a source of hydrogen. In some embodiments, the source of hydrogen is H₂ gas, a source of H atoms, and/or a hydride source. In some embodiments, the source of hydrogen is a borohydride reagent. In some embodiments, the source of hydrogen is formic acid. In some embodiments, the source of hydrogen is a formate salt of ammonium or a protonated amine. In some embodiments, the source of hydrogen is triethylammonium formate (HCOOH·Et₃N). In some embodiments, the source of hydrogen is H₂.

In some embodiment, the molar ratio of the compound of Formula (IV) to the reducing agent (e.g. triethylammonium formate) in step 3.0 is from about 1:6 to about 1:12. In one embodiment, the molar ratio of the compound of Formula (IV) to the reducing agent in step 3.0 is about 1.9.

In some embodiments, step 3.0 occurs in the presence of a base. In some embodiments, step 3.0 occurs in the presence of an organic base. In some embodiments, the organic base is nitrogen containing base. In some embodiments, step 3.0 occurs in the presence of NH₄OH, triethylamine, diisopropylethylamine (DIEA or DIPEA), pyridine, lutidine, 4-dimethylaminopyridine, imidazole, or 1,8-diazabicyclo[5.4.0]undec-7-ene (DBU). In one embodiment, the base is triethylamine (TEA).

In some embodiments, the molar ratio of the compound of Formula (IV) to base in step 3.0 is from about 1:3 to about 1:10. In one embodiment, the molar ratio of the compound of Formula (IV) to base in step 3.0 is about 1:4.5.

In some embodiments, the molar ratio of the source of hydrogen to base in step 3.0 is from about 2:1 to about 1:2. In one embodiment, the molar ratio of the source of hydrogen to base in step 3.0 is about 2:1.

Step 3.0 may occur in a solvent suitable for the reaction. In some embodiments, the solvent is an organic solvent or a mixture of organic solvents. In some embodiments, the solvent is a protic solvent. In some embodiments, the solvent is an alcohol solvent. In some embodiments, the solvent is methanol, ethanol, t-butanol, or 2-propanol. In one embodiment, the solvent is ethanol. In one embodiment, the solvent is ethyl acetate.

In some embodiments, the volume of solvent in step 3.0 is from about 5 vol to about 15 vol. In one embodiment, the volume of the solvent in step 3.0 is 10 vol.

In some embodiments, step 3.0 occurs in an inert atmosphere (i.e. under conditions which eliminate or substantially reduce the presence of atmospheric oxygen). In one embodiment, the solvent is sparged with an inert gas (e.g. dinitrogen or argon) in step 3.0.

In some embodiments, step 3.0 occurs at a reaction temperature of from about 40° C. to about 80° C. In one embodiment, the reaction temperature is about 50-55° C. In one embodiment, the reaction temperature is about 65-70° C.

In some embodiments, step 3.0 occurs at a reaction time of from about 15 hours to about 30 hours. In one embodiment, the reaction time is about 20 hours.

In one embodiment, step 3.0 occurs in the presence of a catalyst, a source of hydrogen, and a base, wherein the catalyst is Pt/C, the source of hydrogen is formic acid, and the base is triethylamine. In one embodiment, the molar ratio of the compound of Formula (IV) to formic acid and triethylamine in step 3.0 is about 1:9 and about 1:4.5, respectively. In one embodiment, step 3.0 occurs in a solvent of ethanol and a solvent volume of 10 vol. at a temperature of about 65-70° C. In one embodiment, the solvent is sparged with nitrogen gas in step 3.0.

In one embodiment, step 3.0 occurs in the presence of a catalyst and a source of hydrogen, wherein the catalyst is Pt/C, the source of hydrogen is H₂. In one embodiment, step 3.0 occurs in the presence of a catalyst and a source of hydrogen, wherein the catalyst is Pt—V/C, the source of hydrogen is H₂. In one embodiment, the ratio of the catalyst to the compound of Formula (IV) is about 3 wt % to 8 wt %. In one embodiment, step 3.0 occurs in a solvent of EtOAc and a solvent weight of about 4 to 5 folds at a temperature of about 20-30° C. In one embodiment, the solvent is sparged with nitrogen gas in step 3.0.

In some embodiments, step 3.0 proceeds to greater than 90%, greater than 95%, greater than 96%, greater than 97%, greater than 98%, or greater than 99% conversion within about 20 hours, as determined by HPLC and/or NMR. In some embodiments, step 3.0 provides less than about 10%, less than about 5%, less than about 4%, less than about 3%, less than about 2%, or less than about 1% of an impurity distinct from the compound of Formula (III). Impurities provided in step 3.0 may include, but are not limited to, the compound of Formula (IV), the compound of Formula (V), a compound of Formula (SP-4), and a compound of Formula (SP-5).

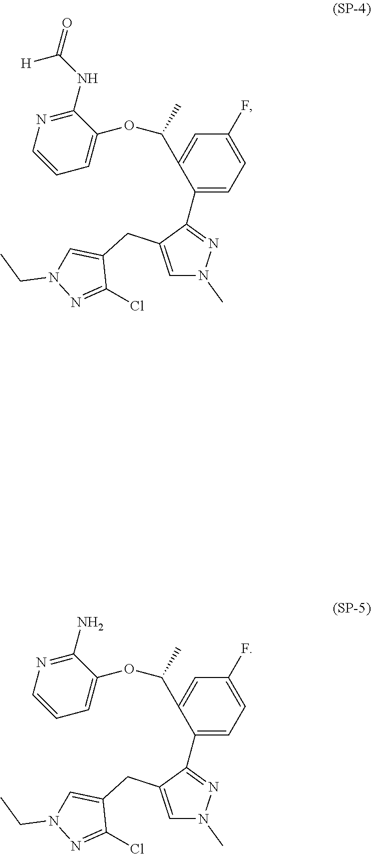 [SMILES: CCn1cc(Cc2cn(C)nc2-c2ccc(F)cc2[C@@H](C)Oc2cccnc2NC=O)c(Cl)n1 ; CCn1cc(Cc2cn(C)nc2-c2ccc(F)cc2[C@@H](C)Oc2cccnc2N)c(Cl)n1]

In some embodiments, the impurity of Formula (SP-4) is formed during the transfer hydrogenation conditions (step 3.0). In some embodiments, the impurity of Formula (SP-4) is formed in the presence of formic acid and triethylamine with Pt/C as a catalyst.

In one embodiment, the total amount of impurities provided in step 3.0 is less than about 10%, less than about 5 wt %, less than about 4 wt %, less than about 3 wt %, less than about 2 wt %, or less than about 1 wt %.

In some embodiments, step 3.0 further comprises purification of the compound of Formula (III). In certain embodiments, the compound of Formula (III) produced in step 3.0 is purified by precipitation from a solvent by an anti-solvent and/or (re)crystallization.

In one embodiment, the compound of Formula (III) is precipitated from an organic solvent. In one embodiment, the compound of Formula (III) is precipitated from ethyl acetate. In one embodiment, the compound of Formula (III) is precipitated from a solvent by addition of an anti-solvent. In one embodiment, the anti-solvent is heptane. In one embodiment, the anti-solvent is methylcyclohexane (MCH). In one embodiment, the solvent is ethyl acetate and the anti-solvent is heptane. In one embodiment, the solvent is ethyl acetate and the anti-solvent is MCH.

In certain embodiments, step 3.0 provides a compound of Formula (III) in a substantially pure form. In certain embodiments, step 3.0 provides a compound of Formula (III) in a substantially chemically pure form. In certain embodiments, step 3.0 provides a compound of Formula (III) in a substantially enantiomerically pure form. In certain embodiments, step 3.0 provides a compound of Formula (III) substantially free of impurities and easy scale up. In certain embodiments, step 3.0 reduces, eliminates or minimizes the amount of impurities carried forward into step 2.0.

In certain embodiments, also provided herein is a process for preparing a compound of Formula (IV), or a stereoisomer, or a mixture of stereoisomers thereof, or a pharmaceutically acceptable salt thereof, comprising:

(step 4.0) reacting a compound of Formula (V):

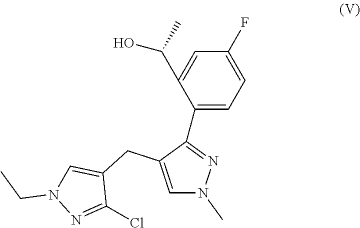 [SMILES: CCn1cc(Cc2cn(C)nc2-c2ccc(F)cc2[C@@H](C)O)c(Cl)n1]

or a stereoisomer, or a mixture of stereoisomers thereof, or a pharmaceutically acceptable salt thereof, with a compound of Formula (VI):

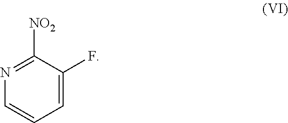

In some embodiments, the molar ratio of the compound of Formula (V) to the compound of Formula (VI) in step 4.0 is from about 1:1 to about 1:1.5. In one embodiment, the molar ratio of the compound of Formula (V) to the compound of Formula (VI) in step 4.0 is about 1:1.1. In one embodiment, the molar ratio of the compound of Formula (V) to the compound of Formula (VI) in step 4.0 is about 1:1.2.

In some embodiments, step 4.0 occurs in the presence of a base. In some embodiments, step 4.0 occurs in the presence of an alkali metal base. In some embodiments, the base is an alkali metal hydride, hydroxide, alkoxide, carbonate, hydrogencarbonate, phosphate, hydrogenphosphate, or dihydrogenphosphate. In some embodiments, the base is NaH, KH, LiOH, NaOH, KOH, NaO^{t}Bu, KO^{t}Bu, Na₂CO₃, K₂CO₃, Cs₂CO₃, NaHCO₃, KHCO₃, Na₃PO₄, K₃PO₄, Na₂HPO₄, K₂HPO₄, NaH₂PO₄, or KH₂PO₄. In one embodiment, the base is potassium t-butoxide (KO^{t}Bu).

In some embodiments, the molar ratio of the compound of Formula (V) to base in step 4.0 is from about 1:1 to about 1:2. In one embodiment, the molar ratio of the compound of Formula (V) to base is about 1:1.5. In one embodiment, the molar ratio of the compound of Formula (V) to base is about 1:1.2 to about 1:1.4.

Step 4.0 may occur in a solvent suitable for the reaction. In some embodiments, the solvent is an organic solvent or a mixture of organic solvents. In one embodiment, step 4.0 occurs in a solvent of toluene, THF, or a mixture thereof. In one embodiment, step 4.0 occurs in a solvent of toluene, THF, or 2-Me THF.

In some embodiments, the weight ratio of solvent to the compound of Formula (V) in step 4.0 is from about 3:1 to 8:1.

In some embodiments, step 4.0 occurs in an inert atmosphere (i.e. under conditions which eliminate or substantially reduce the presence of atmospheric oxygen). In one embodiment, the solvent is sparged with an inert gas (e.g. nitrogen or argon) in step 4.0.

In some embodiments, step 4.0 occurs at a reaction temperature below room temperature. In some embodiments, step 4.0 occurs at a reaction temperature of from about −5° C. to about 5° C. In one embodiment, the reaction temperature is about 0° C. In one embodiment, the reaction temperature is about 5° C.

In some embodiments, step 4.0 occurs at a reaction time of from about 10 minutes to about 2 hours. In one embodiment, the reaction time is from about 30 minutes to about 1 hour.

In one embodiment, step 4.0 occurs in the presence of potassium t-butoxide. In one embodiment, the molar ratios of the compound of Formula (V) to potassium t-butoxide is about 1:1.5. In one embodiment, step 4.0 occurs in a mixed solvent of toluene and THF and a solvent volume of 11 vol. at a temperature of about −5° C. to about 5° C. In one embodiment, step 4.0 occurs in toluene with about 4-7 folds in weight relevant to the compound of Formula (V) at a temperature of about −5° C. to about 5° C.

In some embodiments, step 4.0 proceeds to greater than 90%, greater than 95%, greater than 96%, greater than 97%, greater than 98%, or greater than 99% conversion within about 1 hour, as determined by HPLC and/or NMR. In some embodiments, step 4.0 provides less than about 10%, less than about 5%, less than about 4%, less than about 3%, less than about 2%, or less than about 1% of an impurity distinct from the compound of Formula (IV).

Impurities provided in step 4.0 may include one or more of the following impurities: the compound of Formula (V), the compound of Formula (VI), a compound of Formula (SP-6), a compound of Formula (SP-7), and/or a compound of Formula (SP-8).

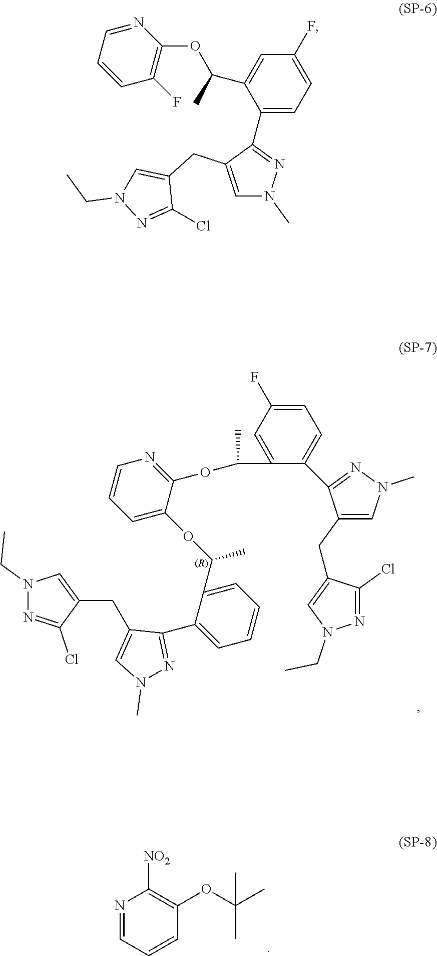

The impurity of Formula (SP-6) is an impurity observed in step 4.0. The impurity of Formula (SP-6) is formed by displacement of the nitro group from the compound of Formula (VI) vis SNAr reaction. In certain embodiments, the impurity of Formula (SP-6) can be controlled by using a non-protic polar solvent such as toluene in step 4.0. The impurity of Formula (SP-7) is formed from the double SNAr reactions of a compound of Formula (V) and a compound of Formula (VI).

In one embodiment, the total amount of impurities provided in step 4.0 is less than about 10%, less than about 5%, less than about 4%, less than about 3%, less than about 2%, or less than about 1%.

In some embodiments, step 4.0 further comprises purification of the compound of Formula (IV). In certain embodiments, the compound of Formula (IV) produced in step 4.0 is purified by treatment with activated charcoal, and/or slurrying in at least one organic solvent. In one embodiment, the at least one organic solvent is ethanol, heptane, or a mixture thereof. In one embodiment, step 4.0 further comprises crystalizing the compound of Formula (IV) from a mixture solvent of isopropanol and methylcyclohexane (MCH).

In certain embodiments, step 4.0 provides a compound of Formula (IV) in a substantially pure form. In certain embodiments, step 4.0 provides a compound of Formula (IV) in a substantially chemically pure form. In certain embodiments, step 4.0 provides a compound of Formula (IV) substantially free of impurities. In certain embodiments, step 4.0 provides a compound of Formula (IV) in a substantially enantiomerically pure form. In certain embodiments, step 4.0 provides a compound of Formula (IV) substantially free of impurities and easy scale up. In certain embodiments, step 4.0 reduces, eliminates or minimizes the amount of impurities carried forward into step 3.0.

In certain embodiments, also provided herein is a process for preparing a compound of Formula (V), or a stereoisomer, or a mixture of stereoisomers thereof, or a pharmaceutically acceptable salt thereof, comprising:

(step 5.0) reacting a compound of Formula (VII):

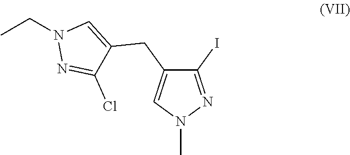 [SMILES: CCn1cc(Cc2cn(C)nc2I)c(Cl)n1]

or a stereoisomer, or a mixture of stereoisomers thereof, or a pharmaceutically acceptable salt thereof, with a compound of Formula (VIII):

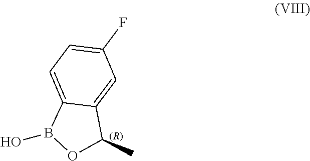

or a stereoisomer, or a mixture of stereoisomers thereof, or a pharmaceutically acceptable salt thereof.

In some embodiments, the molar ratio of the compound of Formula (VII) to the compound of Formula (VIII) in step 5.0 is from about 1:1 to about 1:1.2. In one embodiment, the molar ratio of the compound of Formula (VII) to the compound of Formula (VIII) in step 5.0 is about 1:1.1.

In some embodiments, step 5.0 occurs in the presence of a catalyst. In one embodiment, the catalyst is a palladium catalyst. In one embodiment, the palladium catalyst is Pd₂(dba)₃, Pd(PPh₃)₄, PdCl₂(PPh₃)₂, PdCl₂(Pcy₃)₂, PdCl₂(dppf), PdCl₂(dtbpf), or Pd(Amphos)Cl₂. In one embodiment, the catalyst is PdCl₂(dppf). In one embodiment, the catalyst is Pd(Amphos)Cl₂.

In some embodiments, the molar ratio of the compound of Formula (VII) to catalyst in step 5.0 is from about 1:0.001 (i.e. 0.1 mol %) to about 1:0.04 (i.e. 4 mol %). In some embodiments, the molar ratio of the compound of Formula (VII) to catalyst in step 5.0 is about 1:0.001, about 1:0.002, about 1:0.003, about 1:0.004, about 1:0.005, about 1:0.006, about 1:0.007, about 1:0.008, about 1:0.009, or about 1:0.01. In one embodiment, the molar ratio of the compound of Formula (VII) to catalyst in step 5.0 is about 1:0.005 (i.e. 0.5 mol %). In one embodiment, a catalyst loading of less than about 4 mol %, less than about 1 mol %, or about 0.5 mol %, is employed in step 5.0.

In some embodiments, step 5.0 occurs in the presence of a base. In some embodiments, step 5.0 occurs in the presence of an alkali metal base. In some embodiments, the base is an alkali metal hydroxide, carbonate, hydrogencarbonate, phosphate, hydrogenphosphate, or dihydrogenphosphate. In some embodiments, the base is LiOH, NaOH, KOH, Na₂CO₃, K₂CO₃, Cs₂CO₃, NaHCO₃, KHCO₃, Na₃PO₄, K₃PO₄, Na₂HPO₄, K₂HPO₄, NaH₂PO₄, or KH₂PO₄. In one embodiment, the base is potassium carbonate (K₂CO₃). In another embodiment, the base is potassium phosphate (K₃PO₄).

In some embodiments, the molar ratio of the compound of Formula (VII) to base in step 5.0 is from about 1:1 to about 1:4. In one embodiment, the molar ratio of the compound of Formula (VII) to base in step 5.0 is about 1:3. In one embodiment, the molar ratio of the compound of Formula (VII) to base in step 5.0 is about 1:1.5. In one embodiment, the molar ratio of the compound of Formula (VII) to base in step 5.0 is about 1:2.5.

Step 5.0 may occur in a solvent suitable for the reaction. In one embodiment, the solvent is DMF, DMA, NMP, I, DMSO, 1,4-dioxane, tetrahydrofuran, or water, or a mixture thereof. In one embodiment, the solvent is a mixture of an organic solvent and water. In one embodiment, step 5.0 occurs in a mixture of DMF and water. In one embodiment, step 5.0 occurs in a mixture of toluene and water. In one embodiment, step 5.0 occurs in a mixture of MTBE and water. In one embodiment, the mixture of an organic solvent and water has a weight ratio of organic solvent to water of from about 10:1 to about 4:1. In one embodiment, the weight ratio of organic solvent to water is about 5:1.

In some embodiments, the volume of solvent in step 5.0 is from about 6 vol to about 15 vol. In one embodiment, the volume of the solvent in step 5.0 is about 12 vol.

In some embodiments, step 5.0 occurs at a reaction temperature of from about 40° C. to about 90° C. In some embodiments, step 5.0 occurs at a reaction temperature of from about 60° C. to about 70° C. In one embodiment, the reaction temperature is about 65° C.

In some embodiments, step 5.0 occurs at a reaction time of from about 1 hour to about 4 hours. In one embodiment, the reaction time is from about 2 to about 3 hours.

In one embodiment, the molar ratio of the compound of Formula (VII) to the compound of Formula (VIII) in step 5.0 is about 1:1.1. In one embodiment, the catalyst is Pd(Amphos)Cl₂ and the catalyst loading is about 0.05 mol %. In one embodiment, the base is potassium carbonate (K₂CO₃) and the molar ratio of the compound of Formula (VII) to potassium carbonate in step 5.0 is about 1:1.5. In one embodiment, the solvent in step 5.0 is an about weight ratio 5:1 mixture of dimethylformamide and water. In one embodiment, step 5.0 occurs at a reaction temperature of about 65° C. and a reaction time of about 2 to about 3 hours.

In some embodiments, step 5.0 proceeds to greater than 90%, greater than 95%, greater than 96%, greater than 97%, greater than 98%, or greater than 99% conversion within about 2-3 hours, as determined by HPLC and/or NMR. In some embodiments, step 5.0 provides less than about 10%, less than about 5%, less than about 4%, less than about 3%, less than about 2%, or less than about 1% of an impurity distinct from the compound of Formula (V). Impurities provided in step 5.0 may include, but are not limited to, the compound of Formula (VII) and/or the compound of Formula (VIII). In one embodiment, the total amount of impurities provided in step 5.0 is less than about 10%, less than about 5%, less than about 4%, less than about 3%, less than about 2%, or less than about 1%.

In some embodiments, step 5.0 further comprises purification of the compound of Formula (V). In certain embodiments, the compound of Formula (V) produced in step 5.0 is purified by recrystallization.

In certain embodiments, step 5.0 provides a compound of Formula (V) in a substantially pure form. In certain embodiments, step 5.0 provides a compound of Formula (V) in a substantially chemically pure form. In certain embodiments, step 5.0 provides a compound of Formula (V) substantially free of impurities. In certain embodiments, step 5.0 provides a compound of Formula (V) in a substantially enantiomerically pure form. In certain embodiments, step 5.0 provides a compound of Formula (V) substantially free of impurities and easy scale up. In certain embodiments, step 5.0 reduces, eliminates or minimizes the amount of impurities carried forward into step 4.0.

In certain embodiments, also provided herein is a process for preparing a compound of Formula (XXIX), or a stereoisomer, or a mixture of stereoisomers thereof, or a pharmaceutically acceptable salt thereof, comprising:

(step 5.1) reacting a compound of Formula (VII):

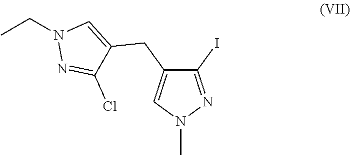 [SMILES: CCn1cc(Cc2cn(C)nc2I)c(Cl)n1]

or a stereoisomer, or a mixture of stereoisomers thereof, or a pharmaceutically acceptable salt thereof, with a compound of Formula (XXXI):

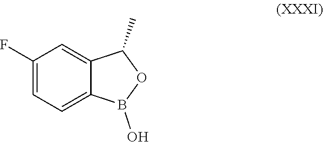

or a stereoisomer, or a mixture of stereoisomers thereof, or a pharmaceutically acceptable salt thereof.

In some embodiments, the molar ratio of the compound of Formula (VII) to the compound of Formula (XXXI) in step 5.1 is from about 1:1 to about 1:1.2. In one embodiment, the molar ratio of the compound of Formula (VII) to the compound of Formula (XXXI) in step 5.0 is about 1:1.1.

In some embodiments, step 5.1 occurs in the presence of a catalyst. In one embodiment, the catalyst is a palladium catalyst. In one embodiment, the palladium catalyst is Pd₂(dba)₃, Pd(PPh₃)₄, PdCl₂(PPh₃)₂, PdCl₂(Pcy₃)₂, PdCl₂(dppf), PdCl₂(dtbpf), or Pd(Amphos)Cl₂. In one embodiment, the catalyst is PdCl₂(dppf). In one embodiment, the catalyst is Pd(Amphos)Cl₂.

In some embodiments, the molar ratio of the compound of Formula (VII) to catalyst in step 5.1 is from about 1:0.001 (i.e. 0.1 mol %) to about 1:0.04 (i.e. 4 mol %). In some embodiments, the molar ratio of the compound of Formula (VII) to catalyst in step 5.0 is about 1:0.001, about 1:0.002, about 1:0.003, about 1:0.004, about 1:0.005, about 1:0.006, about 1:0.007, about 1:0.008, about 1:0.009, or about 1:0.01. In one embodiment, the molar ratio of the compound of Formula (VII) to catalyst in step 5.0 is about 1:0.005 (i.e. 0.5 mol %). In one embodiment, a catalyst loading of less than about 4 mol %, less than about 1 mol %, or about 0.5 mol %, is employed in step 5.1.

In some embodiments, step 5.1 occurs in the presence of a base. In some embodiments, step 5.1 occurs in the presence of an alkali metal base. In some embodiments, the base is an alkali metal hydroxide, carbonate, hydrogencarbonate, phosphate, hydrogenphosphate, or dihydrogenphosphate. In some embodiments, the base is LiOH, NaOH, KOH, Na₂CO₃, K₂CO₃, Cs₂CO₃, NaHCO₃, KHCO₃, Na₃PO₄, K₃PO₄, Na₂HPO₄, K₂HPO₄, NaH₂PO₄, or KH₂PO₄. In one embodiment, the base is potassium carbonate (K₂CO₃). In another embodiment, the base is potassium phosphate (K₃PO₄).

In some embodiments, the molar ratio of the compound of Formula (VII) to base in step 5.1 is from about 1:1 to about 1:4. In one embodiment, the molar ratio of the compound of Formula (VII) to base in step 5.1 is about 1:3. In one embodiment, the molar ratio of the compound of Formula (VII) to base in step 5.1 is about 1:1.5. In one embodiment, the molar ratio of the compound of Formula (VII) to base in step 5.1 is about 1:2.5.

Step 5.1 may occur in a solvent suitable for the reaction. In one embodiment, the solvent is DMF, DMA, NMP, DMSO, 1,4-dioxane, tetrahydrofuran, or water, or a mixture thereof. In one embodiment, the solvent is a mixture of an organic solvent and water. In one embodiment, step 5.1 occurs in a mixture of DMF and water. In one embodiment, step 5.1 occurs in a mixture of toluene and water. In one embodiment, step 5.1 occurs in a mixture of MTBE and water. In one embodiment, the mixture of an organic solvent and water has a weight ratio of organic solvent to water of from about 10:1 to about 4:1. In one embodiment, the weight ratio of organic solvent to water is about 5:1.

In some embodiments, the volume of solvent in step 5.1 is from about 6 vol to about 15 vol. In one embodiment, the volume of the solvent in step 5.0 is about 12 vol.

In some embodiments, step 5.1 occurs at a reaction temperature of from about 40° C. to about 90° C. In some embodiments, step 5.1 occurs at a reaction temperature of from about 60° C. to about 70° C. In one embodiment, the reaction temperature is about 65° C.

In some embodiments, step 5.1 occurs at a reaction time of from about 1 hour to about 4 hours. In one embodiment, the reaction time is from about 2 to about 3 hours.

In one embodiment, the molar ratio of the compound of Formula (VII) to the compound of Formula (XXXI) in step 5.1 is about 1:1.1. In one embodiment, the catalyst is Pd(Amphos)Cl₂ and the catalyst loading is about 0.05 mol %. In one embodiment, the base is potassium phosphate (K₃PO₄) and the molar ratio of the compound of Formula (VII) to potassium phosphate in step 5.1 is about 1:2.5. In one embodiment, the solvent in step 5.1 is a mixture of toluene and water. In one embodiment, the solvent in step 5.1 is a mixture of MTBE and water. In one embodiment, the compound of Formula (XXIX) is purified by crystallization from a solvent of MTBE and/or heptane.

In some embodiments, step 5.1 proceeds to greater than 90%, greater than 95%, greater than 96%, greater than 97%, greater than 98%, or greater than 99% conversion within about 2-3 hours, as determined by HPLC and/or NMR. In some embodiments, step 5.1 provides less than about 10%, less than about 5%, less than about 4%, less than about 3%, less than about 2%, or less than about 1% of an impurity distinct from the compound of Formula (XXIX). Impurities provided in step 5.1 may include, but are not limited to, the compound of Formula (VII) and/or the compound of Formula (XXXI). In one embodiment, the total amount of impurities provided in step 5.1 is less than about 10%, less than about 5%, less than about 4%, less than about 3%, less than about 2%, or less than about 1%.

In some embodiments, step 5.1 further comprises purification of the compound of Formula (XXIX). In certain embodiments, the compound of Formula (XXIX) produced in step 5.1 is purified by recrystallization. In certain embodiments, step 5.1 provides a compound of Formula (XXIX) in a substantially pure form. In certain embodiments, step 5.1 provides a compound of Formula (XXIX) in a substantially chemically pure form. In certain embodiments, step 5.1 provides a compound of Formula (XXIX) substantially free of impurities. In certain embodiments, step 5.1 provides a compound of Formula (XXIX) in a substantially enantiomerically pure form. In certain embodiments, step 5.1 provides a compound of Formula (XXIX) substantially free of impurities and easy scale up. In certain embodiments, step 5.1 reduces, eliminates or minimizes the amount of impurities carried forward into step 2a.1.

In certain embodiments, the compound of Formula (VIII) is prepared from the following scheme:

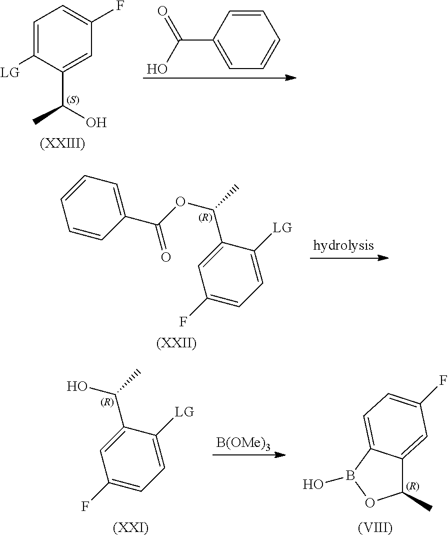

In one embodiment, the compound of Formula (VIII) is prepared by a process comprising reacting the compound of Formula (XXI) with a borate (e.g. trimethyl borate). In one embodiment, the compound of Formula (XXI) is prepared by process comprising hydrolyzing the compound of Formula (XXII). In one embodiment, the compound of Formula (XXII) is prepared by a process comprising reacting the compound of Formula (XXIII) with benzoic acid.

In one embodiment, an alcohol of Formula (XXIII) reacts with benzoic acid in the presence of PPh₃ and DIAD to form an ester of Formula (XXII). In this step, the stereocenter of the compound of Formula (XXIII) is inverted. The ester of Formula (XXII) undergoes hydrolysis in a base (e.g. an inorganic base like NaOH or KOH) to form an alcohol of Formula (XXI). An exemplified hydrolysis condition is aqueous NaOH and MeOH. The compound of Formula (XXI) further reacts with a borate (e.g. trimethyl borate) to provide a compound of Formula (VIII). In one embodiment, the boronation step is carried out in the presence of i-PrMgCl and THF. As used herein, LG is a leaving group, referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6^{th} ed. (501-502). Exemplary leaving groups include, but are not limited to, iodo or —O(SO)₂RLG (e.g., tosyl, mesyl, besyl), wherein RLG is optionally substituted alkyl (e.g. CH₃), optionally substituted aryl (e.g., p-nitrobenzyl- or p-methylphenyl-), or optionally substituted heteroaryl. In some embodiments, the leaving group is iodo.

In other embodiments, the compound of Formula (VIII) is prepared from the following scheme:

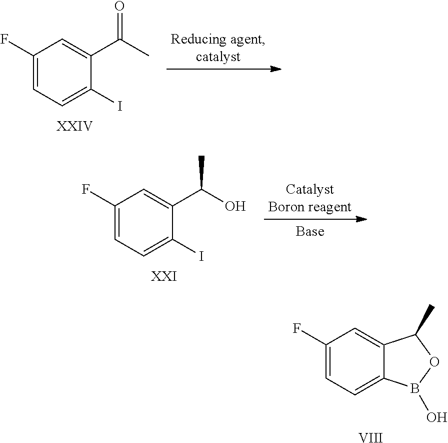

In one embodiment, the compound of Formula (VIII) is prepared by a process comprising reacting the compound of Formula (XXI) with a boron reagent. In one embodiment, the compound of Formula (XXI) is reacted with a boron reagent in the presence of a catalyst and/or base. In one embodiment, the compound of Formula (XXI) is prepared by a process comprising reducing the compound of Formula (XXIV). In one embodiment, the compound of Formula (XXIV) is reduced in the presence of a catalyst.

In one embodiment, a ketone of Formula (XXIV) is reduced in the presence of a catalyst to form an alcohol of Formula (XXI). In certain embodiments, the catalyst is a chiral catalyst, and the reduction affords the alcohol of Formula (XXI) in an enantioenriched form. In certain embodiment, the chiral catalyst is an oxaborolidinone (Corey-Bakshi-Shibata, CBS) reduction catalyst. In certain embodiments, the chiral catalyst is (−),- or (R,R)-DIP-Cl In certain embodiment, the chiral catalyst is Ir—(R)-SprioPAP-3-Me, Dichloro[(r)-(−)-4,12-bis(di(3,5-xylyl)phosphino)-[2,2]-paracyclophane][(1s,2s)-(−)-1,2-diphenylethylenediamine]ruthenium, [((S)-sylyl-PhanePhos)Ru{{R,R)-(DPEN)Cl2, Josiphos, RuCl₂[(S)-Xyl-P-Phos][(S)-DAIPEN], C4-[(S,S)-teth-TsDPEN RuCl], or [Rh(NBD)BF₄)]. In certain embodiments, the chiral catalyst is RuCl(p-cymene)[R,R-Ts-DPEN]. In certain embodiments, the chiral alcohol of Formula (XXI) is prepared by asymmetric Noyori transfer hydrogenation. In certain embodiments, the reducing agent is a hydriodic reagent. In certain embodiments, the hydriodic reagent is a formate salt, such as sodium formate or triethyl amine/formic acid. In certain embodiments, the reduction is performed in a protic solvent, such as an alcohol solvent, for example in methanol. In certain embodiments, the compound of Formula (XXI) is then further reacted with a boron reagent in the presence of a catalyst and optionally base to provide a compound of Formula (VIII). In one embodiment, the boron reagent is trimethyl borate. In another embodiment, the boron reagent is B₂(OH)₄. In certain embodiments, the catalyst is a palladium catalyst. In certain embodiments, the palladium catalyst is Pd(amphos)Cl₂, Xphos Pd G2, Pd(PPh₃)₄, or Pd(dppf)Cl₂. In one embodiment, the palladium catalyst is Xphos Pd G2. In certain embodiments, the boronation reaction is performed in the presence of a carboxylate base. In one embodiment, the carboxylate base is potassium acetate.

In certain embodiments, the compound of Formula (XXXI) is prepared as described in PCT/US2022/077323, which is incorporated herein by reference in its entirety.

In certain embodiments, also provided herein is a process for preparing a compound of Formula (I), or a stereoisomer, or a mixture of stereoisomers thereof, or a pharmaceutically acceptable salt thereof, is prepared by a process comprising:

(step 1.0) cyclizing a compound of Formula (II) or a stereoisomer, or a mixture of stereoisomers thereof, or a pharmaceutically acceptable salt thereof, to provide a compound of Formula (I), or a stereoisomer, or a mixture of stereoisomers thereof, or a pharmaceutically acceptable salt thereof, wherein the compound of Formula (II) is prepared by a process comprising:
(step 2.0) reacting a compound of Formula (III) or a stereoisomer, or a mixture of stereoisomers thereof, or a pharmaceutically acceptable salt thereof, with a brominating reagent; wherein the compound of Formula (III) is prepared by a process comprising:
(step 3.0) reducing a compound of Formula (IV) or a stereoisomer, or a mixture of stereoisomers thereof, or a pharmaceutically acceptable salt thereof, wherein the compound of Formula (IV) is prepared by a process comprising:
(step 4.0) reacting a compound of Formula (V) or a stereoisomer, or a mixture of stereoisomers thereof, or a pharmaceutically acceptable salt thereof, with a compound of Formula (VI); and wherein the compound of Formula (V) is prepared by a process comprising:
(step 5.0) reacting a compound of Formula (VII) or a stereoisomer, or a mixture of stereoisomers thereof, or a pharmaceutically acceptable salt thereof, with a compound of Formula (VIII) or a stereoisomer, or a mixture of stereoisomers thereof, or a pharmaceutically acceptable salt thereof.

In one embodiment, also provided herein is a process for preparing a compound of Formula (I), or a stereoisomer, or a mixture of stereoisomers thereof, or a pharmaceutically acceptable salt thereof, is prepared by a process comprising:

(step 1.0) cyclizing a compound of Formula (II) or a stereoisomer, or a mixture of stereoisomers thereof, or a pharmaceutically acceptable salt thereof, to provide a compound of Formula (I), or a stereoisomer, or a mixture of stereoisomers thereof, or a pharmaceutically acceptable salt thereof, wherein the compound of Formula (II) is prepared by a process comprising:
(step 2a.1) reacting a compound of Formula (XXIX), or a stereoisomer, or a mixture of stereoisomers thereof, or a pharmaceutically acceptable salt thereof, with a compound of Formula (XXX), or a pharmaceutically acceptable salt thereof, in the presence of a diazene and a phosphine; wherein the compound of Formula (XXIX) is prepared by a process comprising:
(step 5.1) reacting a compound of Formula (VII) or a stereoisomer, or a mixture of stereoisomers thereof, or a pharmaceutically acceptable salt thereof, with a compound of Formula (XXXI) or a stereoisomer, or a mixture of stereoisomers thereof, or a pharmaceutically acceptable salt thereof.

In one embodiment, also provided herein is a process for preparing a compound of Formula (I), or a stereoisomer, or a mixture of stereoisomers thereof, or a pharmaceutically acceptable salt thereof, comprising:

(step 1.0) cyclizing a compound of Formula (II):

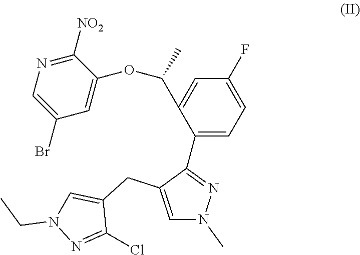 [SMILES: CCn1cc(Cc2cn(C)nc2-c2ccc(F)cc2[C@@H](C)Oc2cc(Br)cnc2[N+](=O)[O-])c(Cl)n1]

or a stereoisomer, or a mixture of stereoisomers thereof, or a pharmaceutically acceptable salt thereof, to provide a compound of Formula (I), or a stereoisomer, or a mixture of stereoisomers thereof, or a pharmaceutically acceptable salt thereof,
wherein step 1.0 occurs in the presence of a base, and wherein the base is potassium pivalate.

In one embodiment, the processes provided herein further comprise a step of providing the compound of Formula (I), or a stereoisomer, or a mixture of stereoisomers thereof, or a pharmaceutically acceptable salt thereof, in a solid form. In one embodiment, the solid form is a crystalline form. In one embodiment, provided herein is a crystalline form of a compound of Formula (I), or a stereoisomer, or a mixture of stereoisomers thereof, or a pharmaceutically acceptable salt thereof, prepared by the process.

In one embodiment, provided herein is a compound of Formula (I) which meets one or more of the following purity criteria: (i) has less than about 1%, about 0.5%, about 0.1%, or about 0.05% of impurity (e.g., as determined by HPLC % area); (ii) has more than about 99%, about 99.5%, or 99.9% of chiral purity, or about 100% chiral purity (e.g., as determined by HPLC % area); (iii) has less than about 1%, about 0.5%, or about 0.1% w/w of water content (e.g., as determined according to USP <921> Karl Fischer); and (iv) has less than about 100 ppm, about 50 ppm, about 20 ppm, or about 10 ppm of palladium (e.g., as determined according to USP <233> ICP-OES). In one embodiment, the compound of Formula (I): (i) has less than about 0.05% of impurity as determined by HPLC % area; (ii) has about 100% chiral purity; (iii) has less than about 0.1% w/w of water content; and (iv) has less about 10 ppm of palladium. In one embodiment, the compound of Formula (I) which meets the purity criteria is manufactured by a process provided herein.

In one embodiment, also provided herein is a compound of Formula (II), (III), (IV), (V) or (VII):

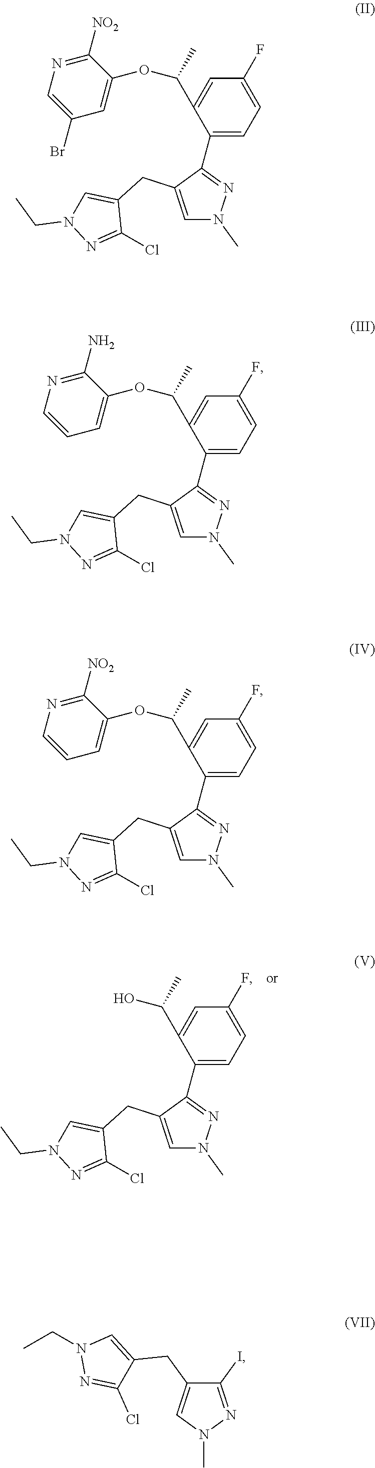 [SMILES: CCn1cc(Cc2cn(C)nc2-c2ccc(F)cc2[C@@H](C)Oc2cc(Br)cnc2[N+](=O)[O-])c(Cl)n1 ; CCn1cc(Cc2cn(C)nc2-c2ccc(F)cc2[C@@H](C)Oc2cccnc2N)c(Cl)n1 ; CCn1cc(Cc2cn(C)nc2-c2ccc(F)cc2[C@@H](C)Oc2cccnc2[N+](=O)[O-])c(Cl)n1 ; CCn1cc(Cc2cn(C)nc2-c2ccc(F)cc2[C@@H](C)O)c(Cl)n1 ; CCn1cc(Cc2cn(C)nc2I)c(Cl)n1]

or a stereoisomer, or a mixture of stereoisomers thereof, or a pharmaceutically acceptable salt thereof.

### 5.4. Pharmaceutical Compositions

In one embodiment, provided herein is a pharmaceutical composition comprising Compound 1:

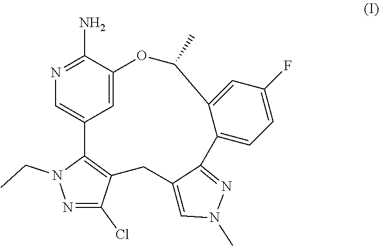 [SMILES: CCn1nc(Cl)c2c1-c1cnc(N)c(c1)O[C@H](C)c1cc(F)ccc1-c1nn(C)cc1C2]

or a stereoisomer, or a mixture of stereoisomers thereof, or a pharmaceutically acceptable salt thereof, a diluent, a disintegrant, a glidant, a binder, and a lubricant.

In one embodiment, Compound 1, or a stereoisomer, or a mixture of stereoisomers thereof, or a pharmaceutically acceptable salt thereof, is free base of Compound 1. In one embodiment, the free base of Compound 1 is amorphous. In one embodiment, the free base of Compound 1 is a crystalline free base of Compound 1. In one embodiment, the free base of Compound 1 is one of the solid forms of free base of Compound 1 provided herein. In one embodiment, the free base of Compound 1 is Form 2 of the free base of Compound 1. In one embodiment, the free base of Compound 1 is characterized by an XRPD pattern comprising peaks at approximately 12.4, 18.9, and 21.1° 2θ (±0.2°). In one embodiment, at least 90% of a representative sample of the particles of Compound 1 has a particle size of about 19 μm to about 106 μm. In one embodiment, at least 50% of a representative sample of the particles of Compound 1 has a particle size of about 10 μm to about 47 μm. In one embodiment, at least 10% of a representative sample of the particles of Compound 1 has a particle size of about 4 μm to about 15 μm.

In one embodiment, Compound 1, or a stereoisomer, or a mixture of stereoisomers thereof, or a pharmaceutically acceptable salt thereof, in the pharmaceutical composition is a pharmaceutically acceptable salt of Compound 1. In one embodiment, the salt is amorphous.

As used herein and unless otherwise specified, the total weight of the pharmaceutical composition (or the w/w based on the total weight of the pharmaceutical composition) does not include coating of the pharmaceutical composition (e.g., an Opadry II coat of a tablet pharmaceutical composition provided herein).

In one embodiment, the amount of Compound 1, or a stereoisomer, or a mixture of stereoisomers thereof, or a pharmaceutically acceptable salt thereof, in the pharmaceutical composition is from about 1% to about 30% w/w. In one embodiment, the amount is from about 3% to about 25% w/w. In one embodiment, the amount is from about 4% to about 25% w/w. In one embodiment, the amount is from about 5% to about 20% w/w. In one embodiment, the amount is from about 3% to about 7% w/w. In one embodiment, the amount is from about 4% to about 6% w/w. In one embodiment, the amount is about 3, about 4, about 5, about 6, or about 7% w/w. In one embodiment, the amount is about 4% w/w. In one embodiment, the amount is about 5% w/w. In one embodiment, the amount is from about 15% to about 25% w/w. In one embodiment, the amount is from about 17% to about 23% w/w. In one embodiment, the amount is from about 18% to about 22% w/w. In one embodiment, the amount is from about 19% to about 21% w/w. In one embodiment, the amount is about 15, about 16, about 17, about 18, about 19, about 20, about 21, about 22, about 23, about 24, or about 25% w/w. In one embodiment, the amount is about 19% w/w. In one embodiment, the amount is about 20% w/w.

In one embodiment, the diluent is microcrystalline cellulose.

In one embodiment, the amount of the diluent in the pharmaceutical composition is from about 50% to about 95% w/w. In one embodiment, the amount is from about 68% to about 83.5% w/w. In one embodiment, the amount is from about 60% to about 75% w/w. In one embodiment, the amount is from about 65% to about 70% w/w. In one embodiment, the amount is from about 67% to about 69% w/w. In one embodiment, the amount is about 65, about 66, about 67, about 68, about 69, or about 70% w/w. In one embodiment, the amount is about 66% w/w. In one embodiment, the amount is about 67% w/w. In one embodiment, the amount is about 68% w/w. In one embodiment, the amount is from about 75% to about 90% w/w. In one embodiment, the amount is from about 80% to about 85% w/w. In one embodiment, the amount is from about 83% to about 84% w/w. In one embodiment, the amount is about 80, about 81, about 82, about 83, about 84, or about 85% w/w. In one embodiment, the amount is about 81% w/w. In one embodiment, the amount is about 82% w/w. In one embodiment, the amount is about 83% w/w. In one embodiment, the amount is about 83.5% w/w. In one embodiment, the amount is about 84% w/w.

In one embodiment, the disintegrant is croscarmellose sodium.

In one embodiment, the amount of the disintegrant in the pharmaceutical composition is from about 10% to about 10% w/w. In one embodiment, the amount is from about 2.50% to about 7.5% w/w. In one embodiment, the amount is about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10% w/w. In one embodiment, the amount is about 5% w/w. In one embodiment, the amount is about 4.5% w/w.

In one embodiment, the glidant is colloidal silica dioxide.

In one embodiment, the amount of the glidant in the pharmaceutical composition is from about 1% to about 5% w/w. In one embodiment, the amount is from about 1% to about 4% w/w. In one embodiment, the amount is from about 2% to about 4% w/w. In one embodiment, the amount is from about 2% to about 3% w/w. In one embodiment, the amount is about 1, about 1.5, about 2, about 2.1, about 2.2, about 2.3, about 2.4, about 2.5, about 2.6, about 2.7, about 2.8, about 2.9, about 3, about 3.5, about 4, about 4.5, or about 5% w/w. In one embodiment, the amount is about 2.5% w/w.

In one embodiment, the binder is hydroxypropyl cellulose (HPC).

In one embodiment, the amount of the binder in the pharmaceutical composition is from about 1% to about 50% w/w. In one embodiment, the amount is from about 1% to about 4% w/w. In one embodiment, the amount is from about 2% to about 4% w/w. In one embodiment, the amount is from about 2% to about 3% w/w. In one embodiment, the amount is about 1, about 1.5, about 2, about 2.1, about 2.2, about 2.3, about 2.4, about 2.5, about 2.6, about 2.7, about 2.8, about 2.9, about 3, about 3.5, about 4, about 4.5, or about 5% w/w. In one embodiment, the amount is about 2.5% w/w.

In one embodiment, the lubricant is magnesium stearate.

In one embodiment, the amount of the lubricant in the pharmaceutical composition is from about 0.5% to about 4% w/w. In one embodiment, the amount is from about 1% to about 3% w/w. In one embodiment, the amount is from about 1.5% to about 2% w/w. In one embodiment, the amount is about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.5, or about 4% w/w. In one embodiment, the amount is about 1.5% w/w. In one embodiment, the amount is about 2% w/w.

In one embodiment, the pharmaceutical composition comprises: Compound 1, or a stereoisomer, or a mixture of stereoisomers thereof, or a pharmaceutically acceptable salt thereof, at an amount of from about 2.5% to about 7.5% w/w of the total weight of the pharmaceutical composition; a diluent at an amount of from about 75% to about 90% w/w of the total weight of the pharmaceutical composition; a disintegrant at an amount of from about 2.5% to about 7.5% w/w of the total weight of the pharmaceutical composition; a glidant at an amount of from about 1% to about 4% w/w of the total weight of the pharmaceutical composition; a binder at an amount of from about 1% to about 4% w/w of the total weight of the pharmaceutical composition; and a lubricant at an amount of from about 1% to about 2% w/w of the total weight of the pharmaceutical composition. In one embodiment, the pharmaceutical composition comprises: Compound 1 at an amount of about 5% w/w of the total weight of the pharmaceutical composition; microcrystalline cellulose at an amount of about 83.5% w/w of the total weight of the pharmaceutical composition; croscarmellose sodium at an amount of about 5% w/w of the total weight of the pharmaceutical composition; colloidal silica dioxide at an amount of about 2.5% w/w of the total weight of the pharmaceutical composition; hydroxypropyl cellulose at an amount of about 2.5% w/w of the total weight of the pharmaceutical composition; and magnesium stearate at an amount of about 1.5% w/w of the total weight of the pharmaceutical composition. In one embodiment, the pharmaceutical composition has a total weight of about 100 mg. As used herein and unless otherwise specified, the total weight of the pharmaceutical composition does not include the weight of any coating of the pharmaceutical composition.

In one embodiment, the pharmaceutical composition comprises: Compound 1, or a stereoisomer, or a mixture of stereoisomers thereof, or a pharmaceutically acceptable salt thereof, at an amount of from about 15% to about 25% w/w of the total weight of the pharmaceutical composition; a diluent at an amount of from about 60% to about 75% w/w of the total weight of the pharmaceutical composition; a disintegrant at an amount of from about 2.5% to about 7.5% w/w of the total weight of the pharmaceutical composition; a glidant at an amount of from about 1% to about 4% w/w of the total weight of the pharmaceutical composition; a binder at an amount of from about 1% to about 4% w/w of the total weight of the pharmaceutical composition; and a lubricant at an amount of from about 1% to about 3% w/w of the total weight of the pharmaceutical composition. In one embodiment, the pharmaceutical composition comprises: Compound 1 at an amount of about 20% w/w of the total weight of the pharmaceutical composition; microcrystalline cellulose at an amount of about 68% w/w of the total weight of the pharmaceutical composition; croscarmellose sodium at an amount of about 5% w/w of the total weight of the pharmaceutical composition; colloidal silica dioxide at an amount of about 2.5% w/w of the total weight of the pharmaceutical composition; hydroxypropyl cellulose at an amount of about 2.5% w/w of the total weight of the pharmaceutical composition; and magnesium stearate at an amount of about 2% w/w of the total weight of the pharmaceutical composition. In one embodiment, the pharmaceutical composition has a total weight of about 125 mg. In one embodiment, the pharmaceutical composition has a total weight of about 250 mg. In one embodiment, the pharmaceutical composition has a total weight of about 375 mg. In one embodiment, the pharmaceutical composition has a total weight of about 500 mg. In one embodiment, the pharmaceutical composition has a total weight of about 625 mg. In one embodiment, the pharmaceutical composition has a total weight of about 750 mg. As used herein and unless otherwise specified, the total weight of the pharmaceutical composition does not include the weight of any coating of the pharmaceutical composition.

The pharmaceutical compositions may conveniently be presented in unit dosage form. In one embodiment, the pharmaceutical composition is an oral dosage form. In one embodiment, the oral dosage form is a tablet. In certain embodiments, the unit dosage form is a tablet of 5 mg (by weight of free base Compound 1) dose strength. In certain embodiments, the unit dosage form is a tablet of 25 mg (by weight of free base Compound 1) dose strength. In certain embodiments, the unit dosage form is a tablet of 50 mg (by weight of free base Compound 1) dose strength. In certain embodiments, the unit dosage form is a tablet of 75 mg (by weight of free base Compound 1) dose strength. In certain embodiments, the unit dosage form is a tablet of 100 mg (by weight of free base Compound 1) dose strength. In certain embodiments, the unit dosage form is a tablet of 125 mg (by weight of free base Compound 1) dose strength. In certain embodiments, the unit dosage form is a tablet of 150 mg (by weight of free base Compound 1) dose strength. The amount of active ingredient which can be combined with a carrier material to produce a single dosage form will vary depending upon the subject being treated, the particular mode of administration. In one embodiment, the oral dosage form is an immediate release tablet. In one embodiment, the pharmaceutical composition is film-coated.

In one embodiment, oral dosage forms (e.g. tablets) comprising Compound 1 (e.g. Form 2 of Compound 1) are stable after storage at 30° C.±2° C./65%±5% RH for at least 12 months. In one embodiment, oral dosage forms (e.g. tablets) comprising Compound 1 (e.g. Form 2 of Compound 1) are stable at 40° C.±2° C./75%±5% RH for at least 6 months. In one embodiment, oral dosage forms (e.g. tablets) comprising Compound 1 (e.g. Form 2 of Compound 1) stored at 30° C.±2° C./65%±5% RH for at least 12 months or at 40° C.±2° C./75%±5% RH for at least 6 months is at least 90 wt % chemically pure.

In certain embodiments, the present disclosure provides a pharmaceutical preparation suitable for use in a human patient, comprising any of the compounds shown above (e.g., a compound of the disclosure, such as a compound of Formula (I), and one or more pharmaceutically acceptable excipients. In certain embodiments, the pharmaceutical preparations may be for use in treating or preventing a condition or disease as described herein. Any of the disclosed compounds may be used in the manufacture of medicaments for the treatment of any diseases or conditions disclosed herein.

In certain embodiments, provided is a pharmaceutical composition comprising Form 2 and a pharmaceutically acceptable carrier. In certain embodiments, provided is a pharmaceutical composition comprising Form 2 substantially free (e.g., less than about 0.2 wt %, about 0.1 wt %, about 0.05 wt %, or about 0.01 wt %) of impurities such as a compound of any one of Formulae (SP-1) to (SP-8). In certain embodiments, provided is a pharmaceutical composition comprising Form 2 substantially free (e.g., less than about 0.2 wt %, about 0.1 wt %, about 0.05 wt %, or about 0.01 wt %) of impurities such as compounds of Formula (SP-1) and/or Formula (SP-2). In certain embodiments, the pharmaceutical composition comprising Form 2 is substantially free of other crystal forms of the compound of Formula (I).

The compositions and methods of the present disclosure may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In certain embodiments, the active compound will be administered once daily.

In certain embodiments, compounds of the disclosure may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the disclosure with one or more additional therapeutic agent(s) (e.g., one or more additional chemotherapeutic agent(s)) provides improved efficacy relative to each individual administration of the compound of the disclosure (e.g., compound of Formula I) or the one or more additional therapeutic agent(s). In certain such embodiments, the conjoint administration provides an additive effect, wherein an additive effect refers to the sum of each of the effects of individual administration of the compound of the disclosure and the one or more additional therapeutic agent(s).

This disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Pharmaceutically acceptable anionic salts include acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, acetate, succinate, sulfate, tartrate, teoclate, and tosylate.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### 5.5. Therapeutic Methods

In one embodiment, provided herein are methods of treating cancer comprising administering a solid form or pharmaceutical composition provided herein. In one embodiment, provided herein are methods of using a solid form or pharmaceutical composition provided herein for treating, preventing or managing solid tumor. In one embodiment, provided herein is a method of treating solid tumor, comprising administering to a patient in need thereof a solid form or pharmaceutical composition provided herein.

Cancer is a disease of uncontrolled cell proliferation that results from alterations in certain genes. Some of these alterations occur in genes that encode receptor tyrosine kinases (RTKs), a family of membrane-bound proteins that transmit signals from outside the cell to promote cell survival, growth, and proliferation. Aberrant RTK activation can lead to excessive cell growth and hence cancer. Generally, RTKs contain an N-terminal domain that binds extracellular ligands, a transmembrane domain, and a C-terminal kinase domain that catalyzes intracellular signal transduction.

In some embodiments, the compound of Formula (I) is an inhibitor of human ROS1. ROS1 is an RTK encoded by the ROS1 gene. The ligands and biological functions of human ROS1 are unknown, but its homologs in some other species have been shown to bind extracellular ligands and stimulate cell differentiation. For example, mouse ROS1 is essential for male gamete maturation and reproduction. In humans, ROS1 chromosomal rearrangements are a well-documented cause of cancer, representing 1-2% of non-small cell lung cancer (NSCLC) and a subset of many other cancers. These rearrangements result in the fusion of the C-terminus of ROS1 with the N-terminus of various partner proteins, the most common of which is CD74. ROS1 fusions have constitutive kinase activity that drives tumor growth through MAPK, PI3K, and JAK/STAT signaling pathways. Small-molecule tyrosine kinase inhibitors (TKIs) have been used to target ROS1 fusions in cancer, including crizotinib and entrectinib. Crizotinib was the first FDA-approved TKI for the treatment of ROS1-positive NSCLC, with an overall response rate of 60-80% and median progression-free survival of 9-19 months. Despite an initial response, most patients acquire resistance to crizotinib and relapse. The predominant mechanism of resistance is the G2032R mutation in the solvent front, which dramatically reduces crizotinib affinity. No inhibitors with activity against ROS1-G2032R fusions have been FDA-approved, indicating a need in the art.

In some embodiments, the compound of Formula (I) is an inhibitor of human anaplastic lymphoma kinase (ALK). ALK, also known as cluster of differentiation 246 (CD246), is an RTK encoded by the ALK gene. ALK and ROS1 are evolutionarily related; both belong to the insulin receptor superfamily, and their kinase domains share around 80% sequence similarity. A few ALK ligands in humans have been identified, including pleiotrophin and midkine growth factors. While the roles of ALK in humans remain inconclusive, much evidence from mouse studies suggests that it is important for the development of the nervous system. Like ROS1, ALK chromosomal rearrangements also lead to constitutively active fusion proteins that promote oncogenic transformation through MAPK, JAK/STAT, or other signaling pathways. ALK rearrangements represent 3-5% of NSCLC, roughly half of anaplastic large-cell lymphoma (ALCL), and a subset of many other cancers, with the predominant fusions being EML4-ALK for NSCLC and NPM1-ALK for ALCL. Oncogenic point mutations and amplification of ALK have also been observed, albeit at a much lower frequency than translocations. Crizotinib, ceritinib, alectinib, brigatinib, and lorlatinib are FDA-approved TKIs for the treatment of ALK-positive NSCLC and other cancers, either in front-line or after prior therapy. Crizotinib, for example, shows an overall response rate of 60-80% and median progression-free survival of 8-11 months, which is comparable to its activity in ROS1-positive NSCLC. Despite an initial response, many resistance mutations have emerged to the aforementioned FDA-approved TKIs. Some of these mutations, such as the combined L1196M gatekeeper and G1202R solvent front mutation, are resistant to all of the approved drugs. New treatments of ALK-positive cancer harboring resistance mutations are a need in the art.

In further embodiments, the compound of Formula (I) is an inhibitor of human tropomyosin receptor kinases (TRKs). The TRK family comprises receptor tyrosine kinases TRKA, TRKB, and TRKC, which are encoded by the NTRK1, NTRK2, and NTRK3 genes, respectively. Each TRK is activated by a different but overlapping set of neurotrophin ligands such as NGF, BDNF, and NT-3. All TRKs modulate similar downstream signaling pathways, consistent with sequence divergence in the ligand-binding domain but convergence in the kinase domain (90% similarity). TRKs play crucial roles in the nervous system of developing and adult mammals by regulating processes such as memory, movement, pain, and proprioception. Like ROS1 and ALK, NTRK rearrangements lead to constitutively active TRK fusions that drive oncogenic transformation through MAPK, PI3K, and other pathways. TRK fusions are found in many cancers and represent over 80% of the cases in secretory breast carcinoma, mammary analogue secretory carcinomas, infantile fibrosarcoma, and congenital mesoblastic nephroma. Thus, inhibition of TRKs is advantageous for treating cancers expressing TRK fusions.

Many ROS1 and ALK inhibitors in the prior art also exhibit potent inhibition of native non-oncogenic TRKs. This is a substantial drawback because native TRKs play important functions in the nervous system, and inadvertent inhibition of native TRKs is associated with adverse reactions including dizziness, ataxia, gait disturbance, paraesthesia, weight gain, and cognitive changes. New therapies that spare TRKs while selectively targeting ROS1 and/or ALK, in their non-mutant and/or mutant forms, are a need in the art.

In one embodiment, provided herein is a method of decreasing a level of ROS1 or ALK in a cell, comprising contacting the cell with a compound or a pharmaceutical composition or a pharmaceutical combination provided herein. In an embodiment, such contact occurs in a cell in a mammal such as a human. In an embodiment, such contact occurs in a cell in human patient having a cancer provided herein.

In one embodiment, a compound provided herein selectively inhibits ROS1.

In one embodiment, the compound selectively inhibits ROS1 over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by ratio of IC50 values, among other means. In one embodiment, the selectivity of ROS1 over TRK is measured by the ratio of the IC50 value against TRK to the IC50 value against ROS1.

In one embodiment, a compound provided herein selectively inhibits ALK. In one embodiment, the compound selectively inhibits ALK over ROS1. By way of non-limiting example, the ratio of selectivity can be greater than a factor of about 1.5, greater than a factor of about 2, than a factor of about 3, greater than a factor of about 4, greater than a factor of about 5, or greater than a factor of about 10, where selectivity can be measured by ratio of IC50 values, among other means. In one embodiment, the selectivity of ALK over ROS1 is measured by the ratio of the IC50 value against ROS1 to the IC50 value against ALK.

In one embodiment, the compound selectively inhibits ALK over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, or greater than a factor of about 10,000, where selectivity can be measured by ratio of IC50 values, among other means. In one embodiment, the selectivity of ALK over TRK is measured by the ratio of the IC50 value against TRK to the IC50 value against ALK.

In one embodiment, the compound selectively inhibits ROS1 and ALK over TRK (e.g., TRKA, TRKB, and/or TRBC). By way of non-limiting example, the ratio of selectivity can be greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by ratio of IC50 values, among other means. In one embodiment, the selectivity of ROS1 and ALK over TRK is measured by the ratio of the IC50 value against TRK to the IC50 value against ROS1 and ALK.

In one embodiment, provided herein is a method for selectively inhibiting ROS1 over TRK (e.g., TRKA, TRKB, and/or TRBC) wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ROS1, said method comprising selectively inhibiting ROS1 over TRK (e.g., TRKA, TRKB, and/or TRBC) by administering an amount of a compound or a pharmaceutical composition provided herein to said subject, wherein said amount is sufficient for selective inhibiting ROS1 over TRK (e.g., TRKA, TRKB, and/or TRBC).

In one embodiment, provided herein is a method for selectively inhibiting ALK over ROS1 wherein the inhibition takes place in a cell. In one embodiment, provided herein is a method for selectively inhibiting ALK over TRK (e.g., TRKA, TRKB, and/or TRBC) wherein the inhibition takes place in a cell. In one embodiment, the method comprises contacting ALK with an effective amount of a compound provided herein. In an embodiment, such contact occurs in a cell. In an embodiment, such contact occurs in a cell in a mammal such as a human. In an embodiment, such contact occurs in a cell in human patient having a cancer provided herein.

In one embodiment, provided herein is a method for selectively inhibiting ALK over ROS1 wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ALK, said method comprising selectively inhibiting ALK over ROS1 by administering an amount of a compound or a pharmaceutical composition provided herein to said subject, wherein said amount is sufficient for selective inhibiting ALK over ROS1.

In one embodiment, provided herein is a method for selectively inhibiting ALK over TRK (e.g., TRKA, TRKB, and/or TRBC) wherein the inhibition takes place in a subject suffering from cancer, said method comprising administering an effective amount of a compound or a pharmaceutical composition provided herein to said subject. In certain embodiments, provided herein is a method of treating a subject suffering from a cancer associated with ALK, said method comprising selectively inhibiting ALK over TRK (e.g., TRKA, TRKB, and/or TRBC) by administering an amount of a compound or a pharmaceutical composition provided herein to said subject, wherein said amount is sufficient for selective inhibiting ALK over TRK (e.g., TRKA, TRKB, and/or TRBC).

As used herein and unless otherwise specified, inhibition of ROS1 includes inhibition of wild type ROS1, a mutation, a rearrangement, or amplification or copy gain thereof, inhibition of ALK includes inhibition of wild type ALK, a mutation, a rearrangement, or amplification or copy gain thereof, or a partially deleted ALK protein; and inhibition of TRK includes inhibition of wild type TRK, or a mutation thereof.

Cancers treated by methods of the present disclosure include, but are not limited to, lung cancer, e.g., non-small cell lung cancer, inflammatory myofibroblastic tumor, ovarian cancer, e.g., serous ovarian carcinoma, melanoma, e.g., spitzoid melanoma, glioblastoma, bile duct cancer, e.g., cholangiocarcinoma, gastric cancer, colorectal cancer, angiosarcoma, anaplastic large cell lymphoma, diffuse large B-cell lymphoma, large B-cell lymphoma, esophageal cancer, e.g., esophageal squamous cell carcinoma, kidney cancer, e.g., renal medullary carcinoma or renal cell carcinoma, breast cancer, e.g., triple negative breast cancer, thyroid cancer, e.g., papillary thyroid cancer, neuroblastoma, epithelioid hemangioendothelioma, colon cancer, and spitzoid tumor.

In one embodiment, cancers treated by methods of the present disclosure include cancers originating from one or more oncogenic proteins selected from ROS1, ALK, TRKA, TRKB, and TRKC. In certain embodiments, cancers treated by methods of the present disclosure include cancers that are drug resistant to treatments directed at one or more oncogenic proteins selected from ROS1, ALK, TRKA, TRKB, and TRKC.

In one embodiment, the cancer in a method provided herein is anaplastic lymphoma kinase positive (ALK+). As used herein and unless otherwise specified, an “ALK positive” (ALK+) cancer, disease, or disorder refers to a cancer, disease, or disorder characterized by inappropriate (e.g., inappropriately high) expression of an ALK gene and/or the presence of a mutation in an ALK gene and/or the presence of a partially deleted ALK protein, and/or a mutation in the ALK protein, and/or is mediated by ALK, and/or that responds to inhibition of ALK. In one embodiment, “ALK positive” (ALK+) cancer, disease, or disorder refers to a cancer, disease, or disorder characterized by inappropriately high expression of an ALK gene and/or the presence of a mutation in an ALK gene, or is mediated by ALK. In one embodiment, “ALK positive” (ALK+) cancer, disease, or disorder refers to a cancer, disease, or disorder characterized by the presence of a partially deleted ALK protein (e.g., NB1, AskaSS). In one embodiment, “ALK positive” (ALK+) cancer is mediated by a genetically altered ALK. In some embodiments, the cancer, disease, or disorder carries ALK wild-type gene or genetically altered ALK gene. In one embodiment, “ALK positive” (ALK+) cancer is mediated by a fusion protein comprising a fragment of a protein encoded by an ALK gene and a fragment of a protein encoded by a gene selected from the group consisting of NPM, EML4, TPR, TFG, ATIC, CLTC1, TPM4, MSN ALO17, and MYH9. In one embodiment, the fusion protein is one or more of an EML4-ALK fusion protein, an NPM-ALK fusion protein, or a TPR-ALK fusion protein. In some embodiments, the genetically altered ALK is an EML4-ALK fusion protein. In some embodiments, the EML4-ALK fusion protein is a wild-type protein. In some embodiments, the EML4-ALK fusion protein comprises at least one resistance mutation. In some embodiments, the EML4-ALK fusion protein comprises at least one mutation selected from the group consisting of L1196M, G1202R, D1203N, L1152P/R, F1174C/L/V, C1156Y, I1171N, G1123S, S1206Y, G1269S/A, and T1151_L1152insT. In some embodiments, the EML4-ALK fusion protein comprises at least one mutation selected from the group consisting of G1202R, G1202K, L1196M, G1269A, G1269V, C1156Y, I1171T, I1171N, I1171S, F1174I, F1174L, F1174S, V1180L, S1206Y, E1129K, E1210K, T1151M, T1151_L1152insT, F1174C, G1202del, D1203N, S1206C, S1206F, L1152R, L1196Q, L1198P, L1198F, L1198H, R1275Q, L1152P, C1156T, F1245C, T1151K, I1268V, F1174V, L1198Q, S1206A, and F1245V.

In one embodiment, “ALK positive” (ALK+) cancer is mediated by a fusion protein comprising a fragment of a protein encoded by an ALK gene and a fragment of a protein encoded by a gene selected from the group consisting of NPM gene. In some embodiments, the genetically altered ALK is an NPM-ALK fusion protein. In some embodiments, the fusion protein comprises a fragment of a protein encoded by an ALK gene and a fragment of a protein encoded by a TPR gene. In some embodiments, the genetically altered ALK is a TPR-ALK fusion protein. In some embodiments, the TPR-ALK fusion protein contains a wild-type kinase domain. In some embodiments, the TPR-ALK fusion protein comprises at least one resistance mutation. In some embodiments, the TPR-ALK fusion protein comprises a L1196M mutation.

In one embodiment, the mutation alters the biological activity of an ALK nucleic acid molecule or polypeptide. As used herein and unless otherwise specified, a “mutation” or “mutant” of ALK comprises one or more deletions, substitutions, insertions, inversions, duplications, translocations, amplifications, or missense mutations, in the amino acid or nucleotide sequences of ALK, or fragments thereof. As used herein and unless otherwise specified, an ALK “rearrangement” refers to genetic translocations involving the ALK gene that may result in ALK fusion genes and/or ALK fusion proteins. The ALK fusion can also include one or more deletions, substitutions, insertions, inversions, duplications, translocations, or amplifications or a fragment thereof, as long as the mutant retains kinase phosphorylation activity.

In some embodiments, provided here is a method of treating a cancer in a subject, comprising identifying a generically altered ALK in the subject and administering to the subject a therapeutically effective amount of Compound 1 or a pharmaceutically acceptable salt thereof.

In one embodiment, the ALK mutation comprises one or more ALK point mutations. In some embodiments, cancers treated by methods of the present disclosure include one or more mutations in ALK kinase. In one embodiment, the one or more ALK point mutations are selected from point mutations at T1151, L1152, C1156, I1171, F1174, V1180, L1196, L1198, G1202, D1203, S1206, E1129, E1210, F1245, G1269, and R1275. In one embodiment, the one or more ALK point mutations is selected from R1060H, F1174C/I/L/S/V, F1245C/I/L/V, R1275L/Q, T1151M, M1166R, I1171N, I1171S, I1171N, I1183T, L1196M, A1200V, L1204F, L1240V, D1270G, Y1278S, R1192P, G1128A, G1286R, and T1343I. In one embodiment, the one or more ALK point mutations are selected from G1202R, G1202K, L1196M, G1269A, G1269V, C1156Y, I1171T, I1171N, I1171S, F1174I, F1174L, F1174S, V1180L, S1206Y, E1129K, E1210K, T1151M, T1151_L1152insT, F1174C, G1202del, D1203N, S1206Y, S1206C, S1206F, L1152R, L1196Q, L1198P, L1198F, L1198H, R1275Q, L1152P, C1156T, F1245C, T1151K, I1268V, F1174V, L1198Q, S1206A, and F1245V. In one embodiment, the ALK mutation is G1202R. In one embodiment, the ALK mutation is L1196M. In one embodiment, the ALK mutation is G1269A. In one embodiment, the ALK mutation is G1269V. In one embodiment, the ALK mutation is L1198F. In one embodiment, the ALK mutation is L1198H. In one embodiment, the ALK mutation is T1151M. In one embodiment, the ALK mutation is F1174L. In one embodiment, the ALK mutation is F1174I. In one embodiment, the ALK mutation is F1174S. In one embodiment, the ALK mutation is I1171N. In one embodiment, the ALK mutation is I1171S. In one embodiment, the ALK mutation is I1171T. In one embodiment, the ALK mutation is I1171N. In one embodiment, the ALK mutation is E1129K. In one embodiment, the ALK mutation is S1206F. In one embodiment, the ALK mutation is E1210K. In one embodiment, the ALK mutation is D1203N. In one embodiment, the ALK mutation is R1275G. In one embodiment, the ALK mutation is F1245C. In one embodiment, the ALK mutation is T1151K. In one embodiment, the ALK mutation is I1268V. In one embodiment, the ALK mutation is F1174V. In one embodiment, the ALK mutation is L1198Q. In one embodiment, the ALK mutation is S1206A.

As used herein and unless otherwise specified, a “co-mutation” refers to co-occurring mutations, i.e. when two or more mutations are present at the same time, for example in the same cell and on the same allele, in the same cell but on different alleles, or in different cells.

As used herein and unless otherwise specified, a “compound mutation” refers two or more mutations located on the same allele. A compound mutation is a subset of co-mutations. Compound mutations are also sometimes referred to as dual mutations if there are two mutations located on the same allele.

In some embodiments, the ALK mutation is co-mutation of G1202R and one or more mutations selected from L1196M, G1269A, T1151M, F1174S, and L1198F. In one embodiment, the ALK mutation is G1202R/L1196M compound mutation. In one embodiment, the ALK mutation is G1202R/G1269A compound mutation. In one embodiment, the ALK mutation is G1202R/L1198F compound mutation. In one embodiment, the ALK mutation is G1202R/T1151M compound mutation. In one embodiment, the ALK mutation is G1202R/F1174S compound mutation. In one embodiment, the ALK mutation is G1202R/F1174L compound mutation. In one embodiment, the ALK mutation is co-mutation of C1156Y and one or more mutations selected from L1256F, S1206F, F1174V, and F1174I. In one embodiment, the ALK mutation is C1156Y/L1256F compound mutation. In one embodiment, the ALK mutation is C1156Y/S1206F compound mutation. In one embodiment, the ALK mutation is C1156Y/F1174V compound mutation. In one embodiment, the ALK mutation is C1156Y/F1174I compound mutation. In one embodiment, the ALK mutation is co-mutation of L1196M and one or more mutations selected from L1198H, I1179V, and L1256F. In one embodiment, the ALK mutation is L1196M/L1198H compound mutation. In one embodiment, the ALK mutation is L1196M/I1179V compound mutation. In one embodiment, the ALK mutation is L1196M/L1256F compound mutation.

In one embodiment, the ALK mutation is G1202R/L1196M dual mutation. In one embodiment, the ALK mutation is G1202R/G1269A dual mutation. In one embodiment, the ALK mutation is G1202R/L1198F dual mutation. In one embodiment, the ALK mutation is G1202R/T1151M dual mutation. In one embodiment, the ALK mutation is G1202R/F1174S dual mutation. In one embodiment, the ALK mutation is G1202R/F1174L dual mutation. In one embodiment, the ALK mutation is C1156Y/L1256F dual mutation. In one embodiment, the ALK mutation is C1156Y/S1206F dual mutation. In one embodiment, the ALK mutation is C1156Y/F1174V dual mutation. In one embodiment, the ALK mutation is C1156Y/F1174I dual mutation. In one embodiment, the ALK mutation is L1196M/L1198H dual mutation. In one embodiment, the ALK mutation is L1196M/I1179V dual mutation. In one embodiment, the ALK mutation is L1196M/L1256F dual mutation.

In one embodiment, the ALK mutation comprises one or more ALK rearrangements (in one embodiment, one rearrangement). In one embodiment, the ALK mutation comprises one or more ALK fusions (in one embodiment, one fusion). In some embodiments, cancers treated by methods of the present disclosure include ALK fusions. In one embodiment, the ALK fusion is with one of the fusion partners described in Ou et al., JTO Clinical and Research Reports, 1(1): 1-10, the entirety of which is incorporated herein by reference. In one embodiment, the ALK fusion is with one of the fusion partners selected from the group consisting of EML4, TFG, KIF5B, KLC1, STRN, HIP1, TPR, BIRC6, DCTN1, SQSTM1, SOCS5, SEC31A, CLTC, PRKAR1A, PPM1B, EIF2AK3, CRIM1, CEBPZ, PICALM, CLIP1, BCL11A, GCC2, LMO7, PHACTR1, CMTR1, VIT, DYSF, ITGAV, PLEKHA7, CUX1, VKORC1L1, FBXO36, SPTBN1, EML6, FBXO11, CLIP4, CAMKMT, NCOA1, MYT1L, SRBD1, SRD5A2, NYAP2, MPRIP, ADAM17, ALK, LPIN1, WDPCP, CEP55, ERC1, SLC16A7, TNIP2, ATAD2B, SLMAP, FBN1, SWAP70, TCF12, TRIM66, WNK3, AKAP8L, SPECC1L, PRKCB, CDK15, LCLAT1, YAP1, PLEKHM2, DCHS1, PPFIBP1, ATP13A4, C12orf75, EPAS1, FAM179A, FUT8, LIMD1, LINC00327, LOC349160, LYPD1, RBM20, TACR1, TANC1, TTC27, TUBBB, SMPD4, SORCS1, LINC00211, SOS1, C9orf3, CYBRD1, MTA3, THADA, TSPYL6, WDR37, and PLEKHH2. In one embodiment, the ALK fusion is with one of the fusion partners selected from the group consisting of EML4, TMP1, WDCP, GTF2IRD1, TPM3, TPM4, CLTC, LMNA, PRKAR1A, RANBP2, TFG, FN1, KLC1, VCL, STRN, HIP1, NPM1, DCTN1, SQSTM1, TPR, CRIM1, PTPN3, FBXO36, ATIC, MSN, ALO17, MYH9 and KIF5B. In one embodiment, the ALK mutation is EML4-ALK, a fusion between the echinoderm microtubule-associated protein-like 4 (EML4) gene and the ALK tyrosine kinase domain. There are many variants of EML4-ALK that differ by breakpoint junctions, with variant 1 (v1) and variant 3 (v3) being the most prevalent clinically. In one embodiment, the ALK mutation is NPM1-ALK. In one embodiment, the ALK mutation is STRN-ALK.

In one embodiment, the ALK mutation comprises one ALK rearrangement and one or more ALK point mutations. In one embodiment, the ALK mutation is EML4-ALK wt (variant 1). In one embodiment, the ALK mutation is EML4-ALK (variant 2). In one embodiment, the ALK mutation is EML4-ALK (variant 3). In one embodiment, the ALK mutation is EML4-ALK wt (variant 4, 5, 6, or 7). In one embodiment, the ALK mutation is EML4-ALK G1202R. In one embodiment, the ALK mutation is EML4-ALK I1171N. In one embodiment, the ALK mutation is EML4-ALK I1171S. In one embodiment, the ALK mutation is EML4-ALK I1171T. In one embodiment, the ALK mutation is EML4-ALK L1196M. In one embodiment, the ALK mutation is EML4-ALK D1203N. In one embodiment, the ALK mutation is EML4-ALK L1196M/G1202R. In one embodiment, the ALK mutation is EML4-ALK G1202R/G1269A. In one embodiment, the ALK mutation is EML4-ALK G1202R/L1196M. In one embodiment, the ALK mutation is EML4-ALK G1202R/L1198F. In one embodiment, the ALK mutation is EML4-ALK G1202R/T1151M. In one embodiment, the ALK mutation is EML4-ALK G1202R/F1174S. In one embodiment, the ALK mutation is EML4-ALK G1202R/F1174L.

In one embodiment, the ALK positive solid tumor is characterized by the presence of a mutation in an ALK gene. In one embodiment, the ALK mutation comprises one or more ALK rearrangement, one or more ALK point mutation, or a combination thereof. In one embodiment, the ALK mutation comprises G1202R, F1174C, F1174L, I1171N, I1171S, I1171T, L1196M, V1180L, C1156Y, G1202del, G1202K, G1269A, F1174S, S1206Y, E1210K, T1151M, T1151_L1152insT, D1203N, S1206C, L1152R, L1196Q, L1198P, L1198F, R1275Q, L1152P, C1156T, or F1245V, or a combination thereof. In one embodiment, the ALK mutation comprises G1202R. In one embodiment, the ALK mutation comprises F1174S or F1174L. In one embodiment, the ALK mutation comprises I1171S. In one embodiment, the ALK mutation comprises I1171T. In one embodiment, the ALK mutation comprises I1171N. In one embodiment, the ALK mutation comprises F1171M. In one embodiment, the ALK mutation comprises D1203N and one selected from I1171S, I1171T, I1171N, and I1171M. In one embodiment, the ALK mutation comprises C1156Y and one selected from I1171S, I1171T, I1171N, and I1171M. In one embodiment, the ALK mutation comprises R1275Q. In one embodiment, the ALK mutation comprises T1151M. In one embodiment, the ALK mutation comprises one or more compound mutations. In one embodiment, the compound mutation is G1202R/L1196M, G1202R/G1269A, G1202R/L1198F, or G1202R/F1174S. In one embodiment, the compound mutation is G1202R/L1196M. In one embodiment, the compound mutation is G1202R/G1269A. In one embodiment, the compound mutation is G1202R/L1198F. In one embodiment, the compound mutation is G1202R/F1174S. In one embodiment, the ALK positive solid tumor is characterized by the presence of a partially deleted ALK protein. In one embodiment, the ALK mutation is Ex2-3del. In one embodiment, the ALK mutation is Ex2-17del.

In one embodiment, partially deleted ALK proteins influence proliferative and metastatic properties of cancer cells. ALK protein can become partially deleted through various mechanisms. The first mechanism is shedding, where the 80-kDa extracellular domain of the ALK protein is post-translationally cleaved near residue Asn654, leaving the 140-kDa C-terminal transmembrane and intracellular domains on the cell. Shedding has been observed in many ALK-expressing cell lines, most notably from a neuroblastoma disease background. Shedding increases cancer cell migration and proliferation in preclinical models of cancer, both in vitro and in vivo (Moog-Lutz, JBC (2005), Huang, Cell Reports (2021)). The second mechanism is alternative transcription initiation (ATI), where transcription of the ALK gene begins at an alternative initiation site downstream of the original site, resulting in the absence of exons 1-18 and part of exon 19. ALK ATIs have been identified in 11% of melanomas as well as a small portion of lung cancers and anaplastic thyroid cancers. Expression of ALK ATI transforms Ba/F3 and NIH3T3 cells, conferring them with oncogenic potential. One patient with ALK ATI has shown clinical response to an ALK inhibitor therapy, suggesting that ALK ATI may be a targetable driver mutation (Wiesner, Nature (2015)). The third mechanism is partial deletion of the ALK gene, for example through a chromosomal rearrangement event. Multiple deletion variants have been identified, including deletion of exons 2-3, exons 1-5, exons 4-11, and exons 2-17, and some of these variants have been shown to activate ALK signaling as well as transform Ba/F3 or NIH3T3 cells. ALK partial deletions have been detected in neuroblastomas, sarcomas, and lymphomas. (Okubo, Oncogene (2012); Cazes, Can Res (2013); Fransson, Genes Chromosomes & Cancer (2014); Fleuren, Can Res (2017); Fukuhara, Hematol Oncol (2017)).

In one embodiment, the ALK+ cancer is determined by an FDA-approved test or other tests known in the art. The tests that can be used include, but not limited to, e.g., FoundationOne CDx™ (F1CDx) (a sequencing based in vitro diagnostic device for detection of substitutions, insertion and deletion alterations (indels), and copy number alterations (CNAs) and selected gene rearrangements, as well as genomic signatures including microsatellite instability (MSI) and tumor mutational burden (TMB) using DNA isolated from formalin-fixed paraffin embedded (FFPE) tumor tissue specimens); VENTANA ALK (D5F3) CDx Assay (qualitative detection of the anaplastic lymphoma kinase (ALK) protein in formalin-fixed, paraffin-embedded (FFPE) non-small cell lung carcinoma (NSCLC) tissue stained with the BenchMark XT or BenchMark ULTRA automated staining instrument); and Vysis ALK Break Apart FISH Probe Kit test (a qualitative test to detect rearrangements involving the ALK gene via fluorescence in situ hybridization (FISH) in formalin-fixed, paraffin-embedded (FFPE) non-small cell lung cancer (NSCLC) tissue specimens). In one embodiment, the test is a fluorescence in situ hybridization (FISH) test, e.g., Vysis ALK Break Apart FISH Probe Kit test. Additional information for FDA-approved tests can be found at, e.g., https://www.fda.gov/MedicalDevices/ProductsandMedicalProcedures/InVitroDiagnostics/ucm303030.htm; and additional information for Vysis ALK Break Apart FISH Probe Kit can be found at, e.g., https://www.molecular.abbott/us/en/products/oncology/vysis-alk-break-apart-fish-probe-kit; the entirety of which are incorporated herein by reference.

In one embodiment of any of the methods described herein, the presence of an ALK mutation in the sample indicates that the subject has or is at increased risk for developing an ALK positive (e.g. ALK-driven) cancer. In other embodiments, the presence of the mutation in the sample indicates that the subject has or is at increased risk for developing an ALK positive (e.g. ALK-driven) cancer refractory to treatment with a TKI. In particular embodiments, the presence of the mutation in the sample indicates that the subject has or is at increased risk for developing an ALK positive (e.g. ALK-driven) cancer refractory to treatment with to one or more of crizotinib, ceritinib, alectinib, brigatinib, lorlatinib, and ASP3026. In particular embodiments, the presence of the mutation in the sample indicates that the subject has or is at increased risk for developing an ALK positive (e.g. ALK-driven) cancer refractory to treatment with to one or more of crizotinib, ceritinib, alectinib, brigatinib, and lorlatinib. In some embodiments, the ALK protein or ALK-fusion protein includes a contiguous sequence of between 30 and 1620 amino acids that has at least 95% identity to the amino acid sequence of the ALK [Homo sapiens] (NCBI Reference Sequence: NP_004295.2 or UniProt Sequence No.: Q9UM73 sequence). In another non-limiting embodiments, the ALK protein or ALK-fusion protein includes a contiguous sequence of between 30 and 1620 amino acids that has at least about 85% identity to the amino acid sequence of the ALK [Homo sapiens] (NCBI Reference Sequence: NP_004295.2 or UniProt Sequence No.: Q9UM73 sequence). In another non-limiting embodiments, where the ALK protein or ALK-fusion protein includes a contiguous sequence of between 30 and 1620 amino acids that has at least about 90% identity to the amino acid sequence of the ALK [Homo sapiens] (NCBI Reference Sequence: NP_004295.2 or UniProt Sequence No.: Q9UM73 sequence). In another non-limiting embodiments, where the ALK protein or ALK-fusion protein includes a contiguous sequence of between 30 and 1620 amino acids that has at least about 95% identity to the amino acid sequence of the ALK [Homo sapiens] (NCBI Reference Sequence: NP_004295.2 or UniProt Sequence No.: Q9UM73 sequence). In another non-limiting embodiments, where the ALK protein or ALK-fusion protein includes a contiguous sequence of between 30 and 1620 amino acids that has at least about 85-90%, 91-93%, 94%, 95%, 96%, 97%, 98%, or 99% identity to the amino acid sequence of ALK [Homo sapiens] (NCBI Reference Sequence: NP_004295.2 or UniProt Sequence No.: Q9UM73 sequence).

Also provided are methods of treating a subject having a cancer (e.g., an ALK positive cancer) that include: determining whether a cancer cell in a sample obtained from a subject having a cancer and previously administered a first ALK inhibitor, has one or more ALK inhibitor resistance mutations; and administering Compound 1 (e.g. Form 2) or a pharmaceutically acceptable salt thereof as a monotherapy or in combination with another anticancer agent to the subject if the subject has a cancer cell that has one or more ALK inhibitor resistance mutations. In some embodiments, the one or more ALK inhibitor resistance mutations confer increased resistance to a cancer cell or tumor to treatment with the first ALK inhibitor. In some embodiments, the one or more ALK inhibitor resistance mutations include one or more ALK inhibitor resistance mutations. For example, the one or more ALK inhibitor resistance mutations can include one or more point mutations at one or more of amino acid positions 1202, 1196, 1269, 1156, 1171, 1174, 1180, 1206, 1210, 1151, 1203, 1152, 1198, 1275, and 1245, e.g., G1202R, L1196M, G1269A, C1156Y, I1171T, I1171N, I1171S, F1174L, F1174S, V1180L, S1206Y, E1210K, T1151M, T1151_L1152insT, F1174C, G1202del, D1203N, S1206Y, S1206C, L1152R, L1196Q, L1198P, L1198F, R1275Q, L1152P, C1156T, and F1245V. In some embodiments, another anticancer agent is any anticancer agent known in the art. For example, another anticancer agent can be another ALK inhibitor (e.g., a second ALK inhibitor).

In one embodiment, the cancer in a method provided herein is ROS1 positive (ROS1+). As used herein and unless otherwise specified, a “ROS1 positive” (ROS1+) cancer, disease, or disorder refers to a cancer, disease, or disorder characterized by inappropriately high expression of a ROS1 gene and/or the presence of a mutation in a ROS1 gene. In one embodiment, the mutation alters the biological activity of a ROS1 nucleic acid molecule or polypeptide. As used herein and unless otherwise specified, a “mutation” or “mutant” of ROS1 comprises one or more deletions, substitutions, insertions, inversions, duplications, translocations, or amplifications in the amino acid or nucleotide sequences of ROS1, or fragments thereof. As used herein and unless otherwise specified, a ROS1 “rearrangement” refers to genetic translocations involving the ROS1 gene that may result in ROS1 fusion genes and/or ROS1 fusion proteins. The ROS1 fusion can also include one or more deletions, substitutions, insertions, inversions, duplications, translocations, or amplifications or a fragment thereof, as long as the mutant retains kinase phosphorylation activity.

In one embodiment, the ROS1 mutation comprises one or more ROS1 point mutations. In some embodiments, cancers treated by methods of the present disclosure include one or more mutations in ROS1 kinase. In one embodiment, the one or more ROS1 point mutations are selected from point mutations at E1935, L1947, L1951, G1971, E1974, L1982, S1986, F2004, E2020, L2026, G2032, D2033, C2060, F2075, L2086, V2089, V2098, G2101, D2113, and L2155. In one embodiment, the one or more ROS1 point mutations are selected from G2032R, G2032K, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, and L2086F. In one embodiment, the ROS1 mutation is G2032R. In one embodiment, the ROS1 mutation is S1986F. In one embodiment, the ROS1 mutation is S1986Y. In one embodiment, the ROS1 mutation is L2026M. In one embodiment, the ROS1 mutation is D2033N. In one embodiment, the ROS1 mutation is L2086F. In one embodiment, the ROS1 mutation is F2004C. In one embodiment, the ROS1 mutation is F2004V. In one embodiment, the ROS1 mutation is G2101A. In one embodiment, the ROS1 mutation is L1982F. In one embodiment, the ROS1 mutation is co-mutation of G2032R and one or more of S1986F, S1986Y, F2004C, F2004V, L2026M, or D2033N.

In one embodiment, the ROS1 mutation comprises one or more ROS1 rearrangements (in one embodiment, one rearrangement). In one embodiment, the ROS1 mutation comprises one or more ROS1 fusions (in one embodiment, one fusion). In some embodiments, cancers treated by methods of the present disclosure include ROS1 fusions. In one embodiment, the ROS1 fusion is with one of the fusion partners selected from SLC34A2, CD74, TPM3, SDC4, EZR, LRIG3, KDELR2, CEP72, CLTL, CTNND2, GOPC (e.g., GOPC-S, GOPC-L), GPRC6A, LIMA1, LRIG3, MSN, MYO5C, OPRM1, SLC6A17 SLMAP, SRSF6, TFG, TMEM106B, TPD52L1, ZCCHC8, CCDC6, CAPRIN1, CEP85L, CHCHD3, CLIP1, EEF1G, KIF21A, KLC1, SART3, ST13, TRIM24, ERC1, FIP1L1, HLAA, KIAA1598, MYO5A, PPFIBP1, PWWP2A, FN1, YWHAE, CCDC30, NCOR2, NFKB2, APOB, PLG, RBP4, and GOLGB1. In one embodiment, the ROS1 fusion is CD74-ROS1 fusion. In one embodiment, the ROS1 fusion is SDC4-ROS1 fusion. In one embodiment, the ROS1 fusion is EZR-ROS1 fusion. In one embodiment, the ROS1 fusion is SLC34A2-ROS1 fusion. In one embodiment, the ROS1 fusion is GOPC-ROS1 fusion (e.g., GOPC-ROS1-S, GOPC-ROS1-L). In one embodiment, the ROS1 fusion is CEP85L-ROS1 fusion.

In one embodiment, the ROS1 mutation comprises one ROS1 rearrangements and one or more ROS1 point mutations. In one embodiment, the ROS1 mutation comprises one or more ROS1 rearrangements from CD74-ROS1, EZR-ROS1, SLC34A2-ROS1, GOPC-ROS1 (e.g., GOPC-ROS1-S, GOPC-ROS1-L), and CEP85L-ROS1, and one or more ROS1 point mutations selected from F2004C, F2004V, and G2032R. In one embodiment, the ROS1 mutation comprises one or more ROS1 rearrangements from CD74-ROS1, EZR-ROS1, and SLC34A2-ROS1, and ROS1 point mutation of G2101A.

In one embodiment, the ROS1 mutation is CD74-ROS1 F2004C. In one embodiment, the ROS1 mutation is CD74-ROS1 F2004V. In one embodiment, the ROS1 mutation is CD74-ROS1 G2101A. In one embodiment, the ROS1 mutation is CD74-ROS1 G2032R. In one embodiment, the ROS1 mutation is CD74-ROS1 S1986F. In one embodiment, the ROS1 mutation is CD74-ROS1 L2026M. In one embodiment, the ROS1 mutation is CD74-ROS1 D2033N. In one embodiment, the ROS1 mutation is EZR-ROS1 F2004C. In one embodiment, the ROS1 mutation is EZR-ROS1 F2004V. In one embodiment, the ROS1 mutation is EZR-ROS1 G2101A. In one embodiment, the ROS1 mutation is EZR-ROS1 G2032R. In one embodiment, the ROS1 mutation is SLC34A2-ROS1 F2004C. In one embodiment, the ROS1 mutation is SLC34A2-ROS1 F2004V. In one embodiment, the ROS1 mutation is SLC34A2-ROS1 G2101A. In one embodiment, the ROS1 mutation is SLC34A2-ROS1 G2032R. In one embodiment, the ROS1 mutation is GOPC-ROS1 F2004C (e.g., GOPC-ROS1-S F2004C, GOPC-ROS1-L F2004C). In one embodiment, the ROS1 mutation is GOPC-ROS1 F2004V (e.g., GOPC-ROS1-S F2004V, GOPC-ROS1-L F2004V). In one embodiment, the ROS1 mutation is GOPC-ROS1 G2032R (e.g., GOPC-ROS1-S G2032R, GOPC-ROS1-L G2032R). In one embodiment, the ROS1 mutation is CEP85L-ROS1 F2004C. In one embodiment, the ROS1 mutation is CEP85L-ROS1 F2004V. In one embodiment, the ROS1 mutation is CEP85L-ROS1 G2032R. In one embodiment, the ROS1 mutation is GOPC-ROS1 L1982F (e.g., GOPC-ROS1-S L1982F, GOPC-ROS1-L L1982F). In one embodiment, the ROS1 mutation is CD74-ROS1 L1982F.

In one embodiment, the ROS1+ cancer is determined by an FDA-approved test or other tests known in the art. The tests that can be used include, e.g., Oncomine™ Dx Target Test by Thermo Fisher Scientific. (a qualitative in vitro diagnostic test that uses targeted high-throughput, parallel-sequencing technology to detect sequence variations in 23 genes in DNA and RNA isolated from formalin-fixed, paraffin-embedded tumor (FFPE) tissue samples from patients with non-small cell lung cancer (NSCLC) using the Ion PGM Dx System); Vysis ROS1 Break Apart FISH Probe Kit (a qualitative test to detect rearrangements involving ROS1 gene rearrangements at 6q22 via fluorescence in situ hybridization (FISH) in formalin-fixed, paraffin-embedded (FFPE) non-small cell lung cancer (NSCLC) tissue specimens) or RTReal Time-Polymerase Chain Reaction (RT-PCR) or NGSNext Generation Sequencing via a local diagnostic test.

Also provided are methods of treating a subject having a cancer (e.g., a ROS1 positive cancer) that include: determining whether a cancer cell in a sample obtained from a subject having a cancer and previously administered a first ROS1 inhibitor, has one or more ROS1 inhibitor resistance mutations; and administering a compound of Formula (I) or a pharmaceutically acceptable salt or solvate thereof as a monotherapy or in conjunction with another anticancer agent to the subject if the subject has a cancer cell that has one or more ROS1 inhibitor resistance mutations. In some embodiments, the one or more ROS1 inhibitor resistance mutations confer increased resistance to a cancer cell or tumor to treatment with the first ROS1 inhibitor. In some embodiments, the one or more ROS1 inhibitor resistance mutations include one or more ROS1 inhibitor resistance mutations. For example, the one or more ROS1 inhibitor resistance mutations can include a substitution at one or more of amino acid positions 2032, 2033, 1986, 2026, 1951, 1935, 1947, 1971, 1974, 1982, 2004, 2020, 2060, 2075, 2089, 2098, 2101, 2113, 2155, 2032, and 2086, e.g., G2032R, D2033N, S1986F, S1986Y, L2026M, L1951R, E1935G, L1947R, G1971E, E1974K, L1982F, F2004C, F2004V, E2020K, C2060G, F2075V, V2089M, V2098I, G2101A, D2113N, D2113G, L2155S, L2032K, and L2086F. In some embodiments, another anticancer agent is any anticancer agent known in the art. For example, another anticancer agent can be another ROS1 inhibitor (e.g., a second ROS1 inhibitor).

In one embodiment, a compound provided herein is a CNS-penetrating compound. In one embodiment, after the administration of an effective amount of a compound provided herein (e.g., orally or intravenously), the compound is able to penetrate CNS (e.g., blood-brain barrier) and achieve a concentration in CNS (e.g., brain) that is still sufficient to inhibit (e.g., selectively inhibit) ROS1 or ALK or both.

In one embodiment, provided herein is a method for treating CNS metastases of a cancer, comprising administering to a subject in need thereof an effective amount of a compound provided herein, e.g., a compound of Formula (I), or an enantiomer, a mixture of enantiomers, or a tautomer thereof, or a pharmaceutically acceptable salt thereof. In one embodiment, the CNS metastases is brain metastases. In one embodiment, the cancer is a ROS1+ cancer. In one embodiment, the cancer is an ALK+ cancer.

In one embodiment, the solid tumor (or cancer) is leukocyte receptor tyrosine kinase (LTK) positive. In one embodiment, the solid tumor is LTK positive breast invasive ductal carcinoma, prostate adenocarcinoma, pancreatic adenocarcinoma, adenocarcinoma of unknown primary, or bladder urothelial carcinoma. In one embodiment, the cancer is LTK positive leukemia. In one embodiment, the solid tumor is LTK positive lung cancer. In one embodiment, the solid tumor is LTK positive NSCLC. In one embodiment, the solid tumor (or cancer) has an LTK mutation. In one embodiment, the LTK mutation is G269A, F218I, N257T, A13fs, or A214fs. In one embodiment, the solid tumor (or cancer) has an LTK fusion. In one embodiment, the LTK fusion is CLIP1-LTK. See Cooper A J, Sequist L V, Johnson T W, Lin J J. LTK fusions: A new target emerges in non-small cell lung cancer. Cancer Cell. 2022 Jan. 10; 40(1):23-25; and Izumi, H., Matsumoto, S., Liu, J. et al. The CLIP1-LTK fusion is an oncogenic driver in non-small-cell lung cancer. Nature 600, 319-323 (2021), each of which are incorporated herein by reference in their entirety.

In some embodiments, the compound is an inhibitor of human tropomyosin receptor kinase A, B, or C. In certain embodiments, the IC50 of the compound for inhibition of mutant or non-mutant ROS1 or ALK is no more than one-fifth of the IC50 of the compound for inhibition of wild-type tropomyosin receptor kinase A, B, or C. TRK inhibition, particularly in the central nervous system (CNS), has been associated with adverse reactions, including dizziness/ataxia/gait disturbance, paraesthesia, weight gain and cognitive changes.

In some embodiments, provided is a method of minimizing adverse events in a subject in need of treatment for cancer (e.g., a ROS1 positive cancer or an ALK positive cancer), the method comprising administering to the subject a therapeutically effective amount of a compound provided herein, e.g., a compound of Formula (I), an enantiomer, a mixture of enantiomers, or a tautomer thereof, or a pharmaceutically acceptable salt thereof, and wherein the method minimizes adverse events associated with TRK inhibitors. In some embodiments, the cancer is a ROS1-associated cancer or an ALK-associated (or ALK+) cancer. In some embodiments, the adverse events are TRK-related CNS adverse events.

As used herein “minimizing” adverse events refers to a reduction in the incidence of adverse events in a subject or patient population compared to the paradigmatic incidence of adverse events in a subject or patient population treated with TRK inhibitors (e.g., entrectinib, repotrectinib, or lorlatinib). In some embodiments, the incidence of an adverse event refers to the frequency or percentage of a specific adverse event over a subject or patient population. In some embodiments, the incidence of an adverse event refers to the total number of adverse events experienced by an individual subject. In some embodiments, minimizing adverse events refers to minimizing TRK-related CNS adverse events. In some embodiments, minimizing TRK-related CNS adverse events means less than 40% of the patient population has a TRK-related CNS adverse event. In some embodiments, minimizing TRK-related CNS adverse events means less than 35%, less than 30%, less than 25%, less than 20%, less than 15%, less than 10% or less than 5% of the patient population has a TRK-related CNS adverse event. In some embodiments, minimizing TRK-related CNS adverse events means less than 12% of the patient population have more than one TRK-related CNS adverse event. In some embodiments, minimizing TRK-related CNS adverse events means less than 11%, less than 10%, less than 9%, less than 8%, less than 7%, less than 6%, less than 5%, less than 4%, or less than 3% of the patient population have more than one TRK-related CNS adverse event.

In some embodiments, TRK-related CNS adverse events refers to one or more of the following: dizziness, ataxia, gait disturbance, paraesthesia, weight gain, hyperphagia, paresthesias, abnormal movement, cognitive changes, speech effects (e.g, dysarthria, slow speech, or speech disorder), mood disorder (e.g., irritability, anxiety, depression, affect lability, personality change, mood swings, affective disorder, aggression, agitation, mood altered, depressed mood, euphoric mood, or mania), and cognitive disorder (e.g., memory impairment, cognitive disorder, amnesia, confusion, disturbance in attention, delirium, mental impairment, attention deficit/hyperactivity disorder, dementia, or reading disorder).

In one embodiment, provided herein is a method for preventing or limiting TRK-related CNS side effect or adverse event in a cancer treatment, comprising administering to a subject in need thereof an effective amount of a compound provided herein, e.g., a compound of Formula (I), or an enantiomer, a mixture of enantiomers, or a tautomer thereof, or a pharmaceutically acceptable salt thereof. In one embodiment, the method prevents the occurrence of the TRK-related CNS adverse event. In one embodiment, the method limits the frequency of occurrence of the TRK-related CNS adverse event. In one embodiment, the method limits the severity of the TRK-related side effect. In one embodiment, provided herein is a method for treating CNS metastases of a cancer with reduced TRK-related side effect, comprising administering to a subject in need thereof an effective amount of a compound provided herein, e.g., a compound of Formula (I), or an enantiomer, a mixture of enantiomers, or a tautomer thereof, or a pharmaceutically acceptable salt thereof. In one embodiment, the reduction/limiting/prevention in CNS side effect or adverse event is determined in a statistical sample, as compared to a standard of care treatment, e.g., an approved ROS1 and/or ALK inhibitor (e.g., crizotinib, entrectinib, lorlatinib, or repotrectinib) for ROS1+ and/or ALK+ cancer. In one embodiment, the TRK-related side effect is a TRKB-related CNS side effect. In one embodiment, the TRK-related CNS side effect or adverse event is dizziness, ataxia, gait disturbance, paraesthesia, weight gain, cognitive impairment, a mood disorder, or sleep disturbance.

In one embodiment, provided herein is a method for treating cancer, comprising administering to a subject in need thereof a therapeutically effective amount of a compound provided herein, e.g., a compound of Formula (I), or an enantiomer, a mixture of enantiomers, or a tautomer thereof, or a pharmaceutically acceptable salt thereof. In one embodiment, the cancer is a ROS1-associated cancer. In one embodiment, the cancer is a ROS1+ cancer. In one embodiment, the cancer is an ALK-associated cancer. In one embodiment, the cancer is an ALK+ cancer. In one embodiment, the cancer is identified to be ROS1+. In one embodiment, the cancer is identified to be ALK+. In one embodiment, the cancer is lung cancer, bile duct cancer, colorectal cancer, angiosarcoma, sarcoma, hemangioendothelioma, esophageal cancer, kidney cancer, breast cancer, colon cancer, thyroid cancer, neuroblastoma, hematological cancer, anaplastic large cell lymphoma (ALCL), atypical meningioma, breast cancer, cholangiocarcinoma, gastric cancer, glioblastoma, inflammatory myofibroblastic tumor (IMT), inflammatory hepatocellular adenoma (HCA), melanoma, pancreatic cancer, papillary thyroid carcinoma, salivary gland carcinoma, serous ovarian carcinoma, or spitzoid neoplasm.

In one embodiment, the cancer is solid tumor. In some embodiments, the solid tumor is advanced solid tumor. In one embodiment, the solid tumor is locally advanced or metastatic solid tumor. In one embodiment, the advanced solid tumor is relapsed after, refractory to, or resistant to the prior treatment by a tyrosine kinase inhibitor (TKI). In one embodiment, the solid tumor is non-small cell lung cancer (NSCLC). In one embodiment, the solid tumor is advanced NSCLC. In one embodiment, the solid tumor is metastatic. In one embodiment, the solid tumor is CNS metastatic. In one embodiment, the solid tumor is metastatic NSCLC. In one embodiment, the solid tumor is CNS metastatic NSCLC. As used herein and unless otherwise specified, “advanced tumor” refers to a tumor that cannot be cured or grows beyond the initial site of origin, either locally advanced or metastatic.

In one embodiment, the solid tumor (or cancer) is ALK positive. In one embodiment, the solid tumor is ALK positive NSCLC. In one embodiment, the solid tumor is advanced ALK positive solid tumor. In one embodiment, the solid tumor is advanced ALK positive NSCLC. In one embodiment, the solid tumor is metastatic ALK positive solid tumor. In one embodiment, the solid tumor is locally advanced ALK positive solid tumor. In one embodiment, the solid tumor is CNS metastatic ALK positive solid tumor. In one embodiment, the solid tumor is metastatic ALK positive NSCLC. In one embodiment, the solid tumor is CNS metastatic ALK positive NSCLC.

In one embodiment, the ALK positive solid tumor or cancer is anaplastic large cell lymphoma, inflammatory myofibroblastic tumors, diffuse large B-cell lymphoma, esophageal squamous cell carcinoma, renal medullary carcinoma, renal cell carcinoma, breast cancer, colorectal cancer, ovarian cancer, papillary thyroid carcinoma, cholangiocarcinoma, spitzoid tumors, neuroblastoma, or anaplastic thyroid cancer. In certain embodiments, the ALK positive solid tumor is anaplastic thyroid cancer. In one embodiment, the subject has not been treated with a prior therapy. In one embodiment, the subject is naïve to (i.e. not receiving) any tyrosine kinase inhibitor (TKI) therapy.

In one embodiment, provided herein is a method for treating a ROS1+ cancer, comprising administering to a subject in need thereof a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating an ALK+ cancer, comprising administering to a subject in need thereof a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating cancer in a subject, comprising: (i) identifying the cancer in the subject to be ROS1+, and (ii) administering to the subject a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating cancer in a subject, comprising: (i) identifying the cancer in the subject to be ALK+, and (ii) administering to the subject a therapeutically effective amount of a compound provided herein, e.g., a compound of Formula (I), or an enantiomer, a mixture of enantiomers, or a tautomer thereof, or a pharmaceutically acceptable salt thereof.

In one embodiment, the cancer (or ROS1+ cancer, or ALK+ cancer) is a solid tumor. In one embodiment, the cancer (or ROS1+ cancer, or ALK+ cancer) is lung cancer, e.g., non-small cell lung cancer (NSCLC), glioblastoma, inflammatory myofibroblastic tumor (IMT), bile duct cancer, e.g., cholangiocarcinoma, ovarian cancer, e.g., serous ovarian carcinoma, gastric cancer, colorectal cancer, angiosarcoma, melanoma, e.g., spitzoid melanoma, epithelioid hemangioendothelioma, esophageal cancer, e.g., esophageal squamous cell carcinoma (ESCC), kidney cancer, e.g., renal medullary carcinoma or renal cell carcinoma, breast cancer, e.g., triple negative breast cancer, colon cancer, thyroid cancer, e.g., papillary thyroid cancer, spitzoid tumor, or neuroblastoma.

In one embodiment, the cancer is lung cancer. In one embodiment, the cancer is non-small cell lung cancer. In one embodiment, the cancer is ROS1+ non-small cell lung cancer. In one embodiment, the cancer is ALK+ non-small cell lung cancer. In one embodiment, the cancer is relapsed or refractory non-small cell lung cancer. In one embodiment, the cancer is relapsed or refractory ROS1+ non-small cell lung cancer. In one embodiment, the cancer is relapsed or refractory ALK+ non-small cell lung cancer. In one embodiment, the cancer is newly diagnosed non-small cell lung cancer. In one embodiment, the cancer is newly diagnosed ROS1+ non-small cell lung cancer. In one embodiment, the cancer is newly diagnosed ALK+ non-small cell lung cancer.

In one embodiment, the cancer is glioblastoma. In one embodiment, the cancer is ROS1+ glioblastoma. In one embodiment, the cancer is ALK+ glioblastoma. In one embodiment, the cancer is relapsed or refractory glioblastoma. In one embodiment, the cancer is relapsed or refractory ROS1+ glioblastoma. In one embodiment, the cancer is relapsed or refractory ALK+ glioblastoma. In one embodiment, the cancer is newly diagnosed glioblastoma. In one embodiment, the cancer is newly diagnosed ROS1+ glioblastoma. In one embodiment, the cancer is newly diagnosed ALK+ glioblastoma.

In one embodiment, the cancer is IMT. In one embodiment, the cancer is ROS1+ IMT. In one embodiment, the cancer is ALK+ IMT. In one embodiment, the cancer is relapsed or refractory IMT. In one embodiment, the cancer is relapsed or refractory ROS1+ IMT. In one embodiment, the cancer is relapsed or refractory ALK+ IMT. In one embodiment, the cancer is newly diagnosed IMT. In one embodiment, the cancer is newly diagnosed ROS1+ IMT. In one embodiment, the cancer is newly diagnosed ALK+ IMT.

In one embodiment, the cancer is bile duct cancer. In one embodiment, the cancer is cholangiocarcinoma. In one embodiment, the cancer is ROS1+ cholangiocarcinoma. In one embodiment, the cancer is ALK+ cholangiocarcinoma. In one embodiment, the cancer is relapsed or refractory cholangiocarcinoma. In one embodiment, the cancer is relapsed or refractory ROS1+ cholangiocarcinoma. In one embodiment, the cancer is relapsed or refractory ALK+ cholangiocarcinoma. In one embodiment, the cancer is newly diagnosed cholangiocarcinoma. In one embodiment, the cancer is newly diagnosed ROS1+ cholangiocarcinoma. In one embodiment, the cancer is newly diagnosed ALK+ cholangiocarcinoma.

In one embodiment, the cancer is ovarian cancer. In one embodiment, the cancer is ROS1+ ovarian cancer. In one embodiment, the cancer is ALK+ ovarian cancer. In one embodiment, the cancer is relapsed or refractory ovarian cancer. In one embodiment, the cancer is relapsed or refractory ROS1+ ovarian cancer. In one embodiment, the cancer is relapsed or refractory ALK+ ovarian cancer. In one embodiment, the cancer is newly diagnosed ovarian cancer. In one embodiment, the cancer is newly diagnosed ROS1+ ovarian cancer. In one embodiment, the cancer is newly diagnosed ALK+ ovarian cancer. In one embodiment, the ovarian cancer is serous ovarian carcinoma. In one embodiment, the ovarian cancer is high grade serous ovarian carcinoma.

In one embodiment, the cancer is gastric cancer. In one embodiment, the cancer is ROS1+ gastric cancer. In one embodiment, the cancer is ALK+ gastric cancer. In one embodiment, the cancer is relapsed or refractory gastric cancer. In one embodiment, the cancer is relapsed or refractory ROS1+ gastric cancer. In one embodiment, the cancer is relapsed or refractory ALK+ gastric cancer. In one embodiment, the cancer is newly diagnosed gastric cancer. In one embodiment, the cancer is newly diagnosed ROS1+ gastric cancer. In one embodiment, the cancer is newly diagnosed ALK+ gastric cancer.

In one embodiment, the cancer is colorectal cancer. In one embodiment, the cancer is ROS1+ colorectal cancer. In one embodiment, the cancer is ALK+ colorectal cancer. In one embodiment, the cancer is relapsed or refractory colorectal cancer. In one embodiment, the cancer is relapsed or refractory ROS1+ colorectal cancer. In one embodiment, the cancer is relapsed or refractory ALK+ colorectal cancer. In one embodiment, the cancer is newly diagnosed colorectal cancer. In one embodiment, the cancer is newly diagnosed ROS1+ colorectal cancer. In one embodiment, the cancer is newly diagnosed ALK+ colorectal cancer.

In one embodiment, the cancer is angiosarcoma. In one embodiment, the cancer is ROS1+ angiosarcoma. In one embodiment, the cancer is ALK+ angiosarcoma. In one embodiment, the cancer is relapsed or refractory angiosarcoma. In one embodiment, the cancer is relapsed or refractory ROS1+ angiosarcoma. In one embodiment, the cancer is relapsed or refractory ALK+ angiosarcoma. In one embodiment, the cancer is newly diagnosed angiosarcoma. In one embodiment, the cancer is newly diagnosed ROS1+ angiosarcoma. In one embodiment, the cancer is newly diagnosed ALK+ angiosarcoma.

In one embodiment, the cancer is sarcoma. In one embodiment, the cancer is soft-tissue sarcoma. In one embodiment, the cancer is synovial sarcoma. In one embodiment, the cancer is one or more selected from the group consisting of inflammatory myofibroblastic tumor, Leiomyosarcoma, and neurofibroma. In one embodiment, the cancer is one or more selected from the group consisting of Ewing sarcoma, fibrosarcoma, osteosarcoma, pulmonary sarcoma, uterine carcinosarcoma, and uterine leiomyosarcoma.

In one embodiment, the cancer is melanoma. In one embodiment, the cancer is spitzoid tumor. In one embodiment, the cancer is spitzoid melanoma. In one embodiment, the cancer is ROS1+ spitzoid melanoma. In one embodiment, the cancer is ALK+ spitzoid melanoma. In one embodiment, the cancer is relapsed or refractory spitzoid melanoma. In one embodiment, the cancer is relapsed or refractory ROS1+ spitzoid melanoma. In one embodiment, the cancer is relapsed or refractory ALK+ spitzoid melanoma. In one embodiment, the cancer is newly diagnosed spitzoid melanoma. In one embodiment, the cancer is newly diagnosed ROS1+ spitzoid melanoma. In one embodiment, the cancer is newly diagnosed ALK+ spitzoid melanoma.

In one embodiment, the cancer is epithelioid hemangioendothelioma. In one embodiment, the cancer is ROS1+ epithelioid hemangioendothelioma. In one embodiment, the cancer is ALK+ epithelioid hemangioendothelioma. In one embodiment, the cancer is relapsed or refractory epithelioid hemangioendothelioma. In one embodiment, the cancer is relapsed or refractory ROS1+ epithelioid hemangioendothelioma. In one embodiment, the cancer is relapsed or refractory ALK+ epithelioid hemangioendothelioma. In one embodiment, the cancer is newly diagnosed epithelioid hemangioendothelioma. In one embodiment, the cancer is newly diagnosed ROS1+ epithelioid hemangioendothelioma. In one embodiment, the cancer is newly diagnosed ALK+ epithelioid hemangioendothelioma.

In one embodiment, the cancer is esophageal cancer. In one embodiment, the cancer is ESCC. In one embodiment, the cancer is ROS1+ ESCC. In one embodiment, the cancer is ALK+ ESCC. In one embodiment, the cancer is relapsed or refractory ESCC. In one embodiment, the cancer is relapsed or refractory ROS1+ ESCC. In one embodiment, the cancer is relapsed or refractory ALK+ ESCC. In one embodiment, the cancer is newly diagnosed ESCC. In one embodiment, the cancer is newly diagnosed ROS1+ ESCC. In one embodiment, the cancer is newly diagnosed ALK+ ESCC.

In one embodiment, the cancer is kidney cancer. In one embodiment, the cancer is renal medullary carcinoma. In one embodiment, the cancer is ROS1+ renal medullary carcinoma. In one embodiment, the cancer is ALK+ renal medullary carcinoma. In one embodiment, the cancer is relapsed or refractory renal medullary carcinoma. In one embodiment, the cancer is relapsed or refractory ROS1+ renal medullary carcinoma. In one embodiment, the cancer is relapsed or refractory ALK+ renal medullary carcinoma. In one embodiment, the cancer is newly diagnosed renal medullary carcinoma. In one embodiment, the cancer is newly diagnosed ROS1+ renal medullary carcinoma. In one embodiment, the cancer is newly diagnosed ALK+ renal medullary carcinoma. In one embodiment, the cancer is renal cell carcinoma. In one embodiment, the cancer is ROS1+ renal cell carcinoma. In one embodiment, the cancer is ALK+ renal cell carcinoma. In one embodiment, the cancer is relapsed or refractory renal cell carcinoma. In one embodiment, the cancer is relapsed or refractory ROS1+ renal cell carcinoma. In one embodiment, the cancer is relapsed or refractory ALK+ renal cell carcinoma. In one embodiment, the cancer is newly diagnosed renal cell carcinoma. In one embodiment, the cancer is newly diagnosed ROS1+ renal cell carcinoma. In one embodiment, the cancer is newly diagnosed ALK+ renal cell carcinoma.

In one embodiment, the cancer is breast cancer. In one embodiment, the cancer is ROS1+ breast cancer. In one embodiment, the cancer is ALK+ breast cancer. In one embodiment, the cancer is relapsed or refractory breast cancer. In one embodiment, the cancer is relapsed or refractory ROS1+ breast cancer. In one embodiment, the cancer is relapsed or refractory ALK+ breast cancer. In one embodiment, the cancer is newly diagnosed breast cancer. In one embodiment, the cancer is newly diagnosed ROS1+ breast cancer. In one embodiment, the cancer is newly diagnosed ALK+ breast cancer. In one embodiment, the breast cancer is triple negative breast cancer.

In one embodiment, the cancer is colon cancer. In one embodiment, the cancer is ROS1+ colon cancer. In one embodiment, the cancer is ALK+ colon cancer. In one embodiment, the cancer is relapsed or refractory colon cancer. In one embodiment, the cancer is relapsed or refractory ROS1+ colon cancer. In one embodiment, the cancer is relapsed or refractory ALK+ colon cancer. In one embodiment, the cancer is newly diagnosed colon cancer. In one embodiment, the cancer is newly diagnosed ROS1+ colon cancer. In one embodiment, the cancer is newly diagnosed ALK+ colon cancer.

In one embodiment, the cancer is thyroid cancer. In one embodiment, the cancer is papillary thyroid cancer. In one embodiment, the cancer is ROS1+ papillary thyroid cancer. In one embodiment, the cancer is anaplastic thyroid cancer (ATC). In one embodiment, the cancer is ALK+ papillary thyroid cancer. In one embodiment, the cancer is relapsed or refractory papillary thyroid cancer. In one embodiment, the cancer is relapsed or refractory ROS1+ papillary thyroid cancer. In one embodiment, the cancer is relapsed or refractory ALK+ papillary thyroid cancer. In one embodiment, the cancer is newly diagnosed papillary thyroid cancer. In one embodiment, the cancer is newly diagnosed ROS1+ papillary thyroid cancer. In one embodiment, the cancer is newly diagnosed ALK+ papillary thyroid cancer.

In one embodiment, the cancer is neuroblastoma. In one embodiment, the cancer is ROS1+ neuroblastoma. In one embodiment, the cancer is ALK+ neuroblastoma. In one embodiment, the cancer is relapsed or refractory neuroblastoma. In one embodiment, the cancer is relapsed or refractory ROS1+ neuroblastoma. In one embodiment, the cancer is relapsed or refractory ALK+ neuroblastoma. In one embodiment, the cancer is newly diagnosed neuroblastoma. In one embodiment, the cancer is newly diagnosed ROS1+ neuroblastoma. In one embodiment, the cancer is newly diagnosed ALK+ neuroblastoma.

In one embodiment, the cancer (or ROS1+ cancer, or ALK+ cancer) is a hematological cancer. In one embodiment, the cancer (or ROS1+ cancer, or ALK+ cancer) is lymphoma. In one embodiment, the lymphoma is non-Hodgkin lymphoma. In one embodiment, the lymphoma is anaplastic large cell lymphoma (ALCL), diffuse large B-cell lymphoma (DLBCL), or large B-cell lymphoma. In addition to hematological cancer, methods for treating other blood disorder or hematologic malignancy that is ROS1+ or ALK+ are also provided herein.

In one embodiment, the cancer is ALCL. In one embodiment, the cancer is ROS1+ ALCL. In one embodiment, the cancer is ALK+ ALCL. In one embodiment, the cancer is relapsed or refractory ALCL. In one embodiment, the cancer is relapsed or refractory ROS1+ ALCL. In one embodiment, the cancer is relapsed or refractory ALK+ ALCL. In one embodiment, the cancer is newly diagnosed ALCL. In one embodiment, the cancer is newly diagnosed ROS1+ ALCL. In one embodiment, the cancer is newly diagnosed ALK+ ALCL.

In one embodiment, the cancer is DLBCL. In one embodiment, the cancer is ROS1+ DLBCL. In one embodiment, the cancer is ALK+ DLBCL. In one embodiment, the cancer is relapsed or refractory DLBCL. In one embodiment, the cancer is relapsed or refractory ROS1+ DLBCL. In one embodiment, the cancer is relapsed or refractory ALK+ DLBCL. In one embodiment, the cancer is newly diagnosed DLBCL. In one embodiment, the cancer is newly diagnosed ROS1+ DLBCL. In one embodiment, the cancer is newly diagnosed ALK+ DLBCL.

In one embodiment, the cancer is large B-cell lymphoma. In one embodiment, the cancer is ROS1+ large B-cell lymphoma. In one embodiment, the cancer is ALK+ large B-cell lymphoma. In one embodiment, the cancer is relapsed or refractory large B-cell lymphoma. In one embodiment, the cancer is relapsed or refractory ROS1+ large B-cell lymphoma. In one embodiment, the cancer is relapsed or refractory ALK+ large B-cell lymphoma. In one embodiment, the cancer is newly diagnosed large B-cell lymphoma. In one embodiment, the cancer is newly diagnosed ROS1+ large B-cell lymphoma. In one embodiment, the cancer is newly diagnosed ALK+ large B-cell lymphoma.

In one embodiment, the cancer is one or more selected from the group consisting of acinar adenocarcinoma, adrenocortical carcinoma, anaplastic astrocytoma, anaplastic large cell lymphoma, B-cell acute lymphocytic leukemia, B-cell lymphoma, breast cancer, cervical squamous cell carcinoma, chromophobe renal cell carcinoma, clear cell renal cell carcinoma, colorectal adenocarcinoma, cutaneous melanoma, diffuse large B-cell lymphoma, diffuse-type gastric cancer, endocervical adenocarcinoma, endometrial adenocarcinoma, epithelial ovarian cancer, esophageal cancer, Ewing sarcoma, fallopian tube serous carcinoma, fibrosarcoma. gallbladder carcinoma, ganglioglioma, gastroesophageal junction adenocarcinoma, glioblastoma, head and neck cancer, head and neck squamous cell carcinoma, hepatocellular carcinoma, high-grade serous carcinoma, inflammatory myofibroblastic tumor, leiomyosarcoma, neurofibroma, soft tissue sarcoma, invasive ductal carcinoma, low-grade serous carcinoma, lung adenocarcinoma, medulloblastoma, melanoma, mucinous cystadenocarcinoma, mucoepidermoid carcinoma, mucoepidermoid carcinoma, multiple myeloma, neuroblastoma, non-Hodgkins lymphoma, osteosarcoma, ovarian serous cystadenocarcinoma, pancreatic adenocarcinoma, pancreatic cancer, pancreatic ductal adenocarcinoma, papillary cell renal cell carcinoma, papillary thyroid carcinoma, pericardial mesothelioma, peritoneal mesothelioma, prostate adenocarcinoma, pulmonary sarcoma, renal cell carcinoma, salivary gland cancer, small cell lung carcinoma, small intestine cancer, smooth muscle tumor of uncertain malignant potential, squamous cell carcinoma, stomach cancer, thyroid cancer, urothelial carcinoma, uterine carcinosarcoma, uterine corpus endometrial carcinoma, uterine leiomyosarcoma, uterine papillary serous carcinoma, and uterine sarcoma.

In one embodiment, the cancer (or ROS1+ cancer, or ALK+ cancer) is newly diagnosed. In one embodiment, the cancer (or ROS1+ cancer, or ALK+ cancer) is previously untreated.

In one embodiment, the cancer (or ROS1+ cancer, or ALK+ cancer) is relapsed or refractory. In one embodiment, the cancer is relapsed. In one embodiment, the cancer (or ROS1+ cancer, or ALK+ cancer) is refractory.

In one embodiment, the subject is previously untreated. In one embodiment, the subject is treatment naïve to tyrosine kinase inhibitor (TKI) therapy. In one embodiment, the subject has received one or more prior lines of therapy. In one embodiment, the subject has received two or more prior lines of therapy. In one embodiment, the subject has developed resistance to one or more of the prior line of therapy. In one embodiment, the prior therapy comprises a tyrosine kinase inhibitor (TKI). In one embodiment, the prior therapy comprises one or more of crizotinib, ceritinib, alectinib, brigatinib, lorlatinib, entrectinib, repotrectinib, cabozantinib, foretinib, taletrectinib, merestinib, masitinib, and ensartinib. In one embodiment, the prior therapy comprises one or more chemotherapies. In one embodiment, the one or more chemotherapies are in addition to the TKI therapy.

In one embodiment, the cancer (or ROS1+ cancer, or ALK+ cancer) is resistant to a tyrosine kinase inhibitor (TKI).

In one embodiment, the cancer is resistant lung cancer. In one embodiment, the cancer is resistant non-small cell lung cancer. In one embodiment, the cancer is non-small cell lung cancer resistant to a TKI. In one embodiment, the cancer is ROS1+ non-small cell lung cancer resistant to a TKI. In one embodiment, the cancer is ALK+ non-small cell lung cancer resistant to a TKI.

In one embodiment, the cancer is lung cancer (e.g., NSCLC), and the cancer is relapsed after, or refractory to, prior treatment by a TKI.

In one embodiment, a compound provided herein is administered as first-line treatment. In one embodiment, a compound provided herein is administered as second-line treatment. In one embodiment, a compound provided herein is administered as third or fourth-line treatment.

In one embodiment, the cancer (or ROS1+ cancer, or ALK+ cancer) is metastatic. In one embodiment, the cancer has CNS metastases. In one embodiment, the cancer has brain metastases. In one embodiment, the cancer is metastatic non-small cell lung cancer (NSCLC). In one embodiment, the cancer is metastatic ROS1+ NSCLC. In one embodiment, the cancer is metastatic ALK+ NSCLC.

In one embodiment, provided herein is a method for treating a patient with metastatic ALK+ non-small cell lung cancer (NSCLC), comprising administering to the patient a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating a patient with metastatic ROS1+ non-small cell lung cancer (NSCLC), comprising administering to the patient a therapeutically effective amount of a compound provided herein, e.g., a compound of Formula (I), or an enantiomer, a mixture of enantiomers, or a tautomer thereof, or a pharmaceutically acceptable salt thereof.

In one embodiment, the patient is an adult patient. In one embodiment, the patient is a pediatric patient.

In one embodiment, provided herein is a method for treating an adult patient with metastatic ROS1+ NSCLC, comprising administering to the patient a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating an adult patient with metastatic ROS1+ NSCLC, comprising administering to the patient a therapeutically effective amount of a compound provided herein, e.g., a compound of Formula (I), or an enantiomer, a mixture of enantiomers, or a tautomer thereof, or a pharmaceutically acceptable salt thereof, wherein the patient has progressed on or is intolerant of at least 1 prior TKI therapy.

In one embodiment, provided herein is a method for treating an adult patient with metastatic NSCLC that is ROS1+ with solvent front mutation G2032R, comprising administering to the patient a therapeutically effective amount of a compound provided herein, e.g., a compound of Formula (I), or an enantiomer, a mixture of enantiomers, or a tautomer thereof, or a pharmaceutically acceptable salt thereof, wherein the patient has progressed on or is intolerant of at least 1 prior TKI therapy.

In one embodiment, provided herein is a method for treating a ROS1-associated (or ROS1+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating a ROS1-associated (or ROS1+) cancer in a subject in need thereof, wherein the cancer has developed resistance to a tyrosine kinase inhibitor (TKI), and wherein the cancer has been identified as having one or more ROS1 inhibitor resistance mutations, the method comprising administering to the subject a therapeutically effective amount of a compound provided herein, e.g., a compound of Formula (I), or an enantiomer, a mixture of enantiomers, or a tautomer thereof, or a pharmaceutically acceptable salt thereof. In one embodiment, the one or more ROS1 inhibitor resistance mutations comprise one or more amino acid substitutions at an amino acid position selected from 1986, 2004, 2026, 2032, and 2033. In one embodiment, the one or more ROS1 inhibitor resistance mutations comprise one or more amino acid substitutions selected from S1986F, S1986Y, F2004C, F2004V, L2026M, G2032R, D2033N, L2086F, and G2101A. In one embodiment, the one or more ROS1 inhibitor resistance mutations is G2032R. In one embodiment, the one or more ROS1 inhibitor resistance mutations comprise G2032R and one or more of S1986F, S1986Y, F2004C, F2004V, L2026M, D2033N, or G2101A. In one embodiment, the ROS1 inhibitor resistance mutation is L2086F.

In one embodiment, provided herein is a method for treating a ALK-associated (or ALK+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (I), or an enantiomer, a mixture of enantiomers, or a tautomer thereof, or a pharmaceutically acceptable salt thereof.

In one embodiment, provided herein is a method for treating a ALK-associated (or ALK+) cancer in a subject in need thereof, wherein the cancer has developed resistance to a tyrosine kinase inhibitor (TKI), and wherein the cancer has been identified as having one or more ALK inhibitor resistance mutations, the method comprising administering o the subject a therapeutically effective amount of a compound provided herein, e.g., a compound of Formula (I), or an enantiomer, a mixture of enantiomers, or a tautomer thereof, or a pharmaceutically acceptable salt thereof. In one embodiment, the one or more ALK inhibitor resistance mutations comprise one or more amino acid substitutions at an amino acid position selected from 1196, 1198, 1202, and 1269. In one embodiment, the one or more ALK inhibitor resistance mutations comprise one or more amino acid substitutions selected from L1196M, L1198F, G1202R, and G1269A. In one embodiment, the one or more ALK inhibitor resistance mutations is G1202R. In one embodiment, the one or more ALK inhibitor resistance mutations comprise G1202R and one or more of L1196M, L1198F, and G1269A.

In one embodiment, provided herein is a method for treating an adult patient with metastatic NSCLC that is ALK+ with mutation G1202R, comprising administering to the patient a therapeutically effective amount of a compound provided herein, e.g., a compound of Formula (I), or an enantiomer, a mixture of enantiomers, or a tautomer thereof, or a pharmaceutically acceptable salt thereof, wherein the patient has progressed on or is intolerant of at least 1 prior TKI therapy.

In one embodiment, provided herein is a method for treating a ALK-associated (or ALK+) cancer in a subject in need thereof, wherein the cancer has developed resistance to a tyrosine kinase inhibitor (TKI), the method comprising administering to the subject a therapeutically effective amount of a compound provided herein, e.g., a compound of Formula (I), or an enantiomer, a mixture of enantiomers, or a tautomer thereof, or a pharmaceutically acceptable salt thereof.

In one embodiment, the TKI is a ROS1 inhibitor. In one embodiment, the TKI is an ALK inhibitor. In one embodiment, the TKI is crizotinib, ceritinib, alectinib, brigatinib, lorlatinib, entrectinib, repotrectinib, cabozantinib, foretinib, merestinib, taletrectinib, masitinib, or ensartinib. In one embodiment, the TKI is crizotinib. In one embodiment, the TKI is entrectinib.

In certain embodiments, the subject has relapsed after first-line treatment of the cancer. In other embodiments, the subject has relapsed after second-line treatment of the cancer.

In one embodiment, the cancer or disease is in a pediatric patient (including an infantile patient). In one embodiment, the cancer is systemic anaplastic large cell lymphoma (ALCL) that is ALK+ in pediatric patients 1 year of age or older, and young adults. In another embodiment, the cancer is relapsed or refractory systemic anaplastic large cell lymphoma (ALCL) that is ALK+ in pediatric patients 1 year of age or older, and young adults. In one embodiment, the cancer is systemic anaplastic large cell lymphoma (ALCL) that is ROS1+ in pediatric patients 1 year of age or older, and young adults. In another embodiment, the cancer is relapsed or refractory systemic anaplastic large cell lymphoma (ALCL) that is ROS1+ in pediatric patients 1 year of age or older, and young adults.

In certain embodiments, the methods for treating or preventing cancer can be demonstrated by one or more responses such as increased apoptosis, inhibition of tumor growth, reduction of tumor metastasis, inhibition of tumor metastasis, reduction of microvessel density, decreased neovascularization, inhibition of tumor migration, tumor regression, and increased survival of the subject.

### 5.6. Combination Therapy

In some embodiments, the method of treating or preventing cancer may comprise administering a solid form or pharmaceutical composition provided herein, such as Form 2 of a compound of Formula (I), conjointly with one or more other chemotherapeutic agent(s).

As used herein and unless otherwise specified, by “conjointly” or “in combination with”, it is not intended to imply that the other agent and the compound of Formula (I) must be administered at the same time and/or formulated for delivery together, although these methods of delivery are within the scope of this disclosure. The compound provided herein can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other agents (e.g., one or more other additional agents). In general, each therapeutic agent is administered at a dose and/or on a time schedule determined for that particular agent. The other therapeutic agent can be administered with the compound provided herein in a single composition or separately in a different composition. Triple therapy is also contemplated herein.

Chemotherapeutic agents that may be conjointly administered with compounds of the disclosure include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, ABT-263, afatinib dimaleate, axitinib, aminoglutethimide, amsacrine, anastrozole, APCP, asparaginase, AZD5363, Bacillus Calmette-Guérin vaccine (bcg), bicalutamide, bleomycin, bortezomib, β-methylene-ADP (AOPCP), buserelin, busulfan, cabazitaxel, cabozantinib, camptothecin, capecitabine, carboplatin, carfilzomib, carmustine, ceritinib, chlorambucil, chloroquine, cisplatin, cladribine, clodronate, cobimetinib, colchicine, crizotinib, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, eribulin, erlotinib, estradiol, estramustine, etoposide, everolimus, exemestane, filgrastim, fludarabine, fludrocortisone, fluorouracil, fluoxymesterone, flutamide, gefitinib, gemcitabine, genistein, goserelin, GSK1120212, hydroxyurea, idarubicin, ifosfamide, imatinib, interferon, irinotecan, ixabepilone, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, mutamycin, N-(4-sulfamoylphenylcarbamothioyl) pivalamide, NF279, NF449, nilutamide, nocodazole, octreotide, olaparib, oxaliplatin, paclitaxel, pamidronate, pazopanib, pemexetred, pentostatin, perifosine, PF-04691502, plicamycin, pomalidomide, porfimer, PPADS, procarbazine, quercetin, raltitrexed, ramucirumab, reactive blue 2, rituximab, rolofylline, romidepsin, rucaparib, selumetinib, sirolimus, sodium 2,4-dinitrobenzenesulfonate, sorafenib, streptozocin, sunitinib, suramin, talazoparib, tamoxifen, temozolomide, temsirolimus, teniposide, testosterone, thalidomide, thioguanine, thiotepa, titanocene dichloride, tonapofylline, topotecan, trametinib, trastuzumab, tretinoin, veliparib, vinblastine, vincristine, vindesine, vinorelbine, and vorinostat (SAHA). In other embodiments, chemotherapeutic agents that may be conjointly administered with compounds of the disclosure include: ABT-263, dexamethasone, 5-fluorouracil, PF-04691502, romidepsin, and vorinostat (SAHA). In other embodiments, chemotherapeutic agents that may be conjointly administered with compounds of the disclosure include: 1-amino-4-phenylamino-9,10-dioxo-9,10-dihydroanthracene-2-sulfonate (acid blue 25), 1-amino-4-[4-hydroxyphenyl-amino]-9,10-dioxo-9,10-dihydroanthracene-2-sulfonate, 1-amino-4-[4-aminophenylamino]-9,10-dioxo-9,10-dihydroanthracene-2-sulfonate, 1-amino-4-[1-naphthylamino]-9,10-dioxo-9,10-dihydroanthracene-2-sulfonate, 1-amino-4-[4-fluoro-2-carboxyphenylamino]-9,10-dioxo-9,10-dihydroanthracene-2-sulfonate, 1-amino-4-[2-anthracenylamino]-9,10-dioxo-9,10-dihydroanthracene-2-sulfonate, APCP, β-methylene-ADP (AOPCP), capecitabine, cladribine, cytarabine, fludarabine, doxorubicin, gemcitabine, N-(4-sulfamoylphenylcarbamothioyl) pivalamide, NF279, NF449, PPADS, quercetin, reactive blue 2, rolofylline sodium 2,4-dinitrobenzenesulfonate, sumarin, and tonapofylline.

Many combination therapies have been developed for the treatment of cancer. In certain embodiments, a solid form or pharmaceutical composition provided herein (e.g., Form 2 of a compound of Formula (I)) may be conjointly administered with one or more combination therapies. Examples of combination therapies with which compounds provided herein may be conjointly administered are included in Table 1.

TABLE 1
Exemplary combinatorial therapies for the treatment of cancer

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, Procarbazine |
| CAP | Cyclophosphamide, Doxorubicin, Cisplatin |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, Doxorubicin, |
| | Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic lymphocytic | Chlorambucil, Prednisone |
| leukemia) | |
| CP (Ovarian Cancer) | Cyclophosphamide, Cisplatin |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, Melphalan |
| MAC-III | Methotrexate, Leucovorin, Dactinomycin, Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PCV | Lomustine, Procarbazine, Vincristine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, Cytarabine, |
| | Bleomycin, Vincristine, Methotrexate, Leucovorin, Cotrimoxazole |
| PROMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, Bleomycin, |
| | Etoposide, Prednisone |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or Mitoxantrone |
| “8 in 1” | Methylprednisolone, Vincristine, Lomustine, Procarbazine, |
| | Hydroxyurea, Cisplatin, Cytarabine, Dacarbazine |

In certain embodiments In certain embodiments, the conjoint therapies of the disclosure comprise conjoint administration with other types of chemotherapeutic agents, such as immuno-oncology agents. Cancer cells often have specific cell surface antigens that can be recognized by the immune system. Thus, immuno-oncology agents, such as monoclonal antibodies, can selectively bind to cancer cell antigens and effect cell death. Other immuno-oncology agents can suppress tumor-mediated inhibition of the native immune response or otherwise activate the immune response and thus facilitate recognition of the tumor by the immune system. Exemplary antibody immuno-oncology agents, include, but are not limited to, abagovomab, adecatumumab, afutuzumab, alemtuzumab, anatumomab mafenatox, apolizumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED 14736, MPDL3280A, nivolumab, obinutuzumab, ocaratuzumab, ofatumumab, olatatumab, pembrolizumab, pidilizumab, rituximab, ticilimumab, samalizumab, and tremelimumab. In some embodiments, the antibody immuno-oncology agents are selected from anti-CD73 monoclonal antibody (mAb), anti-CD39 mAb, anti-PD-1 mAb, and anti-CTLA4 mAb. Thus, in some embodiments, the methods of the disclosure comprise conjoint administration of one or more immuno-oncology agents, such as the agents mentioned above.

In some embodiments, the combination therapy comprises conjoint administration of a compound or solid form of the disclosure, such as Form 2 of a compound of Formula (I), with SH2 inhibitors, such as CGP78850, CPG85793, C90, C126, G7-18NATE, G7-B1, and NSC642056.

In some embodiments, the combination therapy comprises conjoint administration of a compound or solid form of the disclosure, such as Form 2 of a compound of Formula (I), with MEK inhibitors, such as trametinib, cobimetinib, binimetinib, selumetinib, PD-325901, CI-1040, and TAK-733.

In some embodiments, the combination therapy comprises conjoint administration of a compound or solid form of the disclosure, such as Form 2 of a compound of Formula (I), with a MET inhibitor selected from JNJ-38877605, PF-04217903, foretinib, AMG 458, tivantinib, cabozantinib, crizotinib, capmatinib hydrochloride, tepotinib hydrochloride, and savolitinib.

In some embodiments, the combination therapy comprises conjoint administration of a compound or solid form of the discloser, such as Form 2 of a compound of Formula (I), with a SHP2 inhibitor selected from TNO-155, RMC-4630, JAB-3068, or RLY-1971.

In some embodiments, the combination therapy comprises conjoint administration of a compound or solid form of the disclosure, such as Form 2 of a compound of Formula (I), with a RAS inhibitor selected from aliskiren, captopril, losartan, irbesartan, olmesartan, candesartan, valsartan, fimasartan, azilsartan, telmisartan, eprosartan, benazepril, enalapril, lisinopril, perindopril, quinapril, ramipril, and trandolapril.

In some embodiments, the combination therapy comprises administration of a compound or solid form provided herein, e.g., Form 2 of a compound of Formula (I), in combination with a TKI. In one embodiment, the TKI is a ROS1 inhibitor. In one embodiment, the TKI is an ALK inhibitor. In one embodiment, the TKI is crizotinib, ceritinib, alectinib, brigatinib, lorlatinib, entrectinib, repotrectinib, cabozantinib, foretinib, merestinib, taletrectinib, masitinib, or ensartinib. In one embodiment, the TKI is crizotinib. In one embodiment, the TKI is entrectinib. In one embodiment, the TKI is alectinib. In one embodiment, the TKI is brigatinib.

In some embodiments, the combination therapy comprises conjoint administration of a compound or solid form of the disclosure, such as Form 2 of a compound of Formula (I), with anti-PD-1 therapy. In certain embodiments, the combination therapy comprises conjoint administration of a compound or solid form of the disclosure, such as Form 2 of a compound of Formula (I), with oxaliplatin. In other embodiments, the combination therapy comprises conjoint administration of a compound or solid form of the disclosure, such as Form 2 of a compound of Formula (I), with doxorubicin.

In certain embodiments, a compound or solid form of the disclosure may be conjointly administered with non-chemical methods of cancer treatment. In certain embodiments, a compound or solid form of the disclosure may be conjointly administered with radiation therapy. In certain embodiments, a compound or solid form of the disclosure may be conjointly administered with surgery, with thermoablation, with focused ultrasound therapy, with cryotherapy, or with any combination of these.

In certain embodiments, compounds or solid forms of the disclosure may be conjointly administered with one or more other compounds or solid forms of the disclosure. Moreover, such combinations may be conjointly administered with other therapeutic agents, such as other agents suitable for the treatment of cancer, immunological or neurological diseases, such as the agents identified above. In certain embodiments, conjointly administering one or more additional chemotherapeutic agents with a compound of the disclosure provides a synergistic effect. In certain embodiments, conjointly administering one or more additional chemotherapeutic agents provides an additive effect.

### EXAMPLES

The examples and preparations provided below further illustrate and exemplify the compounds as provided herein and methods of preparing such compounds. It is to be understood that the scope of the present disclosure is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers can be obtained by methods known to those skilled in the art.

| Abbreviations/Acronyms | Full Name/Description |
|---|---|
| ACN or MeCN | acetonitrile |
| DCM | dichloromethane |
| DMF | dimethylformamide |
| EtOAc | ethyl acetate |
| IPA | isopropyl alcohol |
| IPAc | isopropyl acetate |
| MEK | methyl ethyl ketone |
| 2-MeTHF | 2-methyltetrahydrofuran |
| MIBK | methyl iso-butyl ketone |
| MTBE or TBME | tert-butyl methyl ether |
| THF | tetrahydrofuran |
| DIPEA | diisopropylethylamine |
| DIPA | diisopropanolamine |
| DMA | dimethylacetamide |
| MeOH | methanol |
| EtOH | ethanol |
| CPME | Cyclopentyl methyl ether |
| XRPD | x-ray powder diffraction |

### Example 1. Preparation of Compound 1

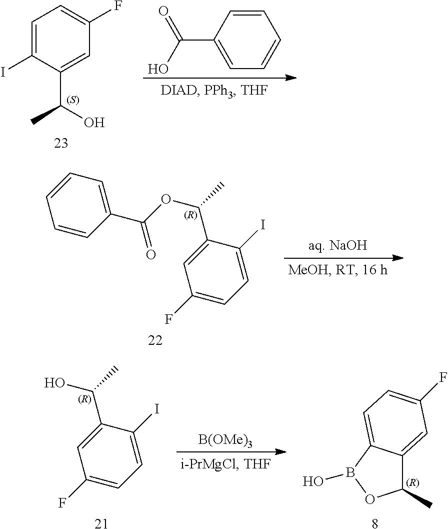

Synthesis of Compound 22. To a 100 L reactor was charged THF (56.8 kg), Compound 23 (8.00 kg), benzoic acid (3.68 kg) and PPh₃ (8.28 kg) at r.t. (20-25° C.) under nitrogen protection. The mixture was cooled to 0° C., and was added DIAD (6.38 kg) drop-wise over 30 min at 0-10° C. After complete addition, the reaction mixture was warmed up to 20-25° C. over 1-1.5 h, then stirred at 20-25° C. for 18 h under nitrogen. The reaction mixture was poured into water (80 kg) below 20° C., and was charged EtOAc (36.0 kg). The mixture was stirred for 30 min and separated. The aqueous phase was extracted with EtOAc (21.6 kg). The combined organic phases were washed with brine (15 w %, 55 kg), and was concentrated in vacuum until around 2V. Then heptane (16.4 kg×2) was charged, and was concentrated in vacuum to around 3V. The resulting slurry was filtered and the filter cake was rinsed with heptane (10.9 kg). The combined filtrates were concentrated in vacuum at 45-50° C. to get crude product (13.1 kg). The crude product (13.1 kg) was recrystallized from MeOH (15.8 kg) at 45-10° C. to afford Compound 22 (8.50 kg, >99.9%/220 nm, 76% yield). ¹H NMR (400 MHz, DMSO-d₆) δ 8.16-8.01 (m, 2H), 7.92 (dd, J=8.7, 5.7 Hz, 1H), 7.75-7.64 (m, 1H), 7.56 (dd, J=10.7, 4.7 Hz, 2H), 7.42 (dd, J=10.0, 3.0 Hz, 1H), 7.03 (td, J=8.5, 3.1 Hz, 1H), 6.06 (q, J=6.4 Hz, 1H), 1.58 (d, J=6.5 Hz, 2H).

Synthesis of Compound 21. To a 100 L reactor was charged MeOH (32.0 kg) and Compound 22 (13.5 kg) at r.t. (20-25° C.). The mixture was cooled to 10° C., and was added aqueous NaOH solution (2.92 kg in 40.5 kg water) drop-wise over 1-2 min at 10-20° C. After complete addition, the reaction mixture was warmed up to 20-25° C., then stirred at 25-30° C. for 18-20 h. The reaction mixture was poured into water (135 kg) below 15° C. The resulting slurry was stirred for 1-2 h at 10-15° C. and filtered. The filter cake was washed with water (30 kg). The filter cake was slurried for 1-2 h in water (67.5 kg) at 10-15° C. and filtered. The filter cake was dried for 24 h in oven at 50° C. to afford Compound 21 as a white solid (9.20 kg). ¹H NMR (400 MHz, DMSO-d₆) δ 7.81 (dd, J=8.6, 5.7 Hz, 1H), 7.31 (dd, J=10.4, 3.2 Hz, 1H), 6.93 (td, J=8.5, 3.2 Hz, 1H), 5.56 (d, J=4.0 Hz, 1H), 4.90-4.64 (m, 1H), 1.27 (d, J=6.4 Hz, 3H).

Synthesis of Compound 8. To a 100 L reactor was charged THF (42.5 kg) and (R)-1-(5-fluoro-2-iodophenyl) ethan-1-ol (Compound 21, 6.00 kg) at 20-25° C. under nitrogen. The mixture was refilled fully with nitrogen. The mixture was cooled to −30-−20° C., and was added i-PrMgCl solution in THF (28.2 L, 2 M) dropwise over 1.5-2 h at −30-−20° C. under nitrogen. After complete addition, the reaction mixture was stirred for 1.5-2 h at −15-−10° C. The reaction mixture was cooled to −30° C., and was charged B(OMe)₃ (5.86 kg) drop-wise over 2 h at −30-−20° C. under nitrogen. After addition, the reaction mixture was stirred for 2-3 h at −10-0° C. The reaction mixture was poured into aqueous NH₄Cl solution (20 w %, 60 kg) at 10-20° C. The mixture was stirred for 1 h at 15-25° C. and separated. The aqueous phase was extracted with EtOAc (27.0 kg×2) twice. The combined organic phases were washed with water (30 kg), then concentrated in vacuum until around 3V, switched to n-heptane (8.2 kg×2) twice and concentrated at 40-45° C. to get crude Compound 8 as a light yellow oil (3.4 kg). Recrystallization of the combined crude Compound 8 from three batches (total 10.2 kg) from n-heptane (14.0 kg) at 40-10° C. afforded Compound 8 as an off-white solid (6.10 kg). ¹H NMR (400 MHz, DMSO-d₆) δ 9.18 (s, 1H), 7.73 (dd, J=8.1, 5.9 Hz, 1H), 7.27 (dd, J=9.5, 2.1 Hz, 1H), 7.17 (td, J=8.1, 4.1 Hz, 1H), 5.19 (q, J=6.6 Hz, 1H), 1.40 (d, J=6.6 Hz, 3H).

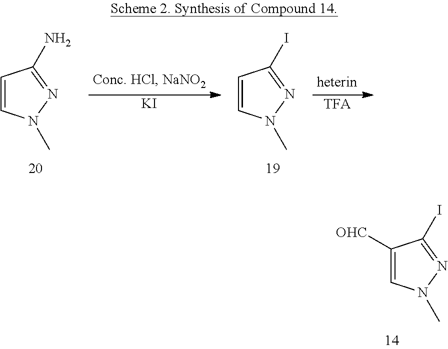

Synthesis of Compound 19. To a 500 L reactor was charged water (59.7 kg) and conc. HCl (36 w %, 59.7 kg) and 1-methyl-1H-pyrazol-3-amine (Compound 20, 19.0 kg, 1.0 eq.) at 15-20° C. Then the mixture was stirred for 30 min at 15-20° C. and cooled to 0° C. Aqueous NaNO₂ solution (20.3 kg) in water (45.0 kg) was charged dropwise while keeping the temperature at −5-5° C. After complete addition, the solution was stirred for another 0.5-1 h at −5-5° C. Aqueous KI solution (64.9 kg) in water (125 kg) was charged in a 1000 L reactor. Then the solution was cooled to 0° C. The yellow solution in the 500 L reactor was charged dropwise into the 1000 L reactor over 1.5-2 h at −5-5° C. After addition, the solution was stirred for another 1-2 h and warmed up slowly to 20° C. Ammonium hydroxide (57.0 kg), aqueous Na₂SO₃ solution (15 w %, 165 kg, 24.7 kg Na₂SO₃ in 140.3 kg water) was charged to the 1000 L reactor dropwise below 30° C. separately. Then EtOAc (86.0 kg) were charged to the 1000 L reactor. The resulting solution was stirred for 30 min and separated. The aqueous phase was extracted with EtOAc (86.0 kg). The combined organic phases were washed with water (95.0 kg) and concentrated in vacuum at 45-50° C. to get crude Compound 19 as a light brown oil (24.2 kg). The crude Compound 19 was used directly for next step.

Synthesis of Compound 14. To a 100 L reactor was charged trifluoroacetic acid (TFA, 47.7 kg), followed by Compound 19 (8.00 kg) at 20-30° C. The resulting solution was cooled to 15-20° C., and was added 1,3,5,7-tetraazatricyclo[3.3.1.1(3,7)]decane (“heterin”, 15.2 kg) in portions over 30 min while keeping the temperature below 50° C. The reaction mixture was heated to 80-90° C. and stirred at 80-90° C. for 16-20 h under nitrogen. The solids were dissolved gradually and the mixture finally became a red solution. The reaction mixture was concentrated to remove most TFA, then cooled to 15-20° C., then neutralized with aq. NaHCO₃ solution (5 w %, 20 kg) below 20° C. The mixture was stirred at 15-25° C. for 1 h and filtered. The filter cake was rinsed with water (8 kg×2). Purification was conducted for three batches, each 8.00 kg scale. The combined wet cakes were slurried in water (100 kg) at r.t (20-25° C.) for 1-2 h and filtered. The filter cake was washed with water (20 kg). The filter cake was dried over in oven at 50° C. for 24 h to get Compound 14 as a yellow solid (21.8 kg). ¹H NMR (400 MHz, CDCl₃) δ 9.64 (s, 1H), 7.82 (s, 1H), 3.98 (s, 3H).

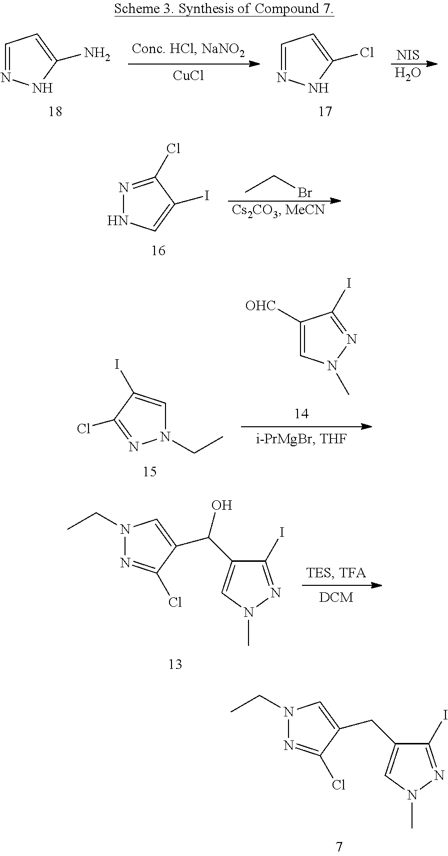

Synthesis of Compound 17. To a 500 L reactor was charged water (90.0 kg) and conc. HCl (36 w %, 55.0 kg) at r.t. The mixture was cooled to 0-10° C., and was charged Compound 18 (15.0 kg) in portions at 20-30° C. The mixture was stirred for 30 min at 20-30° C., then cooled to −5-5° C. A solution of NaNO₂ (13.1 kg) in water (30.0 kg) was charged dropwise over 1.5-2 h at −5-5° C. The reaction mixture was stirred for 0.5-1 h at −5-5° C. Conc. HCl (36 w %, 55.0 kg) and CuCl (26.8 kg) was charged to a second 500 L reactor at 20-30° C. The mixture was heated to 40-45° C. under N₂. The solution in the first 500 L reactor was charged into the second 500 L reactor dropwise over 2-3 h at 40-45° C. under N₂. The reaction mixture was stirred for 1 h at 40-45° C. The reaction mixture was cooled to 20-30° C., and was charged brine (15 w %, 200 kg) and the mixed solvents of EtOAc (225 kg)/THF (44.5 kg). The mixture was stirred for 0.5 h and separated. The aqueous phase was extracted with the mixed solvents of EtOAc (135 kg)/THF (26.5 kg). The combined organic layers were washed with aqueous NaHCO₃ (5 w %, 25 kg) and brine (15 w %, 100 kg) separately. The organic layer was concentrated in vacuum at 40-45° C. until ˜2V, switched to n-heptane (20.5 kg×2) twice and concentrated to get Compound 17 as an oil (12.8 kg). ¹H NMR (400 MHz, DMSO-d₆) δ 6.30 (br s, 1H), 7.80 (br s, 1H), 12.8-13.2 (m, 1H).

Synthesis of Compound 16. To a 1000 L reactor was charged water (240 kg), followed by crude Compound 17 (12.0 kg) in portions at r.t. (15-20° C.) under nitrogen. The reaction mixture was cooled to 0˜10° C., and was charged NIS (24.3 kg) in portions at 0-10° C. over 0.5-1 h. The resulting mixture as an off-white slurry was stirred at 0-10° C. for 0.5-1 h. The reaction mixture was warmed up to 20-30° C. and stirred for 16 h at 20-30° C. The reaction mixture was quenched with around 30 w % aq. Na₂S₂O₃ (26.4 kg Na₂S₂O₃ in 60 kg of water) below 30° C. The mixture was stirred for 1-2 h at 20˜30° C. and separated. The aqueous phase was extracted with EtOAc (108 kg) and the organic phase was washed with brine (15 w %, 80 kg×2) twice and concentrated in vacuum at 40-45° C. until around 2V, switched to heptane (80 kg×2) twice and concentrated to get crude product as a brown solid (92%/220 nm). The crude product was slurried for 1-2 h in the mixed solvents of EtOAc (320 g)/Heptane (24 kg) at 40-45° C. and filtered. The filter cake was rinsed with heptane (13.7 kg) and dried in oven at 50° C. for 16 h to get Compound 16 as an off-white solid (14.6 kg). ¹H NMR (400 MHz, DMSO-d₆) δ 13.40 (s, 1H), 8.00 (s, 1H).

Synthesis of Compound 15. To a 100 L reactor was charged MeCN (37.0 kg), followed by Compound 16 (6.70 kg) at r.t. (15˜20° C.) under nitrogen. The mixture was stirred for 15 min to afford a solution, and was charged Cs₂CO₃ (13.5 kg) at r.t. (15˜20° C.). The reaction mixture was cooled to 0-10° C., and was charged a solution of bromoethane (3.93 kg) in MeCN (5.5 kg) drop-wise at 0-10° C. over 1.5 h with strong stirring. The white slurry was stirred at 0-10° C. for 6-8 h. The mixture was then filtered and the filter cake was washed with EtOAc (12.1 kg). To the combined filtrates were charged EtOAc (48.3 kg) and water (53.6 kg). The mixture was stirred for 30 min and separated. The aqueous layer was extracted with another EtOAc (30.2 kg) and separated. The organic phases were washed with brine (15 w %, 45 kg×2) twice. The organic phase was concentrated in vacuum below 45° C. until ˜2V, switched to n-heptane (13.7 kg×2) and concentrated to get a residue as oil. The combined residue from two batches (each 6.7 kg scale) was purified by silica gel chromatography column eluted with n-heptane/EtOAc (from 500:1 to 50:1) to afford Compound 15 as an off-white solid (7.10 kg). ¹H NMR (400 MHz, DMSO-d₆) δ 7.99 (s, 1H), 4.11 (q, J=7.3 Hz, 2H), 1.35 (t, J=7.3 Hz, 3H).

Synthesis of Compound 13. To a 100 L reactor was charged anhydrous THF (16.0 kg), followed by Compound 15 (4.50 kg) at r.t. (20-45° C.). The reaction mixture was refilled with nitrogen. The reaction solution was cooled to −25-−20° C., and was charged slowly i-PrMgCl solution in THF (8.77 L, 2M) drop-wise over 1.5 h at −25-−15° C. The resulting mixture was stirred at −25-−15° C. for 0.5-1 h under nitrogen. To the mixture was charged a solution of Compound 14 (4.02 kg) in anhydrous THF (35.9 kg) dropwise at −25-−15° C. over 1.0-1.5 h under nitrogen. The reaction mixture was stirred at −25-−15° C. for 1 h. The reaction mixture was quenched with aqueous NH₄Cl solution (20 w %, 9 kg NH₄Cl in 36 kg water) at 0-10° C. and separated. The aqueous phase was extracted with EtOAc (20 kg×2) twice. The combined organic phases was washed with brine (15 w %, 30 kg×2) and concentrated in vacuum at 45-50° C. to get crude Compound 13 as a yellow oil. 24.5 kg of crude Compound 13 from four separate batches was stirred for 18-24 h in the mixed solvents of EtOAc (17.8 kg)/n-heptane (26.9 kg) at 20-30° C. The solids was collected by filtration, and was dried in oven at 45-50° C. to get crude Compound 13 (17.8 kg, ˜96%/220 nm). The crude Compound 13 (17.8 kg) was slurried in MTBE (26.5 kg) at 10-15° C. for 5-8 h and filtered. The filter cake was dried in oven at 40-45° C. to get Compound 13 as a pale yellow solid (16.9 kg). ¹H NMR (400 MHz, DMSO-d₆) δ 7.62 (s, 1H), 7.53 (s, 1H), 5.59 (d, J=4.8 Hz, 1H), 5.29 (d, J=4.8 Hz, 1H), 4.05 (q, J=7.2 Hz, 2H), 3.82 (s, 3H), 1.33 (t, J=7.2 Hz, 3H).

Synthesis of Compound 7. To a 1000 L reactor was charged DCM (219 kg), followed by Compound 13 (16.5 kg) at 10-20° C. The mixture was refilled with nitrogen. Triethylsilane (TES) (15.7 kg) was charged dropwise over 30 min at 10-20° C. The reaction mixture was cooled to −5-−5° C., and was charged TFA (20.0 kg) dropwise over 1.5-2 h at −15-−5° C. The reaction mixture was stirred at −5-5° C. for 2-3 h under nitrogen. The reaction mixture was adjusted to pH=˜7 with aqueous Na₂CO₃ solution (150 kg, 15 w %, 22.5 kg Na₂CO₃ in 127.5 kg water) at 0-20° C. The mixture was stirred for 0.5-1 h at 10-20° C. and separated. The organic phase was washed with water (165 kg) and brine (15 w %, 105 kg) separately. The organic phase was concentrated in vacuum to around 2V, switched to n-heptane (56.4 kg×2), then concentrated until around 4V. The remaining mixture was cooled to 10-15° C., then stirred for another 1-2 h at 10-15° C. and filtered. The filter cake was washed with n-heptane (11.3 kg) and dried in oven at 40-45° C. for 16-20 h to get Compound 7 as an off-white solid (13.6 kg). ¹H NMR (400 MHz, DMSO-d₆) δ 7.55 (s, 1H), 7.43 (s, 1H), 4.03 (q, J=7.2 Hz, 2H), 3.79 (s, 3H), 3.37 (s, 2H), 1.31 (t, J=7.3 Hz, 3H).

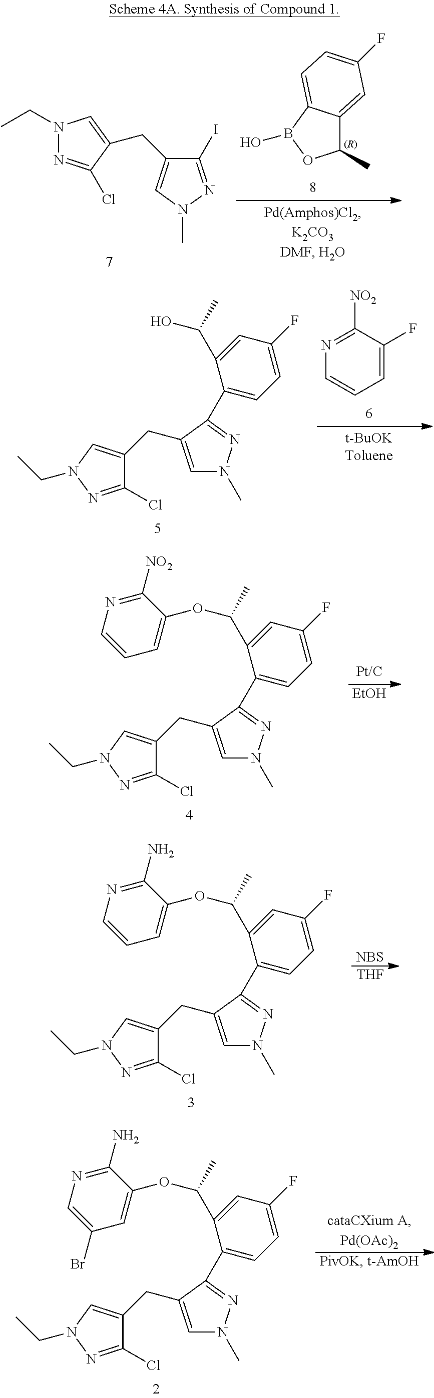

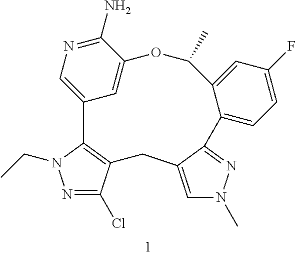 [SMILES: CCn1nc(Cl)c2c1-c1cnc(N)c(c1)O[C@H](C)c1cc(F)ccc1-c1nn(C)cc1C2]

Synthesis of Compound 5. To a 50 L flask was charged DMF (12 L, 10 vol.) and H₂O (2 L, 2 vol.), followed by Compound 7 (1.2 kg, 3.42 mol, 1.0 equiv.) and Compound 8 (624.8 g, 3.76 mol, 1.1 equiv.) in one portion at r.t. (15-20° C.). The reaction mixture was purged with nitrogen, then was charged power K₂CO₃ (1.42 kg, 200-300 meshes) and Pd(Amphos)Cl₂ (12.1 g) under nitrogen. After complete addition, the reaction mixture was purged with nitrogen, then heated to 60-65° C. and stirred for 2-3 h at 60-65° C. Silica Thiopropyl Metal Scavenger (40-63 um, 60 A, 10 w %, 120 g) was added at 60-65° C. and stirred for 2 h at 60-65° C. The reaction mixture was cooled to 20-30° C. and filtered through a celite pad (1.5×). The filter cake was rinsed with MTBE (2.4 L). The combined filtrates were diluted with MTBE (9.6 L), washed with water (24 L) and separated. The aqueous layer was extracted with MTBE (6 L×2). The combined organic phases were washed with brine (15% w/w, 9.6 L×2). The combined organic phases from two batches (around 48 L) were dried over anhydrous Na₂SO₄, filtered and concentrated to get crude Compound 5 as a yellow oil (2.42 kg). The crude Compound 5 was used directly for the next reaction. ¹H NMR (400 MHz, DMSO-d₆) δ 7.52 (s, 1H), 7.39 (s, 1H), 7.36 (dd, J=10.7, 2.6 Hz, 1H), 7.15 (dd, J=8.3, 6.1 Hz, 1H), 7.07 (td, J=8.4, 2.6 Hz, 1H), 5.13 (d, J=4.2 Hz, 1H), 4.86-4.77 (m, 1H), 3.98 (q, J=7.3 Hz, 2H), 3.81 (s, 3H), 3.35 (d, J=2.3 Hz, 2H), 1.28 (t, J=7.3 Hz, 3H), 1.10 (d, J=6.3 Hz, 3H).

Synthesis of Compound 4. To a 50 L reactor was added toluene (16 L, 10 vol.), crude Compound 5 (1.60 kg, 4.4 mol, 1.0 equiv.) and Compound 6 (688.0 g, 4.84 mol, 1.1 equiv.) at r.t. (15-20° C.) under nitrogen. The reaction mixture was purged with nitrogen, then cooled to 0-5° C. A solution of potassium t-butoxide (736.0 g, 6.56 mol, 1.5 equiv.) in THF (1.6 L, 1 vol.) was added dropwise at 0-5° C. The reaction mixture was stirred for 0.5-1 h at 0-5° C. under nitrogen. Aqueous NH₄Cl solution (15 w %, 8 L) at 0-20° C. was charged over 10 min, and was stirred for 0.5 h at 5-15° C. MTBE (16 L) was added to the mixture, then was stirred for 0.5 h at 5-15° C. and separated. The aqueous phase was extracted with MTBE (8 L) and separated. The combined organic phases were washed with brine (15 w %, 8 L×2). The combined organic phases were dried over anhydrous Na₂SO₄, filtered and concentrated to afford crude Compound 4 as a brown oil (2.25 kg). The brown oil was dissolved in MTBE (16 L), and was charged activated charcoal (320 g, 20 w %). The mixture was heated to 45-50° C. and stirred for 1-2 h at 45-50° C. and filtered. The filter cake was washed with MTBE (2.4 L). The filtrate was concentrated in vacuum to afford crude Compound 4 as a yellow oil (1.85 kg). 1.8 kg of crude Compound 4 was slurried in EtOH (1.5 L) at 40-45° C., then stirred for around 30 min at 40-45° C. The oil was converted slowly to the solids. The slurry was cooled slowly to 25° C. over 1 h, then was added heptane (4.5 L) drop-wise over 1.5-2 h at 20-30° C. After addition, the mixture was cooled to 5-10° C. over 0.5-1 h, stirred for 1 h at 0-10° C. and filtered. The filter cake was rinsed with the mixed solvents of heptane/EtOH (1 L, 3 v/1 v) and dried in oven at 45-50° C. to afford Compound 4 as an off-white solid. (1.41 kg). ¹H NMR (400 MHz, DMSO-d₆) δ 8.05 (d, J=4.4 Hz, 1H), 7.63 (d, J=9.1 Hz, 2H), 7.54 (dd, J=8.5, 4.5 Hz, 1H), 7.46 (s, 1H), 7.37-7.31 (m, 1H), 7.22 (t, J=8.9 Hz, 2H), 5.88 (q, J=5.9 Hz, 1H), 3.98 (q, J=7.2 Hz, 2H), 3.87 (s, 3H), 3.45 (s, 2H), 1.44 (d, J=6.3 Hz, 3H), 1.26 (t, J=7.2 Hz, 3H).

The impurity of Formula (SP-6) is formed by displacement of the nitro group from Compound 6 vis SNAr reaction. ¹H NMR (400 MHz, DMSO-d₆) δ 7.76 (d, J=4.6 Hz, 1H), 7.68-7.61 (m, 1H), 7.53 (s, 1H), 7.39 (s, 1H), 7.32 (dd, J=10.2, 2.5 Hz, 1H), 7.23 (dd, J=8.3, 6.1 Hz, 1H), 7.15 (td, J=8.4, 2.6 Hz, 1H), 7.00-6.92 (m, 1H), 6.16 (q, J=6.3 Hz, 1H), 5.75 (s, 1H), 3.95 (q, J=7.2 Hz, 2H), 3.79 (s, 3H), 1.53 (d, J=6.4 Hz, 3H), 1.26 (t, J=7.2 Hz, 3H).

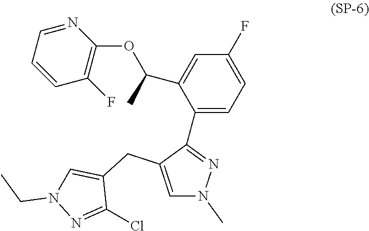 [SMILES: CCn1cc(Cc2cn(C)nc2-c2ccc(F)cc2[C@@H](C)Oc2ncccc2F)c(Cl)n1]

Synthesis of Compound 3. To a 20 L flask was added EtOH (6.75 L, 10 vol.), Compound 4 (675.0 g, 1.39 mol, 1.0 equiv.) and triethylamine (635.0 g, 6.28 mol, 4.5 equiv.) at r.t. (15-20° C.) under nitrogen, followed by Pt/C (81.0 g, 0.12×) in one portion at 15-20° C. The reaction mixture was heated to 65-70° C., and HCOOH (577.0 g, 12.5 mol, 9.0 equiv.) was added dropwise over 30 min at 65-70° C. The mixture was stirred for 20 h at 65-70° C. The reaction mixture was cooled to 20-25° C., filtered through a celite pad (2×) and rinsed with EtOH (1.4 L). The filtrate was concentrated in vacuum at 50-55° C. to get crude Compound 3 as a yellow oil (1.30 kg, 97.1%/220 nm). The crude Compound 3 (1.30 kg) was dissolved in EtOAc (2.6 L), and heptane was added (1.3 L) at 20-30° C. Heptane (1.3 L) was added drop wise at 5-10° C. over round 1 h. The seeds (0.5 g) were added and the solids slowly precipitated. Heptane (3.9 L) was added drop-wise at 0-10° C. over 2 h. After addition, the slurry was stirred at 15-20° C. for 1 h and filtered. The filter cake was rinsed with heptane/EtOAc (1.3 L, 3 v/1 v) and dried in oven at 45° C. for 12 h to afford Compound 3 as an off-white solid (985 g). ¹H NMR (400 MHz, CDCl3) δ 7.62 (s, 1H), 7.48 (s, 1H), 7.41 (dd, J=12.3, 3.5 Hz, 2H), 7.27 (dd, J=8.3, 6.0 Hz, 1H), 7.15 (td, J=8.5, 2.3 Hz, 1H), 6.59 (d, J=7.6 Hz, 1H), 6.20 (dd, J=7.6, 5.1 Hz, 1H), 5.79 (s, 2H), 5.52 (q, J=5.9 Hz, 1H), 3.99 (q, J=7.2 Hz, 2H), 3.87 (s, 3H), 3.42 (d, J=3.2 Hz, 2H), 1.45 (d, J=6.2 Hz, 3H), 1.28 (t, J=7.2 Hz, 3H).

The impurity of Formula (SP-4) is generated when formic acid reacts with the amino group in Compound 3. ¹H NMR (400 MHz, DMSO-d₆) δ 7.70 (d, J=4.0 Hz, 1H), 7.66 (s, 1H), 7.56 (d, J=10.1 Hz, 1H), 7.47 (s, 1H), 7.37 (d, J=7.1 Hz, 2H), 7.28 (dd, J=8.5, 4.7 Hz, 1H), 7.24-7.17 (m, 1H), 5.87 (d, J=5.9 Hz, 1H), 3.97 (dd, J=14.3, 7.1 Hz, 2H), 3.88 (s, 3H), 3.50 (s, 2H), 1.42 (d, J=5.9 Hz, 3H), 1.23 (t, J=6.9 Hz, 3H).

De-chlorination compound of Formula (SP-5) is another impurity generated during this step. ¹H NMR (400 MHz, DMSO-d₆) δ 8.15 (s, 2H), 7.69 (s, 1H), 7.49-7.42 (m, 2H), 7.39 (s, 1H), 7.32 (dd, J=8.4, 6.0 Hz, 1H), 7.20 (td, J=8.5, 2.6 Hz, 1H), 7.14 (s, 1H), 7.10 (d, J=7.9 Hz, 1H), 6.60-6.53 (m, 1H), 5.74 (dd, J=12.5, 6.2 Hz, 1H), 4.01 (dd, J=14.5, 7.3 Hz, 2H), 3.88 (s, 3H), 3.50 (s, 2H), 1.48 (d, J=6.2 Hz, 3H), 1.28 (t, J=7.3 Hz, 3H).

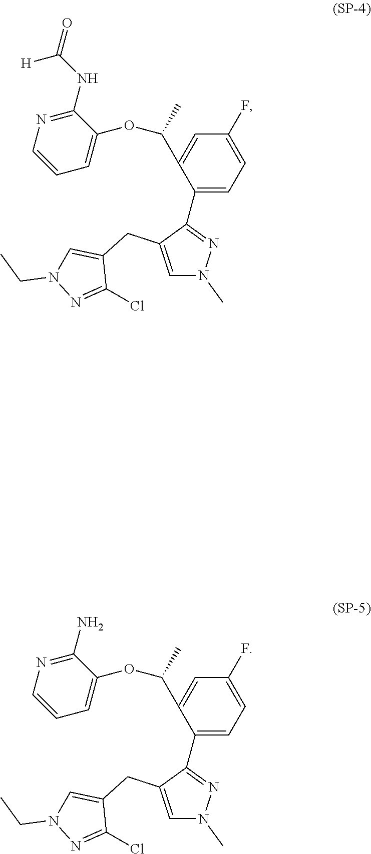 [SMILES: CCn1cc(Cc2cn(C)nc2-c2ccc(F)cc2[C@@H](C)Oc2cccnc2NC=O)c(Cl)n1 ; CCn1cc(Cc2cn(C)nc2-c2ccc(F)cc2[C@@H](C)Oc2cccnc2N)c(Cl)n1]

Synthesis of Compound 2. To a 20 L flask was added THF (1.80 L) and Compound 3 (600.0 g, 1.32 mol, 1.0 equiv.) at r.t. (15-20° C.). The mixture was purged with nitrogen. A solution of NBS (246.0 g, 1.38 mol, 1.05 equiv.) in THF (4.20 L, total=10 vol.) was added at −10˜0° C. over 30 min. The reaction mixture was stirred for 0.5-1 h at −10˜0° C. The reaction mixture was quenched with aqueous Na₂S₂O₃ solution (15 w %, 3.0 L), diluted with EtOAc (6.0 L) and separated. The organic phase was washed with aqueous Na₂CO₃ solution (20 w %, 3.0 L×2) and H₂O (3.0 L×2) respectively, then dried over Na₂SO₄, filtered and concentrated in vacuum at 40-45° C. to get crude Compound 2 as a brown solid. The combined crude Compound 2 from three batches (total 1080 g) was dissolved with EtOAc (10.8 L) at 40-50° C., and activated carbon (324 g, 30 w %) was added. The mixture was stirred for 1-2 h at 40-50° C., cooled to 20-25° C. and filtered off. The filter cake was rinsed with EtOAc (2.20 L) and the filtrate was concentrated in vacuum to get Compound 2 as a brown oil (1042 g). Compound 2 as brown oil (1040 g) was dissolved in DCM (10.4 L), then silica gel (312 g, 30 w %, 100-200 meshes, 30 w %) was added. The mixture was stirred for 1-2 h at 20-30° C. and filtered off. The filter cake was rinsed with DCM (2.00 L) and the filtrate was concentrated in vacuum at 40-45° C. to afford Compound 2 as a pale yellow foamy solid (936 g). ¹H NMR (400 MHz, DMSO-d₆) δ 7.66 (s, 1H), 7.48 (d, J=10.5 Hz, 3H), 7.26 (t, J=6.8 Hz, 1H), 7.17 (t, J=8.3 Hz, 1H), 7.07 (s, 1H), 6.12 (s, 2H), 5.56 (d, J=6.0 Hz, 1H), 4.00 (dd, J=14.0, 7.0 Hz, 2H), 3.90 (s, 3H), 3.46 (dd, J=32.6, 16.3 Hz, 2H), 1.42 (d, J=5.9 Hz, 3H), 1.29 (t, J=6.9 Hz, 3H).

Synthesis of Compound 1. To a 20 L reactor was added Compound 2 (450.0 g, 843 mmol, 1.0 equiv.) and t-AmOH (9.00 L, 20 vol.) at r.t. (20-25° C.) under nitrogen. CatacXium A (72.0 g, 200.6 mmol, 0.24 equiv.), Pd(OAc)₂ (22.5 g, 100.2 mmol, 0.12 equiv.) and potassium pivalate (354.6 g, 2529 mmol, 3 equiv.) were added at 20-30° C. under nitrogen. The reaction mixture was refilled fully with nitrogen and was stirred for 18 h at 100-105° C. The reaction mixture was cooled to 20-30° C., filtered through a celite pad (1.84 kg). The filter cake was washed with t-AmOH (1.82 L). The combined filtrates were stirred for 16 h at 60° C. with thiopropyl silica scavenger (276 g). Then the mixture was cooled to 30° C. and filtered off. The filter cake was rinsed with t-AmOH (1.82 L). The combined filtrates were stirred for 16 h at 60° C. with thiopropyl silica scavenger (276 g). Then the mixture was cooled to 30° C. and filtered off. The filter cake was rinsed with MTBE (4.60 L). The combined filtrates (t-AmOH/MTBE solution) were washed with water twice (4.6 L×2). The organic phase was concentrated in vacuum until no drop, then switched to MTBE (4.60 L) and concentrated in vacuum to get a residue. The residue was dissolved with MTBE (18.4 L). Then the solution was stirred for 1 h at 40° C. with 5 wt % aqueous. L-cysteine solution (3.0 eq., 626.8 g, 11.9 kg in water) and separated. The organic phase was washed with water (4.60 L). The above organic phase was stirred for 1 h at 40° C. with 5 wt % aqueous. L-cysteine solution (3.0 eq., 626.8 g, 11.9 kg in water) and separated. The organic phase was washed with water (4.60 L) and concentrated in vacuum to get crude Compound 1 as a yellow solid (670 g, 94.7%/220 nm). The crude Compound 1 (650 g) was dissolved in the mixed solvents of EtOH/n-heptane (1.82 L, 2V/0.8V) at 45° C. Then n-heptane (8.45 L) was added dropwise over 2 h at 40-45° C. and the seeds of Form 2 (0.5 g) were added in one portion. The slurry was stirred at 40-45° C. for 0.5 h, cooled to 10-15° C. (15° C./hour), then quickly heated to 40-45° C. over 30 min, repeated that cycle for 4 times. The mixture was cooled to 10-15° C. and stirred for 1-2 h at 10-15° C. and filtered. The filter cake was rinsed with n-heptane (1.3 L) and dried in vacuum at 45-50° C. for 16 h to afford Compound 1, Form 2, as an off-white solid (418 g, 99.3 w %, ˜54% yield). ¹H NMR (400 MHz, DMSO-d₆) δ 7.71 (dd, J=10.4, 2.4 Hz, 1H), 7.58 (s, 1H), 7.47 (d, J=1.7 Hz, 1H), 7.27-7.04 (m, 2H), 6.27 (d, J=1.5 Hz, 1H), 6.22 (s, 2H), 5.46-5.10 (m, 1H), 4.01 (qd, J=7.0, 1.9 Hz, 2H), 3.87 (s, 3H), 3.54 (d, J=15.8 Hz, 1H), 2.70 (d, J=15.7 Hz, 1H), 1.71 (d, J=6.2 Hz, 3H), 1.27 (t, J=7.2 Hz, 3H).

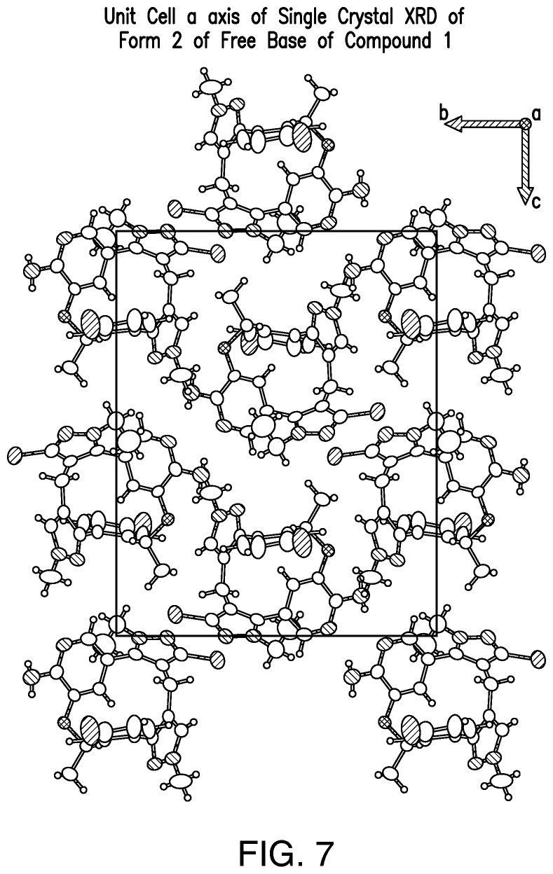

XRPD (FIG. 4), DSC (FIG. 5), DVS (FIG. 6), and Single Crystal XRD (FIG. 7) results were obtained for a sample of Form 2.

One batch of Form 2 of Compound 1 was characterized as having a chemical purity of about 97.8% (% area by HPLC), a chiral purity of about 99.5%, about 0.17% w/w of water content, residual Pt≤5 ppm, and residual Pd≤10 ppm.

Alternatively, Compound 1 can be synthesized according to Scheme 4B.

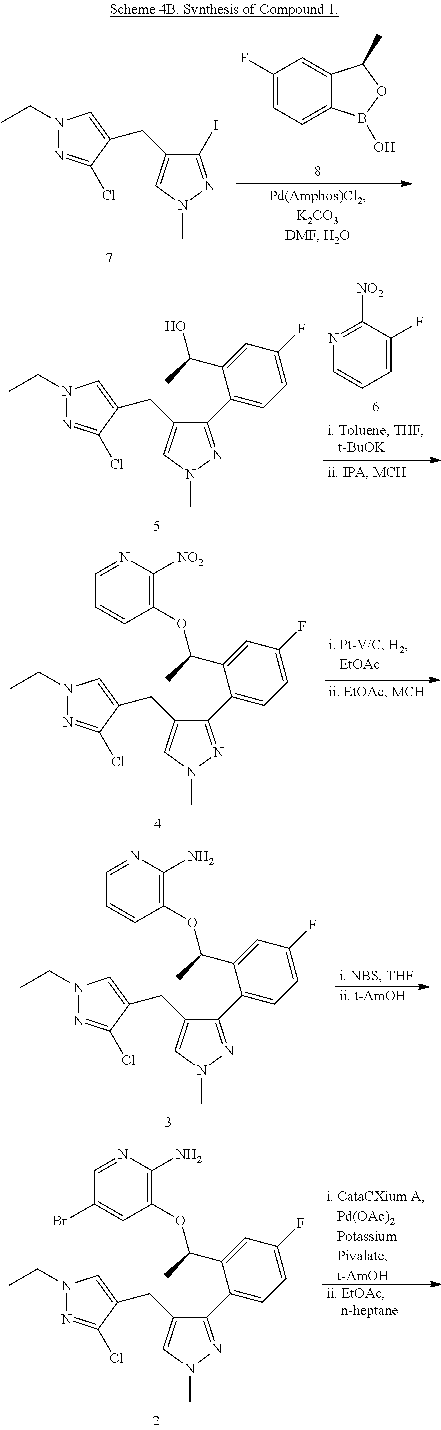

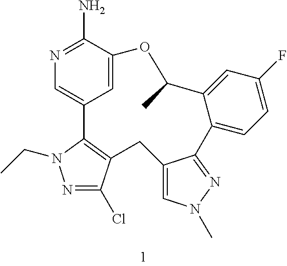 [SMILES: CCn1nc(Cl)c2c1-c1cnc(N)c(c1)O[C@H](C)c1cc(F)ccc1-c1nn(C)cc1C2]

Compound 5 was synthesized following the same procedure as in Scheme 4A. Compound 4 was synthesized using the similar procedure as in Scheme 4A, but subsequently crystallized directly from the reaction mixture with isopropanol (IPA) and methylcyclohexane (MCH). A reaction mixture of Pt—V/C (0.30 kg), Compound 4 (4.95 kg), EtOAc (21.5 kg) was stirred at 20-30° C. for 20-30 min under N₂ and swapped with H₂. The reaction mixture was stirred under H₂ pressure (0.17-0.24 MPa) at 20-30° C. for 21 h. The reactor headspace was swapped from H₂ to N₂, and the solution mixture was filtered to remove the catalyst. The filter was washed with EtOAc to provide about 38 kg solution (Batch 1). Batch 2 of about 37.8 kg solution was prepared using the similar procedure from 5.00 kg of Compound 4. Batch 1 and Batch 2 were combined and concentrated under vacuum to approximately 27 L below 40° C. and subsequently charged with MCH ˜22.4 kg at 20-25° C. A seed amount (about 0.048 kg) of Compound 3 was added and the mixture was stirred for 1-2 h. To the mixture was slowly added MCH (99.6 kg) over 10 h at 20-25° C. The temperature was adjusted to 45-50° C. within 2-3 h and the mixture was stirred for 2-3 h, then adjusted to 25-30° C. within 2-3 h and stirred for 1-2 h. The solution was further adjusted to 45-50° C. within 2-3 h and stirred for 2-3 h, then adjusted to 5-10° C. within 3-4 h and stirred for 4-6 h. The solution was filtered, and the cake was washed with MCH (about 16.8 kg) and dried under vacuum at 40-50° C. to give Compound 3 (8.24 kg, 99.8% purity, Pt<5 ppm).

Compound 3 seed preparation: To a solution of Compound 4 (9.3 g) in EtOH (100 ml) was added triethylamine (11.6 g), followed by Pt/C (1.1 g), and degassed with nitrogen. Subsequently, HCOOH (5.3 g) was added dropwise. The reaction mixture was stirred at 55° C. overnight. The reaction mixture was then cooled to room temperature and filtered through Celite. The filter cake was washed with ethyl acetate and the combined filtrate was concentrated. The residue was diluted with ethyl acetate (about 10V wherein “V” refers to the volume of the starting reagent, Compound 4), washed with aqueous NaHCO₃ (about 5V×2), water (5V), and brine (15%, 5V). The organic phase was dried over anhydrous Na₂SO₄, filtered, and concentrated to provide crude Compound 3. The crude Compound 3 was dissolved in MTBE (about 3V). To the solution was added heptane dropwise (about 1V) at room temperature and solids precipitated. Additional 4V heptane was added dropwise. The precipitate was filtered and the filter cake was rinsed with heptane/MTBE (3:1) and dried over vacuum to give Compound 3 (6.8 g).

Following the same procedure as in Scheme 4A, Compound 3 (6.5 kg) reacted with NBS (2.66 kg) in THF (about 58 kg) under N₂ to give the crude Compound 2 filtrate after work up. The crude Compound 2 filtrate was concentrated to about 26 L under vacuum below 50° C. and t-AmOH (about 26 L) was added, further concentrated to about 35 L, and more t-AmOH (about 21.4 kg) was added to provide a solution of about 42.0 kg to use in next step. Crude Compound 1 was prepared following the similar procedure as in Scheme 4A except the recrystallization was carried out with EtOAc (about 3-4× in weight relative to the crude Compound 1) and n-heptane (about 21 to 23× in weight relative to the crude Compound 1). Form 2 of Compound 1 was synthesized with about 99.9% chemical purity and about 100.0% chiral purity with residual Pt<3 ppm and residue Pd about 15 ppm.

### Example 2. Preparation of Enantiomer of Compound 1

As shown in Scheme 5, enantiomer of Compound 1 was prepared following similar procedure as Compound 1.

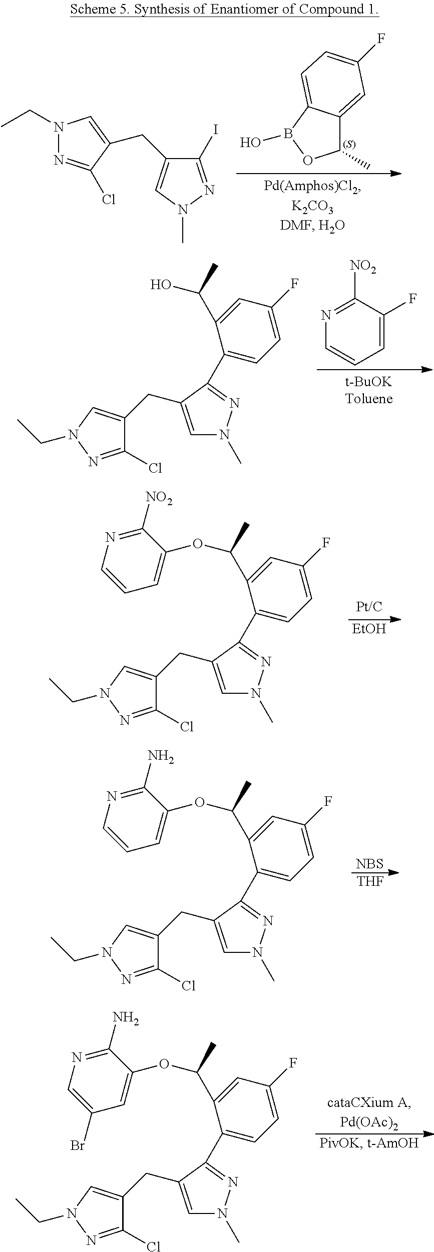

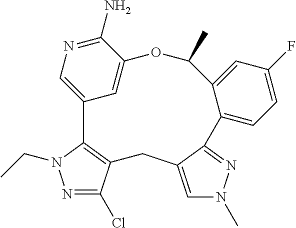 [SMILES: CCn1nc(Cl)c2c1-c1cnc(N)c(c1)O[C@@H](C)c1cc(F)ccc1-c1nn(C)cc1C2]

### Example 3. Solid Form Screening Studies

A solvent solubility screen was carried out and indicated that lyophilized material was highly soluble (>150 mg/mL) in the majority of the solvents investigated. Solids were recovered from these solutions by the addition of anti-solvent to give an indication of solid form, with Form 2 predominantly observed. Amorphous material was returned from water-containing systems, whilst Form 1 was observed in 2-methyl THF:heptane and isopropyl acetate:heptane, Form 8 from THF:heptane, and Form 9 was observed from cyclohexanone.

A solid form screen was carried out in 26 solvent systems and using a range of crystallisation techniques (temperature cycling, evaporation, antisolvent addition, crash cooling, and solvent drop grinding) starting from amorphous Compound 1. Slow evaporation, anti-solvent addition and solvent drop grinding were carried out using single solvent systems, with the slow evaporation and anti-solvent addition experiments carried out at 50° C. and ambient (ca. 20° C.) temperature. Slurrying at ambient (ca. 20° C.) temperature and temperature cycling (5-40° C.) were carried out using binary solvent systems and low solubility neat solvents, and crash cooling of saturated liquors to 5° C. from ambient temperature was also carried out. XRPD analysis indicated Form 2 was observed in most conditions at 50° C., and also after prolonged ambient temperature slurrying and temperature cycling in binary solvent mixtures containing heptane or water. Additional solid forms were observed at ambient temperatures and gums and amorphous materials were predominantly seen in water containing systems.

Desolvation experiments were carried out to investigate potential form changes indicated by the thermal data, with XPRD analysis indicating that all other identified solid forms converted to Form 2 after desolvation.

For slow evaporation experiments at ambient temperature, Form 1 was observed from 2-methyl THF, whilst Form 2 was observed form acetonitrile, tBME, isobutyl acetate and cyclopentyl methyl ether. Form 8 was observed from THF. Pattern 10 was obtained from MIBK. Patterns 12 and 13 were observed from methyl cyclohexane and cyclohexane, respectively. For slow evaporation experiments at 50° C., Form 2 was obtained from the majority of solvent systems, with Form 1 observed from 2-methyl THF, Form 4 from 1-4-dioxane, Form 7 from MIBK, and Form 8 from THF.

For anti-solvent addition experiments at ambient temperature, Form 1 was observed from 2-methyl THF:heptane and isopropyl acetate:heptane, Form 7 was observed from MIBK:heptane, Form 8 was observed from THF:heptane, Form 9 was observed from cyclohexanone:heptane and Form 11 obtained from MEK:heptane. For anti-solvent addition experiments at 50° C., Form 4 was isolated from water addition to 1,4-Dioxane, and Form 2 was observed from all other conditions where solids were recovered.

Using solvent drop grinding, Form 1 was returned from the 2-methyl THF and isopropyl acetate experiments, Form 2 was returned from the acetonitrile experiments, Form 7 was returned from the MIBK experiment, Form 5 was isolated from the 2-propanol, acetone and THF experiments, and Form 14 was observed from bead milling with cyclohexanone.

For slurrying experiments at room temperature, Form 1 was observed in 2-methyl THF:heptane, Form 4 was observed in 1,4-dioxane:water, Form 8 was observed in THF:heptane, Form 9 was observed in cyclohexanone, Form 12 was observed in methyl cyclohexane, and Form 13 was observed in cyclohexane. Form 2 was observed in the remaining solvent systems studied where solids were recovered.

For temperature cycling experiments, Form 1 was returned from 2-methyl THF: heptane, Form 4 was returned from 1,4-Dioxane:heptane, Form 8 was returned from THF:heptane, Form 9 was returned from cyclohexanone, Form 13 was returned from cyclohexane. Form 2 was observed in the remaining solvent systems studied where solids were recovered.

For crash cooling experiments, all the samples formed oils and no solids were observed after 7 days at 5° C., except for following: Form 7 was observed in MIBK:heptane, Form 8 was observed in THF:heptane, and Form 9 was observed in cyclohexanone.

### Example 4. Solid form Preparation

A. Preparation of amorphous material
1. Approximately 2.8 g of Compound 1 was dissolved in 50.4 mL t-butanol. Dissolution was aided by gentle heating to 50° C. on a hot plate
2. The resultant solution was distributed into 28×2 mL HPLC vials (1.8 mL per vial)
3. The samples were frozen at −20° C. and lyophilized over >48 h
4. Analyze by XRPD to check for amorphous content.

B. Form 1 preparation
1. Weigh approximately 100 mg of amorphous Compound 1
2. 1000 μL of isopropyl acetate:heptane (50:50% v/v) was added to the solid.
3. Resulting solution was stirred magnetically and temperature cycled between 40-5° C. over 72 hours.
   Held at 40° C. for 3 hours
   Cooled at 0.1° C./min to 5° C., held for 3 hours,
   Heated at 0.1° C./min to 40° C., held for 3 hours, repeat
4. Solids were isolated by centrifugation
5. XRPD (FIG. 1A) and TGA/DSC (FIG. 2) results were obtained for a sample of Form 1 (2-MeTHF solvate); XRPD (FIG. 1B) and TGA/DSC (FIG. 3) results were obtained for a sample of Form 1 (isopropyl acetate solvate).

C. Form 2 seed preparation
To approximately 100 mg of amorphous Compound 1 in the 4 ml vial, 200 μl of 2-methyl-THF was added to form a clear solution. The sample was left uncapped to let the solvent evaporates overnight at room temperature and pressure. The resulting form was quickly dried by heating up to 200° C. under vacuum in a Buchi glass oven piston.

D. Form 3 preparation
1. Added approximately 150 μL of 2-Methyl THF to 100 mg of Compound 1 Form 2 material.
2. Temperature cycled between ambient temperature (ca. 20° C.) and 40° C. in an incubator/shaker.
3. Isolated solids by centrifugation.
4. XRPD (FIG. 8) and TGA/DSC (FIG. 9) results were obtained for a sample of Form 3.

E. Form 4 preparation
1. Weigh approximately 100 mg of amorphous Compound 1.
2. 400 μL of 1,4-dioxane in 50 μL aliquots were added to the solid at 50° C. in a HPLC vial.
3. The HPLC vial was sealed with a cap and pierced with syringe needle.
4. The solvent was allowed to evaporate slowly at 50° C. over 2 days
5. XRPD (FIG. 10) and TGA/DSC (FIG. 11) results were obtained for a sample of Form 4.

F. Form 5 (t-BuOH and 2-propanol mixed solvate) preparation
1. A drop (10 μL) of 2-propanol was added to approximately 20 mg of amorphous Compound 1 (some residual t-butanol was present in lyophilized solid) in 2 mL bead mill vials along with 3 steel bead mill balls.
2. The vials were then bead milled using the following program: speed: 6000 RPM; cycle: 40×90 s (1 hour of milling time total), pause: 10 s.
3. XRPD (FIG. 12A) and TGA/DSC (FIG. 13) results were obtained for a sample of Form 5 (t-BuOH and 2-propanol mixed solvate).

G. Form 5 (t-BuOH and acetone mixed solvate) preparation
4. A drop (10 μL) of acetone was added to approximately 20 mg of amorphous Compound 1 (some residual t-butanol was present in lyophilized solid) in 2 mL bead mill vials along with 3 steel bead mill balls.
5. The vials were then bead milled using the following program: speed: 6000 RPM; cycle: 40×90 s (1 hour of milling time total), pause: 10 s.
6. XRPD (FIG. 12B) and TGA/DSC (FIG. 14) results were obtained for a sample of Form 5 (t-BuOH and acetone mixed solvate).

H. Form 5 (t-BuOH and THF mixed solvate) preparation
7. A drop (10 μL) of THF was added to approximately 20 mg of amorphous Compound 1 (some residual t-butanol was present in lyophilized solid) in 2 mL bead mill vials along with 3 steel bead mill balls.
8. The vials were then bead milled using the following program: speed: 6000 RPM; cycle: 40×90 s (1 hour of milling time total), pause: 10 s.
9. XRPD (FIG. 12C) and TGA/DSC (FIG. 15) results were obtained for a sample of Form 5 (t-BuOH and THF mixed solvate).

I. Form 6 preparation
1. Weigh approximately 20 of compound 1, and 250 μL of acetone was added to the solid at 20° C.
2. The solvent was allowed to evaporate slowly at ambient (ca. 20° C.) in an uncapped HPLC vial.
3. XRPD (FIG. 16) results were obtained for a sample of Form 6.

J. Form 7 preparation
1. Weigh approximately 20 mg of amorphous Compound 1
2. 10 μL of MIBK was added to the solid at 20° C.
3. Samples were agitated at ambient (ca. 20° C.) in plastic vial with 3 stainless steel ball bearings in place
4. The vials were then bead milled using the following program: speed: 6000 RPM; cycle: 40×90 s (1 hour of milling time total), pause: 10 s
5. XRPD (FIG. 17) and TGA/DSC (FIG. 18) results were obtained for a sample of Form 7.

K. Form 8 preparation
1. Weigh approximately 100 mg of amorphous Compound 1
2. 500 μL of THF and 500 μL of heptane were added to form a slurry
3. Slurry was stirred magnetically over 24 hours at ambient temperature (ca. 20° C.)
4. Solids isolated by centrifugation (0.22 μm, nylon)
5. XRPD (FIG. 19) and TGA/DSC (FIG. 20) results were obtained for a sample of Form 8.

L. Form 9 preparation
1. Weigh approximately 100 mg of amorphous Compound 1
2. 500 μL of cyclohexanone was added to the solid
3. Temperature of the resulting solution was cycled between 40-5° C. over 72 hours and stirred magnetically.
   Held at 40° C. for 3 hours
   Cooled at 0.1° C./min to 5° C., held for 3 hours,
   Heated at 0.1° C./min to 40° C., held for 3 hours, repeat
4. Isolated solids by centrifugation.
5. XRPD (FIG. 21) and TGA/DSC (FIG. 22) results were obtained for a sample of Form 9.

M. Form 10 preparation
1. Weigh approximately 100 mg of amorphous Compound 1
2. 1000 μL of MIBK in 100 μL aliquots was added to the solid
3. Resulting solution was left to evaporate through a HPLC vial Screwcap (pierced cap with syringe needle) at 20° C. for 2 days
4. XRPD (FIG. 23) and TGA/DSC (FIG. 24) results were obtained for a sample of Form 10.

N. Form 11 preparation
1. Weigh approximately 100 mg of amorphous Compound 1
2. 250 μL of MEK in 50 μL aliquots was added to compound 1 at ambient temperature (20° C.)
3. Transferred 150 μL of solution into separate vial, stirred solution magnetically (ca. 20° C.).
4. Added 150 μL of heptane to solution, stirred overnight magnetically at ambient (ca. 20° C.).
5. Isolated solids by centrifugation XRPD (FIG. 25) and TGA/DSC (FIG. 26) results were obtained for a sample of Form 11.

O. Form 12 preparation
1. Weigh approximately 20 mg of amorphous Compound 1
2. 2000 μL of methyl cyclohexane were added to form a slurry
3. Slurry was stirred magnetically over 24 hours at ambient temperature (ca. 20° C.).
4. Solids isolated by centrifugation (0.22 μm, nylon)
5. XRPD (FIG. 27) and TGA/DSC (FIG. 28) results were obtained for a sample of Form 12.

P. Form 13 preparation
1. Weigh approximately 20 mg of lyophilized amorphous Compound 1
2. 2000 μL of cyclohexane were added to form a slurry
3. Slurry was stirred magnetically over 24 hours at ambient temperature (ca. 20° C.)
4. Solids isolated by centrifugation (0.22 μm, nylon)
5. XRPD (FIG. 29) and TGA/DSC (FIG. 30) results were obtained for a sample of Form 13.

Q. Form 14 preparation
1. A drop (10 μL) of cyclohexanone was added to approximately 20 mg of amorphous Compound 1 (some residual t-butanol was present in lyophilized solid) in 2 mL bead mill vials along with 3 steel bead mill balls.
2. The vials were then bead milled using the following program: speed: 6000 RPM; cycle: 40×90 s (1 hour of milling time total), pause: 10 s
3. XRPD (FIG. 31) and TGA/DSC (FIG. 32) results were obtained for a sample of Form 14.

R. Form 15 preparation
1. Weigh approximately 20 mg of Form 2 into HPLC vial
2. Acetone was added in 2×50 μL aliquots (total 100 μL)
3. An additional 8.1 mg of Form 2 was then added, resulting in a slurry forming which thickened immediately upon manual agitation
4. Slurry stirred for 3 days
5. Solids was filtered by centrifugation (0.22 μm, nylon)
6. XRPD (FIG. 33) and TGA/DSC (FIG. 34) results were obtained for a sample of Form 15.

### Example 5: Desolvation Experiments

Desolvation experiments were carried out on the solvated forms identified during the polymorph screen to establish the relationships between the forms. Samples were heated to a temperature at which the solids fully desolvated (based on TG/DSC characterisation data) and where exothermic events were observed after desolvation a separate experiment was carried out to assess for additional solid forms. Solids were analysed by XRPD after heating, with the analysis indicating that all solids converted to Form 2 upon heating to the temperatures investigated. The results are summarized in the following table.

| | | Target | | |
|---|---|---|---|---|
| Input | | Temperature | Preceding | Solid Form |
| Form | Solvate | (° C.) | event | post-heating |
| Form 1 | 2-Methyl THF | 105 | Desolvation | Form 2 |
| Form 1 | Isopropyl acetate | 150 | Desolvation | Form 2 |
| Form 4 | 1,4-Dioxane | 132 | Desolvation | Form 2 |
| Form 4 | 1,4-Dioxane | 170 | Exothermic | Form 2 |
| | | | event | |
| Form 7 | MIBK | 134 | Desolvation | Form 2 |
| Form 8 | THF | 150 | Desolvation | Form 2 |
| Form 8 | THF | 200 | Exothermic | Form 2 |
| | | | event | |
| Form 9 | Cyclohexanone | 150 | Desolvation | Form 2 |
| Form 10 | MIBK | 150 | Desolvation | Form 2 |
| Form 11 | MEK | 130 | Desolvation | Form 2 |
| Form 11 | MEK | 200 | Exothermic | Form 2 |
| | | | event | |
| Form 12 | Methyl | 150 | Desolvation | Form 2 |
| | Cyclohexane | | | |
| Form 13 | Cyclohexane | 150 | Desolvation | Form 2 |
| Form 15 | Acetone | 150 | Desolvation | Amorphous |
| Form 15 | Acetone | 200 | Exothermic | Form 2 |
| | | | event | |

### Example 6: Single Crystal X-Ray Diffraction Characterization of Form 2

A full crystal structure of Form 2 was collected and solved, See FIG. 7 for a representative depiction of the structure determined from single-crystal X-ray diffraction studies of Form 2. A summary of structural data for Compound 1 Form 2 is provided in the table below. Form 2 crystallizes in the orthorhombic system, space group P2₁2¹2₁ with the final R1 [I>2σ(I)]=3.76%.

TABLE 2
Crystallographic parameters and refinement
indicators for Compound 1 Form 2

| Parameter | Value |
|---|---|
| Empirical formula | C₂₃H₂₂ClFN₆O |
| Formula weight | 452.92 |
| Temperature/K | 296.0 |
| Crystal system | Orthorhombic |
| Space group | P2₁2₁2₁ |
| a/Å | 8.1686(13) |
| b/Å | 14.750(3) |
| c/Å | 18.694(3) |
| α/° | 90 |
| β/° | 90 |
| γ/° | 90 |
| Volume/Å³ | 2252.4(7) |
| Z | 4 |
| ρ_{calc}g/cm³ | 1.336 |
| μ/mm⁻¹ | 1.805 |
| F(000) | 944.0 |
| Crystal size/mm³ | 0.18 × 0.16 × 0.12 |
| Radiation | CuKα (λ = 1.54178) |
| 2Θ range for data collection/° | 7.634 to 140.152 |
| Index ranges | −9 ≤ h ≤ 9, −15 ≤ |
| | k ≤ 17, −22 ≤ 1 ≤ 22 |
| Reflections collected | 16936 |
| Independent reflections | 4272 [Rᵢₙₜ = 0.0491, R_{sigma} = 0.0393] |
| Data/restraints/parameters | 4272/0/293 |
| Goodness-of-fit on F² | 1.083 |
| Final R indexes [I >= 2σ (I)] | R₁ = 0.0376, wR₂ = 0.0865 |
| Final R indexes [all data] | R₁ = 0.0462, wR₂ = 0.0927 |
| Largest diff. peak/hole/e Å⁻³ | 0.14/−0.26 |
| Flack parameter | 0.001(11) |

R₁ = (Σ|Fₒ|−|F_{c}|)/Σ|Fₒ|); wR₂ = {Σ[w(Fₒ² − F_{c}²)²]/Σ[w(Fₒ²)²]}^{1/2}; S = {Σ [w(Fₒ² − F_{c}²)²]/(n-p)}^{1/2}.

The asymmetric unit of Form 2 contains one fully ordered molecule of Compound 1. Anisotropic atomic displacement ellipsoids for the non-hydrogen atoms are shown at the 50% probability level. Hydrogen atoms are displayed with an arbitrarily small radius.

The absolute configuration of Compound 1 has been determined as depicted below with the Flack parameter=0.001(11). See Parsons, Sand Flack, H., Acta Cryst. 2004, A60, s61. The chiral center has an R configuration.

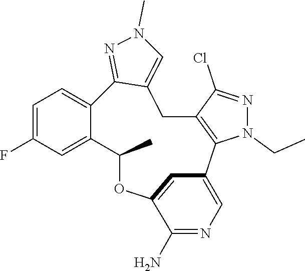

### Example 7. Salt Screen

A salt screen was carried out on Compound 1 using 6 counterions and 4 solvents, to identify suitable conditions for successful salt formation. The acid counterions investigated are: hydrochloric acid (37 wt %, 12M), methane sulfonic acid, benzene sulfonic acid, maleic acid, phosphoric acid (85% wt, 15M), and citric acid.

The first set of salt formation experiments were carried out using 2-propanol. Approximately 20 mg of Compound 1 was weighed into 6×1.5 mL glass vials. The material was dissolved in 200 μL of 2-propanol. 1.05 equivalents of selected acid counterion ion was added to each sample (46.4 μL of 1M aqueous acid stock solution added). Initial observations were recorded, and the samples were temperature cycled between ambient temperature and 40° C. in 4-hour cycles for ca. 72 hours. The samples were collected, and observations made. Clear, colorless solutions were observed in all samples. The vials were uncapped and stored under ambient conditions to allow evaporation. Post-evaporation clear, glassy solids were observed in all samples. The aliquots of the solids were analyzed by XRPD and ¹H NMR to assess crystallinity and salt formation. The results are shown in the following table.

| | | Observations | Observations | | | | |
|---|---|---|---|---|---|---|---|
| | Volume of | Post- | Post- | | ¹H NMR | | Salt |
| Counterion | Solvent (μL) | Maturation | Evaporation | XRPD | Counterion | Solvent | Formation |
| Hydrochloric | 200 | Clear colorless | Clear, colorless | Amorphous | Unknown | 0.27 eq. IPA | Yes |
| acid | | solution | glassy solid | | | | |
| Methane | 200 | Clear colorless | Clear, colorless | Amorphous | 1 eq. acid | 0.08 eq. IPA | Yes |
| sulfonic acid | | solution | glassy solid | | | | |
| Benzene | 200 | Clear colorless | Clear, colorless | Amorphous | 1 eq. acid | 0.52 eq. IPA | Yes |
| sulfonic acid | | solution | glassy solid | | | | |
| Maleic acid | 200 | Clear colorless | Clear, colorless | Amorphous | 1 eq. acid | 0.53 eq. IPA | Yes |
| | | solution | glassy solid | | | | |
| Phosphoric | 200 | Clear colorless | Clear, colorless | Amorphous | Unknown | 0.42 eq. IPA | No evidence |
| acid | | solution | glassy solid | | | | of salt |
| | | | | | | | formation |
| Citric acid | 200 | Clear colorless | Clear, colorless | Amorphous | 1 eq. acid | 1.2 eq. IPA | Yes |
| | | solution | glassy solid | | | | |

A broad water peak was observed along with shifting peaks in ¹H NMR for hydrochloric acid, methane sulfonic acid, benzene sulfonic acid, maleic acid, and citric acid samples, indicating successful sat formation. For phosphoric acid sample, the ¹H NMR spectrum appeared consistent with Compound 1 spectrum, indicating no salt formation (in ¹H NMR sample). It is possible that salt formation likely had occurred, however the salt may have disproportionated in DMSO. This is because the free base would be expected to crystallize to Form 1 in 2-methyl-THF (in next set of salt formation experiments).

The second set of salt formation experiments were carried out using 2-methyl THF. The glassy solids previously observed were collected and 50 μL of 2-methyl THF was added to each vial. The samples were then temperature cycled between ambient temperature and 40° C. in 4-hour cycles for ca. 2 hours. After 24 hours all samples appeared as clear, colorless solutions. The vials were uncapped and stored under ambient conditions to allow evaporation. Glassy solids were observed in all samples post-evaporation. All solids appeared non-birefringent by PLM. All samples appeared amorphous by PLM.

The third set of salt formation experiments were carried out using isopropyl acetate. The glassy solids previously observed were collected and 100 μL of isopropyl acetate was added to each vial. The vials were stored uncapped under ambient conditions to allow evaporation. Glassy solids were observed in all samples post-evaporation. The glassy solids were analyzed by PLM. All solids appeared non-birefringent. The solids were then re-dissolved in 100 μL of acetone and stored under ambient conditions to allow evaporation. Glassy solids were observed post-evaporation. All samples appeared amorphous by PLM.

The fourth set of salt formation experiments were carried out using acetone. The glassy solids previously observed were dissolved in 100 μL of acetone and stored under ambient conditions to allow evaporation. Glassy solids were observed post-evaporation. An aliquot of each glassy solid was analyzed by DSC to assess crystallization upon heating the amorphous solids. Samples were heated at 10° C./min from 20-300° C.

DSC analysis of the hydrochloric acid, methane sulfonic acid, and benzene sulfonic acid samples showed a large broad endotherm from the onset of the heating to ca. 140° C., likely related to the loss of surface moisture/solvent. Thermal degradation was noted after ca. 200° C. No other significant thermal events were noted.

DSC analysis of the maleic acid sample showed two large broad endotherms from the onset of the heating to ca. 150° C., likely related to the loss of surface moisture/solvent. Thermal degradation was noted after ca. 150° C. No other significant thermal events were noted.

DSC analysis of the phosphoric acid sample showed a large broad endotherm from the onset of the heating to ca. 125° C., likely related to the loss of surface moisture/solvent. Thermal degradation was noted after ca. 175° C. No other significant thermal events were noted.

DSC analysis of the citric acid sample showed a large broad endotherm from the onset of the heating to ca. 125° C., likely related to the loss of surface moisture/solvent. Thermal degradation was noted after ca. 150° C. No other significant thermal events were noted.

### Example 8. Solid Form Characterization

### X-Ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Mylar polymer film to support the sample. The multi-well plate was then placed into the diffractometer and analyzed using Cu K radiation (α₁ λ=1.54060 Å; α₂=1.54443 Å; β=1.39225 Å) running in transmission mode (step size 0.0130° 2θ, step time 18.87 s) using 40 kV/40 mA generator settings. Data were visualized and images generated using the HighScore Plus 4.7 desktop application (PANalytical, 2017). Alternatively, XRPD was performed using a Bruker D8 Advance equipped with LYNXEYE detector in reflection mode (i.e. Bragg-Brentano geometry). Samples were prepared on Si zero-return wafers. Radiation source: Cu Kα₁=1.5406 Å; Kα₂=1.5444 Å; the ratio of Kα₁:Kα₂ was about 2:1 (2.1:1.0).

### Single Crystal X-Ray Diffraction (SCXRD)

Data were collected on an Oxford Diffraction Supernova Dual Source, Cu at Zero, Atlas CCD diffractometer equipped with an Oxford Cryosystems Cobra cooling device. The data was collected using CuKα radiation. Structures were typically solved using either the SHELXS or SHELXD programs and refined with the SHELXL program as part of the Bruker AXS SHELXTL suite (V6.10). Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

### Nuclear Magnetic Resonance (NMR)

NMR experiments were performed on a Bruker AVIIIHD spectrometer equipped with a PRODIGY cryoprobe operating at 500.23 MHz for protons. Experiments were performed in deuterated dimethyl sulfoxide and each sample was prepared to ca. 10 mM concentration.

### Differential Scanning Calorimetry (DSC)

Approximately, 1-5 mg of material was weighed into an aluminium DSC pan and sealed nonhermetically with an aluminium lid. The sample pan was then loaded into a TA Instruments Discovery DSC 2500 differential scanning calorimeter equipped with a RC90 cooler. The sample and reference were heated to 300° C. at a scan rate of 10° C./min and the resulting heat flow response monitored. The sample was re-cooled to 20° C. and then reheated again to 300° C. all at 10° C./min. Nitrogen was used as the purge gas, at a flow rate of 50 cm3/min.

### Thermogravimetric Differential Thermal Analysis (TG/DTA)

Approximately, 5-10 mg of material was weighed into an open aluminium pan and loaded into a simultaneous thermogravimetric/differential thermal analyser (TG/DTA) and held at room temperature. The sample was then heated at a rate of 10° C./min from 20° C. to 400° C. during which time the change in sample weight was recorded along with any differential thermal events (DTA). Nitrogen was used as the purge gas, at a flow rate of 300 cm3/min.

### Thermogravimetric Analysis Differential Scanning Calorimetry (TGA/DSC)

Approximately, 5-10 mg of material was added into a pre-tared open aluminum pan and loaded into a TA Instruments Discovery SDT 650 Auto-Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm3/min.

### Dynamic Vapour Sorption (DVS)

Approximately, 10-20 mg of sample was placed into a mesh vapour sorption balance pan and loaded into a DVS Advantage dynamic vapour sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### Example 9. Preparation of Tablets of Compound 1

Compound 1 tablets are manufactured for oral administration at 5 mg and 50 mg strengths. The 5 mg tablets are manufactured as immediate release, film-coated, orange, round tablets. The 50 mg tablets are manufactured as immediate release, film-coated, orange, oblong tablets. The quantitative composition of the tablets is provided in the following table.

TABLE 3
Composition of Compound 1 Tablets, 5 mg and 50 mg

| | | Target Quantity | |
|---|---|---|---|
| | | (mg/tablet) | |
| Component | Function | 5 mg | 50 mg |
| Compound 1 Drug Substance^{a} | Active | 5.00 | 50.00 |
| Microcrystalline Cellulose^{a} | Diluent | 83.50 | 170.00 |
| Croscarmellose Sodium | Disintegrant | 5.00 | 12.50 |
| Colloidal Silica Dioxide | Glidant | 2.50 | 6.25 |
| Hydroxypropyl Cellulose | Binder | 2.50 | 6.25 |
| Magnesium Stearate | Lubricant | 1.50 | 5.00 |
| Opadry II Orange^{b} | Cosmetic film-coat | 3.00 | 6.25 |
| Purified Water^{c} | Coating Agent | qs | qs |

| | | |
|---|---|---|
| Total | 103.00 | 256.25 |

^{a}The amount of drug substance and microcrystalline cellulose may be adjusted depending on the potency of the drug substance
^{b}Non-functional, cosmetic film coating, added to target theoretical weight gain of 3% and 2.5% for the 5 mg and 50 mg tablets, respectively
^{c}Removed during processing.
qs: quantity sufficient

### Example 10. Stability Studies of Compound 1 and Compound 1 Tablets

A stability study is conducted for Compound 1. Samples were stored at: 30° C.±2° C./65%±5% Relative Humidity (RH), or 40° C.±2° C./75%±5% RH. At regular intervals (e.g., initial, 1, 3, 6, 9, 12, 18, 24, and 36 months), tests are performed for description, assay, related substances, water content, polymorphic form, and chiral purity.

Stability results show the chemical and physical stability of Compound 1 drug substance stored for 1 month at the proposed long-term condition of 30° C.±2° C./65%±5% RH and accelerated condition of 40° C.±2° C./75%±5% RH. No meaningful changes were observed in description, assay, related substances, water content, polymorphic form, and chiral purity for three months.

A stability study is conducted for Compound 1 tablets, 5 mg and 50 mg, having the compositions in Example 9. Stability samples were stored at: 30° C.±2° C./65%±5% RH, or 40° C.±2° C./75%±5% RH. At regular intervals (e.g., initial, 1, 2, 3, 6, 9, 12, 18, 24, and 36 months), tests are performed for description, assay and degradation products, dissolution, water content, and chiral purity.

Stability study show the chemical and physical stability of Compound 1 tablets upon storage for up to 1 month under long-term ICH condition of 30° C.±2° C./65%±5% RH and accelerated condition of 40° C.±2° C./75%±5% RH.

Another stability study is conducted for Compound 1 tablets, 5 mg and 50 mg, having the compositions in Example 9. Stability samples were stored at: 30° C.±2° C./65%±5% RH, or 40° C.±2° C./75%±5% RH. At regular intervals (e.g., initial, 1, 3, 6, 9, 12, 18, 24, and 36 months), tests are performed for description, assay and degradation products, dissolution, water content, and microbial content.

### Example 11. Alternative Synthesis of Compound 8

This example presents an alternative to the synthesis of compound 8 depicted in Scheme 1.

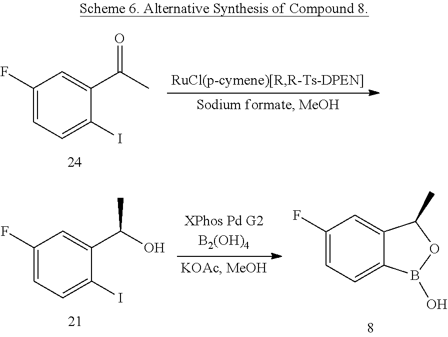

Synthesis of Compound 21. Compound 24 (1.0 kg, 1.0 eq) was charged to an inert reactor and was dissolved in about 10 L of methanol. Sodium formate (0.258 kg, 1.0 eq) was charged, and the reactor stirred and degassed. RuCl(p-cymene)[(R,R)-TsDPEN] (0.0241 kg, 0.01 eq) was charged to the reactor, and the reaction was stirred at 25° C. Reaction conversion was monitored via HPLC analysis. When the reaction was complete, the reaction was filtered through a celite pad, and the filtrate concentrated under reduced pressure. The resultant material was dissolved in MTBE, and was washed with aqueous L-cysteine, sodium bicarbonate, and water. The organic fraction was decolorized with activated carbon and silica. The reaction mass was filtered and concentrated under reduced pressure. Crystallization from MTBE and heptane afforded the Compound 21 as a beige solid (89% yield). ¹H NMR (400 MHz, DMSO-d₆) δ 7.78-7.75 (m, 1H), 7.28-7.52 (m, 1H), 6.91-6.86 (m, 1H), 5.54-5.53 (d, J=4 Hz, 1H), 4.72-4.66 (m, 1H), 1.22-1.21 (d, J=6.4 Hz, 3H). HPLC Chiral purity: 99.85%.

Synthesis of Compound 8. Methanol (10 L/kg) and Compound 21 (1.0 kg, 3.758 moles, 1.0 eq) were charged to the reactor at 25±5° C. under argon atmosphere and stirred for 10-15 min under an argon atmosphere. Tetrahydroxydiboron (0.573 kg, 6.389 moles, 1.7 eq) was charged followed by potassium acetate (0.738 kg, 7.515 moles, 2.0 eq) and XPhos Pd G2 (0.0074 kg, 0.0093 moles, 0.0025 eq) at 25±5° C. and stirred for 10 min under argon atmosphere. The reaction mixture was stirred and purged with argon for 1 h, and then the temperature raised to 55±5° C. The reaction was stirred for 8 hours at 55±5° C. and reaction progress monitored by HPLC. Upon reaction completion, the reaction mixture was concentrated under reduced pressure at 45° C. The reaction mixture was chased three time with MTBE (two volumes each time), and was concentrated to 2 volumes with respect to starting material. The resulting mixture was cooled to 25±5° C. and MTBE (5 L/kg) was charged, along with L-cysteine solution (20% wt/vol in water). Stirring speed was reduced during this step in order to avoid emulsion formation, and the biphasic system was stirred for 2 h at 40±5° C. The separation was allowed to cool to 25±5° C., stirring was discontinued to allow the layers to settle, and the layers were separated. The aqueous layer was extracted with MTBE (2 vol). The organic fractions were combined and washed with ˜4% Sodium bicarbonate solution (5 L/kg) followed by ˜20% w/w sodium chloride solution (5 L/kg). DARCO G60, (0.20 kg/kg) and silica gel 60-120 (0.20 kg/kg) were charged to the organic layer and stirred for 1 hour at 25±5° C. The silica and carbon were removed via filtration through a celite bed. The celite bed was washed with MTBE (˜15 L/kg, checking the last filtrate of each wash for absence of product by TLC) and the combined filtrates were concentrated to ˜1.5 vol under reduced pressure at 40° C. Purified water (1.5 L/kg) was charged into the concentrated mixture and distilled to 1.5 volumes at 40° C. (twice). The resulting mixture was gradually cooled to 25±5° C. and stirred for 2 h at temperature. The reaction mixture was further cooled to 5±5° C. and stirred for 2 hours at 5±5° C. to complete precipitation. The precipitated solids were filtered, washed with cold purified water (0.5 L/kg) and dried under reduced pressure at 40±5° C. to obtain crude Compound 8. Chemical purity and chiral purity by HPLC was evaluated in comparison to a known standard.

MTBE (5 L/kg) and crude Compound 8 (1 kg, 6.025 moles, 1.0 eq) were charged to the reactor at 25±5° C. Darco G60 (0.2 kg/kg) and silica gel (60-120) (0.2 kg/kg) was charged to the reactor at 25±5° C. and stirred for 1 hr at 25±5° C. The reaction mixture was filtered through celite pad and washed with MTBE (15 L/kg). Combined filtrates was concentered to about 2 vol level based on crude at 45° C. The reaction mixture was chased with n-Heptane (2 L/kg) two times at 45° C. n-Heptane (3 L/kg) was charged and heated to 45° C. to get clear solution. Gradually the reaction mixture was allowed to attain a temperature of 25±5° C. and stirred for 2 h at 25±5° C. The reaction mixture was further cooled to 0±5° C. over a period of 2 h and stirred at 0±5° C. for 3 h. The reaction mixture was filtered while maintaining the reactor contents at a temperature of 0±5° C. and the solids washed with pre-cooled n-heptane (0±5° C.) (0.1 L/kg). The resulting Compound 8 was analyzed for chemical purity and chiral purity by HPLC in comparison to a known standard.

If HPLC purity of Compound 8 complies with desired levels, then the compound was dried at 45° C. and unloaded into double LLDPE transparent polythene bags. If purity did not comply, then the following operations were performed. n-Heptane (3 L/kg) was charged to the reactor, followed by isolated Compound 8, and heated to 45° C. to get a clear solution. Gradually, the reaction mixture was allowed to attain 25±5° C. and stirred for 2 h at 25±5° C. The reaction mixture was further cooled to 0±5° C. over period of 2 h and stirred at 0±5° C. for 3 h. The reaction mixture was filtered by maintaining the reactor contents temperature at 0±5° C. and the solids then washed with pre-cooled n-heptane (0±5° C.) (0.1 L/kg). The compound was analyzed for chemical purity and chiral purity by HPLC. ¹H NMR (400 MHz, DMSO-d₆) δ 9.16 (s, 1H), 7.73-7.70 (m, 1H), 7.27-7.25 (m, 1H), 7.18-7.13 (m, 1H), 5.20-5.16 (dd, J=6.8 Hz, 1H), 1.40-1.38 (d, J=6.8 Hz, 3H). ¹³C NMR (100 MHz, DMSO-d₆) δ 165.59, 163.13, 161.39-161.31 (d), 132.77-132.68 (d), 114.77-114.56 (d), 101.53-101.31 (d), 76.21-76.18 (d), 22.31. HPLC Chemical purity: 99.57%. HPLC Chiral purity: 99.96%.

### Example 12. Alternative Synthesis of Compound 14

This example presents an alternative to the synthesis of Compound 14 depicted in Scheme 2.

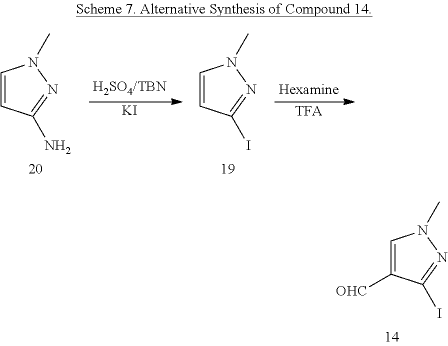

Synthesis of Compound 19. Compound 20 (1.0 kg, 1.0 eq.) and about 4 L of purified water were charged to an inert reactor at 10° C. To this mixture, concentrated H₂SO₄ (2 L, 3.53 eq.) was slowly added to reaction mixture at a rate to maintain the temperature, and was subsequently stirred for 1 h. t-Butyl nitrite (1.6 L, 1.15 eq) was added to the reactor and stirring continued. The reactor was heated to 30° C., and a solution of potassium iodide (2.14 kg, 1.25 eq) in 4.4 L of water was prepared and slowly added to the reaction vessel. The reaction progress was monitored via HPLC analysis. Upon completion of the reaction, the reaction mixture was diluted with ethyl acetate, and washed first with aqueous thiosulfate and then water. The organic layer was concentrated under reduced pressure to give Compound 19 in 86% yield. ¹H NMR (400 MHz, CDCl₃): δ 7.16 (d, J=2.4 Hz, 1H), 6.36 (d, J=2.0 Hz, 1H), 3.89 (s, 3H). LCMS: m/z [M+H]⁺ 208.97.

Synthesis of Compound 14. Compound 19 (1.0 kg, 1.0 eq) was charged to an inert reactor. TFA was added to the reactor (3.0 kg, 3.6 eq), and the reactor was heated to 35° C. Hexamine (2.0 kg, 2.9 eq) was charged to the reactor portion-wise over a 3 h period. When addition was complete, the reaction was heated to 80° C., and the reaction progress was monitored via HPLC analysis. Once complete, the reaction was cooled to 25° C., and was neutralized with aqueous sodium carbonate, followed by a solution of aqueous sodium bisulfite. The resulting mixture was stirred at 25° C. for 3 h, and was filtered. The filtered reaction mixture was washed with water and dried under reduced pressure to give Compound 14 in 70% yield. ¹H NMR (400 MHz, CDCl₃): δ 9.62 (s, 1H), 7.78 (s, 1H), 3.95 (s, 3H). LCMS: m/z [M+H]⁺ 236.9.

### Example 13. Alternative Synthesis of Compound 15

This example presents an alternative to the synthesis of Compound 7 depicted in Scheme 3.

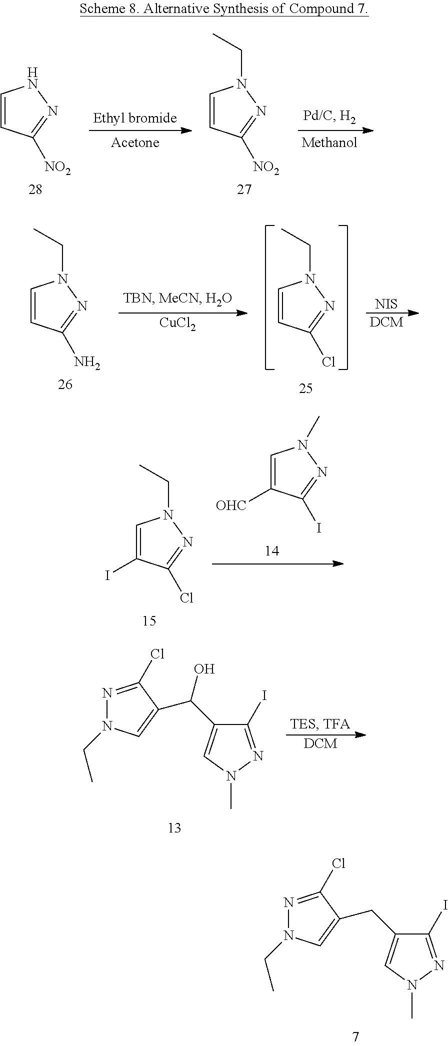

Synthesis of Compound 27. Compound 28 (2.5 kg, 1.0 eq) was charged to an inert reactor at atmospheric pressure and was dissolved in about 23 L of acetone. To this solution, K₂CO₃ (4.1 kg, 1.38 eq) was charged, and the reaction stirred for 15 minutes. Ethyl bromide (3.35 kg, 1.4 eq) was charged to the reactor, and the reaction allowed to stir at 25° C. Reaction progress was followed via HPLC analysis. Upon completion, the reaction mixture was filtered, and concentrated under reduced pressure. The resultant material was dissolved in DCM, and was washed with water and brine. The material was concentrated under reduced pressure, and crystallized from TMBE/heptane to afford Compound 27 (73% yield). ¹H NMR (400 MHz, CDCl₃): δ 7.44 (d, J=2.8 Hz, 1H), 6.84 (d, J=2.4 Hz, 1H), 4.22 (q, J=7.2 Hz, 2H), 1.49 (t, J=7.2 Hz, 3H). LCMS: m/z [M+H]⁺ 142.2.

Synthesis of Compound 26. Compound 27 (1.0 kg, 1.0 eq) was charged to a hydrogenation reactor and dissolved in about 10 L of methanol under an inert atmosphere. To this mixture, a slurry of Pd on C (0.005 kg, 0.5 wt %) was introduced. The reactor was flushed with nitrogen, followed by pressurizing with hydrogen to 45 psi. The reaction was stirred at a temperature of 40° C., and reaction progress monitored via HPLC analysis. After completion of the reaction, the organic fraction was inertly filtered through celite in order to remove catalyst, and was concentrated under reduced pressure to give Compound 26 in 98% yield. ¹H NMR (400 MHz, CDCl₃): δ 7.07 (d, J=2.4 Hz, 1H), 5.51 (d, J=2.4 Hz, 1H), 3.90 (q, J=7.2 Hz, 2H), 3.34 (bs, 2H), 1.35 (t, J=7.2 Hz, 3H). LCMS: m/z [M+H]⁺ 112.09.

Synthesis of Compound 15. To an inert reactor containing about 2.5 L of water, CuCl₂ (1.5 kg, 1.5 eq) was charged portion-wise and allowed to dissolve. The reactor temperature was lowered to 10° C., and a solution of Compound 26 (0.84 kg, 1 eq) in MeCN was charged slowly while maintaining temperature. To this mixture, t-butyl nitrite (1.13 kg, 1.5 eq) was added over the course of 1 h, while maintaining the temperature at 10° C. The reaction was stirred at this temperature, and the reaction progress was monitored via GC analysis. Upon completion of the reaction, the reaction was quenched by the slow addition of aqueous ammonium chloride. The reaction mass was filtered, and extracted with DCM. The combined organic extracts were washed with brine, affording a solution of Compound 25 which was carried to the next step.

The solution of intermediate Compound 25 was recharged to an inert reactor, and NIS (1.7 kg, 1.0 eq) was charged portion-wise at room temperature. Reaction progress was monitored via HPLC analysis. Upon reaction completion, the reaction was quenched with aqueous thiosulfate, washed with brine, and concentrated under reduced pressure. The crude product was purified by column chromatography with hexane and ethyl acetate, to give Compound 15 in 35% yield over two steps as a tan liquid. ¹H NMR (400 MHz, CDCl₃): δ 7.07 (d, J=2.4 Hz, 1H), 5.51 (d, J=2.4 Hz, 1H), 3.90 (q, J=7.2 Hz, 2H), 3.34 (bs, 2H), 1.35 (t, J=7.2 Hz, 3H). LCMS: m/z [M+H]⁺ 257.03.

Synthesis of Compound 7. Compound 15 (1.0 kg, 1.0 eq) was charged to an inert reactor and was dissolved in about 5 L of anhydrous THF at 25° C. The reactor was cooled to −10° C., and a solution of i-PrMgCl in THF (2.0 L, 2 M, 1 eq) was slowly added. The formation of the intermediate material was followed by HPLC analysis. Once fully formed, a solution of Compound 14 (0.9 kg, 0.95 eq), in THF was slowly charged to the reactor. The reaction was monitored via HPLC analysis. Upon completion, the reaction was warmed to 2° C., and was quenched by the addition of aqueous ammonium chloride. The organic fraction was isolated. The aqueous fraction was extracted with DCM. The organic fractions were combined and washed with aqueous bisulfite, brine, and water. Concentration of the organic layers afforded the crude Compound 13 as an intermediate.

The crude intermediate Compound 13 was dissolved in about 1.5 L of DCM, and recharged to an inert reactor at 25° C. The solution was degassed, and triethylsilane (1.3 kg, 3.0 eq) was charged to the reactor. The temperature was lowered to 5° C., and TFA (1.7 kg, 3.9 eq) was slowly added while maintaining the reaction temperature. Once addition was complete, the reaction was stirred at the same temperature, and progress was monitored via HPLC analysis. Upon completion, the reaction was carefully quenched via aqueous sodium carbonate, and the organic layer was isolated. The aqueous fraction was back extracted with DCM, and the organic fractions were combined. The combined organic fractions were washed with brine and water, and concentrated under reduced pressure. Crystallization from heptane afforded the final product in 60% yield over two steps.

### Example 14. Salt Screen for Compound 2

A salt screen was carried out on Compound 2 using 21 counterions to identify suitable conditions for successful salt formation. The acid counterions investigated are: benzenesulfonic acid, ethanedisulfonic acid dihydrate, citric acid, fumaric acid, hydrochloric acid, L-malic acid, maleic acid, methanesulfonic acid, naphthalene-1,5-disulfonic acid, sulfuric acid, succinic acid, L-tartaric acid, phosphoric acid, toluenesulfonic acid, oxalic acid, camphorsulfonic acid, ethanesulfonic acid, 2-naphthalenesulfonic acid, 2-hydroxyethanesulfonic acid, trifluoroacetic acid, and hydrobromic acid.

Separately, a polymorph screen of amorphous Compound 2 free base did not identify any crystalline forms.

Screening Procedure: The equivalent of 25 mg of Compound 2 was added to each 2 mL vial in the form of a stock solution (c.a. 52.6 mg/mL) in ethanol, followed by the appropriate volume of counter ion likewise from a stock solution in ethanol. The vials were left uncapped to evaporate while stirring overnight at 40° C. in atmosphere. Vials where full evaporation did not occur were further evaporated under a gentle stream of nitrogen the following morning. The vials were then placed under active vacuum (˜−29 inHg) at 50° C. for 3 h to dry thoroughly.

For the first round of screening experiments, approximately 10 vol. (0.25 mL) of solvent (methyl tert-butyl ether; MtBE:heptane (1:1 vol.), IPA:water (7:3 vol.), or methyl acetate; MeOAc:heptane (1:1 vol.)) was added to each vial, and the samples were heated to 45° C. while stirring at 500 rpm. The vials that demonstrated significant precipitation were vortexed and sonicated often to ensure thorough mixing. After 2 h, the temperature was reduced to RT and the samples were left to stir overnight prior to sampling. Similar steps were taken for the second and third round of screening experiments. The solvents selected for the second round of screening were acetonitrile (ACN), ethyl acetate (EtOAc):heptane (75:25 vol.), and toluene:cyclohexane (75:25 vol.). The third round of solvents were 2-methyltetrahydrofuran (2-MeTHF) and EtOH.

A drop was taken from each vial that remained in solution and transferred to a 2 mL vial. The vials were then capped and placed in the freezer at −20° C. After 3 days, the vials were checked visually or under a microscope to see if a solid was generated. After seven 7 days, no precipitation was observed from the vials at −20° C. Vials that produced gums or oils were sonicated for approximately 3 h and stirred for another 1-3 days.

XRPD analysis was done in three stages. First, XRPD analysis of the wet cake was completed for all samples where solids were observed. Unique solids were then left on XRPD plates and dried under vacuum (˜−29 inHg) at 50° C. for at least 3 h, after which they were analyzed by XRPD. Finally, solids were exposed to >95% RH overnight and XRPD was carried out on the resulting solids. The humid environment was generated by placing a beaker of saturated potassium sulfate in water in a sealed chamber. All XRPD patterns were compared to counter ion XRPD patterns and any known patterns of the freebase of Compound 2.

Results: Throughout the salt screening, 11 unique crystalline salt patterns were observed, of which five remained physically stable upon both drying and humidification. Crystalline salts were formed with 7 counter ions, mainly sulfonic acids such as benzenesulfonic acid (“BSA”; from EtOAc:heptane or MeOAc:heptane), methanesulfonic acid (“MSA”; from MeCN, EtOAc:heptane, or toluene:cyclohexane), toluenesulfonic acid (“TSA”; from EtOAc:heptane), camphorsulfonic acid (“CSA”; from MtBE:heptane, EtOAc:heptane, or MeOAc:heptane), ethanesulfonic acid (ESA, from MeCN, EtOAc:heptane, or toluene:cyclohexane; 3 crystalline salt patterns), and 2-naphthalenesulfonic acid (from 2-MeTHF). A crystalline salt was also obtained with sulfuric acid using ethanol as solvent; however, gumming was observed at vial scale. The crystalline salts were further assessed by the following physicochemical properties: stability to drying/humidification, polymorphism, solubility in water, purity, stoichiometry, crystallinity, and residual solvent.

Three crystalline salt patterns were observed for the besylate salt of Compound 2, with Form A isolated after drying solids obtained from crystallization in MeOAc:heptane and characterized by XRPD as shown in FIG. 52 and by DSC as shown in FIG. 53. Form B of the besylate salt was isolated following humidification (as described in this section) of the solids obtained from crystallization in MeOAc:heptane and characterized by XRPD as shown in FIG. 54.

The 2-naphthalenesulfonate salt of Compound 2 obtained by crystallization from 2-MeTHF was characterized by XRPD as shown in FIG. 55 and by DSC as shown in FIG. 56.

### Example 15. Preparation of Salts of Compound 2

Five crystalline salt patterns were identified during the salt screen of Example 14 as stable to drying and humidification (mesylate, camsylate, esylate, sulfate, and tosylate), and the synthesis of four of the patterns were scaled-up and further characterized. The mesylate, camsylate, esylate, and sulfate were all determined to be anhydrous forms with defined melts above 120° C. Stoichiometry of the salts was also well-defined, and minimal mass loss was observed by thermogravimetric analysis (TGA). Although synthesis of the tosylate salt was not successfully scaled-up, material sufficient for analysis was produced using the screening conditions identified in Example 14 (i.e. crystallization from EtOAc:heptane).

A. Preparation of Form A of Mesylate Salt of Compound 2
1. Added approximately 1.3 mL of MeCN to 1307.2 mg of amorphous Compound 2 freebase.
2. Mixture stirred at 40° C. for about 15 minutes to obtain hazy solution.
3. 59.5 μL liquid MSA was added dropwise, then stirred for 15 min.
4. Solution seeded with a spatula tip (˜1-2 mg) of Form A of Mesylate Salt of Compound 2.
5. The mixture was stirred at 40° C. for 15 min.
6. Remaining 119 μL of MSA added to the slurry dropwise.
7. Additional 2.4 mL of MeCN was added to increase flowability.
8. Slurry was allowed to stir at 40° C. for approximately 2 h before cooling to RT and stirring overnight.
9. Slurry was filtered and washed three times with 1 vol. of MeCN.
10. Wet cake was sampled for XRPD.
11. Solids was transferred to a tared 20 mL vial and dried overnight under vacuum (−29 inHg) at 50° C.
12. Yield of 1173.2 mg of Form A of Mesylate Salt of Compound 2 was recovered
13. Summary of results obtained for a sample of Form A of Mesylate Salt of Compound 2 is provided in Table 4.

B. Preparation of Form A of Camsylate (CSA) Salt of Compound 2
1. In a 4 mL vial, 498.4 mg of CSA powder was weighed and slurried in 1 mL of MeOAc:heptane (1:1 vol.).
2. In a 20 mL vial, 1.0 mL of MeOAc:heptane (1:1 vol.) was added to 1028.7 mg of amorphous Compound 2 freebase.
3. The mixture was stirred with a stir bar at 40° C. for 10 min, resulting in amber solution.
4. Approximately 0.33 mL of the CSA stock slurry was added dropwise, then stirred for 15 min.
5. The amber slurry was then seeded with a spatula tip (˜1-2 mg) of Form A of Camsylate (CSA) Salt of Compound 2.
6. The mixture was stirred at 40° C. for 15 min.
7. The remaining CSA stock slurry was added to the amber slurry dropwise. The vial containing the CSA
stock slurry and the transfer pipette were washed with three aliquots of 0.2 mL MeOAc:heptane (1:1 vol.).
8. An additional 0.4 mL of MeOAc and 2.4 mL of MeOAc:heptane (1:1 vol.) were added to increase flowability.
9. The slurry was allowed to stir at 40° C. for approximately 2 h before cooling to RT and stirring overnight.
10. The resulting beige slurry was filtered and the solids washed three times with 1 vol. of MeOAc:heptane (1:1 vol.).
11. The wet cake was sampled for XRPD.
12. The solid was transferred to a tared 20 mL vial and dried overnight under vacuum (−29 inHg) at 50° C.
13. Yield of 1328 mg of Form A of Camsylate (CSA) Salt of Compound 2 was recovered.
14. Summary of results obtained for a sample of Form A of Camsylate (CSA) Salt of Compound 2 is provided in Table 4.

C. Preparation of Form A of Esylate Salt of Compound 2
1. In a 20 mL vial, 1.3 mL of EtOAc:heptane (1:1 vol.) was added to 1325.9 mg of amorphous Compound 2 freebase.
2. The mixture was stirred with a stir bar at 40° C. for 15 min, resulting in amber solution.
3. 78.7 μL liquid ESA was added dropwise to the solution, then the mixture was stirred for 15 min.
4. The amber slurry was then seeded with a spatula tip (˜1-2 mg) of Form A of Esylate Salt of Compound 2.
5. The mixture was stirred at 40° C. for 15 min.
6. The remaining 156.4 μL of ESA was added to the slurry dropwise.
7. An additional 2.8 mL of EtOAc:heptane (1:1 vol.) was added to increase flowability.
8. The slurry was allowed to stir at 40° C. for approximately 2 h before cooling to RT and stirring overnight.
9. The slurry was filtered and washed three times with 1 vol. of EtOAc:heptane (1:1 vol.).
10. The wet cake was sampled for XRPD.
11. The solid was transferred to a tared 20 mL vial and dried overnight under vacuum (−29 inHg) at 50° C.
12. Yield of 1425.8 mg of Form A of Esylate Salt of Compound 2 was recovered.
13. Summary of results obtained for a sample of Form A of Esylate Salt of Compound 2 is provided in Table 4.

D. Preparation of Form A of Sulfate Salt of Compound 2
1. In a 20 mL vial, 2.0 mL (3.3 vol.) of EtOH:heptane (1:1 vol.) was added to 494.0 mg of amorphous Compound 2 freebase.
2. The mixture was stirred with a stir bar at 40° C. for 10 min, resulting in amber solution.
3. 476 μL of sulfuric acid stock solution (0.074 g/mL in EtOH:heptane (1:1 vol.)) was added dropwise, then stirred for 15 min.
4. The amber slurry was then seeded with a spatula tip (˜1-2 mg) of Form A of Sulfate Salt of Compound 2.
5. The remaining 952 μL of sulfuric acid stock solution was added dropwise.
6. The slurry was allowed to stir at 40° C. for approximately 2 h before cooling to RT and stirring overnight.
7. The slurry was filtered and washed twice with 0.6 mL (1.0 vol.) of EtOH:heptane (1:1 vol.).
8. The wet cake was sampled for XRPD.
9. The solid was transferred to a tared 20 mL vial and dried overnight under vacuum (−29 inHg) at 50° C.
10. Yield of 503.9 mg of Form A of Sulfate Salt of Compound 2 was recovered.
11. Summary of results obtained for a sample of Form A of Sulfate Salt of Compound 2 is provided in Table 4.

TABLE 4
Summary of Data for Crystalline Salt Forms of Compound 2

| Form A | Mesylate Salt | Sulfate Salt | Camsylate Salt | Esylate Salt | Tosylate Salt |
|---|---|---|---|---|---|
| XRPD Pattern | FIG. 35 | FIG. 46 | FIG. 38 | FIG. 42 | FIG. 50 |
| TGA Pattern | FIG. 37 | FIG. 48 | FIG. 40 | FIG. 44 | ND |
| [mass loss | [None up to | [0.58 up to | [0.16 between | [0.46 between | |
| (wt %)] | 210° C.] | 200° C.] | 185-215° C.] | 170-235° C.] | |
| DSC Pattern | FIG. 36 | FIG. 47 | FIG. 39 | FIG. 43 | FIG. 51 |
| [Onsets (° C.); | [193.97; 83.15] | [183.21; 76.53] | [200.54; 55.54] | [188.79; 71.01] | 143.37 [40.30] |
| Enthalpy (J/g)] | | | | | |

BDL, below detection limit;
ND, not determined

### Example 16. Solid Form Stability Study of Salts of Compound 2

Solid-form stability studies were completed by weighing approximately 30 mg each of Form A of the camsylate, esylate, mesylate, and sulfate salts of Compound 2 into 4 mL vials and covering with a Kimwipe. The vials were placed inside a humidity chamber set to 75% RH at 40° C. for 7 days. The HPLC and XRPD data was collected to confirm the purity and identity of the solid forms. The four salts were physically and chemically stable. No decrease in purity (i.e. less than 0.1%) or form change was observed after a seven-day stress test at 75% relative humidity (RH) and 40° C. (Table 5).

TABLE 5
Summary of Data for Solid Form Stability
Study of Salts of Compound 2

| Salt of | XRPD Pattern | XRPD Pattern | Purity (%) | Purity (%) |
|---|---|---|---|---|
| Compound 2 | Before | After | Before | After |
| Camsylate | Form A | Form A | 99.59 | 99.65 |
| Esylate | Form A | Form A | 99.75 | 99.75 |
| Mesylate | Form A | Form A | 99.92 | 99.94 |
| Sulfate | Form A | Form A | 99.97 | 99.97 |

### Example 17. Solubility of Salts of Compound 2

Solubility of the scaled-up salts of Compound 2 and free base Compound 2 was assessed in distilled water at RT. Approximately 30 mg of solid was weighed directly into 2 mL vials, and a 6 mm stir bar was added to each vial. Water was then incrementally added to the vials (starting with 1 vol.) and the resulting slurries were left to stir for 10-15 min prior to the next addition. As all solids remained in slurries upon the addition of 1 mL of water, the gravimetric method was adopted, and the vials were stirred at RT for 3 days before centrifuging. Supernatant solutions were recovered and evaporated to dryness at 50° C. in atmosphere on a hot plate, then placed at 50° C. under vacuum (˜−29 inHg) for 3 h before final weighing. The results are outlined in Table 6.

TABLE 6
Summary of Solubility Data for Salts of Compound 2

| | Starting | | Solubility | XRPD |
|---|---|---|---|---|
| Form of | XRPD | Mass | (mg solid/ | Pattern |
| Compound 2 | Pattern | (mg) | mL water) | Recovered |
| Free Base | Amorphous | 31.0 | <3 | Amorphous |
| Camsylate Salt | Form A | 29.1 | <3 | Form A |
| Esylate Salt | Form A | 32.6 | 5 | Form A |
| Mesylate Salt | Form A | 34.4 | 8 | Form A |
| Sulfate Salt | Form A | 22.4 | <3 | Form A |

### Example 18. Alternative Synthesis of Compound 1

This example presents an alternative to the syntheses of Compound 1 depicted in Schemes 4A and 4B.

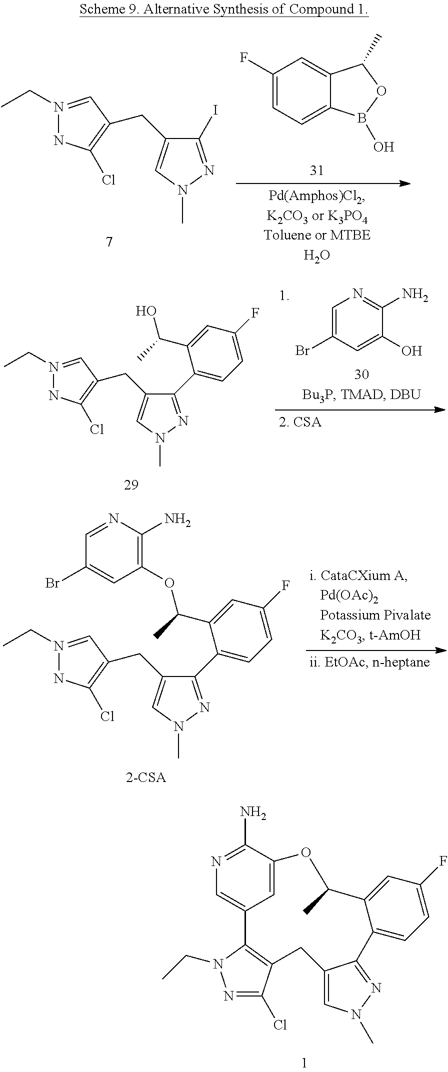

Synthesis of Compound 29. Compound 7 (100 g, 1 eq) was dissolved in 200 mL of MTBE, followed by Compound 31 (the S-enantiomer of Compound 8) (52 g, 1.1 eq) at 25° C. with stirring. The reaction mixture was degassed with nitrogen, and a solution of aqueous potassium phosphate (2.5 eq, 200 mL) was added. The mixture was again degassed, and Pd(Amphos)Cl₂ (1 g, 0.005 eq) was charged. The reaction mixture was heated to 50° C., and was monitored by HPLC analysis. When the reaction was complete, the organic layer was isolated, washed with water, and was concentrated under reduced pressure. Crystallization from MTBE and heptane afforded Compound 29 (the (S)-enantiomer of Compound 5) in 90% yield.

Synthesis of Compound 2. In a 250 mL three-necked RBF, equipped with mechanical stirrer and thermal probe, Compound 30 (6.77 g, 1.3 eq.) was dissolved in anhydrous THF, and DBU (5.47 mL, 1.3 eq.) was added dropwise. The brown solution was stirred at room temperature for 30 min. Compound 29 (10 g, 1.0 eq.) was added to the reaction mixture followed by PBu₃ (9.04 mL, 1.3 eq.). A solution of TMAD (6.17 g, 1.3 eq.) in THF was added dropwise using a dropping funnel over 25 min then stirred for 1 h. Reaction completion was monitored via HPLC analysis. Upon reaction completion, the mixture was filtered, and concentrated under reduced pressure. The resulting material was dissolved in i-PrOAc, and washed with water. The reaction mixture was warmed to 40° C., and camphorsulfonic acid (7.11 g, 1.1 eq) was added to the mixture in one portion. The reaction was cooled to room temperature, and filtered to give Form A of the camsylate salt of Compound 2 (57% yield).

Synthesis of Compound 1. The camsylate salt of Compound 2 is used directly to prepare Compound 1 in a ring closing procedure similar to that described in Example 1, but with 1 equiv. of an inorganic base, in particular potassium carbonate, added.

Alternatively, the camsylate salt of Compound 2 is converted to the free base form of Compound 2 prior to palladium mediated ring closing. The camsylate salt of Compound 2 is suspended in an organic solvent (e.g. isopropyl acetate, EtOAc, toluene) and is washed with an aqueous solution of base (e.g. potassium carbonate or potassium bicarbonate). Once the free base is generated, the solvent is swapped to t-amyl alcohol for the ring closing reaction. No additional inorganic base is needed in the ring closing reaction if the camsylate salt of Compound 2 is free based prior to use.

### Example 19. Preparation of Salts of Compound 1

Preparation of a Salicylate Salt of Compound 1: Following the steps of synthesizing Compound 1 as shown in Scheme 4A or Scheme 4B, 5 g (1 eq) of crude Compound 1, which had been treated by cysteine/silica thiol and citric acid, was dissolved in ˜50 mL of IPA, and was heated to 50° C. Salicylic acid (0.46 g, 0.3 eq) was added to the mixture to induce crystallization. An additional 1.52 g (1 eq) of salicylic acid was slowly added in portions, and the mixture stirred at temperature for 1 h. The mixture was cooled to 15° C. over a 2 h period, and stirred at this temperature for an additional 42 h. The resulting solids were isolated by vacuum filtration. Filtration followed by drying at 45° C. under reduced pressure afforded the salicylate salt of Compound 1 as a white solid (5.5 g, 85% yield). XRPD characterization was obtained as shown in FIG. 58. ¹H NMR (400 MHz, DMSO-d₆) δ=7.75 (dd, J=1.8, 7.8 Hz, 1H), 7.71 (dd, 1H), 7.58 (s, 1H), 7.48 (d, 1H), 7.41-7.32 (m, 1H), 7.22-7.10 (m, 1H), 6.89-6.74 (m, 2H), 6.28 (br d, 1H), 5.39-5.25 (m, 1H), 4.10-3.94 (m, 1H), 3.87 (s, 1H), 3.55 (d, 1H), 2.70 (d, 1H), 1.71 (d, 1H), 1.27 (t, 1H). LCMS 453.93 (M+1).

The salicylate salt of Compound 1 is further treated with base, such as aq. K₂CO₃ or aq. Na₂CO₃, to liberate the free base of Compound 1. The resulting organic phase is extracted with EtOAc and crystallized with EtOAc/heptane to provide Form 2 of the free base of Compound 1.

Preparation of a Maleate Salt of Compound 1: Following the steps of synthesizing Compound 1 as shown in Scheme 4A or Scheme 4B, 5 g (1 eq) of crude Compound 1, which had been treated by cysteine/silica thiol and citric acid, was dissolved in ˜20 mL of IPA, and was heated to 50° C. Maleic acid (1.66 g, 1.3 eq) was added to induce crystallization of the salt. The resulting slurry was stirred at 50° C. for 1 h, and was cooled to 0° C. After stirring for 1 h at this temperature, the solution was filtered and dried in a vacuum oven to yield the maleate salt of Compound 1 as a white compound (4.3 g, 70% yield). XRPD characterization was obtained as shown in FIG. 59. ¹H NMR (400 MHz, DMSO-d₆) δ=7.70 (dd, 1H), 7.59 (s, 1H), 7.51 (d, 1H), 7.24-7.12 (m, 2H), 6.98-6.41 (m, 1H), 6.35 (d, 1H), 6.27-6.18 (m, 2H), 5.42-5.29 (m, 1H), 4.10-3.94 (m, 2H), 3.88 (s, 3H), 3.83-3.72 (m, 1H), 3.56 (d, 1H), 2.72 (d, 1H), 1.73 (d, 3H), 1.27 (t, 3H), 1.11 (s, 1H), 1.04 (d, 1H), 1.01 (br s, 1H), 1.07 (s, 1H). LCMS 453.93 (M+1).

The maleate salt of Compound 1 is further treated with base, such as aq. K₂CO₃ or aq. Na₂CO₃, to liberate the salt. The resulting organic phase is extracted with EtOAc and crystalized with EtOAc/heptane to provide Form 2 of the free base of Compound 1.

Preparation of Additional Salts: Additional salts shown in the Table below were prepared using procedures similar to those described above and the solubility of the resulting salts of Compound 1 was evaluated.

TABLE 7
Solubility of Compound 1 Salts

| | Volume (at 10-20° C.) | | | | | | | | | | | | | | | | | |
|---|---|---|---|---|---|---|---|---|---|---|---|---|---|---|---|---|---|---|
| | Maleic | | L- | | Fumaric | | Citric | | | | | | | | Salicylic | | | |
| Acid Salt | Acid | | Tartaric | | Acid | | Acid | | HCl | | H3PO4 | | TsOH | | acid | | MSA | |
| of Cpd 1 | salt | | Acid Salt | | salt | | salt | | salt | | salt | | salt | | salt | | salt | |
| EtOAc | 20-30 | V | 12-14 | V | 80-90 | V | >400 | V | >400 | V | >100 | V | 18-20 | V | 26-30 | V | 300-400 | V |
| IPAc | 20-30 V | | 52-56 | V | 90-100 | V | >400 | V | >400 | V | >400 | V | 18-20 | V | 56-60 | V | 260-300 | V |
| | (dissolved in 4 V | | | | | | | | | | | | | | | | | |
| | at first, then after | | | | | | | | | | | | | | | | | |
| | 10 min, turned | | | | | | | | | | | | | | | | | |
| | into suspension) | | | | | | | | | | | | | | | | | |
| IPA | 70-80 | V | 18-20 | V | 8-9 | V | 24-28 | V | 8-10 | V | 140-150 | V | >400 | V | 300-400 | V | 56-60 | V |
| t-AmOH | 6-8 | V | 18-20 | V | 22-24 | V | 86-90 | V | 18-20 | V | >400 | V | >400 | V | >400 | V | 340-360 | V |
| 2- | 50-60 V | | 4-6 | V | 8-10 | V | 8-10 | V | >400 | V | >160 | V | >400 | V | 18-20 | V | >400 | V |
| MeTHF | (dissolved in 4 V | | | | | | | | | | | | | | | | | |
| | at first, then after | | | | | | | | | | | | | | | | | |
| | 10 min, turned | | | | | | | | | | | | | | | | | |
| | into suspension) | | | | | | | | | | | | | | | | | |
| THF | 2-4 | V | 8-10 | V | 3-4 | V | 10-12 | V | 300-400 | V | >160 | V | 280-300 | V | 4-6 | V | 60-100 | V |
| Acetone | 2-4 | V | 2-4 | V | 8-10 | V | 6-8 | V | 22-24 | V | 58-60 | V | 10-12 | V | 26-28 | V | 56-60 | V |
| Toluene | >400 | V | >400 | V | >400 | V | >400 | V | >400 | V | >400 | V | >400 | V | 320-360 | V | >400 | V |
| MeCN | 16-20 | V | 30-32 | V | 150-200 | V | 300-400 | V | 8-10 | V | >400 | V | 8-10 | V | 90-100 | V | 6-8 | V |
| CPME | 220 | V | 60-70 | V | 50-60 | V | 330 | V | >500 | V | >500 | V | >400 | V | 100-140 | V | >400 | V |

### Example 20. Preparation of Tablets of Compound 1

Compound 1 tablets were manufactured for oral administration at 5 mg, 25 mg, 50 mg, 75 mg, 100 mg, 125 mg, and 150 mg strengths. Tablets at all dosage strengths were manufactured as immediate release, film-coated tablets. The quantitative composition of the tablets is provided in the following table.

TABLE 8
Composition of Compound 1 Tablets, 5 mg, 25
mg, 50 mg, 75 mg, 100 mg, 125 mg, and 150 mg

| | | Target Quantity (mg/tablet) | | | | | | |
|---|---|---|---|---|---|---|---|---|
| Component | Function | 5 mg | 25 mg | 50 mg | 75 mg | 100 mg | 125 mg | 150 mg |
| Compound 1 | Active | 5.00 | 25.00 | 50.00 | 75.00 | 100.00 | 125.00 | 150.00 |
| Drug Substance^{a} | | | | | | | | |
| Microcrystalline | Diluent | 83.50 | 85.00 | 170.00 | 255.0 | 340.00 | 425.00 | 510.00 |
| Cellulose^{a} | | | | | | | | |
| Croscarmellose | Disintegrant | 5.00 | 6.25 | 12.50 | 18.75 | 25.00 | 31.25 | 37.50 |
| Sodium | | | | | | | | |
| Colloidal Silica | Glidant | 2.50 | 3.125 | 6.25 | 9.375 | 12.50 | 15.625 | 18.75 |
| Dioxide | | | | | | | | |
| Hydroxypropyl | Binder | 2.50 | 3.125 | 6.25 | 9.375 | 12.50 | 15.625 | 18.75 |
| Cellulose | | | | | | | | |
| Magnesium | Lubricant | 1.50 | 2.50 | 5.00 | 7.50 | 10.00 | 12.5 | 15.00 |
| Stearate | | | | | | | | |
| Opadry II^{b} | Cosmetic | 3.00 | 3.125 | 6.25 | 9.375 | 12.50 | 15.625 | 18.75 |
| | film-coat | | | | | | | |
| Purified Water^{c} | Coating | qs | qs | qs | qs | qs | qs | qs |
| | Agent | | | | | | | |

| | | | | | | | |
|---|---|---|---|---|---|---|---|
| Total | 103.00 | 128.125 | 256.25 | 384.375 | 512.5 | 640.625 | 768.75 |

^{a}The amount of drug substance and microcrystalline cellulose may be adjusted depending on the potency of the drug substance
^{b}Non-functional, cosmetic film coating, added to target theoretical weight gain of 2.5-3%.
^{c}Removed during processing.
qs: quantity sufficient

### Example 21. Stability Studies of Compound 1 and Compound 1 Tablets

A stability study was conducted for Compound 1 crystalline free base Form 2. Samples were stored at: (1) 30° C.±2° C./65%±5% Relative Humidity (RH), with data collected at 0, 1, 3, 6, 9, and 12 months; and (2) 40° C.±2° C./75%±5% RH, with data collected at 0, 1, 3, and 6 months. At the indicated time points, tests were performed for description, assay, related substances, water content, polymorphic form, and chiral purity.

Stability results demonstrated the chemical and physical stability of Compound 1 crystalline free base Form 2 stored for 12 months at the condition of 30° C.±2° C./65%±5% RH and 6 months at the accelerated condition of 40° C.±2° C./75%±5% RH. There was no meaningful change observed in description, assay, related substances, polymorphic form, and chiral purity. All results complied with the acceptance criteria (Assay: 97%-103% w/w anhydrous and solvent free, individual unspecified impurities: each less than or equal to 0.3% by area, total amount of impurities: less than or equal to 2% area, enantiomeric purity: greater than or equal to 99%, solid form: consistent with reference Form 2) at all time points.

A stability study is conducted for Compound 1 film coated tablets, 5 mg and 50 mg, having the compositions in Example 9 or Example 18. The tablets were packaged in 60 cc bottle with coil and induction sealed at two storage conditions: (1) 30° C.±2° C./65%±5% RH, with data collected at 0, 1, 2, 3, 6, 9, and 12 months and (2) 40° C.±2° C./75%±5% RH, with data collected at 0, 1, 2, 3, and 6 months. At the indicated time points, tests were performed for description, assay, degradation, dissolution, water content, and chiral purity.

Photostability study of the tablets was also performed by exposing the tablets to ICH Q1B Option 2 condition with total exposure of not less than 1.2 million-lux-hours and 200 Watt-hours/m². Additionally, a study with tablets in open dish was performed at 40° C.±2° C., 75%±5% RH, with data collected at 0, 1, 2, 3, and 6 months.

Stability results demonstrated the chemical and physical stability of Compound 15 mg and 50 mg film-coated tablets packaged in 60 cc bottle stored for 12 months at the condition of 30° C.±2° C./65%±5% RH and 6 months at the accelerated condition of 40° C.±2° C./75%±5% RH, as well as tablets in open dish at 40° C.±2° C./75%±5% RH condition for six months, and tablets exposed to not less than 1.2 million-lux-hours and 200 Watt-hours/m². No meaningful changes were observed in description, assay, degradation products, and dissolution. For the open dish study, there was some increase in water content observed but there were no meaningful changes in the other attributes. All results complied with the acceptance criteria at all time points (Assay: 90.0%-110.0% w/w, individual unspecified degradation products: each less than or equal to 0.3% by area, total amount of degradation products: less than or equal to 3.0% area, chiral purity about 100%, dissolution: Conforms to USP <711>, NLT 80% (Q) of Label Claim at 30 mins).

### Example 22. Tablet Dissolution Measurements

Three batches of Compound 1 crystalline free base Form 2 each having different particle size (Table 9) were used to prepare 50 mg tablets of Compound 1. Dissolution was performed on the tablets by using USP II paddle method (Table 10) in 900 mL dissolution media (pH 3.0 Na₂HPO₄-0.4% CTAB) at 37.0±0.5° C. with paddle speed at 75 rpm (Infinity 250 rpm m after 60 minutes). Samples were taken at different time intervals (5, 10, 15, 20, 30, 45, 60 and 90 min) to be analyzed by HPLC. FIG. 57 shows the tablet dissolution profiles carried out on Agilent 708-DS Dissolution Apparatus with 850-DS auto sampling system or equivalent. As can be seen from the figure, the particle size profile had no significant impact on the dissolution profile.

TABLE 9
Particle Size Batches

| Batch No. | D10 (μm) | D50 (μm) | D90 (μm) |
|---|---|---|---|
| 1 | 4 | 10 | 19 |
| 2 | 15 | 47 | 106 |
| 3 | 5.6 | 19.4 | 45.7 |

TABLE 10
Dissolution Method

| PARAMETER | SETTING |
|---|---|
| Dissolution Medium | pH 3.0 Na₂HPO₄-0.4% CTAB |
| Dissolution Method | Paddle, USP II |
| Media Volume | 900 mL |
| Rotational Speed | 75 rpm |
| Temperature | 37.0 ± 0.5° C. |
| Sampling Times | 5, 10, 15, 20, 30, 45, 60 and 90 |
| | min (Infinity 250 rpm after 60 mins) |
| Sample Volume | For Automated Sampling: 1.5 mL; |
| | For manual Sampling: 5.0 mL |

While exemplary embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein can be employed in practicing the subject matter provided herein. All such equivalents are considered to be within the scope of the claimed subject matter and are encompassed by the appended claims.

1. A solid form comprising a compound of Formula (I), or a pharmaceutically acceptable salt thereof:

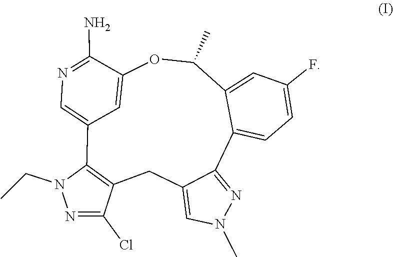 [SMILES: CCn1nc(Cl)c2c1-c1cnc(N)c(c1)O[C@H](C)c1cc(F)ccc1-c1nn(C)cc1C2]

2. The solid form of claim 1, which is crystalline.

3. The solid form of claim 1, comprising a free base of a compound of formula (I).

4. The solid form of claim 3, which is characterized by an XRPD pattern, when measured using Cu Kα radiation, comprising at least three peaks selected from the group consisting of 11.2, 12.4, 13.2, 14.3, 18.9, 21.1, 21.6, 21.8, 22.5, 22.7, 23.0, and 27.0° 2θ±0.2° 2θ.

5. The solid form of claim 4, which is characterized by an XRPD pattern comprising at least four peaks selected from the group consisting of 11.2, 12.4, 13.2, 14.3, 18.9, 21.1, 21.6, 21.8, 22.5, 22.7, 23.0, and 27.0° 2θ±0.2° 2θ.

6. The solid form of claim 5, which is characterized by an XRPD pattern comprising at least five peaks selected from the group consisting of 11.2, 12.4, 13.2, 14.3, 18.9, 21.1, 21.6, 21.8, 22.5, 22.7, 23.0, and 27.0° 2θ±0.2° 2θ.

7. The solid form of claim 4, which is characterized by an XRPD pattern comprising peaks at 12.4, 18.9, and 21.1° 2θ±0.2° 2θ.

8. The solid form of claim 7, wherein the XRPD pattern further comprises peaks at 13.2 and 22.5° 2θ±0.2° 2θ.

9. The solid form of claim 8, wherein the XRPD pattern further comprises peaks at 11.2 and 22.7° 2θ±0.2° 2θ.

10. The solid form of claim 4, which is characterized by an XRPD pattern that matches the XRPD pattern depicted in FIG. 4.

11. The solid form of claim 4, which exhibits an endothermic event, as characterized by DSC, with an onset temperature at 260° C.±2° C. and/or a peak temperature at 261° C.±2° C.

12. The solid form of claim 4, which exhibits a weight increase of about 0.3% when subjected to an increase in relative humidity from about 0 to about 90% relative humidity.

13. The solid form of claim 4, having approximately unit cell dimensions of: a=8.2 Å, b=14.8 Å, c=18.7 Å, α=90°, β=90°, and γ=90°.

14. The solid form of claim 4, which is anhydrous.

15.-17. (canceled)

18. The solid form of claim 3, which is:
(i) characterized by an XRPD pattern comprising peaks at 6.0, 18.5 and 20.6° 2θ±0.2° 2θ;
(iii) characterized by an XRPD pattern comprising peaks at 9.4, 12.8, and 15.3° 2θ±0.2° 2θ;
(iv) characterized by an XRPD pattern comprising peaks at 6.1, 17.2, and 18.2° 2θ±0.2° 2θ;
(v) characterized by an XRPD pattern comprising peaks at 6.8, 10.0, and 18.9° 2θ±0.2° 2θ;
(vi) characterized by an XRPD pattern comprising peaks at 5.8, 10.0, and 18.1° 2θ±0.2° 2θ;
(vii) characterized by an XRPD pattern comprising peaks at 5.9, 9.1, and 19.6° 2θ±0.2° 2θ;
(viii) characterized by an XRPD pattern comprising peaks at 6.0, 17.0, and 19.7° 2θ±0.2° 2θ;
(ix) characterized by an XRPD pattern comprising peaks at 5.9, 17.2, and 19.4° 2θ±0.2° 2θ;
(x) characterized by an XRPD pattern comprising peaks at 5.9, 8.4, and 8.6° 2θ±0.2° 2θ;
(xi) characterized by an XRPD pattern comprising peaks at 5.9, 10.7, and 20.1° 2θ±0.2° 2θ;
(xii) characterized by an XRPD pattern comprising peaks at 5.8, 19.2, and 22.1° 2θ±0.2° 2θ;
(xiii) characterized by an XRPD pattern comprising peaks at 5.9, 9.2, and 19.2° 2θ±0.2° 2θ;
(xiv) characterized by an XRPD pattern comprising peaks at 6.7, 16.9, and 18.9° 2θ±0.2° 2θ; or
(xv) characterized by an XRPD pattern comprising peaks at 6.7, 22.0, and 22.8° 2θ±0.2° 2θ.

19.-122. (canceled)

123. A hydrochloric acid salt (hydrochloride salt), methane sulfonic acid salt (mesylate salt), benzene sulfonic acid salt (besylate salt), maleic acid salt (maleate salt), phosphoric acid salt (phosphate salt), citric acid salt (citrate salt), L-tartaric acid salt (L-tartarate salt), fumaric acid salt (fumarate salt), toluenesulfonic acid (tosylate), or salicylic acid salt (salicylate salt) of a compound of Formula (I):

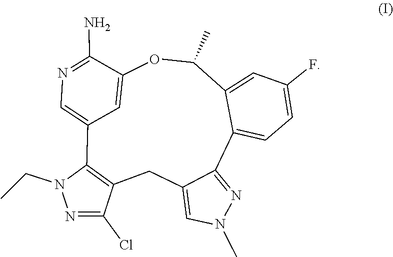 [SMILES: CCn1nc(Cl)c2c1-c1cnc(N)c(c1)O[C@H](C)c1cc(F)ccc1-c1nn(C)cc1C2]

124. (canceled)

125. A solid form comprising the hydrochloric acid salt (hydrochloride salt), methane sulfonic acid salt (mesylate salt), benzene sulfonic acid salt (besylate salt), maleic acid salt (maleate salt), phosphoric acid salt (phosphate salt), citric acid salt (citrate salt), L-tartaric acid salt (L-tartarate salt), fumaric acid salt (fumarate salt), toluenesulfonic acid (tosylate), or salicylic acid salt (salicylate salt) of a compound of Formula (I) of claim 123.

126. (canceled)

127. The solid form of claim 125, which is crystalline.

128. The solid form of claim 127, comprising a salicylic acid salt (salicylate salt) of a compound of Formula (I).

129. The solid form of claim 128 which is characterized by an XRPD pattern comprising peaks at 9.7, 11.7, and 14.7° 2θ±0.2° 2θ.

130.-131. (canceled)

132. The solid form of claim 127, comprising a maleic acid salt (maleate salt) of a compound of Formula (I).

133. The solid form of claim 132 which is characterized by an XRPD pattern comprising peaks at 6.0, 13.8, and 21.3° 2θ±0.2° 2θ.

134.-135. (canceled)

136. A pharmaceutical composition comprising the solid form of claim 1, and a pharmaceutically acceptable excipient.

137. A method of treating cancer comprising administering a therapeutically effective amount of the solid form of claim 1 to a subject in need thereof.

138. A process for preparing Form 2 of a compound of Formula (I): 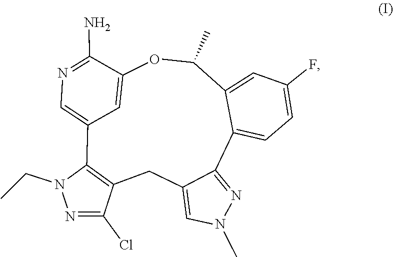 [SMILES: CCn1nc(Cl)c2c1-c1cnc(N)c(c1)O[C@H](C)c1cc(F)ccc1-c1nn(C)cc1C2]
comprising
(i) dissolving the compound of Formula (I) in a solvent;
(ii) adding an anti-solvent; and
(ii) recovering said Form 2.

139.-141. (canceled)

142. A salt of a compound of Formula (II):

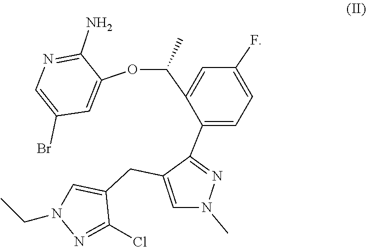

143-144. (canceled)

145. A solid form comprising the salt of a compound of Formula (II) of claim 142.

146.-176. (canceled)

177. A process for preparing a compound of Formula (II): 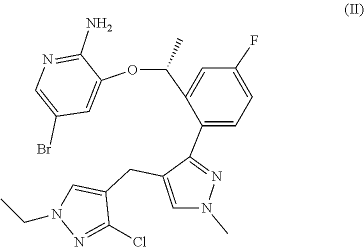
or a stereoisomer, or a mixture of stereoisomers thereof, or a pharmaceutically acceptable salt thereof, comprising:
(step 2.0) reacting a compound of Formula (III): 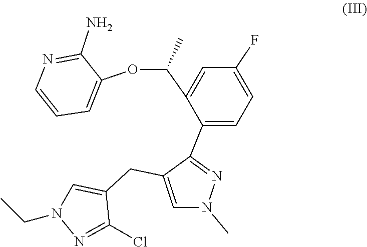
or a stereoisomer, or a mixture of stereoisomers thereof, or a pharmaceutically acceptable salt thereof, with a brominating reagent.

178. A process for preparing a compound of Formula (II): 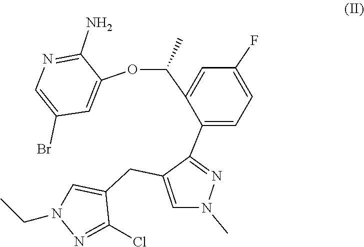
or a stereoisomer, or a mixture of stereoisomers thereof, or a pharmaceutically acceptable salt thereof, comprising:
(step 2a.1) reacting a compound of Formula (XXIX): 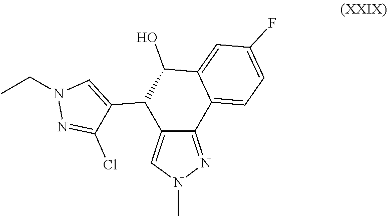 [SMILES: CCn1cc(Cc2cn(C)nc2-c2ccc(F)cc2[C@H](C)O)c(Cl)n1]
or a stereoisomer, or a mixture of stereoisomers thereof, or a pharmaceutically acceptable salt thereof, with a compound of Formula (XXX): 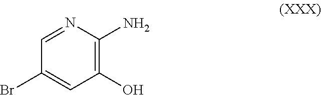
or a pharmaceutically acceptable salt thereof.

179. The process of claim 177, further comprising:
(step 1.0) cyclizing the compound of Formula (II), or a stereoisomer, or a mixture of stereoisomers thereof, or a pharmaceutically acceptable salt thereof, to provide a compound of Formula (I): 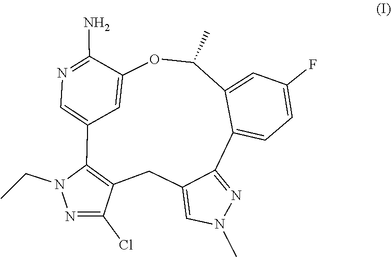 [SMILES: CCn1nc(Cl)c2c1-c1cnc(N)c(c1)O[C@H](C)c1cc(F)ccc1-c1nn(C)cc1C2]
or a stereoisomer, or a mixture of stereoisomers thereof, or a pharmaceutically acceptable salt thereof.

180.-229. (canceled)

230. A process for preparing a compound of Formula (I): 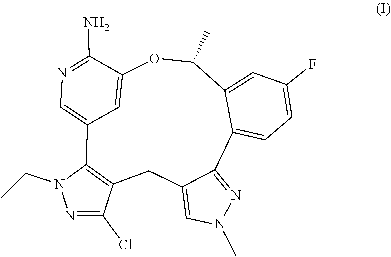 [SMILES: CCn1nc(Cl)c2c1-c1cnc(N)c(c1)O[C@H](C)c1cc(F)ccc1-c1nn(C)cc1C2]
or a stereoisomer, or a mixture of stereoisomers thereof, or a pharmaceutically acceptable salt thereof, comprising:
(step 1.0) cyclizing a compound of Formula (II): 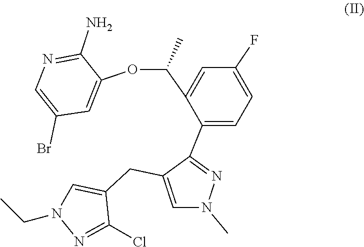
or a stereoisomer, or a mixture of stereoisomers thereof, or a pharmaceutically acceptable salt thereof, to provide a compound of Formula (I), or a stereoisomer, or a mixture of stereoisomers thereof, or a pharmaceutically acceptable salt thereof,
wherein step 1.0 occurs in the presence of a base, and wherein the base is potassium pivalate.

231.-232. (canceled)

233. A solid form of a compound of Formula (I), or a stereoisomer, or a mixture of stereoisomers thereof, or a pharmaceutically acceptable salt thereof, prepared by the process of claim 179.

234.-236. (canceled)

237. A compound of Formula (III) or (IV): 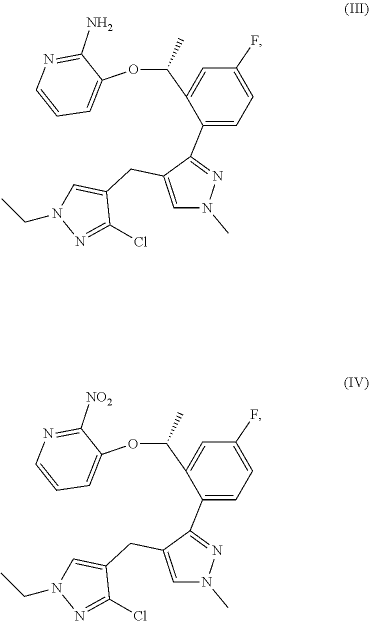
a compound of Formula (SP-1), (SP-2), (SP-3), (SP-4), (SP-5), (SP-6), (SP-7), or (SP-8), or a stereoisomer, or a mixture of stereoisomers thereof, or a pharmaceutically acceptable salt thereof.

238. (canceled)

239. A pharmaceutical composition comprising Compound 1: 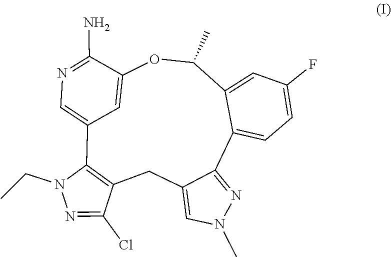 [SMILES: CCn1nc(Cl)c2c1-c1cnc(N)c(c1)O[C@H](C)c1cc(F)ccc1-c1nn(C)cc1C2]
or a stereoisomer, or a mixture of stereoisomers thereof, or a pharmaceutically acceptable salt thereof, a diluent, a disintegrant, a glidant, a binder, and a lubricant.

240.-267. (canceled)

268. A method of treating cancer comprising administering a therapeutically effective amount of the pharmaceutical composition of claim 239 to a subject in need thereof.
